SEQUENCE LISTING (11 sequences)

SEQ ID NO: 1 - protein, 707 aa, Homo sapiens [sequence omitted]

SEQ ID NO: 2 - protein, 427 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGP

SEQ ID NO: 3 - protein, 417 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYG

SEQ ID NO: 4 - protein, 242 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 5 - protein, 204 aa, Homo sapiens
MLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 6 - protein, 251 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 7 - protein, 268 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGP

SEQ ID NO: 8 - protein, 167 aa, Homo sapiens
MFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGP

SEQ ID NO: 9 - protein, 7 aa, Homo sapiens
FQTFEGD

SEQ ID NO: 10 - protein, 15 aa, Homo sapiens
CGVPDLGRFQTFEGD

SEQ ID NO: 11 - protein, 708 aa, Rattus norvegicus
MSPWQPLLLVLLALGYSFAAPHQRQPTYVVFPRDLKTSNLTDTQLAEDYLYRYGYTRAAQMMGEKQSLRPALLMLQKQLSLPQTGELDSETLKAIRSPRCGVPDVGKFQTFDGDLKWHHHNITYWIQSYTEDLPRDVIDDSFARAFAVWSAVTPLTFTRVYGLEADIVIQFGVAEHGDGYPFDGKDGLLAHAFPPGPGIQGDAHFDDDELWSLGKGAVVPTYFGNANGAPCHFPFTFEGRSYLSCTTDGRNDGKPWCGTTADYDTDRKYGFCPSENLYTEHGNGDGKPCVFPFIFEGHSYSACTTKGRSDGYRWCATTANYDQDKADGFCPTRADVTVTGGNSAGEMCVFPFVFLGKQYSTCTSEGRSDGRLWCATTSNFDADKKWGFCPDQGYSLFLVAAHEFGHALGLDHSSVPEALMYPMYHYHEDSPLHEDDIKGIHHLYGRGSKPDPRPPATTAAEPQPTAPPTMCSTAPPMAYPTGGPTVAPTGAPSPGPTGPPTAGPSEAPTESSTPDDNPCNVDVFDAIADIQGALHFFKDGRYWKFSNHGGNQLQGPFLIARTWPAFPSKLNSAFEDPQPKKIFFFLWAQMWVYTGQSVLGPRSLDKLGLGSEVTLVTGLLPRRGGKALLISRERIWKFDLKSQKVDPQSVTRLDNEFSGVPWNSHNVFQYQDKAYFCHDKYFWRVSFHNRVNQVDHVAYVTYDLLQCP

(19) United States
(12) Patent Application Publication
(10) Pub. No.: US 2012/0129842 A1
(43) Pub. Date: May 24, 2012

(54) BISTHIAZOLE INHIBITORS OF PRO-MATRIX METALLOPROTEINASE ACTIVATION

(76) Inventors: Paul Francis JACKSON, New Hope, PA (US); Brett Andrew Tounge, Blue Bell, PA (US); Kristi Anne Leonard, Lansdale, PA (US); Yan Zhang, Fort Washington, PA (US); Aihua Wang, Jamison, PA (US); Michael Hawkins, Ambler, PA (US); Umar S.M. Maharoof, North Wales, PA (US); Joseph Kent Barbay, Flourtown, PA (US)

(21) Appl. No.: 13/297,400

(22) Filed: Nov. 16, 2011

Related U.S. Application Data
(60) Provisional application No. 61/414,967, filed on Nov. 18, 2010.

(51) Int. Cl. A61K 31/551; C07D 417/14; A61K 31/427; A61K 31/454; A61K 31/496; A61K 31/4709; A61K 31/5377; A61P 35/00; A61P 19/02; A61P 19/08; A61P 9/00; A61P 1/04; A61P 9/12; A61P 11/00; A61P 1/00; A61P 29/00; A61P 17/02; A61P 1/16; A61P 25/00; A61P 11/06; A61P 13/12; A61P 25/06; A61P 9/10; C07D 417/04

(52) U.S. Cl. 514/218; 548/194; 548/195; 548/197; 548/181; 546/209; 544/369; 546/171; 544/133; 540/575; 514/370; 514/371; 514/326; 514/254.02; 514/314; 514/236.8

(57) ABSTRACT

This invention relates to bisthiazole I and its therapeutic and prophylactic uses, wherein the variables A, R⁵, R⁶, and R⁷ are defined in the specification. Disorders treated and/or prevented include rheumatoid arthritis.

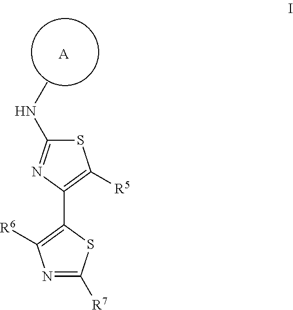

### CROSS-REFERENCE TO RELATED APPLICATIONS

The present application claims the benefits of the filing of U.S. Provisional Application No. 61/414,967 filed Nov. 18, 2010. The complete disclosures of the aforementioned related patent applications are hereby incorporated herein by reference for all purposes.

### TECHNICAL FIELD

The present invention relates to novel bisthiazole compounds and their therapeutic and prophylactic uses. Disorders treated and/or prevented include inflammation related disorders and disorders ameliorated by inhibiting the proteolytic activation of pro-matrix metalloproteinases.

### BACKGROUND OF THE INVENTION

Matrix metalloproteinases (MMPs) are a family of structurally related zinc-dependent proteolytic enzymes that digest extracellular matrix proteins such as collagen, elastin, laminin and fibronectin. Currently, at least 28 different mammalian MMP proteins have been identified and they are grouped based on substrate specificity and domain structure. Enzymatic activities of the MMPs are precisely controlled, not only by their gene expression in various cell types, but also by activation of their inactive zymogen precursors (proMMPs) and inhibition by endogenous inhibitors and tissue inhibitors of metalloproteinases (TIMPs). The enzymes play a key role in normal homeostatic tissue remodeling events, but are also considered to play a key role in pathological destruction of the matrix in many connective tissue diseases such as arthritis, periodontitis, and tissue ulceration and also in cancer cell invasion and metastasis.

A role for MMPs in oncology is well established, as up-regulation of any number of MMPs are one mechanism by which malignant cells can overcome connective tissue barriers and metastasize (Curr Cancer Drug Targets 5(3): 203-20, 2005). MMPs also appear to have a direct role in angiogenesis, which is another reason they have been an important target for oncology indications (Int J Cancer 115(6): 849-60, 2005; J Cell Mol Med 9(2): 267-85, 2005). Several different classes of MMPs are involved in these processes, including MMP9. Other MMP mediated indications include the cartilage and bone degeneration that results in osteoarthritis and rheumatoid arthritis. The degeneration is due primarily to MMP digestion of the extracellular matrix (ECM) in bone and joints (Aging Clin Exp Res 15(5): 364-72, 2003). Various MMPs, including MMP9 and MMP13 have been found to be elevated in the tissues and body fluids surrounding the damaged areas.

Elevated MMP levels, including MMP9 and MMP13 are also believed to be involved in atherosclerotic plaque rupture, aneurysm and vascular and myocardial tissue morphogenesis (Expert Opin Investig Drugs 9(5): 993-1007, 2000; Curr Med Chem 12(8): 917-25, 2005). Elevated levels of MMPs, including MMP9 and MMP13, have often been associated with these conditions. Several other pathologies such as gastric ulcers, pulmonary hypertension, chronic obstructive pulmonary disease, inflammatory bowel disease, periodontal disease, skin ulcers, liver fibrosis, emphysema, and Marfan syndrome all appear to have an MMP component as well (Expert Opinion on Therapeutic Patents 12(5): 665-707, 2002). Within the central nervous system, altered MMP expression has been linked to several neurodegenerative disease states (Expert Opin Investig Drugs 8(3): 255-68, 1999), most notably in stroke (Glia 50(4): 329-39, 2005). MMPs, including MMP9, have been shown to have an impact in propagating the brain tissue damage that occurs following an ischemic or hemorrhagic insult. Studies in human stroke patients and in animal stroke models have demonstrated that expression levels and activity of MMPs, including MMP9, increase sharply over a 24 hour period following an ischemic event. Administration of MMP inhibitors has been shown to be protective in animal models of stroke (Expert Opin Investig Drugs 8(3): 255-68, 1999; J Neurosci 25(27): 6401-8, 2005). In addition, MMP9 knockout animals also demonstrate significant neuroprotection in similar stroke models (J Cereb Blood Flow Metab 20(12): 1681-9, 2000). In the US, stroke is the third leading cause of mortality, and the leading cause of disability. Thus stroke comprises a large unmet medical need for acute interventional therapy that could potentially be addressed with MMP inhibitors.

It has also been suggested that MMP9 may play a role in the progression of multiple sclerosis (MS). Studies have indicated that serum levels of MMP9 are elevated in active patients, and are concentrated around MS lesions (Lancet Neurol 2(12): 747-56, 2003). Increased serum MMP9 activity would promote infiltration of leukocytes into the CNS, a causal factor and one of the hallmarks of the disease. MMPs may also contribute to severity and prolongation of migraines. In animal models of migraine (cortical spreading depression), MMP9 is rapidly upregulated and activated leading to a breakdown in the BBB, which results in mild to moderate edema (J Clin Invest 113(10): 1447-55, 2004). It is this brain swelling and subsequent vasoconstriction which causes the debilitating headaches and other symptoms associated with migraine. In the cortical spreading depression model, MMP inhibitors have been shown to prevent the opening of the BBB (J Clin Invest 113(10): 1447-55, 2004). Related research has shown that MMP9 is specifically upregulated in damaged brain tissues following traumatic brain injury (J Neurotrauma 19(5): 615-25, 2002), which would be predicted to lead to further brain damage due to edema and immune cell infiltration. MMPs may also have additional roles in additional chronic CNS disorders. In an animal model of Parkinson's disease, MMP9 was found to be rapidly upregulated after striatal injection of a dopaminergic neuron poison (MPTP).

With regard to structure and activation of the inactive zymogen form, a prototypical MMP is matrix metalloproteinase 9 (MMP9). MMP9 is also known as macrophage gelatinase, gelatinase B, 92 kDa gelatinase, 92 kDa type IV collagenase, and type V collagenase. The inactive form of MMP9, proMMP9, is expressed with several different domains including a signal sequence for secretion, a propeptide domain which inhibits activity of proMMP9, a catalytic domain for protein cleavage, a fibronectin type-II (FnII) domain consisting of three fibronectin-type II repeats, and a hemopexin-like domain thought to assist in substrate docking. The hemopexin-like domain also serves as a binding domain for interaction with tissue inhibitors of metalloproteinases (TIMPs). The inactive zymogen form of MMP9, proMMP9, is maintained through a cysteine-switch mechanism, in which a Cys in the propeptide forms a complex with the catalytic zinc in the catalytic domain and occludes the active site (Proc Natl Acad Sci USA 87(14): 5578-82, 1990). Activation of proMMP9 occurs in a two-step process. A protease cleaves an initial site after Met60, disrupting the zinc coordination and destabilizing the propeptide interaction with the catalytic domain. This initial cleavage allows access to the second cleavage site at Phe107, after which the propeptide is removed and the mature active form of the enzyme is released (Biol Chem 378(3-4): 151-60, 1997). The identity of the proMMP9 activating proteases is unknown in vivo, although there is evidence that activation can occur through the actions of MMP3, chymase and trypsin (J Biol Chem 267(6): 3581-4, 1992; J Biol Chem 272(41): 25628-35, 1997; J Biol Chem 280(10): 9291-6, 2005).

Based on the demonstrated involvement in numerous pathological conditions, inhibitors of matrix metalloproteases (MMPs) have therapeutic potential in a range of disease states. However, non-selective active site MMP inhibitors have performed poorly in clinical trials. The failures have often been caused by dose-limiting toxicity and the manifestation of significant side effects, including the development of musculoskeletal syndrome (MSS). It has been suggested that development of more selective MMP inhibitors might help to overcome some of the problems that hindered clinical success in the past, but there are a number of obstacles to developing more selective MMP active site inhibitors. MMPs share a catalytically important Zn2+ ion in the active site and a highly conserved zinc-binding motif. In addition, there is considerable sequence conservation across the entire catalytic domain for members of the MMP family.

A novel approach to developing more selective MMP inhibitors is to target the pro domain of the inactive zymogens, proMMPs, with allosteric small-molecule inhibitors that bind and stabilize the inactive pro form of the protein and inhibit processing to the active enzyme. There is significantly less sequence identity within the pro domains of MMP proteins, no catalytically important Zn2+ ion, and no highly conserved zinc-binding motif. Thus targeting the pro domain of proMMPs is an attractive mechanism of action for inhibiting the activity of the MMP proteins Inhibition of proMMP9 activation has been observed with a specific monoclonal antibody (Hybridoma 12(4): 349-63, 1993). The activation of proMMP9 by trypsin has also been shown to be inhibited by Bowman-Birk inhibitor proteins and derived peptide inhibitors (Biotechnol Lett 26(11): 901-5, 2004). There are no reports, however, of allosteric small-molecule inhibitors that bind the pro domain and inhibit activation of proMMP9, proMMP13, or any other proMMP. The present invention provides tricyclic compounds as allosteric small-molecule inhibitors of the proteolytic activation of proMMP9, proMMP13, and methods of treatment using such inhibitors.

### SUMMARY OF THE INVENTION

The invention comprises the compounds of Formula I

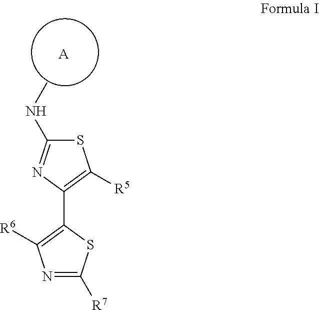

### Wherein:

A is a ring selected from the group consisting of

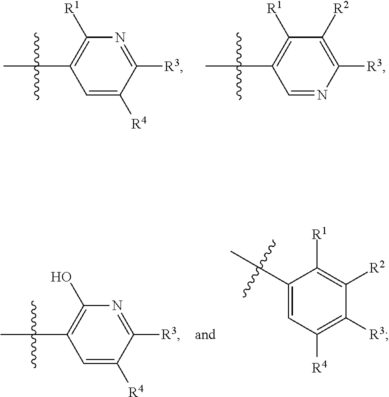

or R¹ and R² may be taken together with the ring to which they are attached to make a fused ring system selected from the group consisting of: quinolinyl, isoquinolinyl, quinazolinyl, quinoxalinyl, benzimidazolyl, benzothiazolyl, napthalyl, benzofuranyl, benzo[1,3]dioxol-4-yl, 2,3-dihydro-benzo[1,4]dioxin-5-yl, 2,3-dihydro-benzofuranyl, benzothiophenyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, benzo[1,3]dioxolyl, 2,3-dihydro-benzo[1,4]dioxinyl, 2,3-dihydro-benzofuranyl, benzothiophenyl, benzotriazolyl, indolyl, indolinyl, and indazolyl are optionally substituted with one methyl group or up to two fluorine atoms;
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### R² is H, CH₃, or F;

R³ is Cl, SO₂NH₂, SO₂CH₃, CO₂H, CONH₂, NO₂, —CN, CH₃, CF₃, or H;
R⁴ is NH₂, NHC₍₁₋₃₎alkyl, N(C₍₁₋₃₎alkyl)₂, C₍₁₋₃₎alkyl, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)C₍₁₋₄₎alkyl, C₍₁₋₄₎alkyl, F, Cl, Br, —CN, OC₍₁₋₄₎alkyl;

### R⁵ is H, F, Br, Cl, CF₃, or CH₃;

### R⁶ is H, CH₃, or CF₃;

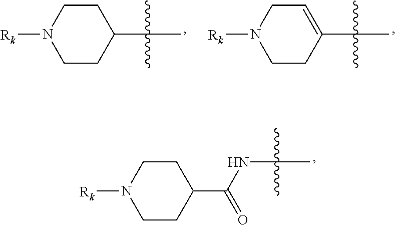

R⁷ is H, CF₃, CH₂CF₃, Cl, Br, C₍₁₋₆₎alkyl, CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A²; wherein said

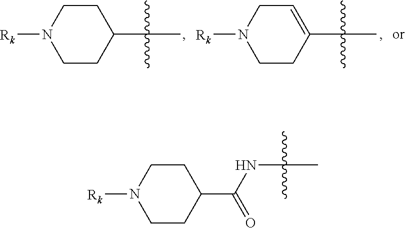

is optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl,

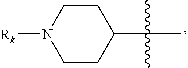

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

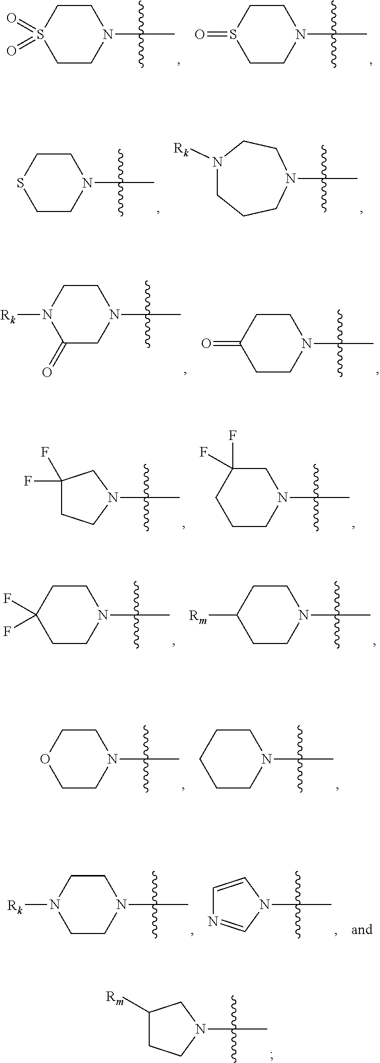

wherein any said A¹ and A² ring, except imidazolyl, may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoropyridyl, and C₍₃₋₆₎cycloalkyl;

Rₘ is H, OCH₃, CH₂OH, NH(C₍₁₋₄₎alkyl), N(C₍₁₋₄₎alkyl)₂, NH₂, C₍₁₋₆₎alkyl, F, or OH; and solvates, hydrates, tautomers, and pharmaceutically acceptable salts thereof.

### BRIEF DESCRIPTION OF THE DRAWINGS

Embodiments of the present invention will now be described, by way of an example only, with reference to the accompanying drawings wherein:

FIG. 1: Shown are western blots with two different antibodies illustrating the effects of a small molecule allosteric processing inhibitor, Compound α, on the activation of proMMP9 in synoviocytes harvested from female Lewis rats after inducing arthritis with i.p. administration of Streptococcal cell wall peptidoglycan polysaccharides. A mouse monoclonal antibody, mAb L51/82, detected pro and processed forms of MMP9. The mouse monoclonal antibody showed that Compound α caused a dose-dependent reduction in the appearance of the 80 kD active form of MMP9 and the appearance of an 86 kD form of the protein (FIG. 1A, lanes 3-6). A rabbit polyclonal antibody, pAb-1246, detected the 80 kD active form of MMP9, but did not recognize the 100 kD form of proMMP9. The rabbit polyclonal antibody showed that the small molecule allosteric processing inhibitor caused a dose-dependent reduction in the appearance of the 80 kD active form of MMP9 (FIG. 1B, lanes 2-6).

FIG. 2: Shown are western blots illustrating increased proMMP9 and increased active MMP9 in tibia-tarsus joints (ankles) from female Lewis rats after inducing arthritis with i.p. administration of Streptococcal cell wall peptidoglycan polysaccharides (SCW). In healthy ankles of rats administered saline, mAb-L51/82 detected small amounts of an approximately 100 kD proMMP9 and an approximately 80 kD form of active MMP9 (FIG. 2A, lanes 1 and 2). The amount of proMMP9 increased markedly in ankle homogenates 5 and 18 days after SCW-administration (FIG. 2A, lanes 3-5 and 6-8, respectively). The amount of active 80 kD MMP9 increased mildly 5 days after SCW-administration (FIG. 2A, lanes 3-5) and increased markedly 18 days after SCW-administration (FIG. 2A, lanes 6-8). In healthy ankles of rats administered saline, mAb-1246 detected small amounts active 80 kD MMP9 (FIG. 2B, lanes 1 and 2). The 80 kD active MMP9 increased mildly 5 days after SCW-administration (FIG. 2A, lanes 3-5) and increased markedly 18 days after SCW-administration (FIG. 2A, lanes 6-8).

FIG. 3: Shown are western blots with two different antibodies illustrating the effects of a small molecule allosteric processing inhibitor, Compound α, on the activation of proMMP9 in tibia-tarsus joints (ankles) from female Lewis rats after inducing arthritis with i.p. administration of Streptococcal cell wall peptidoglycan polysaccharides (SCW). Both proMMP9 and active MMP9 were abundantly present in ankles of SCW-induced vehicle-treated rats (FIGS. 3A and 3B, lanes 1-3). Treatment of rats with Compound α did not reduce the abundance of proMMP-9 (FIG. 3A, lanes 4-9). However, treatment of rats with Compound α resulted in a notable reduction in the active 80 kD form of MMP9 detected with pAb-1246 (FIG. 3B, lanes 4-9) and also with mAb-L51/82 (FIG. 3A, lanes 4-9).

### DETAILED DESCRIPTION OF THE INVENTION

The invention comprises the compounds of Formula I

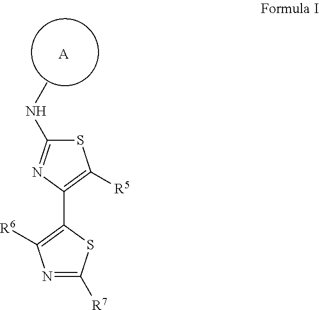

wherein:
A is a ring selected from the group consisting of

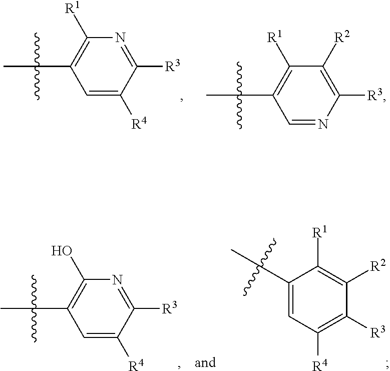

or R¹ and R² may be taken together with the ring to which they are attached to make a fused ring system selected from the group consisting of: quinolinyl, isoquinolinyl, quinazolinyl, quinoxalinyl, benzimidazolyl, benzothiazolyl, napthalyl, benzofuranyl, benzo[1,3]dioxol-4-yl, 2,3-dihydro-benzo[1,4]dioxin-5-yl, 2,3-dihydro-benzofuranyl, benzothiophenyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, benzo[1,3]dioxolyl, 2,3-dihydro-benzo[1,4]dioxinyl, 2,3-dihydro-benzofuranyl, benzothiophenyl, benzotriazolyl, indolyl, indolinyl, and indazolyl are optionally substituted with one methyl group or up to two fluorine atoms;
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃; R² is H, CH₃, or F;
R³ is Cl, SO₂NH₂, SO₂CH₃, CO₂H, CONH₂, NO₂, —CN, CH₃, CF₃, or H;
R⁴ is NH₂, NHC₍₁₋₃₎alkyl, N(C₍₁₋₃₎alkyl)₂, C₍₁₋₃₎alkyl, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)C₍₁₋₄₎alkyl, C₍₁₋₄₎alkyl, F, Cl, Br, —CN, OC₍₁₋₄₎alkyl;

### R⁵ is H, F, Br, Cl, CF₃, or CH₃;

### R⁶ is H, CH₃, or CF₃;

R⁷ is H, CF₃, CH₂CF₃, Cl, Br, C₍₁₋₆₎alkyl,

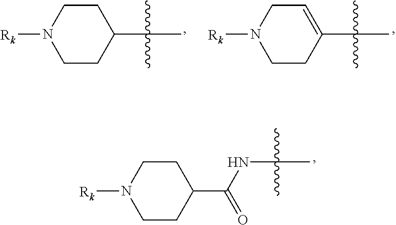

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A²; wherein said

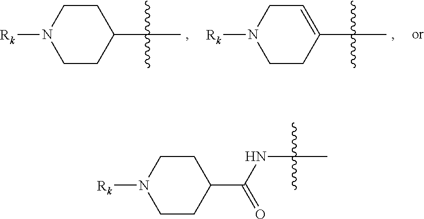

is optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl,

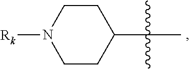

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

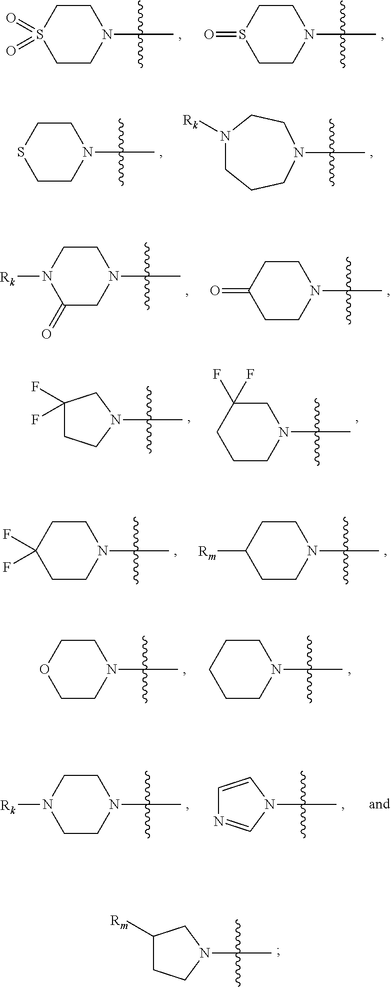

wherein any said A¹ and A² ring, except imidazolyl, may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoropyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH; and solvates, hydrates, tautomers, and pharmaceutically acceptable salts thereof.

In another embodiment of the invention

A is a ring selected from the group consisting of

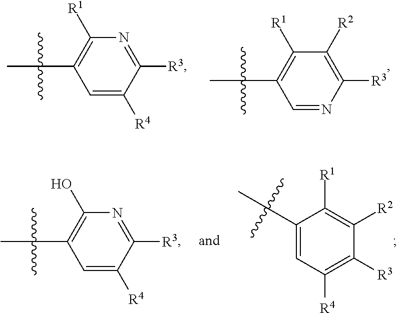

or R¹ and R² may be taken together with the ring to which they are attached to make a fused ring system selected from the group consisting of: quinolinyl, isoquinolinyl, quinazolinyl, benzimidazolyl, napthalyl, benzofuranyl, benzo[1,3]dioxol-4-yl, 2,3-dihydro-benzo[1,4]dioxin-5-yl, 2,3-dihydro-benzofuranyl, and indazolyl, wherein said quinolinyl, isoquinolinyl, quinazolinyl, benzimidazolyl, napthalyl, benzofuranyl, benzo[1,3]dioxolyl, 2,3-dihydro-benzo[1,4]dioxinyl, 2,3-dihydro-benzofuranyl, and indazolyl are optionally substituted with one methyl group or up to two fluorine atoms;
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### R² is H, or F;

R³ is Cl, SO₂NH₂, SO₂CH₃, CO₂H, CONH₂, NO₂, —CN, CH₃, CF₃, or H;
R⁴ is NH₂, CH₃, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)C₍₁₋₄₎alkyl, C₍₁₋₄₎alkyl, F, Cl, Br, —CN, or OCH₃;

### R⁵ is H, F, Br, Cl, CF₃, or CH₃;

### R⁶ is H, CH₃, or CF₃;

R⁷ is H, CF₃, CH₂CF₃, Cl, Br, C₍₁₋₆₎alkyl,

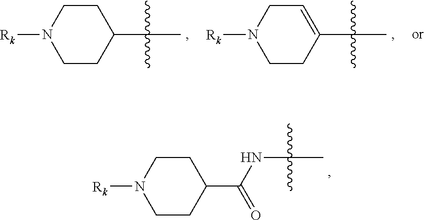

CO₂H, CO₂C₍₁₋₄₎alkyl, SO₂C₍₁₋₄₎alkyl, pyridinyl, pyrimidinyl, pyrazinyl, NA¹A², C(O)NA¹A², SO₂NA¹A², SONA¹A², C(O)N(CH₃)C₍₂₋₆₎alkylNA¹A², C(O)NHC₍₂₋₆₎alkylNA¹A², NHC(O)C₍₁₋₆₎alkylNA¹A², N(CH₃)C(O)C₍₁₋₆₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², NHC₍₂₋₆₎alkylNA¹A², N(CH₃)C₍₂₋₆₎alkylNA¹A², or CH₂NA¹A²;
A¹ is H, or C₍₁₋₃₎alkyl;

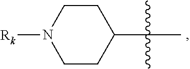

A² is H, C₍₁₋₆₎alkyl, C₍₃₋₆₎cycloalkyl, C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl; wherein said pyridyl may be optionally substituted with two substituents selected from the group consisting of CF₃ and pyrrolidinyl; or A¹ and A² are taken together with their attached nitrogen to form a ring selected from the group consisting of:

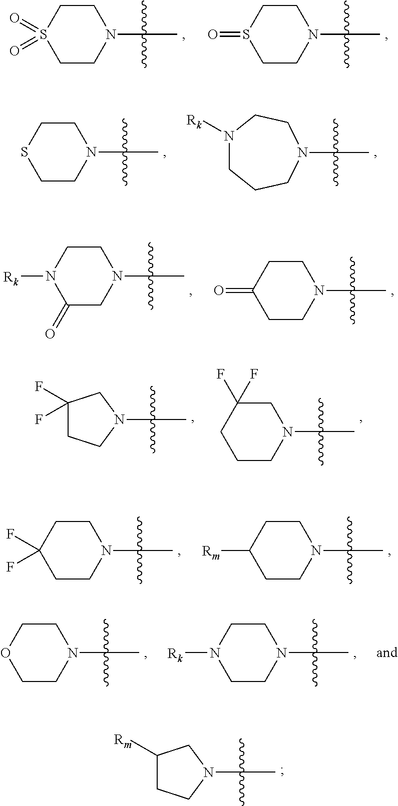

wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₃₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, CH₃, F, or OH; and solvates, hydrates, tautomers, and pharmaceutically acceptable salts thereof

In another embodiment of the invention

A is a ring selected from the group consisting of

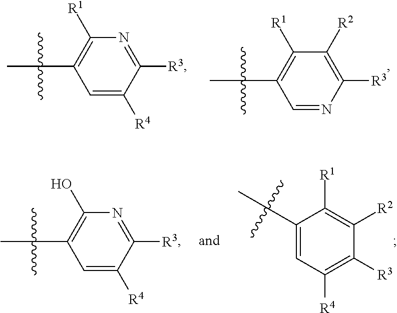

or R¹ and R² may be taken together with the ring to which they are attached to make a fused ring system selected from the group consisting of: quinolinyl, napthalyl, benzofuranyl, benzo[1,3]dioxol-4-yl, 2,3-dihydro-benzo[1,4]dioxin-5-yl, and 2,3-dihydro-benzofuranyl, wherein said quinolinyl, napthalyl, benzofuranyl, benzo[1,3]dioxolyl, 2,3-dihydro-benzo[1,4]dioxinyl, and 2,3-dihydro-benzofuranyl are optionally substituted with one methyl group or up to two fluorine atoms;
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### R² is H, or F;

R³ is Cl, SO₂NH₂, SO₂CH₃, CO₂H, CONH₂, NO₂, —CN, CH₃, CF₃, or H;
R⁴ is NH₂, CH₃, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)CH₃, C₍₁₋₄₎alkyl, F, or Cl;

### R⁵ is H, F, CH₃, or Br;

### R⁶ is H, CH₃, or CF₃;

R⁷ is H, CF₃, CH₂CF₃, C₍₁₋₄₎alkyl,

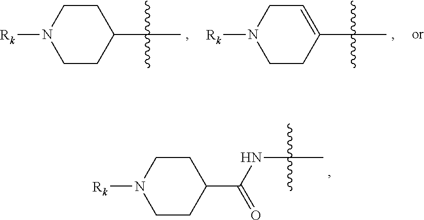

CO₂H, CO₂C₍₁₋₄₎alkyl, SO₂C₍₁₋₄₎alkyl, pyridinyl, pyrimidinyl, pyrazinyl, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₆₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², or CH₂NA¹A²;
A¹ is H, or C₍₁₋₃₎alkyl;
A² is H, C₍₁₋₃₎alkyl,

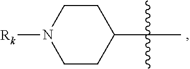

SO₂C₍₁₋₄₎alkyl, C(O)Ph, C(O)C₍₁₋₄₎alkyl, pyrazinyl, or pyridyl; wherein said pyridyl may be optionally substituted with two substituents selected from the group consisting of CF₃ and pyrrolidinyl; or A¹ and A² are taken together with their attached nitrogen to form a ring selected from the group consisting of:

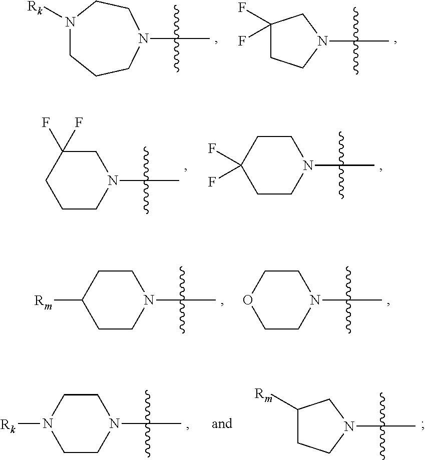

Rₖ is selected from the group consisting of H, CH₂CF₃, CH₂CH₂CF₃, C₍₁₋₃₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, F, or OH; and solvates, hydrates, tautomers, and pharmaceutically acceptable salts thereof.

In another embodiment of the invention

A is a ring selected from the group consisting of

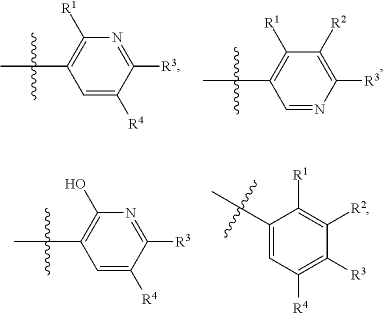

or R¹ and R² may be taken together with the ring to which they are attached to make a fused ring system selected from the group consisting of: quinolinyl, napthalyl, benzofuranyl, benzo[1,3]dioxol-4-yl, 2,3-dihydro-benzo[1,4]dioxin-5-yl, and 2,3-dihydro-benzofuranyl, wherein said quinolinyl, napthalyl, benzofuranyl, benzo[1,3]dioxolyl, 2,3-dihydro-benzo[1,4]dioxinyl, 2,3-dihydro-isoindolonyl, and 2,3-dihydro-benzofuranyl are optionally substituted with one methyl group or up to two fluorine atoms;
R¹ is C₍₁₋₄₎alkoxy, C₍₁₋₄₎alkyl, SC₍₁₋₄₎alkyl, Cl, OC₍₃₋₆₎cycloalkyl, OCH₂CF₃, or OCF₃;

### R² is H, or F;

### R³ is SO₂NH₂, SO₂CH₃, CO₂H, CONH₂, —CN, CH₃, or H;

R⁴ is NH₂, CH₃, —CN, —CH═CH₂, —CONH₂, —CO₂H, —NO₂, —CONHC₍₁₋₄₎alkyl, CH₂CONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHCH₂CH₂-piperidinyl, —CONHCH₂Ph, or R⁴ is selected from the group consisting of: phenyl, pyridyl, pyrazolyl, imidazolyl, oxazolyl, isoxazolyl, thiazolyl, and thiophenyl, wherein said phenyl, pyridyl, pyrazolyl, imidazolyl, oxazolyl, isoxazolyl, thiazolyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)CH₃, C₍₁₋₄₎alkyl, F, or Cl;

### R⁵ is H, F, CH₃, or Br;

### R⁶ is H, CH₃, or CF₃;

R⁷ is C₍₁₋₄₎alkyl,

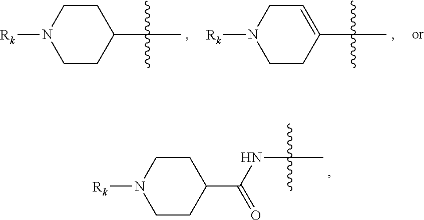

CO₂H, CO₂C₍₁₋₄₎alkyl, SO₂C₍₁₋₄₎alkyl, pyridyl, pyrimidinyl, pyrazinyl, NA¹A², C(O)NA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², or CH₂NA¹A²;
A¹ is H, or C₍₁₋₃₎alkyl;
A² is H, C₍₁₋₃₎alkyl,

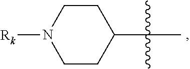

SO₂C₍₁₋₄₎alkyl, C(O)Ph, C(O)C₍₁₋₄₎alkyl, pyrazinyl, or pyridyl; wherein said pyridyl may be optionally substituted with two substituents selected from the group consisting of CF₃ and pyrrolidinyl; or A¹ and A² are taken together with their attached nitrogen to form a ring selected from the group consisting of:

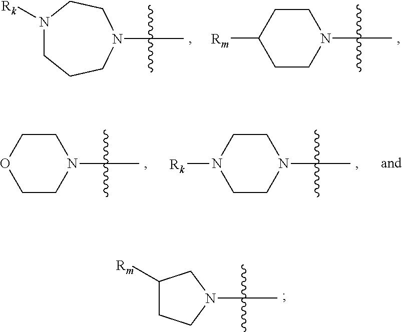

Rₖ is selected from the group consisting of H, C₍₁₋₃₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, N(C₍₁₋₄₎alkyl)₂, NH₂, F, or OH;
and solvates, hydrates, tautomers, and pharmaceutically acceptable salts thereof.

In another embodiment of the invention

A is a ring selected from the group consisting of

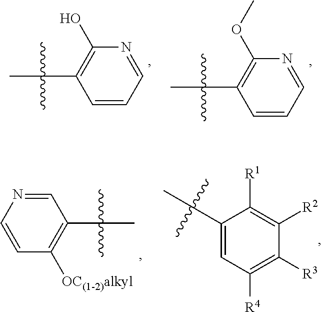

quinolin-8-yl, 6-methoxy-quinolin-8-yl, napthal-1-yl, 2-methyl-benzofuran-7-yl, 2,2-difluoro-benzo[1,3]dioxol-4-yl, 2,3-dihydro-benzo[1,4]dioxin-5-yl, 5-methoxy-2,3-dihydro-isoindolon-6-yl, and 5-sulfonic acid amide-2,3-dihydro-benzofuran-7-yl;
wherein
R¹ is C₍₁₋₄₎alkoxy, C₍₁₋₄₎alkyl, SCH₂CH₃, OCF₃, Cl, OCH₂CF₃, or O-cyclopentyl;

### R² is H, or F;

### R³ is H, CONH₂, or CH₃;

R⁴ is NH₂, CH₃, —CN, —CH═CH₂, —CONH₂, —CO₂H, —NO₂, —CONHCH₃, CH₂CONH₂, —NHCOCH₃, —CO₂CH₃, —CO₂CH₂CH₃, CF₃, SO₂C₍₁₋₂₎alkyl, —SO₂NH₂, —SO₂N(CH₂CH₃)₂, —CONHCH₂CH₂-piperidinyl, —CONHCH₂Ph, or R⁴ is selected from the group consisting of:

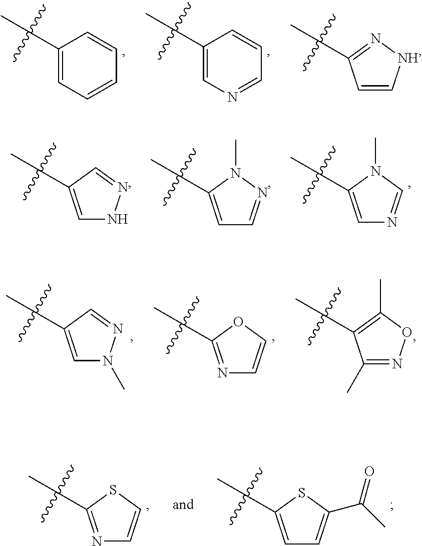

provided that R⁴ is not NHC(O)CH₃, if R⁷ is NHC(O)CH₃; further provided that R⁴ is neither phenyl, nor pyridyl, if R⁷ is CH₃; or R⁴ may be H, provided that R³ is CONH₂;

### R⁵ is H, F, CH₃, or Br;

### R⁶ is H, CH₃, or CF₃;

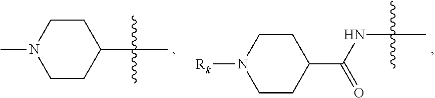

R⁷ is CH₃, CO₂H, CO₂C₍₁₋₂₎alkyl, SO₂CH₃, pyridinyl, NA¹A², C(O)NA¹A², CH₂NHCH₂CH₂NA¹A², CH₂N(CH₃)CH₂CH₂NA¹A², C(O)N(CH₃)CH₂CH₂NA¹A², or CH₂NA¹A²; wherein
A¹ is H, or C₍₁₋₂₎alkyl;

A² is H, C₍₁₋₃₎alkyl, C(O)CH₃,

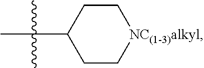

pyrazinyl, or pyridyl, wherein said pyridyl is optionally substituted with up to two substituents selected from the group consisting of CF₃ and pyrrolidinyl; alternatively, A¹ and A² may be taken together with their attached nitrogen, to form a ring selected from the group consisting of

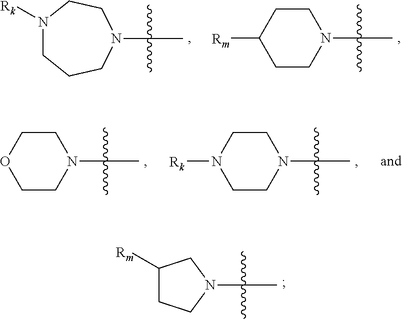

Rₖ is selected from the group consisting of C₍₁₋₃₎alkyl, trifluoromethylpyridyl, and cyclohexyl;

### Rₘ is H, N(CH₃)₂, or OH;

and solvates, hydrates, tautomers, and pharmaceutically acceptable salts thereof.

Another embodiment of the invention is a compound which is selected from the group consisting of:

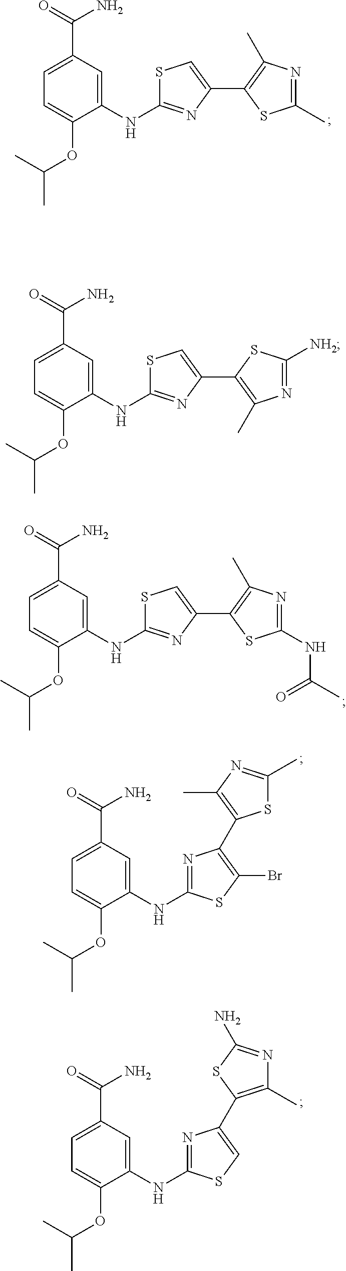

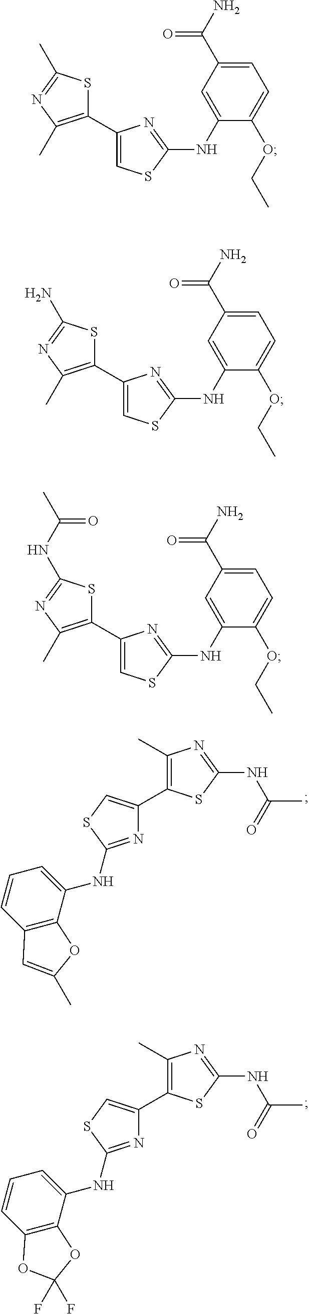

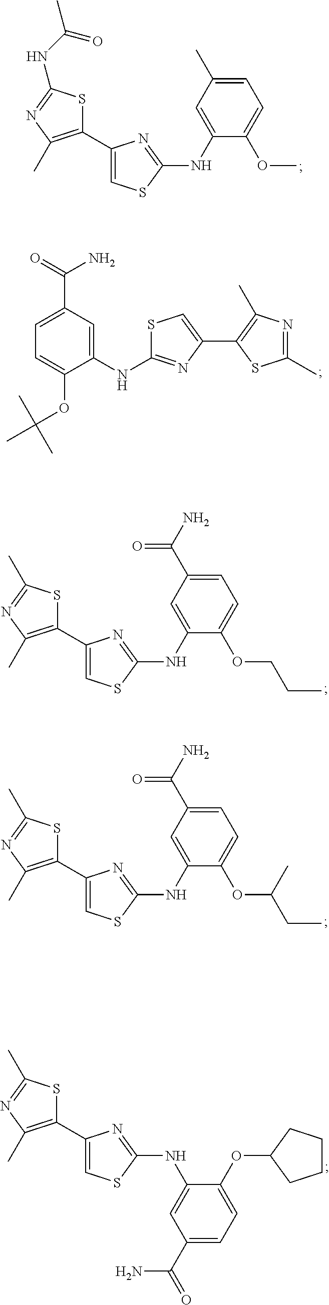

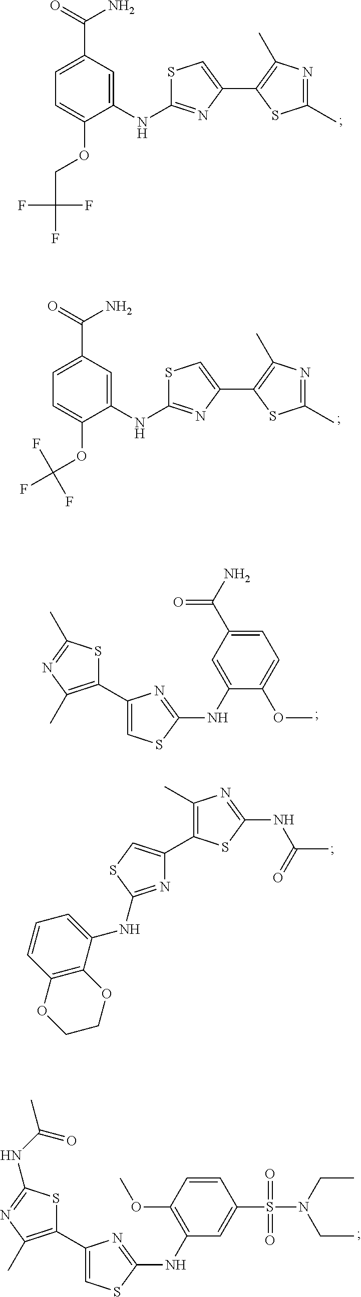

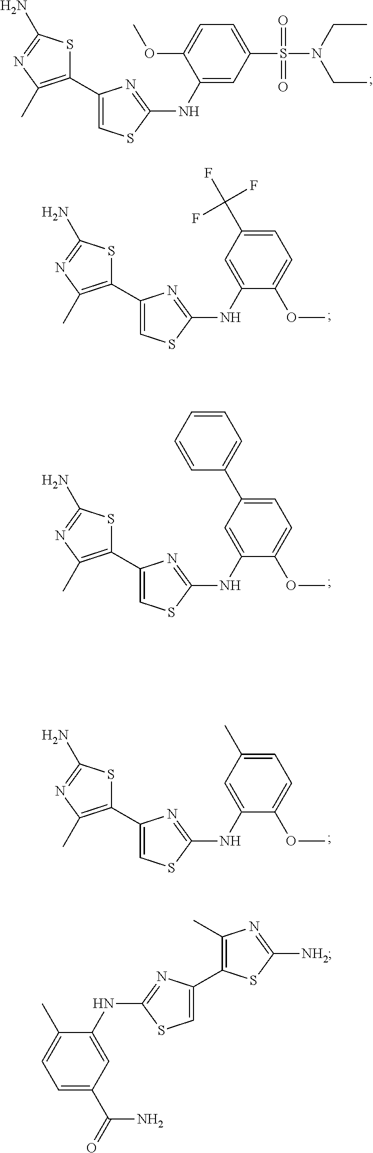

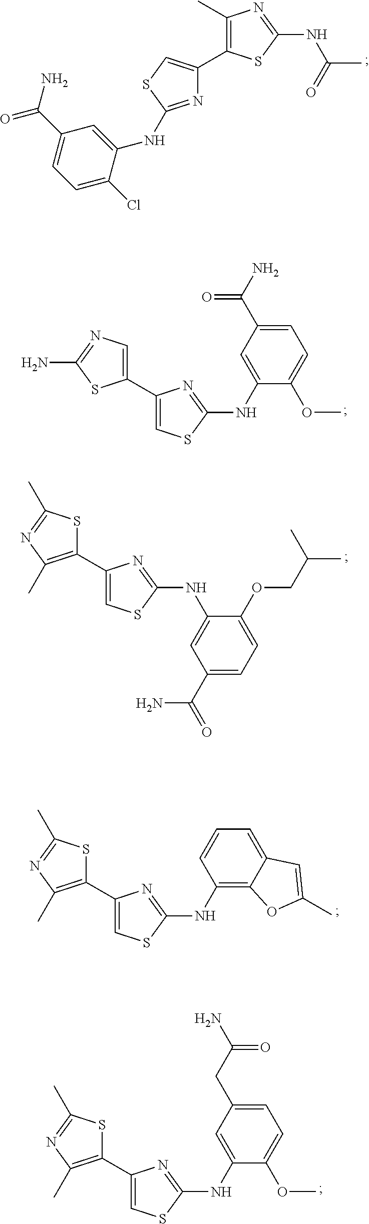

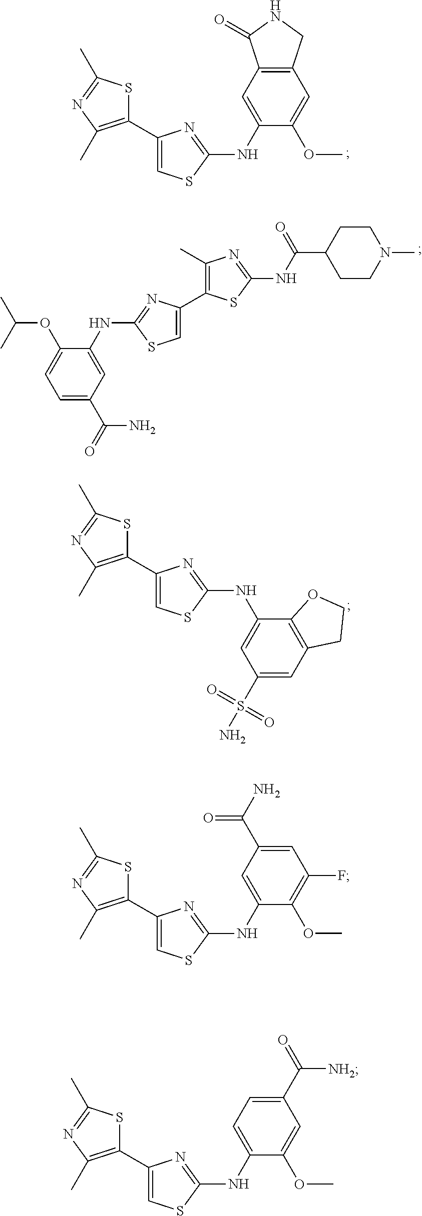

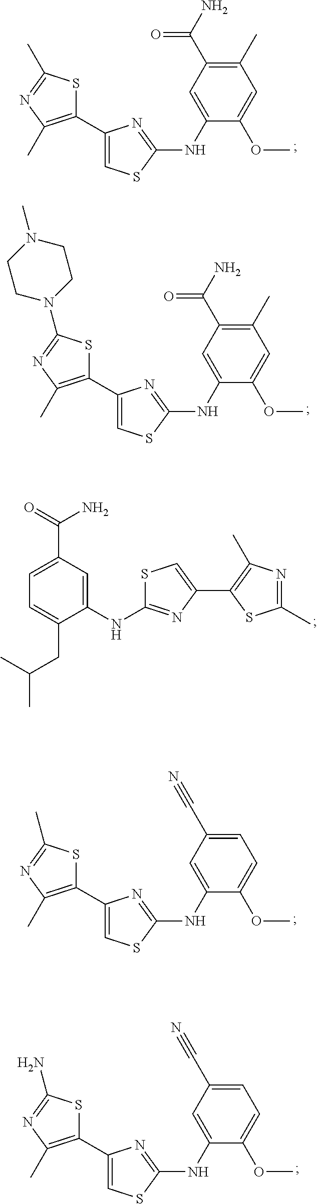

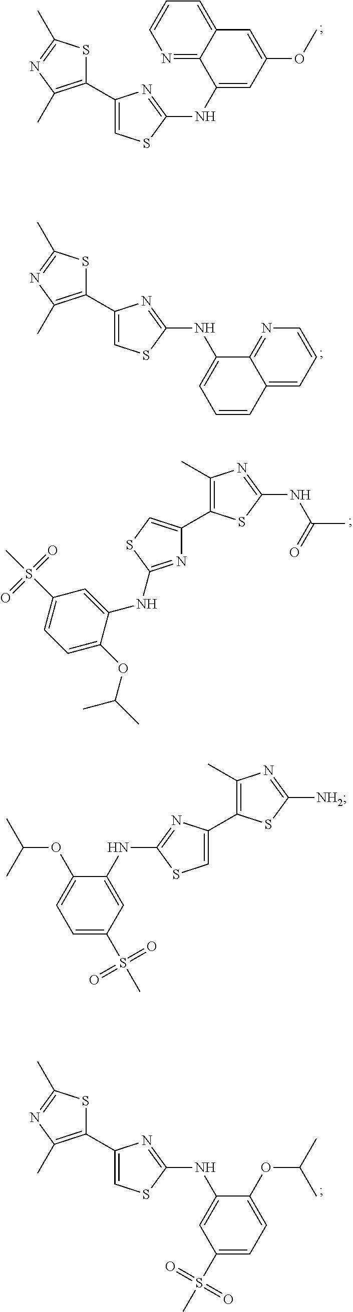

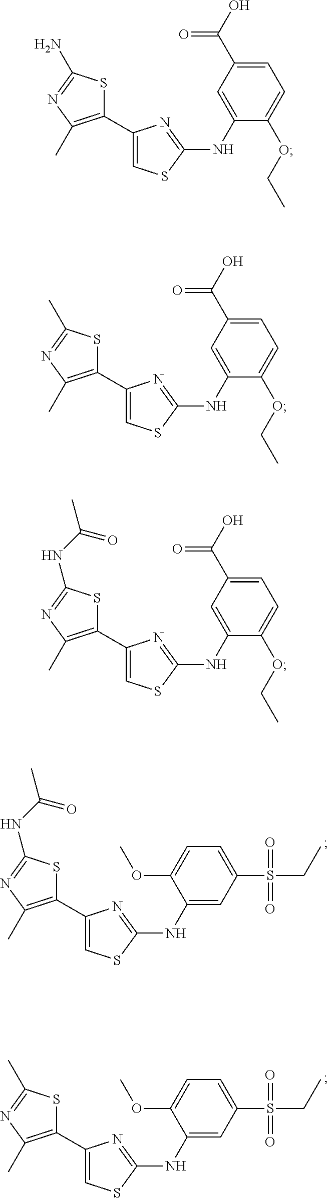

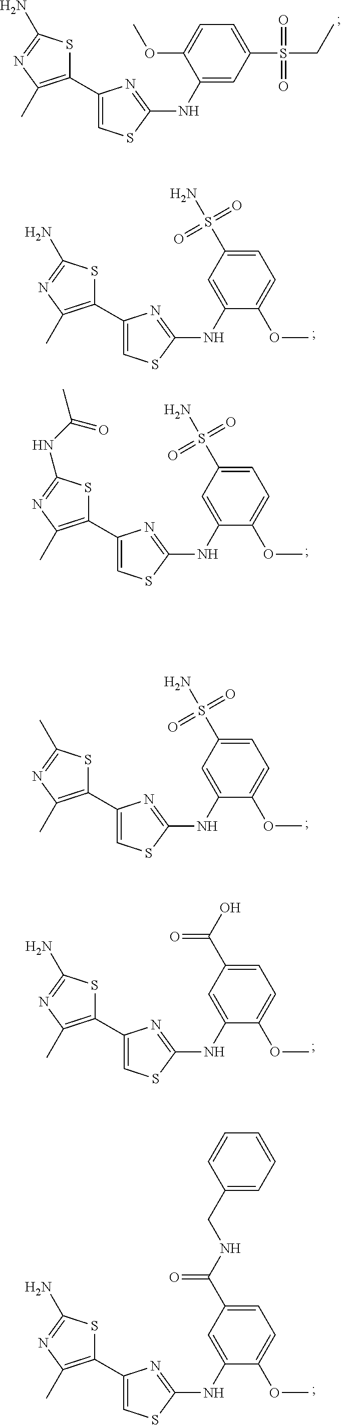

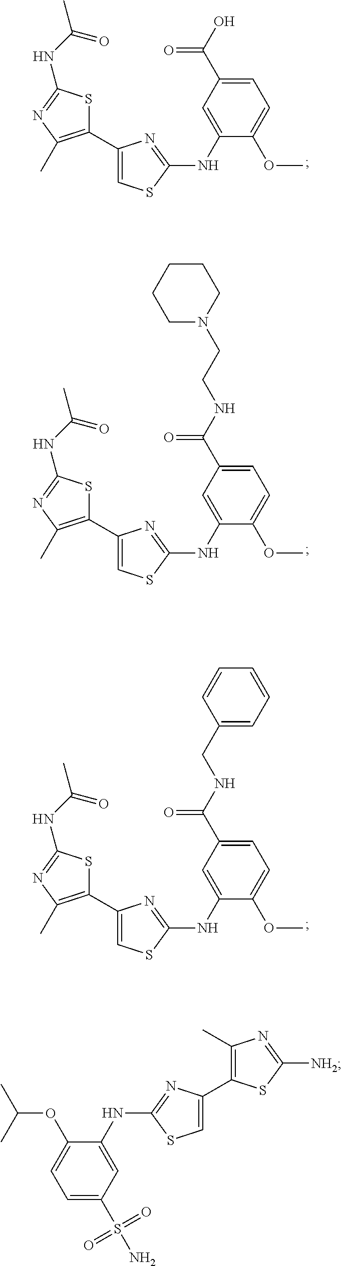

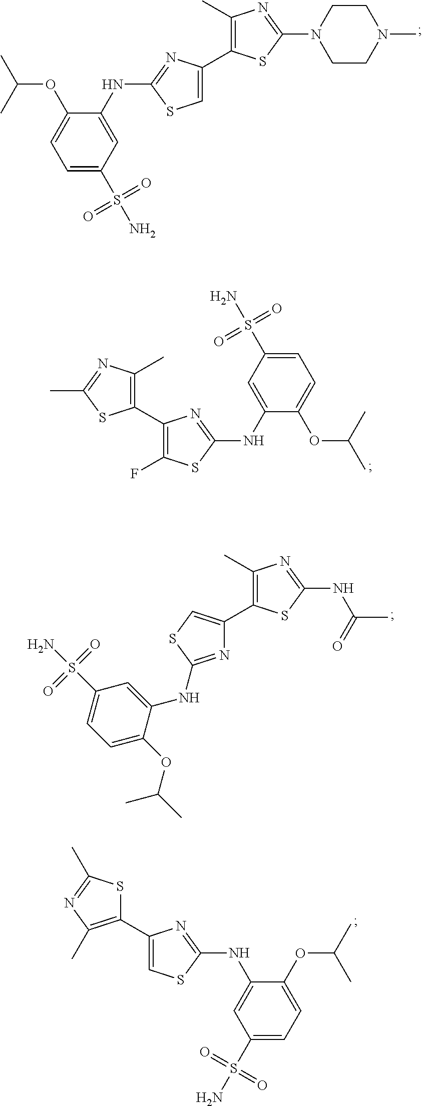

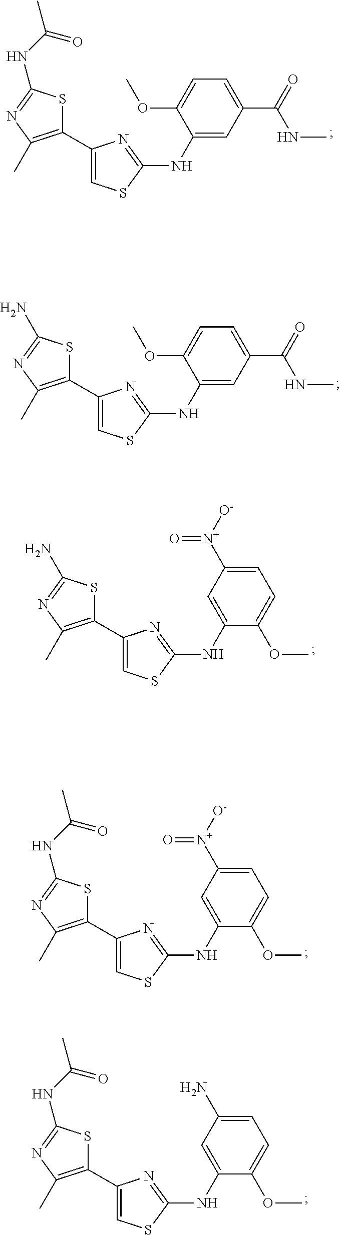

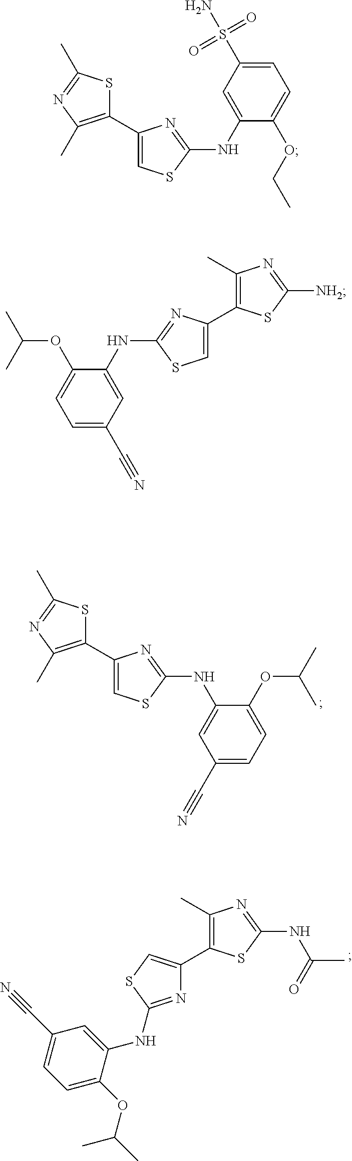

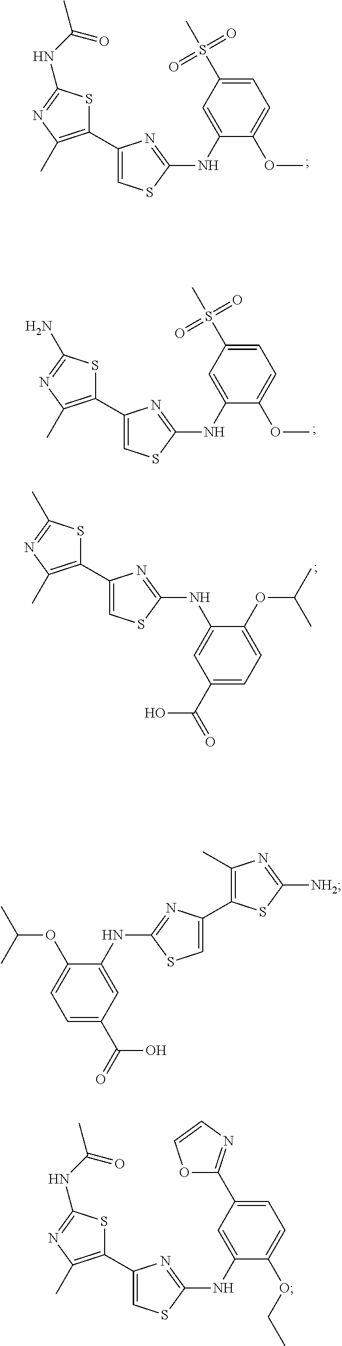

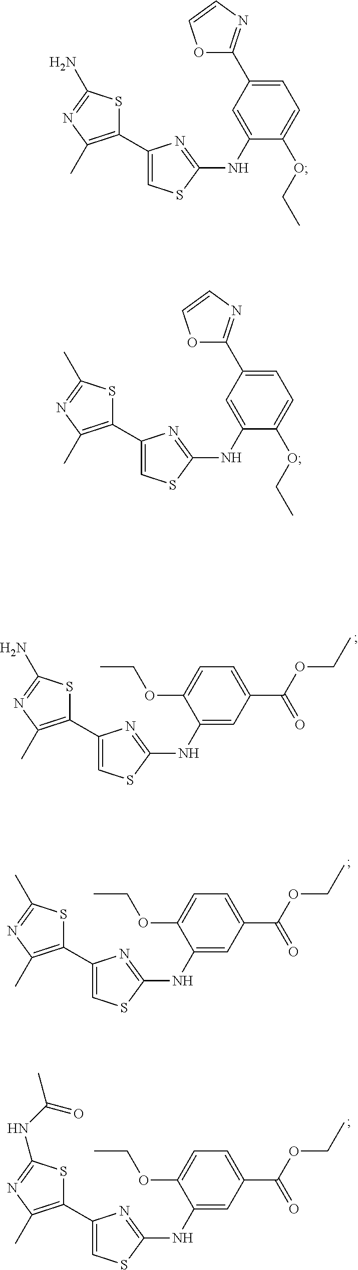

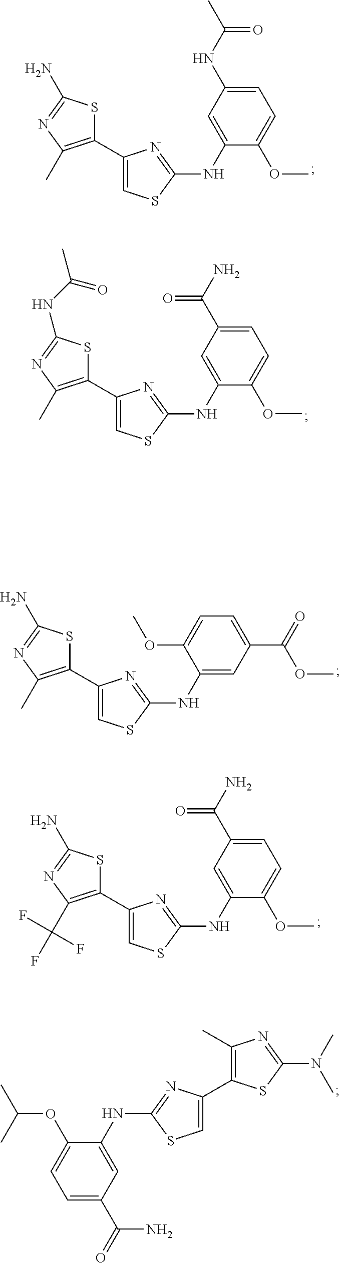

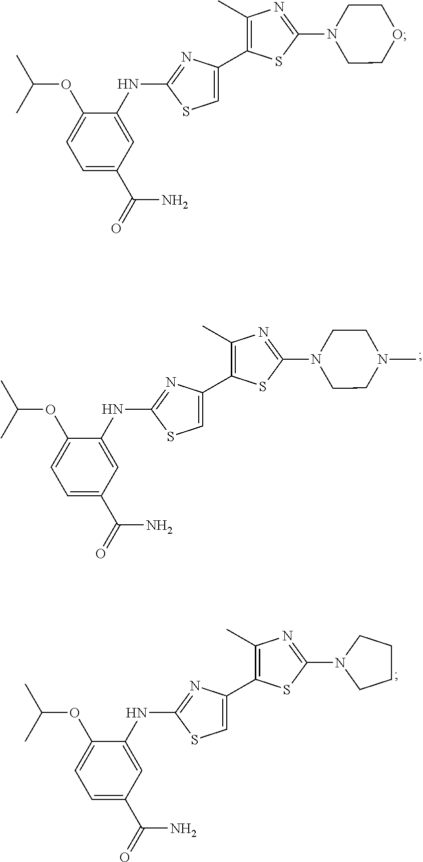

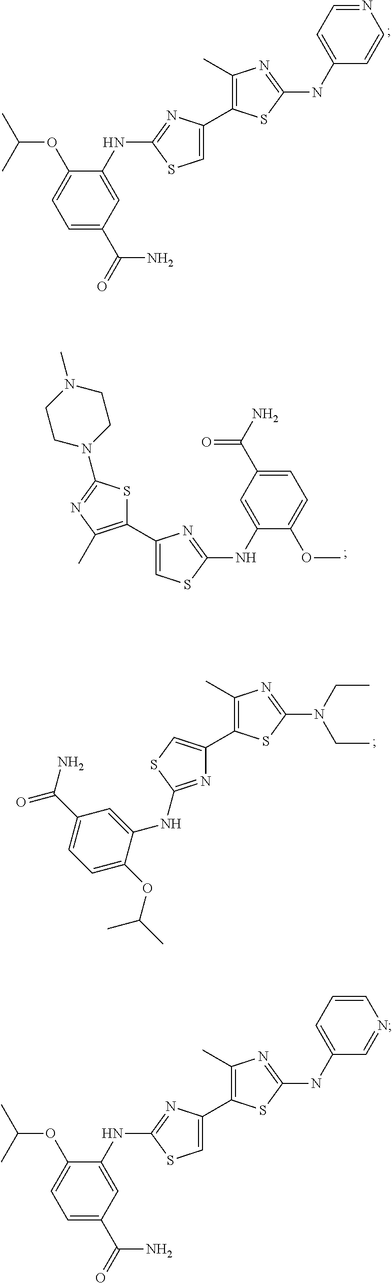

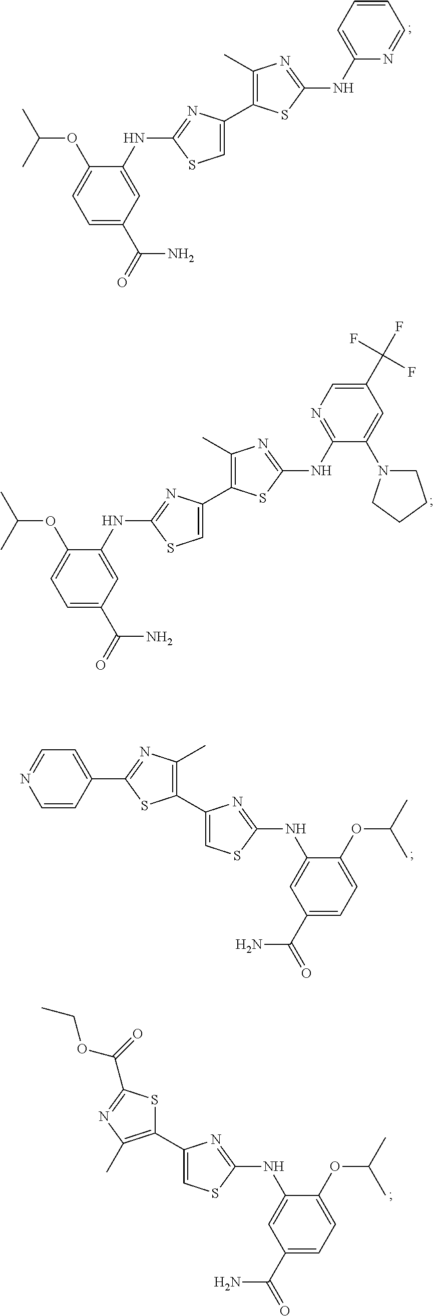

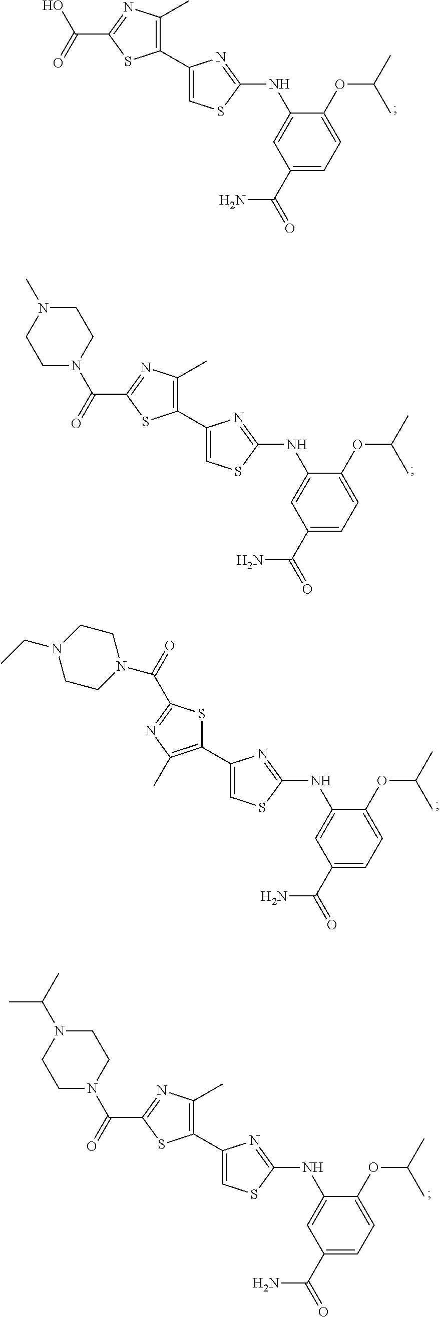

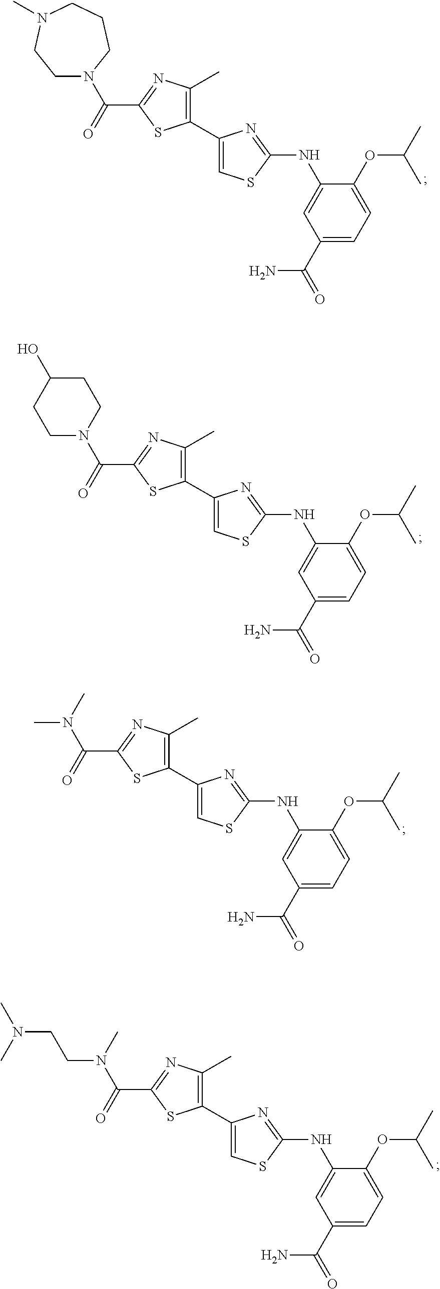

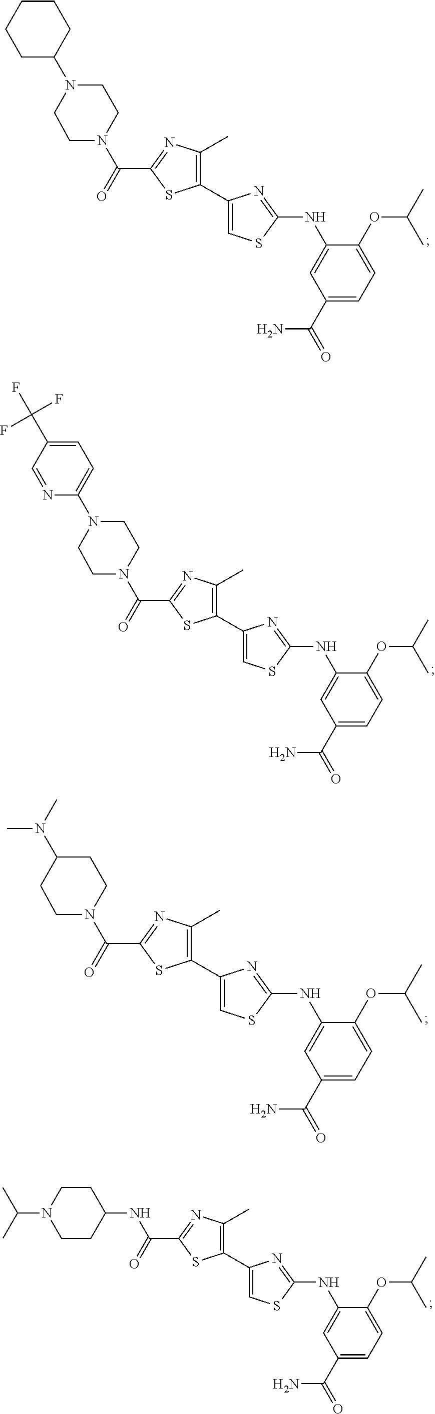

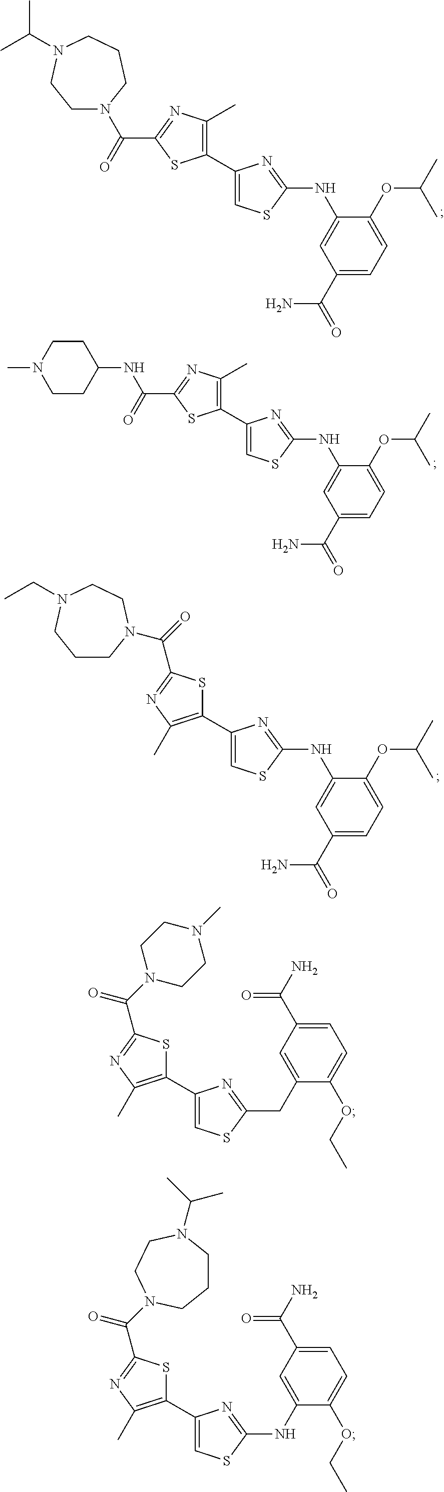

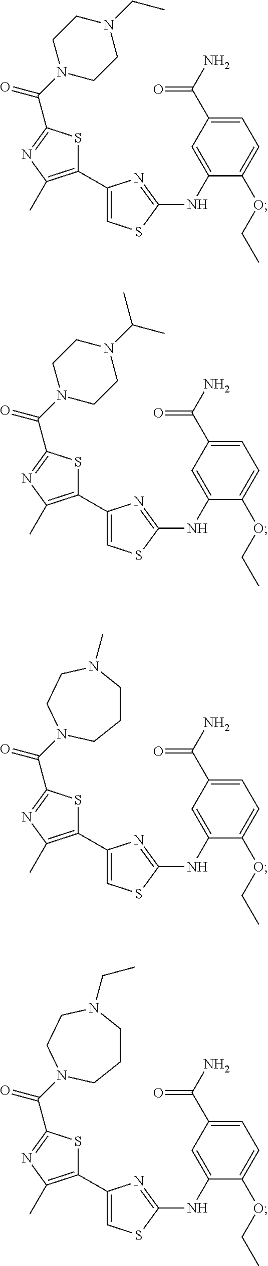

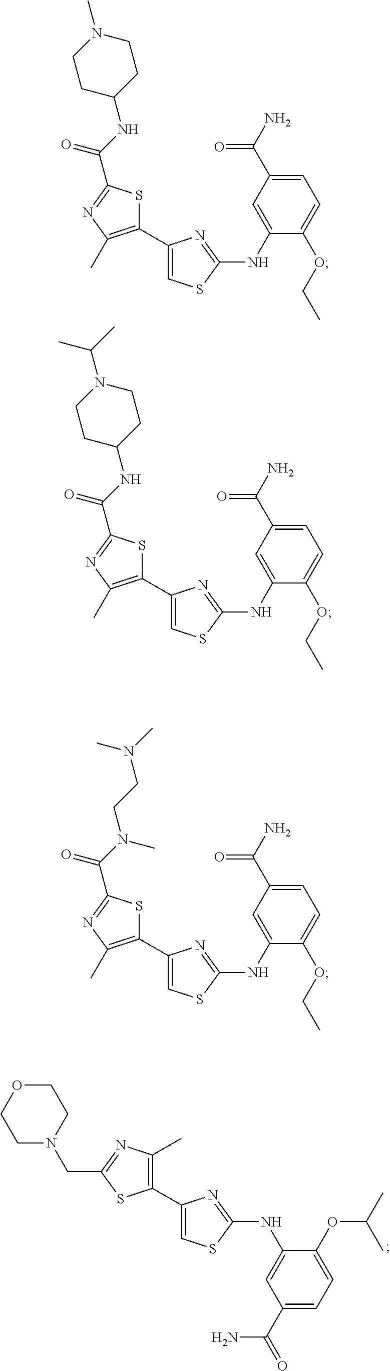

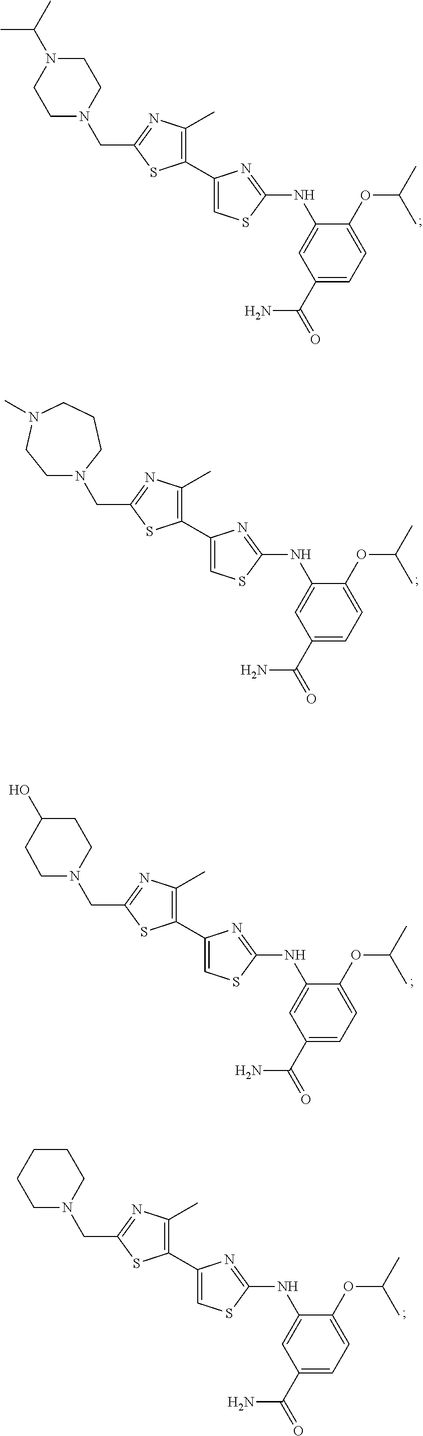

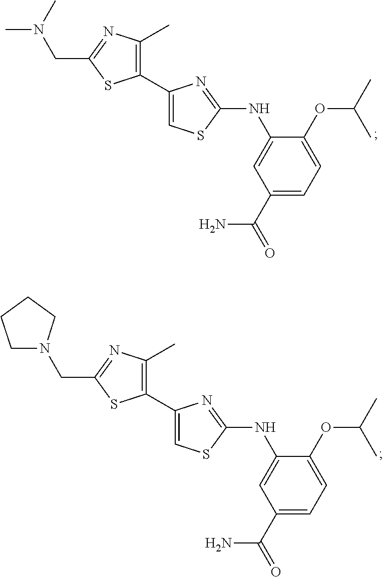

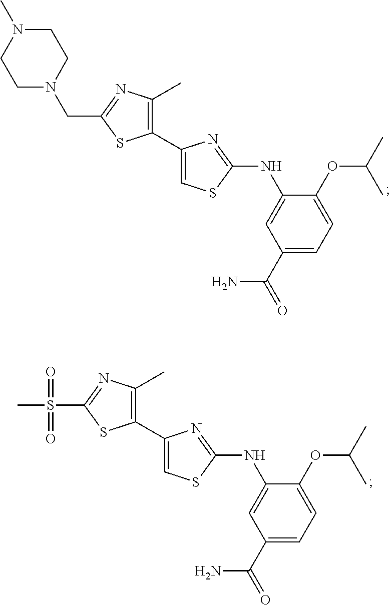

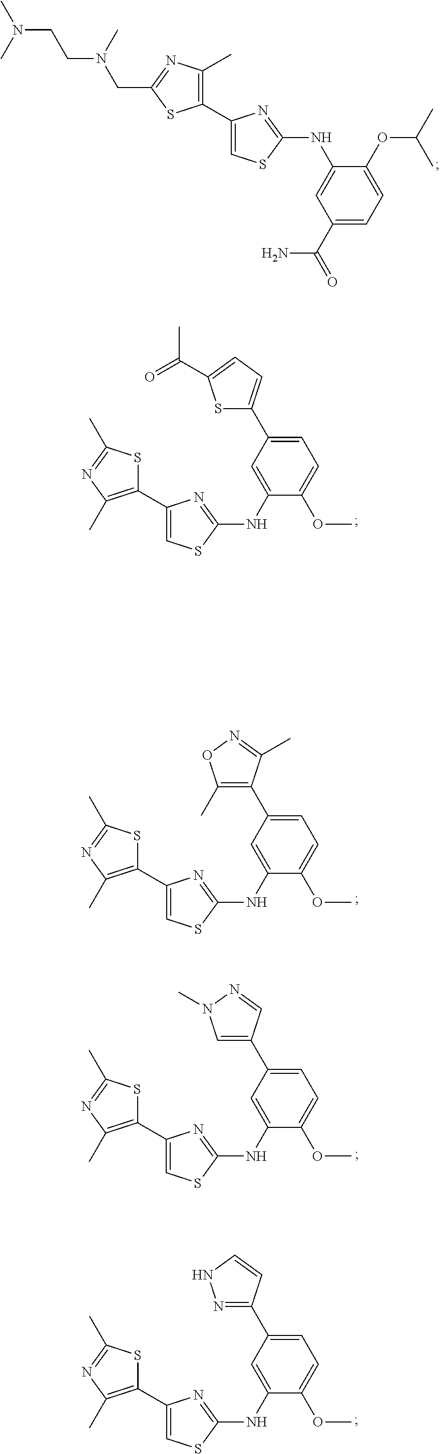

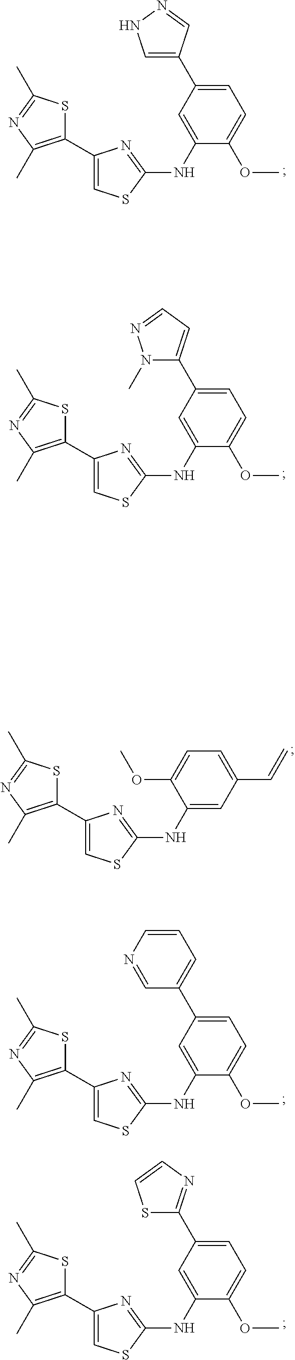

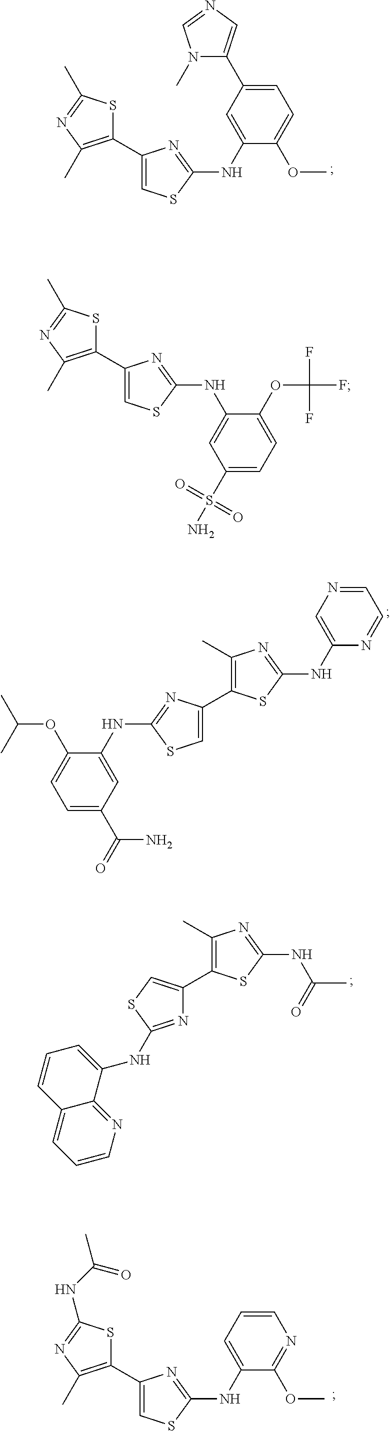

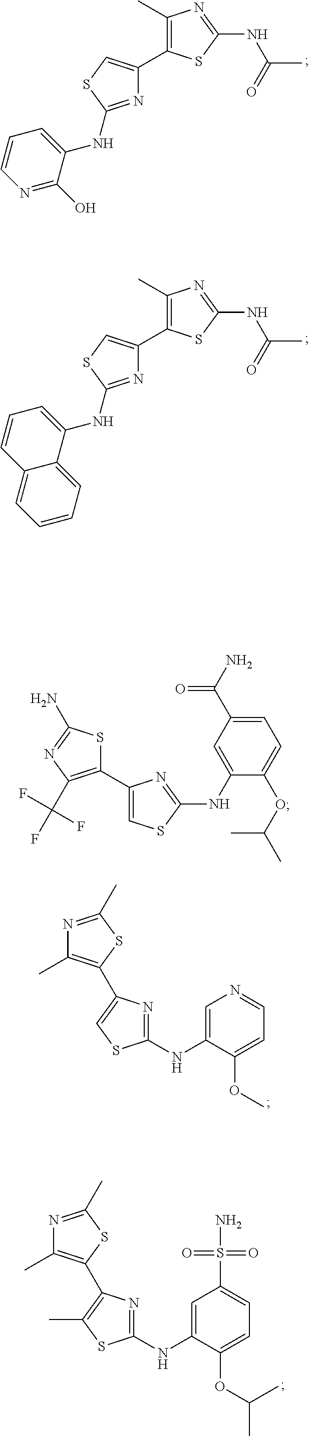

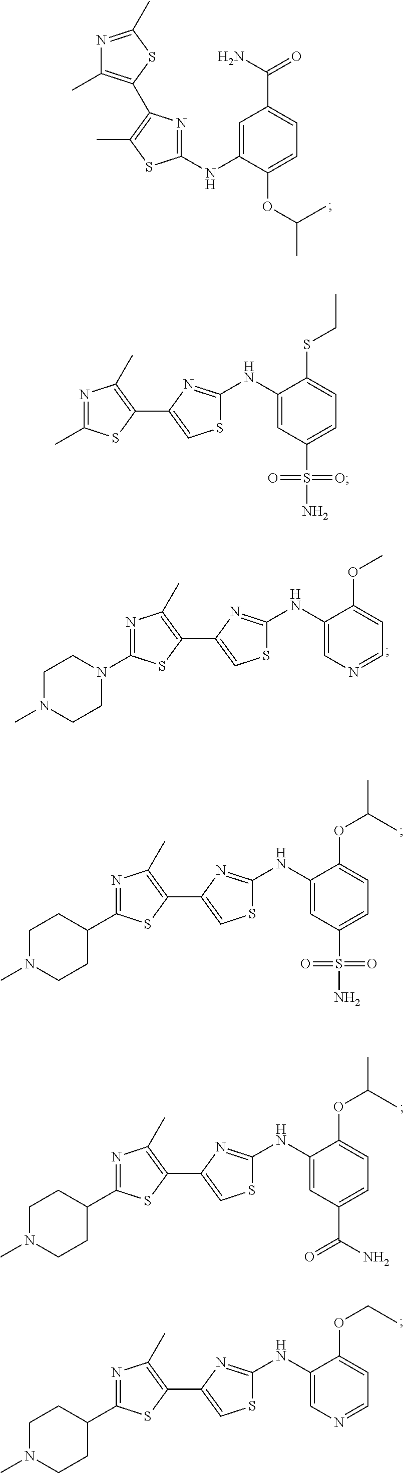

and solvates, hydrates, tautomers, and pharmaceutically acceptable salts thereof.

In another embodiment of the invention:

### A is

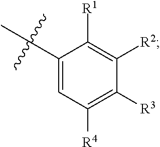

### R¹ is OCH(CH₃)₂;

### R² is H;

### R³ is H;

### R⁴ is —CONH₂, —CO₂H, or —SO₂NH₂;

### R⁵ is H;

### R⁶ is CH₃;

R⁷ is H, CF₃, CH₂CF₃, Cl, Br, C₍₁₋₆₎alkyl,

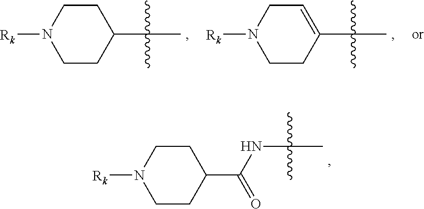

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A²; wherein said

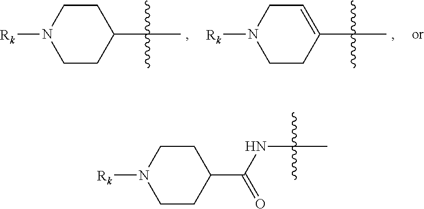

is optionally substituted with up to four methyl groups on two or more ring carbon atoms or optionally substituted with up to two CF₃ groups on any two ring carbon atoms;
A¹ is H, or C₍₁₋₃₎alkyl;

A² is H, C₍₁₋₆₎alkyl, C₍₃₋₆₎cycloalkyl,

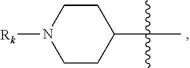

C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

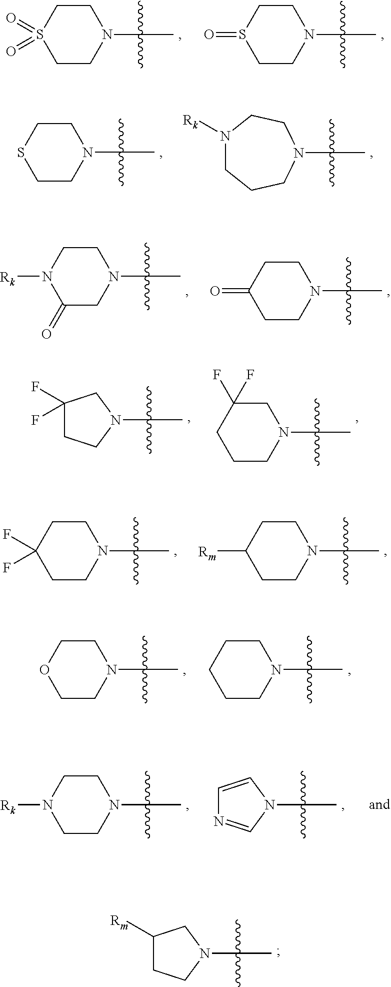

wherein any said A¹ and A² ring, except imidazolyl, may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoropyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH; and solvates, hydrates, tautomers, and pharmaceutically acceptable salts thereof.

Another embodiment of the invention is a pharmaceutical composition, comprising a compound of Formula I and a pharmaceutically acceptable carrier.

Another embodiment of the invention is a pharmaceutical composition, comprising a compound listed in the Examples section of this specification and a pharmaceutically acceptable carrier.

The present invention also provides a method for preventing, treating or ameliorating an MMP9 mediated syndrome, disorder or disease comprising administering to a subject in need thereof an effective amount of a compound of Formula I or a form, composition or medicament thereof.

The present invention also provides a method for preventing, treating or ameliorating an MMP13 mediated syndrome, disorder or disease comprising administering to a subject in need thereof an effective amount of a compound of Formula I or a form, composition or medicament thereof.

The present invention also provides a method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound of Formula I or a form, composition or medicament thereof.

The present invention also provides a method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound of Formula I or a form, composition or medicament thereof.

The present invention provides a method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound of Formula I or a form, composition or medicament thereof.

The present invention provides a method of preventing, treating or ameliorating a neoplastic disorder, wherein said neoplastic disorder is ovarian cancer, comprising administering to a subject in need thereof an effective amount of a compound of Formula I or a form, composition or medicament thereof.

The present invention provides a method of preventing, treating or ameliorating a cardiovascular disease, wherein said cardiovascular disease is selected from the group consisting of: atherosclerotic plaque rupture, aneurysm, vascular tissue morphogenesis, coronary artery disease, and myocardial tissue morphogenesis, comprising administering to a subject in need thereof an effective amount of a compound of Formula I or a form, composition or medicament thereof.

The present invention provides a method of preventing, treating or ameliorating atherosclerotic plaque rupture, comprising administering to a subject in need thereof an effective amount of a compound of Formula I or a form, composition or medicament thereof.

The present invention provides a method of preventing, treating or ameliorating rheumatoid arthritis, comprising administering to a subject in need thereof an effective amount of a compound of Formula I or a form, composition or medicament thereof.

The present invention provides a method of preventing, treating or ameliorating asthma, comprising administering to a subject in need thereof an effective amount of a compound of Formula I or a form, composition or medicament thereof.

The present invention provides a method of preventing, treating or ameliorating chronic obstructive pulmonary disease, comprising administering to a subject in need thereof an effective amount of a compound of Formula I or a form, composition or medicament thereof.

The present invention provides a method of preventing, treating or ameliorating inflammatory bowel syndrome, comprising administering to a subject in need thereof an effective amount of a compound of Formula I or a form, composition or medicament thereof.

The present invention provides a method of preventing, treating or ameliorating abdominal aortic aneurism, comprising administering to a subject in need thereof an effective amount of a compound of Formula I or a form, composition or medicament thereof.

The present invention provides a method of preventing, treating or ameliorating osteoarthritis, comprising administering to a subject in need thereof an effective amount of a compound of Formula I or a form, composition or medicament thereof.

The present invention provides a method of preventing, treating or ameliorating idiopathic pulmonary fibrosis, comprising administering to a subject in need thereof an effective amount of a compound of Formula I or a form, composition or medicament thereof.

The invention also relates to methods of inhibiting MMP9 activity in a mammal by administration of an effective amount of at least one compound of Formula I.

The invention also relates to methods of inhibiting MMP13 activity in a mammal by administration of an effective amount of at least one compound of Formula I.

In another embodiment, the invention relates to a compound as described in the Examples section for use as a medicament, in particular, for use as a medicament for treating a MMP9 mediated syndrome, disorder or disease.

In another embodiment, the invention relates to the use of a compound as described in the Examples section for the preparation of a medicament for the treatment of a disease associated with an elevated or inappropriate MMP9 activity.

In another embodiment, the invention relates to a compound as described in the Examples section for use as a medicament, in particular, for use as a medicament for treating a MMP13 mediated syndrome, disorder or disease.

In another embodiment, the invention relates to the use of a compound as described in the Examples section for the preparation of a medicament for the treatment of a disease associated with an elevated or inappropriate MMP13 activity.

### DEFINITIONS

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl. Any alkyl group may be optionally substituted with one OCH₃, one OH, or up to two fluorine atoms.

The term “alkoxy” refers to a saturated branched or straight chain monovalent hydrocarbon alcohol radical derived by the removal of the hydrogen atom from the hydroxide oxygen substituent on a parent alkane. Examples include C₍₁₋₆₎alkoxy or C₍₁₋₄₎alkoxy groups. Any alkoxy group may be optionally substituted with one OCH₃, one OH, or up to two fluorine atoms.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₍₃₋₆₎cycloalkyl, C₍₅₋₈₎cycloalkyl, decahydronaphthalenyl, and 2,3,4,5,6,7-hexahydro-1H-indenyl. Any cycloalkyl group may be optionally substituted with one OCH₃, one OH, or up to two fluorine atoms.

### ABBREVIATIONS

Herein and throughout this application, the following abbreviations may be used.
Ac —C(O)CH₃
aq. aqueous
Bu butyl
conc. concentrated
DCM dichloromethane
DME dimethoxyethane
DMF dimethylformamide
DMSO dimethylsulfoxide
Et ethyl
g gram
h hours
d days
HATU 2-(7-aza-1H-benzotriazole-1-yl)-1,1,3,3-tetramethyluronium hexafluorophosphate
hept heptanes
HPLC high pressure liquid chromatography
KHMDS ((CH₃)₃Si)₂NK
M molar
Me methyl
mL milliliter
mmol millimole
mg milligram
min minutes
MsCl mesyl chloride
N normal
NMR nuclear magnetic resonance
OXONE® potassium peroxymonosulfate
Pd(dppf)Cl_{2 [}1,1′-bis(diphenylphosphino)ferrocene]dichloropalladium(II)
Ph phenyl
iPr isopropyl
RP reverse phase
Selectfluor® 1-chloromethyl-4-fluoro-1,4-diazoniabicyclo[2.2.2]octane bis-(tetrafluoroborate)
sat. saturated
TFA trifluoroacetic acid
THF tetrahydrofuran
OTs tosylate
TLC thin layer chromatography
TsOH para-toluenesulfonic acid
v volume

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine or zinc.

### Methods of Use

The present invention is directed to a method for preventing, treating or ameliorating a MMP9 and/or MMP13 mediated syndrome, disorder or disease comprising administering to a subject in need thereof an effective amount of a compound of Formula I or a form, composition or medicament thereof.

Examples of a MMP9 and/or MMP13 mediated syndrome, disorder or disease for which the compounds of Formula I are useful include angiogenesis, osteoarthritis, rheumatoid arthritis, gastric ulcers, pulmonary hypertension, chronic obstructive pulmonary disorder, inflammatory bowel syndrome, periodontal disease, skin ulcers, liver fibrosis, emphysema, Marfan syndrome, stroke, multiple sclerosis, abdominal aortic aneurysm, coronary artery disease, idiopathic pulmonary fibrosis, renal fibrosis, migraine, and cardiovascular disorders including: atherosclerotic plaque, ruptive aneurysm, vascular tissue morphogenesis, and myocardial tissue morphogenesis.

The term “administering” with respect to the methods of the invention, means a method for therapeutically or prophylactically preventing, treating or ameliorating a syndrome, disorder or disease as described herein by using a compound of Formula I or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment. In one aspect of the invention, the subject is at risk of (or susceptible to) developing a syndrome, disorder or disease that is associated with elevated MMP9 and/or MMP13 expression or MMP9 and/or MMP13 overexpression, or a patient with an inflammatory condition that accompanies syndromes, disorders or diseases associated with elevated MMP9 and/or MMP13 expression or MMP9 and/or MMP13 overexpression.

The term “therapeutically effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

When employed as inhibitors of pro-matrix metalloproteinase activation, the compounds of the invention may be administered in an effective amount within the dosage range of about 0.5 mg to about 10 g, preferably between about 0.5 mg to about 5 g, in single or divided daily doses. The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The compounds of Formula I may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

The present invention also encompasses a method of making a pharmaceutical composition comprising mixing a pharmaceutically acceptable carrier with any of the compounds of the present invention. Additionally, the present invention includes pharmaceutical compositions made by mixing a pharmaceutically acceptable carrier with any of the compounds of the present invention. As used herein, the term “composition” is intended to encompass a product comprising the specified ingredients in the specified amounts, as well as any product which results, directly or indirectly, from combinations of the specified ingredients in the specified amounts.

### Polymorphs and Solvates

Furthermore, the compounds of the present invention may have one or more polymorph or amorphous crystalline forms and as such are intended to be included in the scope of the invention. In addition, the compounds may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the compounds of the present invention. Thus, in the methods of treatment of the present invention, the term “administering” shall encompass the means for treating, ameliorating or preventing a syndrome, disorder or disease described herein with the compounds of the present invention or a polymorph or solvate thereof, which would obviously be included within the scope of the invention albeit not specifically disclosed.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution.

The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

### General Schemes:

Compounds of Formula I can be prepared by methods known to those who are skilled in the art. The following reaction schemes are only meant to represent examples of the invention and are in no way meant to be a limit of the invention.

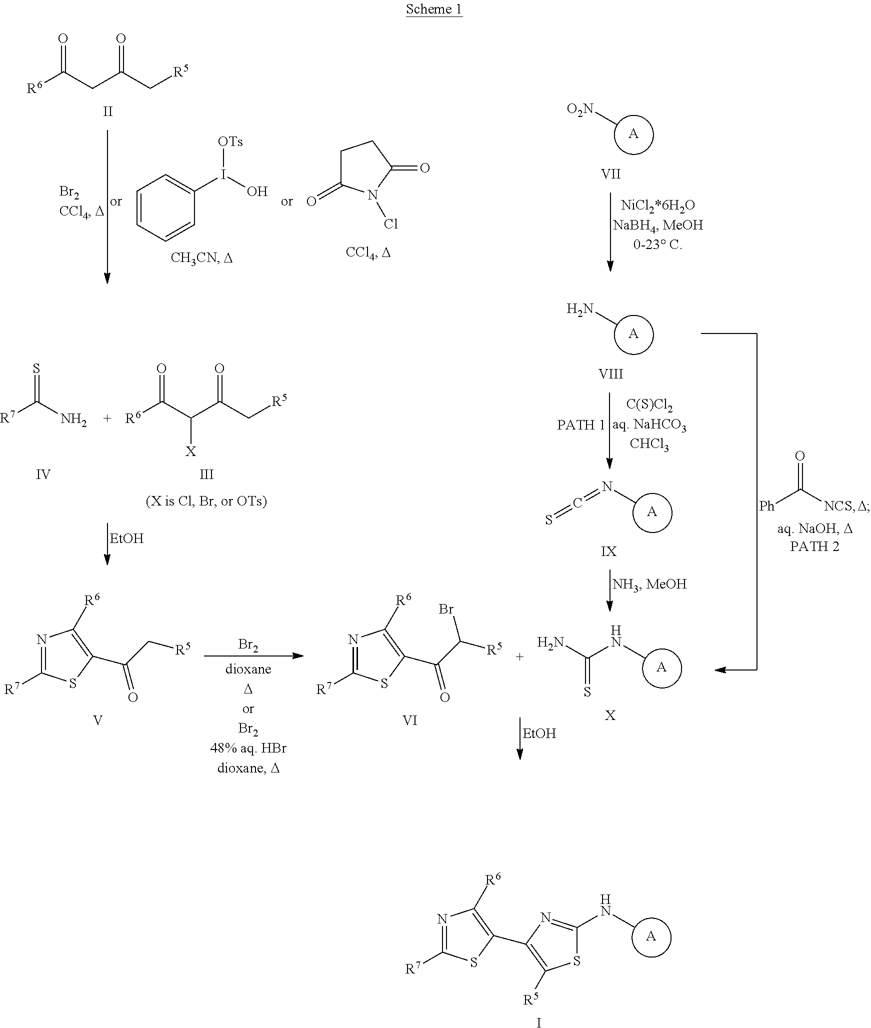

Scheme 1 illustrates synthetic routes leading to compounds of Formula I. Starting with dicarbonyl II, chlorination, bromination, or tosylation results in formation of III, which is condensed with IV to form thiazole V. Thiazole V is treated with bromine to yield bromide VI. Condensation of VI with thiourea X, where A is as defined in Formula I, affords compounds of Formula I. The required thiourea X may be prepared starting with nitro aromatic VII by reduction to the corresponding aniline VIII. The aniline VIII is converted to an isothiocyanate by reaction with thiophosgene and a base, and the isothiocyanate IX is treated with ammonia to provide thiourea X (path 1). Alternatively, following path 2, aniline VIII can be directly converted to thiourea X by reaction with benzoyl isothiocyanate followed by hydrolysis under basic aqueous conditions.

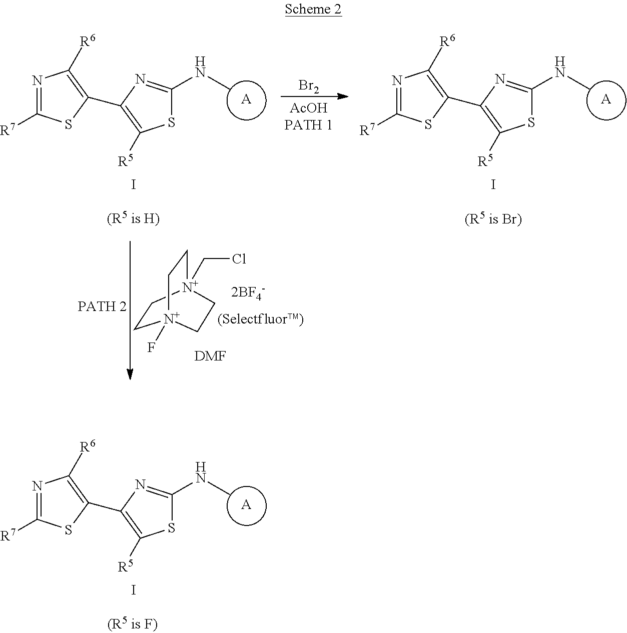

Scheme 2 depicts synthetic routes to compounds of Formula I, where R⁵ is bromo or fluoro. A compound of Formula I, where R⁵ is hydrogen, can be brominated by treatment with bromine in acetic acid to give a compound of Formula I, where R⁵ is bromo (path 1). Alternatively, according to path 2, I, where R⁵ is hydrogen, can be treated with Selectfluor™ to form a compound of Formula I, where R⁵ is fluoro.

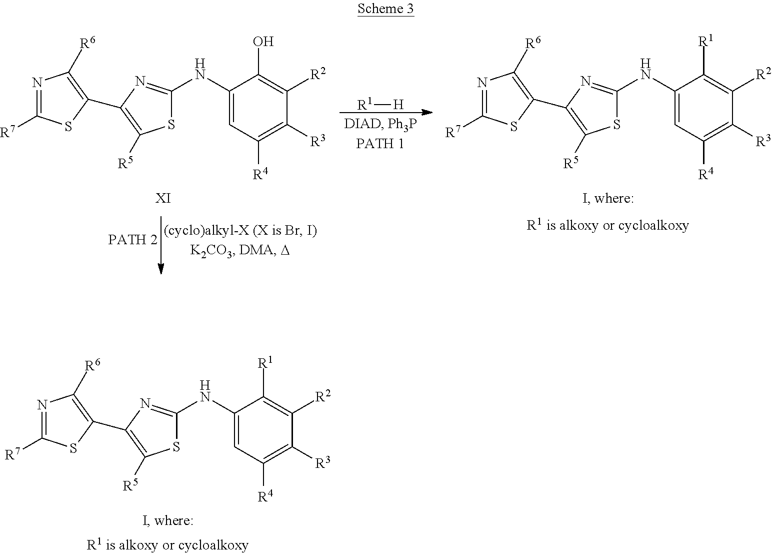

Alternative routes to compounds of Formula I, where R¹ is alkoxy or cycloalkoxy are illustrated in Scheme 3. As path 1 illustrates, phenol XI, prepared as described in Scheme 1, can be converted to a compound of Formula I, where R¹ is alkoxy or cycloalkoxy, by reaction with an alcohol, triphenylphosphine and diisopropyl azodicarboxylate (DIAD). Alternatively, by the route shown in path 2, the compound of formula XI may also be alkylated with an electrophile, such as an alkyl or cycloalkyl bromide or iodide, by heating in the presence of a base to provide I, where R¹ is alkoxy or cycloalkoxy.

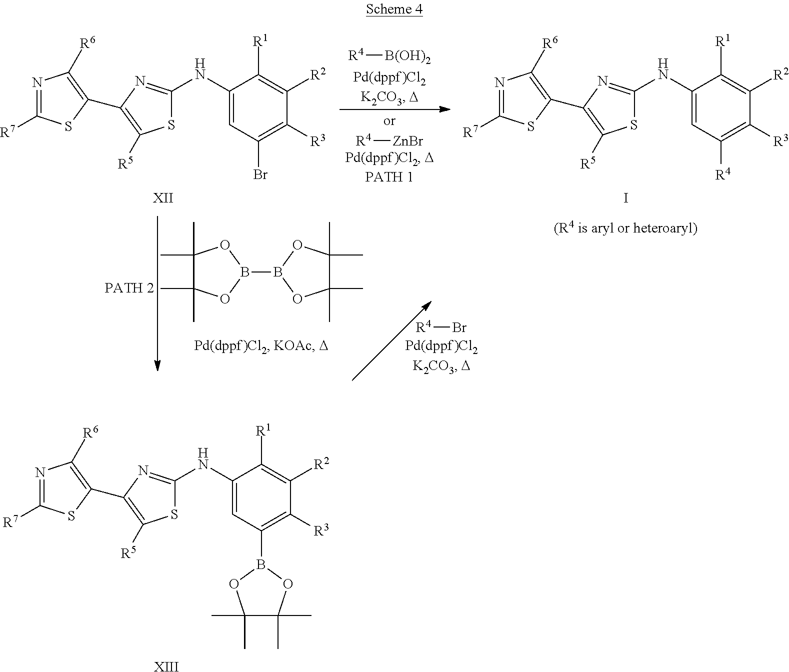

Additional routes to compounds of Formula I, where R⁴ is aryl or heteroaryl, are depicted in Scheme 4. An aryl bromide XII, prepared as described in Scheme 1, can react with a boronic acid (or ester), or a zinc reagent, in the presence of a palladium catalyst to yield I, where R⁴ is aryl or heteroaryl, as shown in path 1. Alternatively, as path 2 illustrates, the compound of formula XII can be converted to the corresponding boronate ester XIII by treatment with bis(pinacolato)diboron and a palladium catalyst. The boronate ester XIII may be converted to a compound of Formula I, where R⁴ is aryl or heteroaryl, by reaction with an aryl or heteroaryl bromide under palladium catalyzed conditions.

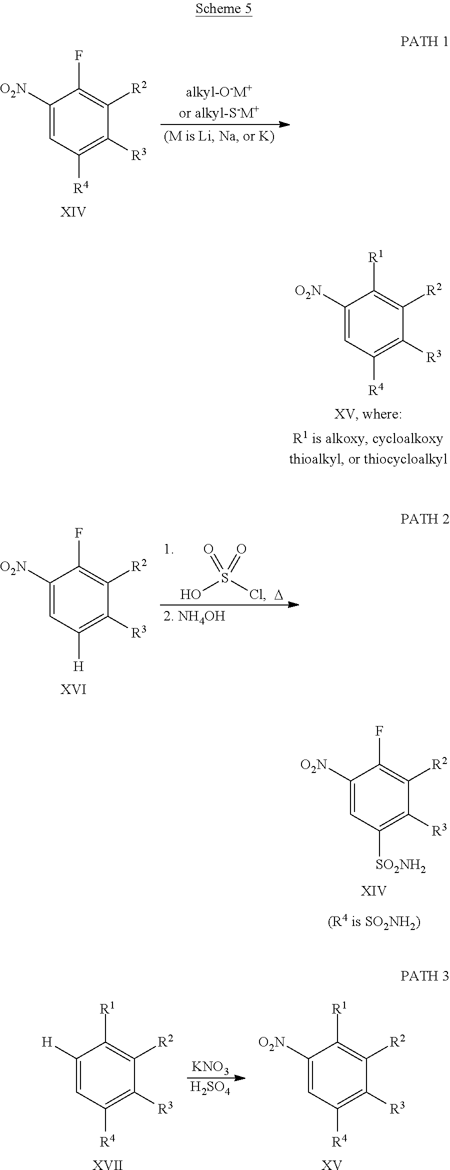

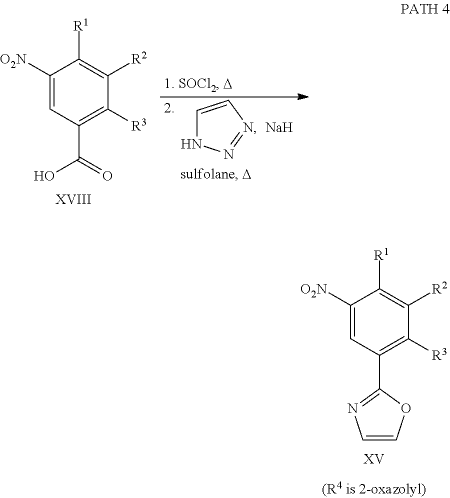

Many intermediates of formulae VII, VIII, IX, and X (as used in Scheme I) are commercially available. Scheme 5 illustrates synthetic routes (paths 1 to 4) to aryl nitro compounds of formula XV, which may be converted to compounds of Formula I as described in Scheme 1. A 2-nitrofluoro benzene XIV can be reacted with a metal alkoxide or thiolate to yield XV, where R¹ is alkoxy, cycloalkoxy, thioalkyl, or thiocycloalkyl (path 1). As shown in path 2, where R⁴ is SO₂NH₂, the required starting material XIV may be obtained by heating 2-fluoronitro benzene XVI (unsubstituted para to the fluorine) in neat chlorosulfonic acid, followed by treatment of the aryl sulfonyl chloride intermediate with ammonium hydroxide solution. Additional aryl nitro compounds XV may be obtained by treatment of substituted aryls XVII with a nitrating reagent, such as KNO₃/H₂SO₄, HNO₃/H₂SO₄, or HNO₃/Ac₂O (path 3). Those skilled in the art will recognize that path 3 is preferably employed when nitration is desired to occur at a position ortho or para to electron-donating substituents, such as alkoxy or alkyl, and meta to electron-withdrawing substituents, such as CONH₂. As depicted in path 4, nitro compounds of formula XV, where R⁴ is 2-oxazolyl, may be prepared by conversion of aryl carboxylic acid XVIII to the corresponding acid chloride, for instance by heating in thionyl chloride, followed by reaction with a metal salt of 1,2,3-1H-triazole.

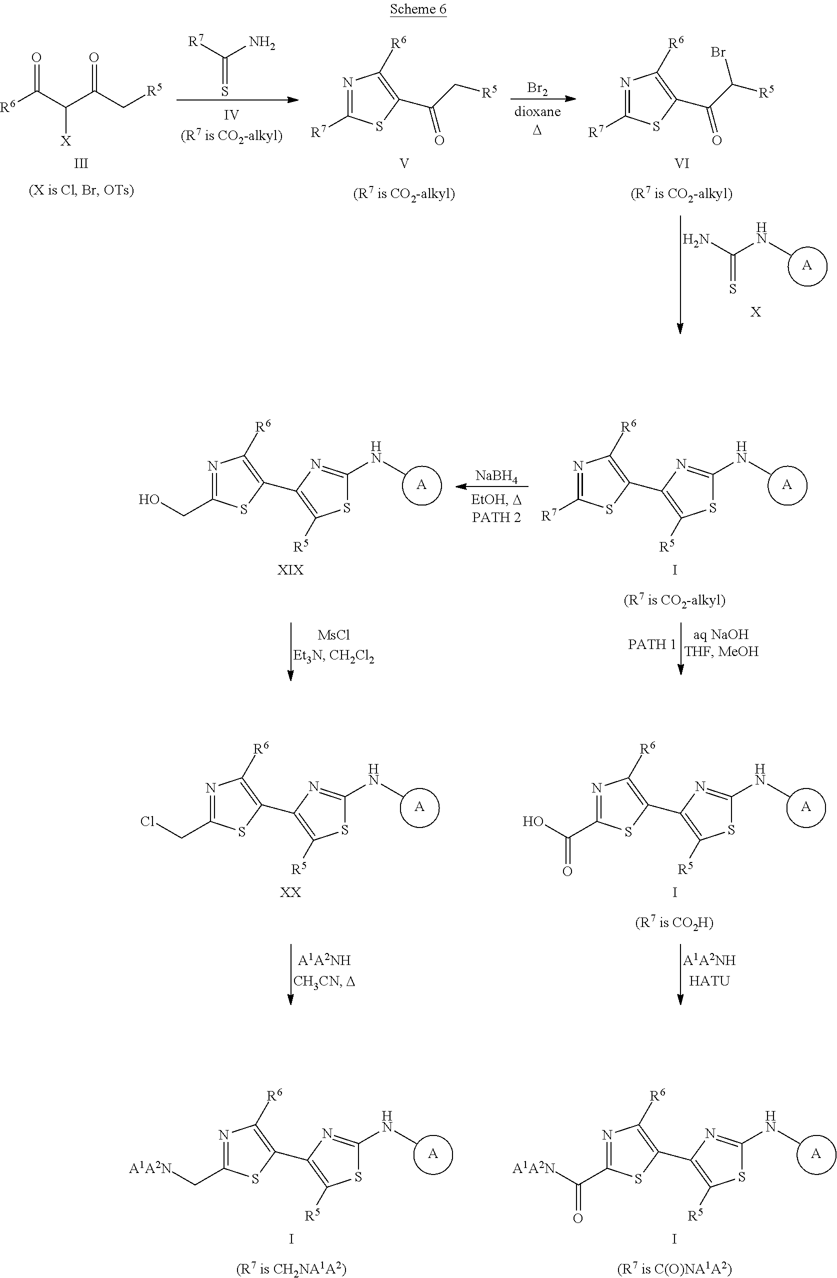

Synthetic routes to compounds of Formula I, where R⁷ is CO₂-alkyl, CO₂H, C(O)NA¹A², or CH₂NA¹A² are shown in Scheme 6. Condensation of III with an alkyl thiooxamate (IV, where R⁷ is CO₂-alkyl) provides thiazole V, where R⁷ is CO₂-alkyl. Following conversion to bromide VI and condensation with thiourea X, hydrolysis under basic aqueous conditions yields compounds of Formula I, where R⁷ is CO₂H (path 1). Compounds of Formula I, where R⁷ is CO₂H, are reacted with an amine A¹A²NH in the presence of a coupling agent, such a HATU, to form compounds of Formula I, where R⁷ is C(O)NA¹A². Alternatively, following path 2, a compound of Formula I, where R⁷ is CO₂-alkyl, can be reduced by reaction with sodium borohydride to alcohol XIX. The alcohol is reacted with methanesulfonyl chloride (MsCl) and a tertiary amine base to yield the chloride XX. Displacement of the chloride with an amine A¹A²NH affords compounds of Formula I, where R⁷ is CH₂NA¹A².

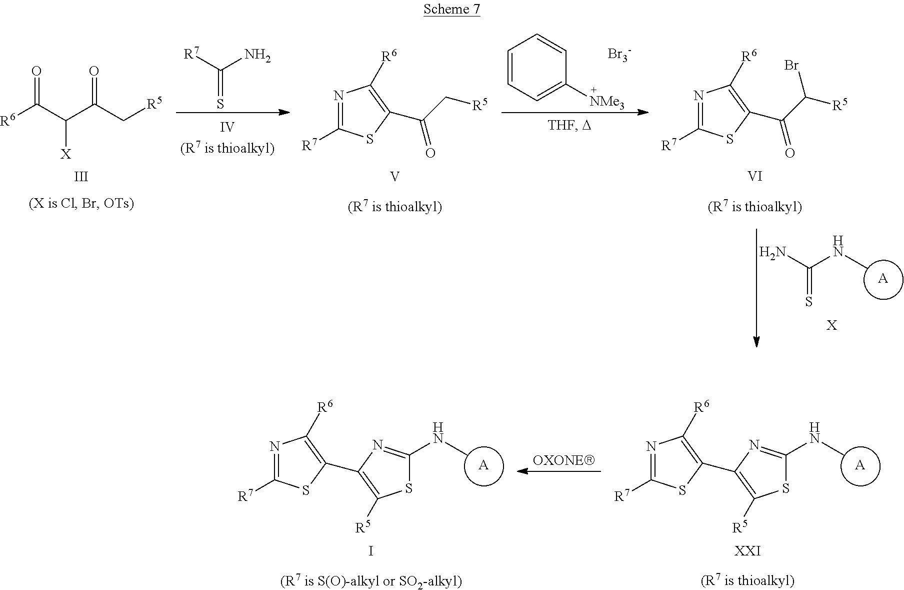

Scheme 8 depicts synthetic routes to compounds of Formula I, where R⁷ is S(O)-alkyl or SO₂-alkyl. Condensation of III with a dithiocarbamic acid alkyl ester (IV, where R⁷ is thioalkyl) affords thiazole V. Bromination of V by treatment with phenyltrimethylammonium tribromide yields bromide VI. Condensation with thiourea X then provides bisthiazole XXI. Bisthiazole XXI can be oxidized with OXONE®, or another oxidant such as hydrogen peroxide or meta-chloroperbenzoic acid, to afford compounds of Formula I, where R⁷ is S(O)-alkyl or SO₂-alkyl. Those skilled in the art will recognize that the reaction can be controlled to favor formation of the sulfinyl-substituted (R⁷ is S(O)-alkyl) or sulfonyl-substituted (R⁷ is SO₂-alkyl)thiazole products by varying factors such as the amount of oxidant used and the time of reaction.

### EXAMPLES

### Intermediate 1

### 1-(2-Amino-thiazol-5-yl)-2-bromo-ethanone

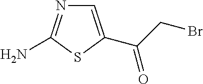

Bromine (0.115 mL, 2.24 mmol) in dioxane (10 mL) was added to a solution of 1-(2-amino-thiazol-5-yl)-ethanone (500 mg, 2.24 mmol, prepared as described in J. Org. Chem. 1984, 49, 566) in 48% aq. HBr (10 mL) at 60° C. The resulting orange solution was stirred at this temperature for 1 h, then was added dropwise to stirred ice-cold sat. aq. NaHCO₃. The mixture was extracted with EtOAc. The organic phase was dried (Na₂SO₄), filtered, and concentrated. The residue was purified by flash column chromatography (Silica gel, 20-60% EtOAc-Hept), affording the title compound as a white solid.

### Intermediate 2: Step a

### 1-Methyl-piperidine-4-carboxylic acid (5-acetyl-4-methyl-thiazol-2-yl)-amide

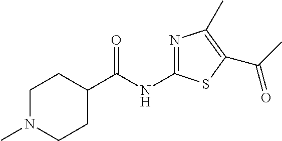

A suspension of 1-methyl-piperidine-4-carbonyl chloride.HCl (691.7 mg, 3.49 mmol, prepared as described in WO 2003/084949) in THF (10 mL) was added to an ice-cold mixture of 1-(2-amino-4-methyl-thiazol-5-yl)-ethanone (545.5 mg, 3.49 mmol, Aldrich) and N,N-diisopropylethylamine (0.73 mL, 4.19 mmol) in THF (5 mL). The mixture was allowed to slowly warm to room temperature and was stirred for 2 d. The reaction mixture was partitioned between EtOAc and sat. aq. NaHCO₃. The separated aq. phase was saturated with NaCl and was exhaustively extracted with CH₂Cl₂ followed by EtOAc. The organic extracts were dried (Na₂SO₄), filtered, and concentrated and the residue was purified by flash column chromatography (Biotage KP-NH column, 0-2% MeOH-EtOAc), affording the title compound as a cream-colored solid.

### Intermediate 2: Step b

### 1-Methyl-piperidine-4-carboxylic acid [5-(2-bromo-acetyl)-4-methyl-thiazol-2-yl]-amide.HBr

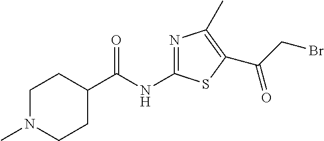

Bromine (0.0232 mL, 0.452 mmol) was added to a colorless solution of 1-methyl-piperidine-4-carboxylic acid (5-acetyl-4-methyl-thiazol-2-yl)-amide (115.5 mg, 0.410 mmol, intermediate 2, step a) in 48% aq. HBr (2 mL) and the resulting mixture was stirred in a 60° C. oil bath for 1.5 h. The reaction mixture was concentrated from toluene three times, affording the crude title compound as a partially solidified orange oil which was used without purification in the next step.

### Intermediate 3

### 2-Bromo-1-(2,4-dimethyl-thiazol-5-yl)-ethanone.HBr

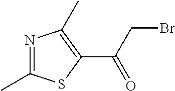

A suspension of bromine (11.9 mL, 231.5 mmol) in 1,4-dioxane (200 mL) was added to a stirred solution of 1-(2,4-dimethyl-thiazol-5-yl)-ethanone (28.75 g, 185.2 mmol, Alfa) in 1,4-dioxane (200 mL). The mixture was stirred for 25 h at 50° C. and the resulting cream-colored suspension was allowed to cool to room temperature and was filtered and washed with 2:1 heptane:EtOAc (v/v). The resulting white powder was recrystallized from EtOH, affording the title compound.

### Intermediate 4

### 1-(2-Amino-4-methyl-thiazol-5-yl)-2-bromo-ethanone

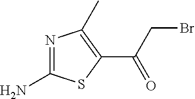

1-(2-Amino-4-methyl-thiazol-5-yl)-2-bromo-ethanone.HBr was prepared as described in WO 2005/068444. To convert to the corresponding free base, the crude reaction mixture was slowly added to ice-cold sat. aq. NaHCO₃ solution. The precipitate was collected by vacuum filtration and washed with Et₂O. The crude product was recrystallized from EtOH, affording the title compound as an orange powder.

### Intermediate 5

### N-[5-(2-Bromo-acetyl)-4-methyl-thiazol-2-yl]-acetamide.HBr

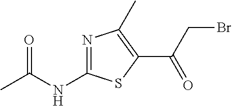

The title compound was prepared as described in WO 2005/068444.

### Intermediate 6: Step a

### Toluene-4-sulfonic acid 1-acetyl-3,3,3-trifluoro-2-oxo-propyl ester

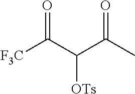

A solution of 1,1,1-trifluoro-2,4-pentanedione (3.00 g, 19.47 mmol) and [hydroxyl(tosyloxy)iodo]benzene (9.16 g, 23.36 mmol) in acetonitrile (100 mL) was heated to 45° C. for 45 minutes. The reaction mixture was then cooled to room temperature, evaporated and purified via column chromatography eluting with heptanes: ethyl acetate to give the title compound.

### Intermediate 6: Step b

### 1-(2-Amino-4-trifluoromethyl-thiazol-5-yl)-ethanone.TsOH

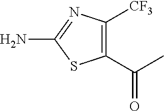

A solution of toluene-4-sulfonic acid 1-acetyl-3,3,3-trifluoro-2-oxo-propyl ester (0.100 g, 0.308 mmol, intermediate 6, step a) and thiourea (0.028 g, 0.370 mmol) in acetonitrile (5 mL) were heated to reflux for several hours. The reaction mixture was then cooled to room temperature and evaporated. Ethyl acetate was added and the suspension was filtered. The filtrate was evaporated, dichloromethane was added and the solution was filtered to give the title compound as a solid.

### Intermediate 6: Step c

### 1-(2-Amino-4-trifluoromethyl-thiazol-5-yl)-2-bromo-ethanone

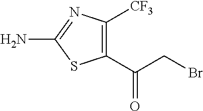

A solution of bromine (0.024 mL, 0.475 mmol) in dioxane (3 mL) was added dropwise to a solution of 1-(2-amino-4-trifluoromethyl-thiazol-5-yl)-ethanone (0.100 g, 0.475 mmol, intermediate 6, step b) in 48% aqueous HBr (3 mL) at 60° C. and stirred for 2 hours. The reaction mixture was cooled to room temperature, saturated aqueous NaHCO₃ was added slowly and the pH was adjusted to 7 with 2 N aqueous Na₂CO₃. Ethyl acetate was added and the product was extracted, dried with sodium sulfate and evaporated to give the title compound.

### Intermediate 7: Step a

### 1-[4-Methyl-2-(4-methyl-piperazin-1-yl)-thiazol-5-yl]-ethanone.HCl

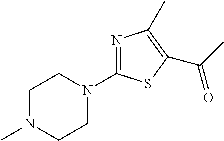

A mixture of 3-chloro-pentane-2,4-dione (400 mg, 2.97 mmol) and 4-methyl-piperazine-1-carbothioic acid amide (473 mg, 2.97 mmol) in EtOH (5 mL) was heated at 100° C. for 30 min. Upon cooling down, precipitate formed. The solid was filtered, washed with heptanes and air dried to yield the title compound.

### Intermediate 7: Step b

### 1-[4-Methyl-2-(4-methyl-piperazin-1-yl)-thiazol-5-yl]-ethanone

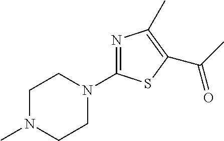

1-[4-Methyl-2-(4-methyl-piperazin-1-yl)-thiazol-5-yl]-ethanone.HCl (intermediate 7, step a) was partitioned between 2 N Na₂CO₃ and CH₂Cl₂, the organic layer was dried (Na₂SO₄), filtered, concentrated and dried under vacuum to provide the title compound as a free base.

### Intermediate 7: Step c

### 2-Bromo-1-[4-methyl-2-(4-methyl-piperazin-1-yl)-thiazol-5-yl]-ethanone.HBr

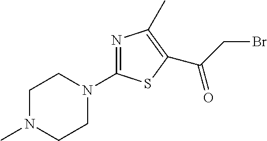

Bromine (0.0492 mL, 0.958 mmol) was added to a colorless solution of 1-[4-methyl-2-(4-methyl-piperazin-1-yl)-thiazol-5-yl]-ethanone (208.5 mg, 0.871 mmol, intermediate 7, step b) in 48% aq. HBr (2 mL) and the resulting mixture was stirred in a 60° C. oil bath for 1.5 h. The reaction mixture was concentrated from toluene three times, affording the crude title compound as a partially solidified orange oil which was used without purification in the next step.

### Intermediate 8: Step a

### 1-(2-Dimethylamino-4-methyl-thiazol-5-yl)-ethanone.HCl

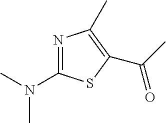

The title compound was prepared using 1,1-dimethyl-thiourea in place of 4-methyl-piperazine-1-carbothioic acid amide according to the procedure described for intermediate 7, step a.

### Intermediate 8: Step b

### 2-Bromo-1-(2-dimethylamino-4-methyl-thiazol-5-yl)-ethanone.HBr

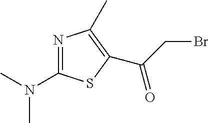

To 1-(2-dimethylamino-4-methyl-thiazol-5-yl)-ethanone.HCl (intermediate 8, step a) (146 mg, 0.663 mmol) in 48% HBr (3 mL) at 60° C. was added Br₂ (0.02 mL, 0.398 mmol) in 1,4-dioxane (3 mL). The mixture was stirred for 18 h at 60° C. and the resulting cream-colored suspension was allowed to cool to room temperature and was filtered, washed with 2:1 heptane:EtOAc (v/v) and air dried to give the title compound.

### Intermediate 9: Step a

### 1-(4-Methyl-2-morpholin-4-yl-thiazol-5-yl)-ethanone.HCl

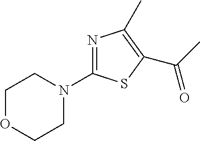

The title compound was prepared using morpholine-4-carbothioic acid amide in place of 4-methyl-piperazine-1-carbothioic acid amide according to the procedure described for intermediate 7, step a.

### Intermediate 9: Step b

### 2-Bromo-1-(4-methyl-2-morpholin-4-yl-thiazol-5-yl)-ethanone.HBr

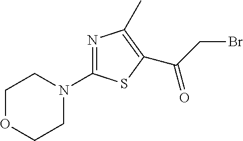

The title compound was prepared using 1-(4-methyl-2-morpholin-4-yl-thiazol-5-yl)-ethanone.HCl (intermediate 9, step a) in place of 1-(2-dimethylamino-4-methyl-thiazol-5-yl)-ethanone.HCl according to the procedure described for intermediate 8, step b.

### Intermediate 10: Step a

### 1-(4-Methyl-2-pyrrolidin-1-yl-thiazol-5-yl)-ethanone.HCl

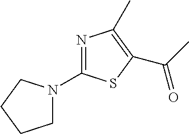

The title compound was prepared using pyrrolidine-1-carbothioic acid amide in place of 4-methyl-piperazine-1-carbothioic acid amide according to the procedure described for intermediate 7, step a.

### Intermediate 10: Step b

### 2-Bromo-1-(4-methyl-2-pyrrolidin-1-yl-thiazol-5-yl)-ethanone.HBr

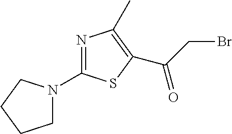

The title compound was prepared using 1-(4-methyl-2-pyrrolidin-1-yl-thiazol-5-yl)-ethanone.HCl (intermediate 10, step a) in place of 1-(2-dimethylamino-4-methyl-thiazol-5-yl)-ethanone.HCl according to the procedure described for intermediate 8, step b.

### Intermediate 11: Step a

### 1-(2-Diethylamino-4-methyl-thiazol-5-yl)-ethanone.HCl

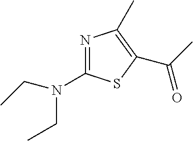

The title compound was prepared using 1,1-diethyl-thiourea in place of 4-methyl-piperazine-1-carbothioic acid amide according to the procedure described for intermediate 7, step a.

### Intermediate 11: Step b

### 2-Bromo-1-(2-diethylamino-4-methyl-thiazol-5-yl)-ethanone.HBr

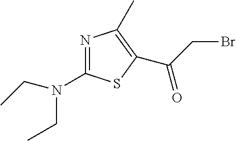

The title compound was prepared using 1-(2-diethylamino-4-methyl-thiazol-5-yl)-ethanone.HCl (intermediate 11, step a) in place of 1-(2-dimethylamino-4-methyl-thiazol-5-yl)-ethanone.HCl according to the procedure described for intermediate 8, step b.

### Intermediate 12: Step a

### 1-[4-Methyl-2-(pyridin-4-ylamino)-thiazol-5-yl]-ethanone.HCl

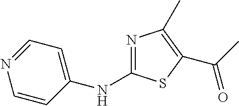

The title compound was prepared using pyridin-4-yl-thiourea in place of 4-methyl-piperazine-1-carbothioic acid amide according to the procedure described for intermediate 7, step a.

### Intermediate 12: Step b

### 2-Bromo-1-[4-methyl-2-(pyridin-4-ylamino)-thiazol-5-yl]-ethanone.HBr

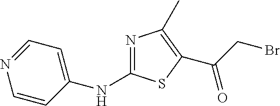

The title compound was prepared using 1-[4-methyl-2-(pyridin-4-ylamino)-thiazol-5-yl]-ethanone.HCl (intermediate 12, step a) in place of 1-(2-dimethylamino-4-methyl-thiazol-5-yl)-ethanone.HCl according to the procedure described for intermediate 8, step b.

### Intermediate 13: Step a

### 1-[4-Methyl-2-(pyridin-3-ylamino)-thiazol-5-yl]-ethanone.HCl

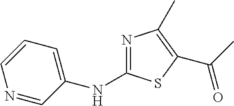

The title compound was prepared using pyridin-3-yl-thiourea in place of 4-methyl-piperazine-1-carbothioic acid amide according to the procedure described for intermediate 7, step a.

### Intermediate 13: Step b

### 2-Bromo-1-[3-methyl-2-(pyridin-4-ylamino)-thiazol-5-yl]-ethanone.HBr

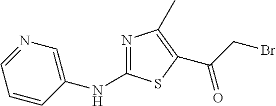

The title compound was prepared using 1-[4-methyl-2-(pyridin-3-ylamino)-thiazol-5-yl]-ethanone.HCl (intermediate 13, step a) in place of 1-(2-dimethylamino-4-methyl-thiazol-5-yl)-ethanone.HCl according to the procedure described for intermediate 8, step b.

### Intermediate 14: Step a

### 1-[4-Methyl-2-(pyridin-2-ylamino)-thiazol-5-yl]-ethanone.HCl

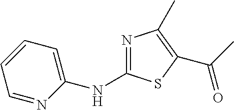

The title compound was prepared using pyridin-2-yl-thiourea in place of 4-methyl-piperazine-1-carbothioic acid amide according to the procedure described for intermediate 7, step a.

### Intermediate 14: Step b

### 2-bromo-1-(4-methyl-2-(pyridin-2-ylamino)thiazol-5-yl)ethanone.HBr

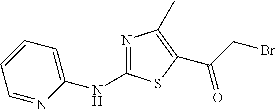

The title compound was prepared using 1-[4-methyl-2-(pyridin-2-ylamino)-thiazol-5-yl]-ethanone.HCl (intermediate 14, step a) in place of 1-(2-dimethylamino-4-methyl-thiazol-5-yl)-ethanone.HCl according to the procedure described for intermediate 8, step b.

### Intermediate 15: Step a

### 1-[4-Methyl-2-(3-pyrrolidin-1-yl-5-trifluoromethyl-pyridin-2-yl)-thiazol-5-yl]-ethanone.HCl

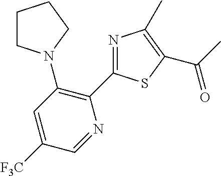

The title compound was prepared using 3-pyrrolidin-1-yl-5-trifluoromethyl-pyridine-2-carbothioic acid amide (Maybridge) in place of 4-methyl-piperazine-1-carbothioic acid amide according to the procedure described for intermediate 7, step a.

### Intermediate 15: Step b

### 2-Bromo-1-[4-methyl-2-(3-pyrrolidin-1-yl-5-trifluoromethyl-pyridin-2-yl)-thiazol-5-yl]-ethanone.HBr

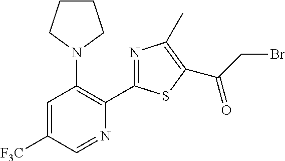

The title compound was prepared using 1-[4-methyl-2-(3-pyrrolidin-1-yl-5-trifluoromethyl-pyridin-2-yl)-thiazol-5-yl]-ethanone.HCl (intermediate 15, step a) in place of 1-(2-dimethylamino-4-methyl-thiazol-5-yl)-ethanone.HCl according to the procedure described for intermediate 8, step b.

### Intermediate 16: Step a

### 5-Acetyl-4-methyl-thiazole-2-carboxylic acid ethyl ester

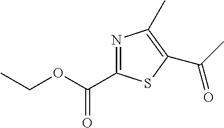

A mixture of 3-chloro-pentane-2,4-dione (2.02 g, 15 mmol) and amino-thioxo-acetic acid ethyl ester (2 g, 15 mmol) in EtOH (20 mL) was heated at 100° C. for 2 h. After cooling down the reaction mixture was concentrated and the residue was partitioned between 2 N Na₂CO₃ and EtOAc, the organic layer was dried (Na₂SO₄), filtered, concentrated and purified through column chromatography yielding the title compound as a colorless oil.

### Intermediate 16: Step b

### 5-(2-Bromo-acetyl)-4-methyl-thiazole-2-carboxylic acid ethyl ester

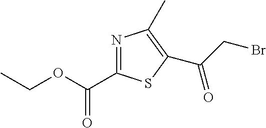

A suspension of bromine (0.337 mL, 6.56 mmol) in 1,4-dioxane (5 mL) was added to a stirred solution of 5-acetyl-4-methyl-thiazole-2-carboxylic acid ethyl ester (1.4 g, 6.56 mmol, intermediate 16, step a) in 1,4-dioxane (15 mL). The mixture was stirred at 50° C. for 6 h and concentrated. The residue was partitioned between 2 N Na₂CO₃ and CH₂Cl₂, the organic layer was dried (Na₂SO₄), filtered, concentrated and purified through column chromatography yielding the title compound as a white solid.

### Intermediate 17: Step a

### 2-(5-Carbamoyl-2-ethoxy-phenylamino)-4′-methyl-[4,5′]bithiazolyl-2′-carboxylic acid ethyl ester.HBr

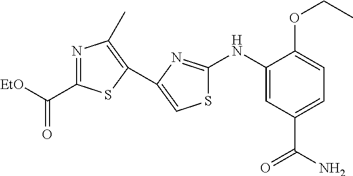

The title compound was prepared using 4-ethoxy-3-thioureido-benzamide (intermediate 25, step c) in place of 4-isopropoxy-3-thioureido-benzamide according to the procedure described in Example 99.

### Intermediate 17: Step b

### 2-(5-Carbamoyl-2-ethoxy-phenylamino)-4′-methyl-[4,5′]bithiazolyl-2′-carboxylic acid

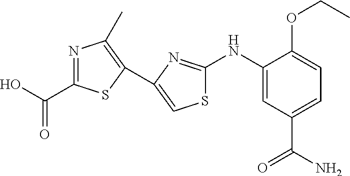

The title compound was prepared using 2-(5-carbamoyl-2-ethoxy-phenylamino)-4′-methyl-[4,5′]bithiazolyl-2′-carboxylic acid ethyl esterHBr (intermediate 17, step a) in place of 2-(5-carbamoyl-2-isopropoxy-phenylamino)-4′-methyl-[4,5′]bithiazolyl-2′-carboxylic acid ethyl ester.HBr according to the procedure described in Example 100.

### Intermediate 18: Step a

### 3-(2′-Hydroxymethyl-4′-methyl-[4,5′]bithiazolyl-2-ylamino)-4-isopropoxy-benzamide

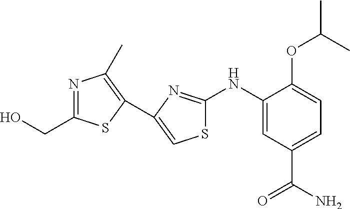

NaBH₄ (299 mg, 7.9 mmol) was added portion by portion to a suspension of 2-(5-carbamoyl-2-isopropoxy-phenylamino)-4′-methyl-[4,5′]bithiazolyl-2′-carboxylic acid ethyl esterHBr (833 mg, 1.58 mmol, Example 99) in EtOH (15 mL). The mixture was heated at 70° C. for 4 h and cooled down. To the reaction mixture was added silica gel (300 mesh, 10 g) and the mixture was concentrated and purified by solid loading on column chromatography (15% MeOH/DCM) to yield the title compound as a white solid.

### Intermediate 18: Step b

### 3-(2′-Chloromethyl-4′-methyl-[4,5′]bithiazolyl-2-ylamino)-4-isopropoxy-benzamide

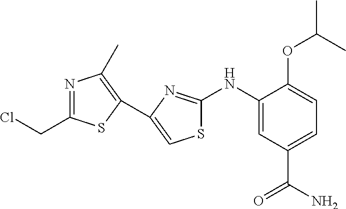

To a suspension of 3-(2′-hydroxymethyl-4′-methyl-[4,5′]bithiazolyl-2-ylamino)-4-isopropoxy-benzamide (639 mg, 1.58 mmol, intermediate 18, step a) in CH₂Cl₂ (10 mL) was added Et₃N (240 mg, 2.37 mmol) and MsCl (235 mg, 2.05 mmol). The reaction mixture was stirred for 6 h, diluted with CH₂Cl₂ (20 mL), and washed with saturated NaHCO₃ solution followed by water. The organic layer was dried (Na₂SO₄), filtered and concentrated to give the crude product as a white solid.

### Intermediate 19: Step a

### Dithiocarbamic acid methyl ester

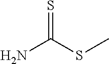

The title compound was prepared as described in Synthesis, 1985, 948.

### Intermediate 19: Step b

### 1-(4-Methyl-2-methylsulfanyl-thiazol-5-yl)-ethanone

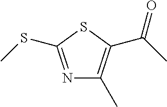

A mixture of dithiocarbamic acid methyl ester (1.7 g, 15.9 mmol, intermediate 19, step a) and 3-chloro-pentane-2,4-dione (2.56 g, 19.3 mmol) in EtOH (30 mL) was heated at 100° C. for 1.5 h. After cooling down the reaction mixture was concentrated and the residue was partitioned between 2 N Na₂CO₃ and EtOAc, the organic layer was dried (Na₂SO₄), filtered, concentrated and purified through column chromatography to yield the title compound as a thick oil.

### Intermediate 19: Step c

### 2-Bromo-1-(4-methyl-2-methylsulfanyl-thiazol-5-yl)-ethanone

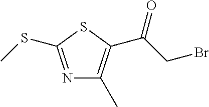

To a solution of 1-(4-methyl-2-methylsulfanyl-thiazol-5-yl)-ethanone (500 mg, 2.67 mmol, intermediate 19, step b) in THF (20 mL) was added phenyltrimethylammonium tribromide (1 g, 2.67 mmol). The reaction mixture was heated at 66° C. for 1 h. After cooling down the reaction mixture was partitioned between saturated NaHCO₃ and EtOAc, the organic layer was dried (Na₂SO₄), filtered, concentrated and purified through column chromatography to yield the title compound.

### Intermediate 19: Step d

### 4-Isopropoxy-3-(4′-methyl-2′-methylsulfanyl-[4,5′]bithiazolyl-2-ylamino)-benzamide.HBr

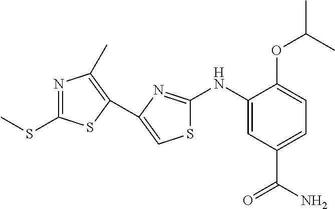

The title compound was prepared using 2-bromo-1-(4-methyl-2-methylsulfanyl-thiazol-5-yl)-ethanone (intermediate 19, step c) in place of 5-(2-bromo-acetyl)-4-methyl-thiazole-2-carboxylic acid ethyl ester according to the procedure described in Example 99.

### Intermediate 20: Step a

### 4-Bromo-2-isothiocyanato-1-methoxy-benzene

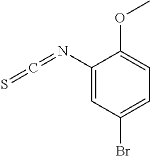

A solution of sodium bicarbonate (3.74 g, 44.5 mmol) in water (75 mL) was added to commercially available 5-bromo-2-methoxy-phenylamine (3 g, 14.8 mmol) in chloroform (75 mL). Thiophosgene (1.42 mL, 18.6 mmol) was then added. The biphasic solution was stirred at room temperature for 1 h. The phases were separated and the aqueous phase was extracted with CH₂Cl₂. The organic phase was dried (Na₂SO₄), filtered, and concentrated, yielding the crude title compound as an off-white solid.

### Intermediate 20: Step b

### (5-Bromo-2-methoxy-phenyl)-thiourea

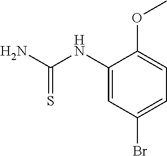

Crude 4-bromo-2-isothiocyanato-1-methoxy-benzene (3.6 g, 14.7 mmol, intermediate 20, step a) was suspended in MeOH (10 mL). A 2 M solution of ammonia in MeOH (56.5 mL) was added and the resulting yellow solution was stirred at room temperature for 16 h. The reaction mixture was concentrated to afford the title compound as a white powder.

### Intermediate 20: Step c

### (5-Bromo-2-methoxy-phenyl)-(2′,4′-dimethyl-[4,5′]bithiazolyl-2-yl)-amine

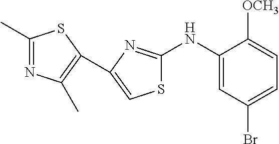

A mixture of 2-bromo-1-(2,4-dimethyl-thiazol-5-yl)-ethanone.HBr (2.41 g, 7.66 mmol, intermediate 3) and (5-bromo-2-methoxy-phenyl)-thiourea (2 g, 7.66 mmol, intermediate 20, step b) in EtOH (20 mL) was stirred at room temperature for 48 h. The mixture was filtered, washed sequentially with EtOH and heptanes, and air-dried. The filtered solid was then partitioned between saturated NaHCO₃ solution and EtOAc, the organic layer was dried (Na₂SO₄), filtered, concentrated and vacuum dried affording the title compound as a white solid.

### Intermediate 21

### (2′,4′-Dimethyl-[4,5′]bithiazolyl-2-yl)-[2-methoxy-5-(4,4,5,5-tetramethyl[1,3,2]dioxaborolan-2-yl)-phenyl]-amine

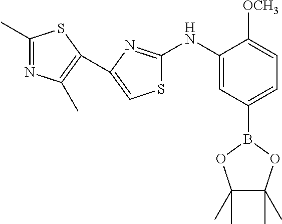

A mixture of (5-bromo-2-methoxy-phenyl)-(2′,4′-dimethyl-[4,5′]bithiazolyl-2-yl)-amine (300 mg, 0.757 mmol, intermediate 20, step c), 4,4,5,5,4′,4′,5′,5′-octamethyl-[2,2′]bi[[1,3,2]dioxaborolanyl] (231 mg, 0.908 mmol), PdCl₂(dppf).CH₂Cl₂ (62 mg, 0.076 mmol) and KOAc (209 mg, 1.51 mmol) in 1,4-dioxane (5 mL) was heated at 86° C. for 4 h. After cooling down the reaction mixture was partitioned between water and EtOAc, the organic layer was dried (Na₂SO₄), filtered, concentrated and purified through column chromatography to afford the title compound.

### Intermediate 22: Step a

### 1-(4-Methyl-2-pyridin-4-yl-thiazol-5-yl)-ethanone.HCl

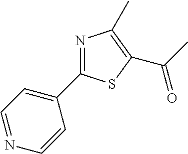

The title compound was prepared using thioisonicotinamide in place of 4-methyl-piperazine-1-carbothioic acid amide according to the procedure described for intermediate 7, step a.

### Intermediate 22: Step b

### 2-Bromo-1-(4-methyl-2-pyridin-4-yl-thiazol-5-yl)-ethanone.HBr

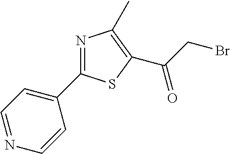

The title compound was prepared using 1-(4-methyl-2-pyridin-4-yl-thiazol-5-yl)-ethanone.HCl (intermediate 22, step a) in place of 1-(2-dimethylamino-4-methyl-thiazol-5-yl)-ethanone.HCl according to the procedure described for intermediate 8, step b.

### Intermediate 23: Step a

### 4-Fluoro-3-nitro-benzamide

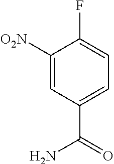

A round bottom flask fitted with a reflux condenser vented through an aqueous sodium hydroxide solution was charged with 4-fluoro-3-nitro-benzoic acid (5.0 g, 27.0 mmol, Aldrich). Thionyl chloride (20 mL) was added and the resulting suspension was heated in an 80° C. oil bath for 3 h. The mixture was concentrated and the residual oil was dissolved in THF (20 mL) and added slowly via pipette to an ice-cold solution of concentrated aqueous NH₄OH (20 mL). The resulting bright yellow mixture was stirred at 0° C. for 35 min. The mixture was partially concentrated to remove THF and the residual solution was extracted with EtOAc. The organic phase was dried (Na₂SO₄), filtered, and concentrated. The residue was purified by flash column chromatography (Silica gel, 1-3% EtOH—CH₂Cl₂) to afford the title compound as a white solid.

### Intermediate 23: Step b

### 4-Isopropoxy-3-nitro-benzamide

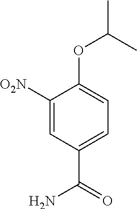

To a solution of iPrOH (0.619 mL, 8.09 mmol) in THF (25 mL) at 0° C., added a 0.5 M solution of KHMDS in toluene (16.2 mL, 8.09 mmol) followed by 4-fluoro-3-nitro-benzamide (993 mg, 5.39 mmol, intermediate 23, step a). The resulting brown suspension was stirred at 0° C. for 1 h, then was allowed to warm to 23° C. and was stirred for an additional 4 h. The mixture was partially concentrated to remove THF and was diluted with water and extracted with EtOAc. The organic phase was dried (Na₂SO₄), filtered, and concentrated, affording the crude title compound as an orange solid which was used without further purification in the next reaction.

### Intermediate 23: Step c

### 3-Amino-4-isopropoxy-benzamide

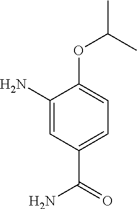

Sodium borohydride (250 mg, 6.60 mmol) was added slowly to a solution of nickel (II) chloride hexahydrate (567 mg, 2.20 mmol) in MeOH (30 mL) at 0° C. and the resulting black suspension was stirred for 30 min at 23° C. The mixture was cooled to 0° C. and to it was added a suspension of crude 4-isopropoxy-3-nitro-benzamide (0.987 g, 4.40 mmol, intermediate 23, step b) in MeOH (20 mL), followed by sodium borohydride (583 mg, 15.4 mmol). The mixture was stirred for 1 hour at 23° C. The mixture was partially concentrated to remove most of the MeOH, water was added to quench excess NaBH₄, and the mixture was partitioned between EtOAc and water. The aqueous phase was extracted with EtOAc. The organic phase was dried (Na₂SO₄), filtered, and concentrated. The residue was purified by flash column chromatography (Silica gel, 1-6% MeOH—CH₂Cl₂), yielding the title compound as a white powder.

### Intermediate 23: Step d 4-Isopropoxy-3-isothiocyanato-benzamide

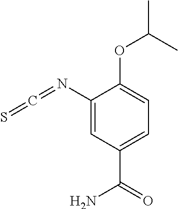

A solution of sodium bicarbonate (645 mg, 7.68 mmol) in water (15 mL) was added to 3-amino-4-isopropoxy-benzamide (497 mg, 2.56 mmol, intermediate 23, step c) in a mixture of chloroform (15 mL) and water (15 mL). Thiophosgene (0.206 mL, 2.69 mmol) was then added. The biphasic solution was stirred at room temperature for 2.5 h. TLC analysis indicated slight remaining starting material, so an additional 0.030 mL portion of thiophosgene was added and the mixture was stirred for 40 min. The phases were separated and the aqueous phase was extracted with CH₂Cl₂. The organic phase was dried (Na₂SO₄), filtered, and concentrated, yielding the crude title compound as an off-white solid.

### Intermediate 23: Step e

### 4-Isopropoxy-3-thioureido-benzamide

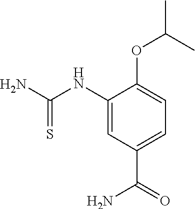

Crude 4-isopropoxy-3-isothiocyanato-benzamide (608 mg, intermediate 23, step d) was suspended in MeOH (2 mL). A 2 M solution of ammonia in MeOH (2 mL) was added and the resulting yellow solution was stirred at room temperature for 16 h. The reaction mixture was concentrated and the residue was purified by flash column chromatography (Silica gel, 3-8% MeOH—CH₂Cl₂), affording the title compound as a white powder.

### Intermediate 24

### 4-Methoxy-3-thioureido-benzamide

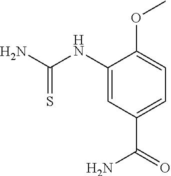

To a solution of 3-amino-4-methoxybenzamide (2.49 g, 15.0 mmol, Alfa) in acetone (30 mL) at reflux was added benzoyl isothiocyanate (2.22 mL, 16.5 mmol) and the mixture was stirred at reflux for 30 min, then was poured into water. The precipitate was collected by vacuum filtration and was treated with 10% aq. NaOH (15 mL). The mixture was heated to reflux for 40 min, cooled to room temperature, and poured into a mixture of ice and 6 N aq. HCl. The mixture was basified to pH 10 with conc. aq. NH₄OH and the resulting white solid precipitate was collected by vacuum filtration, affording the crude title compound, which was used without further purification.

### Intermediate 25: Step a

### 4-Ethoxy-3-nitro-benzamide

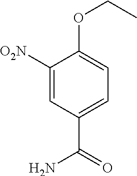

A mixture of 4-fluoro-3-nitro-benzamide (1.18 g, 6.39 mmol, intermediate 23, step a) and EtOH (5 mL) was treated with a solution of sodium ethoxide in EtOH (21 wt. %, 4.77 mL, 12.78 mmol, Aldrich) and the resulting brown suspension was stirred at room temperature for 15 min. The mixture was partitioned between EtOAc and water and the aqueous phase was extracted with EtOAc. The organic phase was dried (Na₂SO₄), filtered, and concentrated and the residue was purified by flash column chromatography (Silica gel, 60-100% EtOAc-Hept) affording the title compound as a yellow powder.

### Intermediate 25: Step b

### 3-Amino-4-ethoxy-benzamide

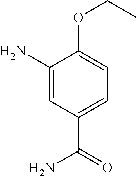

The title compound was prepared using 4-ethoxy-3-nitro-benzamide (intermediate 25, step a) in place of 4-isopropoxy-3-nitro-benzamide using the procedure described for intermediate 23, step c.

### Intermediate 25: Step c

### 4-Ethoxy-3-thioureido-benzamide

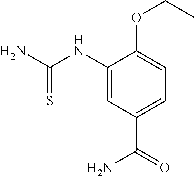

To a suspension of 3-amino-4-ethoxy-benzamide (597 mg, 3.31 mmol, intermediate 25, step b) in acetone (10 mL), was added benzoyl isothiocyanate (0.490 mL, 3.64 mmol) and the resulting white suspension was heated to reflux for 20 min. The mixture was poured into water and the white solid precipitate was collected by vacuum filtration. The solid was heated with 10% aq. NaOH solution (15 mL) in a 90° C. oil bath for 25 min. The reaction mixture was diluted with water and was extracted with EtOAc. The organic phase was dried (Na₂SO₄), filtered, and concentrated. The residue was purified by flash column chromatography (Silica gel, 1-6% MeOH—CH₂Cl₂), yielding the title compound as a white powder.

### Intermediate 26

### (2-Methyl-benzofuran-7-yl)-thiourea

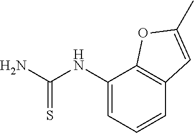

2-Methyl-benzofuran-7-ylamine.HCl (Chembridge) was partitioned between sat. aq. NaHCO₃ and CH₂Cl₂ to convert it to the corresponding free base, and the organic phase was dried (Na₂SO₄), filtered, and concentrated. The resulting brown oil (768 mg, 5.22 mmol) was heated to reflux in acetone (30 mL) and benzoyl isothiocyanate (0.772 mL, 5.74 mmol) was slowly added. The mixture was stirred at reflux for 30 min, was poured into ice water, and the precipitated solid was collected by vacuum filtration, and washed with water. The solid was stirred at reflux in 10% aq. NaOH (15 mL) for 40 min. The reaction mixture was poured into a mixture of ice and 6 N aq. HCl, resulting in formation of a white precipitate. The mixture was basified to pH 10 by addition of conc. aq. NH₄OH and was filtered. The title compound was isolated as a white solid by column chromatography (Silica gel, 20-60% EtOAc-Hept).

### Intermediate 27

### (2,2-Difluoro-benzo[1,3]dioxol-4-yl)-thiourea

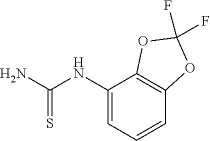

The title compound was prepared using 2,2-difluoro-benzo[1,3]dioxol-4-ylamine (Alfa Aesar) in place of 2-methyl-benzofuran-7-ylamine as described for intermediate 26 and was purified by column chromatography (Silica gel, 20-60% EtOAc-Hept).

### Intermediate 28

### (2-Methoxy-5-methyl-phenyl)-thiourea

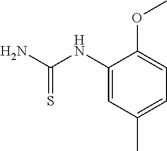

The title compound was prepared using 2-methoxy-5-methyl-phenylamine (TCI) in place of 2-methyl-benzofuran-7-ylamine as described for intermediate 26, except that the crude product was purified by recrystallization (toluene), rather than by column chromatography.

### Intermediate 29: Step a

### 4-tert-Butoxy-3-nitro-benzamide

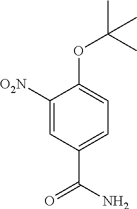

To a solution of 4-fluoro-3-nitro-benzamide (770 mg, 4.18 mmol, intermediate 23, step a) in THF (20 mL) was added a solution of potassium tert-butoxide (938.5 mg, 8.36 mmol) in THF (20 mL). The resulting red solution was stirred at 23° C. for 3 h. The reaction mixture was concentrated and was partitioned between water and EtOAc (sat. aq. NaCl was added to aid phase separation). The separated aq. phase was extracted with EtOAc and the combined organic extracts were washed with sat. aq. NaCl. The organic phase was dried (Na₂SO₄), filtered, and concentrated. The residue was partially purified by flash column chromatography (Silica gel, 0-5% MeOH—CH₂Cl₂), yielding impure title compound which was used as is in the next reaction.

### Intermediate 29: Step b

### 3-Amino-4-tert-butoxy-benzamide

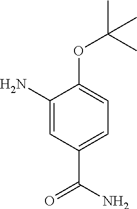

The title compound was prepared according to the procedure of intermediate 23, step c, using impure 4-tert-butoxy-3-nitro-benzamide (intermediate 29, step a) in place of 4-isopropoxy-3-nitro-benzamide. After a reaction time of 3 h, the reaction mixture was filtered through Celite and the filtrate was concentrated. The residue was partitioned between EtOAc and water and the aq. phase was extracted with EtOAc. The organic phase was dried (Na₂SO₄), filtered, and concentrated. The residue was purified by flash column chromatography (Silica gel, 50-100% EtOAc-Hept) to afford the title compound.

### Intermediate 29: Step c

### 4-tert-Butoxy-3-isothiocyanato-benzamide

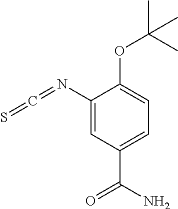

The title compound was prepared according to the procedure of intermediate 23, step d, using 3-amino-4-tert-butoxy-benzamide (intermediate 29, step b) in place of 3-amino-4-isopropoxy-benzamide. The crude product was used without purification.

### Intermediate 29: Step d

### 4-tert-Butoxy-3-thioureido-benzamide

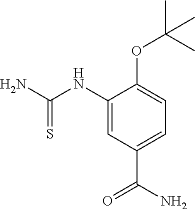

The title compound was prepared according to the procedure of intermediate 23, step e, using 4-tert-butoxy-3-isothiocyanato-benzamide (intermediate 29, step c) in place of 4-isopropoxy-3-isothiocyanato-benzamide, with a reaction time of 1 h and purification by flash column chromatography (Silica gel, 0-6% MeOH—CH₂Cl₂).

### Intermediate 30: Step a

### 4-Hydroxy-3-nitro-benzamide (ammonia complex)

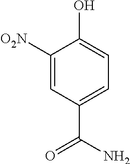

The title compound was prepared by a modification of the procedure described in J. Med. Chem. 2006, 49, 5080: A suspension of 4-hydroxy-3-nitro-benzoic acid methyl ester (20.38 g, 103 mmol, Alfa) in concentrated aqueous ammonium hydroxide solution (28-30%, 75 mL) was heated in a sealed tube (55° C., 4 d). The suspension was allowed to cool to room temperature and the yellow precipitate was collected by vacuum filtration, washing with water. NMR analysis indicated incomplete conversion, so the solid was re-subjected to the reaction conditions (60° C., 3 d). Filtration as above afforded the title compound as a yellow powder.

### Intermediate 30: Step b

### 3-Amino-4-hydroxy-benzamide

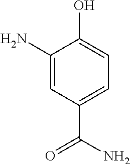

To a green solution of nickel(II) chloride hexahydrate (3.18 g, 12.3 mmol) at 0° C. was slowly added sodium borohydride (1.40 g, 37.0 mmol) (caution: gas evolution). The resulting black suspension was stirred at room temperature for 30 min, then was cooled to 0° C. before addition of 4-hydroxy-3-nitro-benzamide, ammonia complex (4.50 g, 24.7 mmol, intermediate 30, step a) and a second portion of sodium borohydride (3.27 g, 86.4 mmol). The resulting black suspension was stirred at room temperature for 1.5 h and was filtered through Celite. The filtrate was concentrated and the solid residue was partitioned between EtOAc and water (250 mL each). The aqueous phase was neutralized to pH 7 by addition of 1 N aq. HCl, then saturated with NaCl and extracted with EtOAc. TLC indicated product remaining in the aq. phase, so the pH was adjusted to 1 with 1 N aq. HCl, then to 5 with sat. aq. NaHCO₃ and the solution was further extracted with EtOAc. The combined organic extracts were dried (Na₂SO₄), filtered, and concentrated, yielding the title compound.

### Intermediate 30: Step c

### 4-Hydroxy-3-thioureido-benzamide

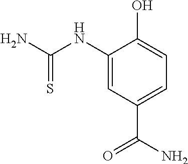

A suspension of 3-amino-4-hydroxy-benzamide (2.06 g, 13.5 mmol, intermediate 30, step b) in acetone (30 mL) at reflux was removed from heating and treated with benzoyl isothiocyanate (2.00 mL, 14.9 mmol) cautiously dropwise via syringe, with stirring. During the addition, a solution briefly formed, then the reaction mixture solidified. Acetone was added to achieve a suspension, which was heated at reflux for 30 min. Water was added and the mixture was allowed to cool to room temperature. The suspension was filtered, washing with water, and the collected tan solid was air-dried. The solid was stirred in 10% aq. NaOH (15 mL) at 85° C. for 30 min. The solution was allowed to cool to room temperature and was poured into a stirred mixture of 6 N aq. HCl and ice, resulting in precipitation of a white solid. The pH was adjusted to 8 with conc. aq. NH₄OH. The precipitated solid was collected by vacuum filtration and was dried by concentrating twice from toluene, affording the crude title compound as an off-white powder, which was used without purification in the next reaction.

### Intermediate 30: Step d

### 3-(2′,4′-Dimethyl-[4,5′]bithiazolyl-2-ylamino)-4-hydroxy-benzamide

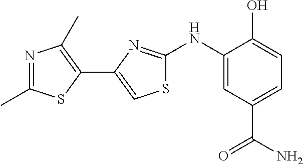

A suspension of 4-hydroxy-3-thioureido-benzamide (511 mg, 2.42 mmol, intermediate 30, step c) and 2-bromo-1-(2,4-dimethyl-thiazol-5-yl)-ethanone.HBr (762 mg, 2.42 mmol, intermediate 3) in EtOH (10 mL) was stirred at room temperature overnight. The reaction mixture was poured into sat. aq. NaHCO₃ and the pH was adjusted from 9 to 7 with 1 N aq. HCl before extraction with EtOAc. The organic extracts were dried (Na₂SO₄), filtered, and concentrated. The yellow solid residue was purified by flash column chromatography (Silica gel, first column: gradient 50-100% EtOAc-Hept, then 10% MeOH-EtOAc. Second column: 1-10% MeOH—CH₂Cl₂), affording the title compound. (The compound should be free of MeOH for the next reaction; trace residual MeOH was removed by concentration from toluene.)

### Intermediate 31: Step a

### 3-Nitro-4-trifluoromethoxy-benzamide

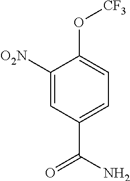

To concentrated aqueous sulfuric acid (3 mL) was slowly added 90% aqueous nitric acid (3 mL) and the resulting solution was cooled in an ice-bath. Solid 4-trifluoromethoxy-benzamide (1.0 g, 4.88 mmol, Alfa) was slowly added and the reaction mixture was stirred at room temperature for 10 min, then was poured into a stirred ice/water mixture. The white precipitate was collected by vacuum filtration and washed with water, affording the crude title compound, which was used without further purification.

### Intermediate 31: Step b

### 3-Amino-4-trifluoromethoxy-benzamide

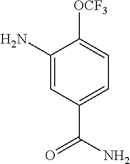

To a solution of nickel(II) chloride hexahydrate (470 mg, 1.98 mmol) in MeOH (10 mL) at 0° C. was slowly added sodium borohydride (225 mg, 5.94 mmol) (caution: gas evolution). The resulting black suspension was stirred at room temperature for 30 min, then was cooled to 0° C. before addition of crude 3-nitro-4-trifluoromethoxy-benzamide (0.99 g, 3.96 mmol, intermediate 31, step a) and a second portion of sodium borohydride (524 mg, 13.9 mmol). The resulting black suspension was stirred at room temperature for 0.5 h before addition of a small amount of water to quench remaining borohydride. The mixture was diluted with sat. aq. NaHCO₃ and extracted with CH₂Cl₂. To facilitate extraction of the polar product, the aqueous phase was saturated with NaCl, then was further extracted with CH₂Cl₂. The organic phase was washed with saturated aqueous NaCl and was dried (Na₂SO₄), filtered, and concentrated. The residual white solid was purified by flash column chromatography (Silica gel, 20-100% EtOAc-Hept), affording the title compound as an off-white solid.

### Intermediate 31: Step c

### 3-Isothiocyanato-4-trifluoromethoxy-benzamide

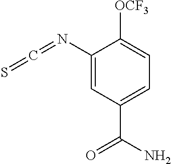

The title compound was prepared using 3-amino-4-trifluoromethoxy-benzamide (intermediate 31, step b) in place of 3-amino-4-isopropoxy-benzamide by the procedure described for intermediate 23, step d, affording the crude title compound as a white solid which was used without purification.

### Intermediate 31: Step d

### 3-Thioureido-4-trifluoromethoxy-benzamide

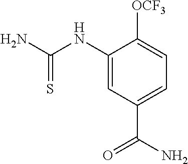

Crude 3-isothiocyanato-4-trifluoromethoxy-benzamide (444 mg, 1.69 mmol, intermediate 31, step c) was treated with a solution of ammonia in methanol (2 M, 10 mL, 20 mmol) and the resulting yellow solution was stirred at 40° C. for 30 min. The reaction mixture was concentrated onto Silica gel for purification by column chromatography (Silica gel, 20-100% EtOAc-Hept), which afforded the title compound as a white solid.

### Intermediate 32

### (2,3-Dihydro-benzo[1,4]dioxin-5-yl)-thiourea

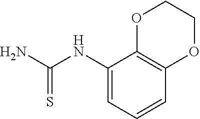

To a solution of commercially available 2,3-dihydro-benzo[1,4]dioxin-5-ylamine (1.00 g, 6.62 mmol) at reflux in acetone (30 mL) was slowly added benzoyl isothiocyanate (0.98 mL, 7.28 mmol). The mixture was stirred at reflux for 50 min and poured into a mixture of ice and water. The resulting precipitate was collected by vacuum filtration and treated with 10% aq. NaOH (15 mL). The mixture was heated to reflux for 1 h and cooled to room temperature. A white solid precipitated and was collected by vacuum filtration, affording the crude title compound which was used without further purification.

### Intermediate 33

### N,N-Diethyl-4-methoxy-3-thioureido-benzenesulfonamide

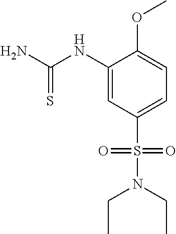

The title compound was prepared using 3-amino-N,N-diethyl-4-methoxy-benzenesulfonamide (TCI) in place of 3-amino-4-methoxybenzamide according to the procedure described for intermediate 24, and was purified by flash column chromatography (Silica gel, 40-80% EtOAc-Hept).

### Intermediate 34

### (2-Methoxy-5-trifluoromethyl-phenyl)-thiourea

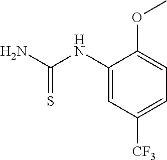

The title compound was prepared using 2-methoxy-5-trifluoromethyl-phenylamine (Aldrich) in place of 2,3-dihydro-benzo[1,4]dioxin-5-ylamine according to the procedure described for intermediate 32.

### Intermediate 35

### (4-Methoxy-biphenyl-3-yl)-thiourea

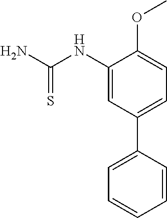

The title compound was prepared using 4-methoxy-biphenyl-3-ylamine (TCI) in place of 2,3-dihydro-benzo[1,4]dioxin-5-ylamine according to the procedure described for intermediate 32.

### Intermediate 36: Step a

### 3-Isothiocyanato-4-methyl-benzamide

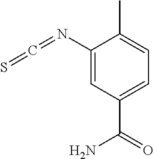

The title compound was prepared using 3-amino-4-methyl-benzamide (Alfa) in place of 3-amino-4-isopropoxy-benzamide according to the procedure described for intermediate 23, step d, except that the reaction was allowed to run overnight and the reaction mixture was filtered to afford the crude title compound.

### Intermediate 36: Step b

### 4-Methyl-3-thioureido-benzamide

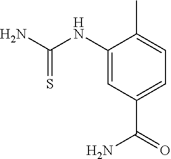

Crude 3-isothiocyanato-4-methyl-benzamide (intermediate 36, step a) was suspended in MeOH (10 mL) and treated with 2 N NH₃ in MeOH (30 mL). The mixture was stirred overnight at room temperature and the tan precipitate was collected by vacuum filtration, affording the title compound.

### Intermediate 37: Step a

### 4-Chloro-3-isothiocyanato-benzamide

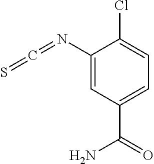

The title compound was prepared using 3-amino-4-chloro-benzamide (Aldrich) in place of 3-amino-4-isopropoxy-benzamide according to the procedure described for intermediate 23, step d, except that the reaction was allowed to run for 3 d and the reaction mixture was filtered to afford the crude title compound.

### Intermediate 37: Step b

### 4-Chloro-3-thioureido-benzamide

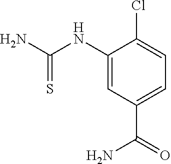

The title compound was prepared using crude 4-chloro-3-isothiocyanato-benzamide (intermediate 37, step a) in place of 3-isothiocyanato-4-methyl-benzamide by the method of intermediate 36, step b. The crude product was purified by trituration from EtOH/water.

### Intermediate 38: Step a

### 2-(4-Methoxy-3-nitro-phenyl)-acetamide

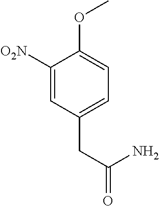

Nitric acid (90%, 6 mL) was added to an ice-cold suspension of (4-methoxy-phenyl)-acetic acid (3.02 g, 18.2 mmol, Aldrich) in acetic anhydride (20 mL). The resulting brown solution was stirred at 0° C. for 15 min, then at room temperature for 15 min, then was poured into ice. When melted, the mixture was extracted with CH₂Cl₂. The organic phase was dried (Na₂SO₄), filtered, and concentrated and the residue was re-concentrated from toluene, affording a brown oil. The crude oil was heated at 80° C. in thionyl chloride (15 mL) for 1 h. The reaction mixture was concentrated, and the residue was dissolved in THF (15 mL) and added to ice-cold concentrated aq. NH₄OH (15 mL). The resulting mixture was stirred at 0° C. for 30 min, was partially concentrated to remove THF, was diluted with water, and was extracted with EtOAc. The organic phase was dried (Na₂SO₄), filtered, and concentrated. The residue was purified by flash column chromatography (Silica gel, 75-100% EtOAc-Hept; then 0-3% MeOH-EtOAc), affording the title compound as a yellow powder.

### Intermediate 38: Step b

### 2-(3-Amino-4-methoxy-phenyl)-acetamide

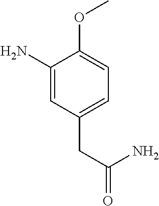

The title compound was prepared using 2-(4-methoxy-3-nitro-phenyl)-acetamide (intermediate 38, step a) in place of 4-isopropoxy-3-nitro-benzamide according to the procedure described for intermediate 23, step c (reaction time 15 min after addition of 2-(4-methoxy-3-nitro-phenyl)-acetamide), except that the crude product was used in the next reaction.

### Intermediate 38: Step c

### 2-(3-Isothiocyanato-4-methoxy-phenyl)-acetamide

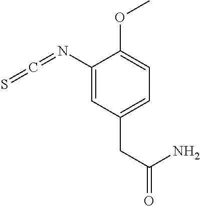

The title compound was prepared using 2-(3-amino-4-methoxy-phenyl)-acetamide (intermediate 38, step b) in place of 3-amino-4-isopropoxy-benzamide according to the procedure described for intermediate 23, step d.

### Intermediate 38: Step d

### 2-(4-Methoxy-3-thioureido-phenyl)-acetamide

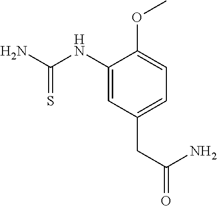

Crude 2-(3-isothiocyanato-4-methoxy-phenyl)-acetamide (369 mg, 1.66 mmol, intermediate 38, step c) was treated with a solution of ammonia in methanol (2 M, 10 mL, 20 mmol) and the resulting mixture was stirred at 40° C. for 40 min. The reaction mixture was allowed to cool to 23° C. and was filtered to collect the title compound as an off-white solid.

### Intermediate 39: Step a

### 5-Methoxy-6-nitro-2,3-dihydro-isoindol-1-one

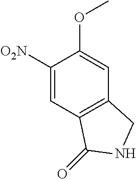

Solid 5-methoxy-2,3-dihydro-isoindol-1-one (1.17 g, 7.15 mmol, J&W PharmLab) was cooled to 0° C. and concentrated sulfuric acid (20 mL) was added. To the resulting red solution was added potassium nitrate (795 mg, 7.87 mmol) and the resulting brown solution was stirred at room temperature for 1 h, then was slowly added to ice. A cream-colored precipitate was collected by vacuum filtration, was dried by concentration from toluene, and was triturated from a heptane/toluene mixture, affording the crude title compound, which was used without further purification.

### Intermediate 39: Step b

### 6-Amino-5-methoxy-2,3-dihydro-isoindol-1-one

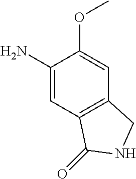

The title compound was prepared using crude 5-methoxy-6-nitro-2,3-dihydro-isoindol-1-one (intermediate 39, step a) in place of 4-isopropoxy-3-nitro-benzamide according to the procedure described for intermediate 23, step c, and was separated from the regioisomeric product 4-amino-5-methoxy-2,3-dihydro-isoindol-1-one and other impurities by column chromatography (Silica gel, 75-100% EtOAc-Hept followed by 1-8% MeOH-EtOAc), affording the title compound as a tan powder.

### Intermediate 39: Step c

### 6-Isothiocyanato-5-methoxy-2,3-dihydro-isoindol-1-one

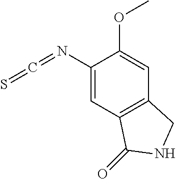

The title compound was prepared using 6-amino-5-methoxy-2,3-dihydro-isoindol-1-one (intermediate 39, step b) in place of 3-amino-4-isopropoxy-benzamide according to the procedure described for intermediate 23, step d (the extraction step employed multiple extractions with either EtOAc or CH₂Cl₂ containing slight THF until the solid material was completely dissolved).

### Intermediate 39: Step d

### (6-Methoxy-3-oxo-2,3-dihydro-1H-isoindol-5-yl)-thiourea

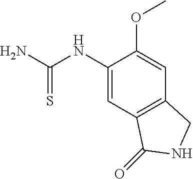

The title compound was prepared using 6-isothiocyanato-5-methoxy-2,3-dihydro-isoindol-1-one (intermediate 39, step c) in place of 4-isopropoxy-3-isothiocyanato-benzamide according to the procedure described for intermediate 23, step e, except that the crude product obtained from concentration of the reaction mixture was used in the next reactions without further purification.

### Intermediate 40: Step a

### 7-Isothiocyanato-2,3-dihydro-benzofuran-5-sulfonic acid amide

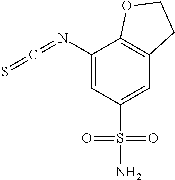

The title compound was prepared using 7-amino-2,3-dihydro-benzofuran-5-sulfonic acid amide (Butt Park Ltd) in place of 3-amino-4-isopropoxy-benzamide according to the procedure described for intermediate 23, step d.

### Intermediate 40: Step b

### 7-Thioureido-2,3-dihydro-benzofuran-5-sulfonic acid amide

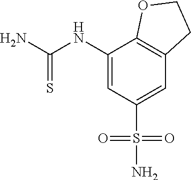

The title compound was prepared using 7-isothiocyanato-2,3-dihydro-benzofuran-5-sulfonic acid amide (intermediate 40, step a) in place of 4-isopropoxy-3-isothiocyanato-benzamide according to the procedure described for intermediate 23, step e.

### Intermediate 41: Step a

### 3-Fluoro-4-methoxy-5-nitro-benzamide

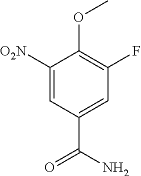

3-Fluoro-4-methoxy-benzamide (3.27 g, 19.3 mmol, JRD Fluorochemicals) was cooled to 0° C. and concentrated sulfuric acid (30 mL) was added followed by potassium nitrate (2.15 g, 21.3 mmol) and the resulting brown solution was stirred at room temperature for 1.5 h, then was slowly added to ice. The precipitate was collected by vacuum filtration affording the crude title compound as a sticky white solid.

### Intermediate 41: Step b

### 3-Amino-5-fluoro-4-methoxy-benzamide

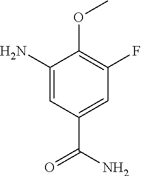

Sodium borohydride (911 mg, 24.1 mmol) was added slowly to a solution of nickel (II) chloride hexahydrate (1.91 mg, 8.03 mmol) in MeOH (50 mL) at 0° C. and the resulting black suspension was stirred for 30 min at 23° C. The mixture was cooled to 0° C. and to it was added crude 3-fluoro-4-methoxy-5-nitro-benzamide (5.73 g, intermediate 41, step a) followed by sodium borohydride (2.13 g, 56.2 mmol). The mixture was stirred for 4 h at 23° C. TLC analysis indicated incomplete reduction, so the mixture was cooled to 0° C. and an additional portion of sodium borohydride (600 mg, 15.9 mmol) was added. The reaction was allowed to proceed for another 3 h at room temperature. A small amount of water was added to quench excess NaBH₄ and the mixture was diluted with sat. aq. NaHCO₃ and was filtered through Celite. The filtrate was saturated with NaCl and was extracted with EtOAc. The organic phase was dried (Na₂SO₄), filtered, and concentrated, affording the crude title compound.

### Intermediate 41: Step c

### 3-Fluoro-5-isothiocyanato-4-methoxy-benzamide

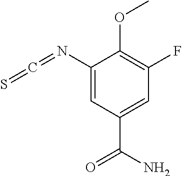

The title compound was prepared using 3-amino-5-fluoro-4-methoxy-benzamide (intermediate 41, step b) in place of 3-amino-4-isopropoxy-benzamide according to the procedure described for intermediate 23, step d. The crude product was purified by flash column chromatography (Silica gel, 50% EtOAc-CH₂Cl₂), affording the title compound as an off-white powder.

### Intermediate 41: Step d 3-Fluoro-4-methoxy-5-thioureido-benzamide

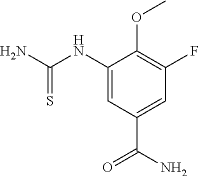

The title compound was prepared using 3-fluoro-5-isothiocyanato-4-methoxy-benzamide (intermediate 41, step c) in place of 4-isopropoxy-3-isothiocyanato-benzamide according to the procedure described for intermediate 23, step e (reaction time 3 d). The reaction mixture was concentrated to approximately half its original volume and crystallization was induced by scraping the walls of the reaction vessel with a spatula. The precipitated white solid was collected by vacuum filtration and washed with MeOH to afford the title compound.

### Intermediate 42: Step a

### 3-Methoxy-4-nitro-benzamide

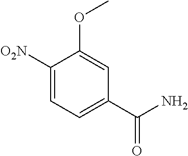

A round bottom flask fitted with a reflux condenser vented through an aqueous sodium hydroxide solution was charged with 3-methoxy-4-nitro-benzoic acid (3.07 g, 15.6 mmol, Aldrich). Thionyl chloride (10 mL) was added and the resulting suspension was heated in an 80° C. oil bath for 30 min. The mixture was concentrated and the residue was dissolved in THF (10 mL) and added slowly via pipette to an ice-cold solution of concentrated aqueous NH₄OH (10 mL). The resulting yellow suspension was stirred at 0° C. for 30 min. The suspension was partially concentrated to remove THF and was filtered to afford the crude title compound as a light yellow solid.

### Intermediate 42: Step b

### 4-Amino-3-methoxy-benzamide

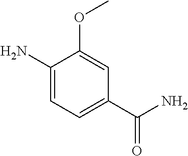

Sodium borohydride (0.764 g, 20.2 mmol) was added slowly to a solution of nickel (II) chloride hexahydrate (1.60 g, 6.73 mmol) in MeOH (25 mL) at 0° C. and the resulting black suspension was stirred for 20 min at 23° C. The mixture was cooled to 0° C. and to it was added crude 3-methoxy-4-nitro-benzamide (2.64 g, 13.5 mmol, intermediate 42, step a) followed by sodium borohydride (1.78 g, 47.1 mmol). The mixture was stirred for 30 min at 23° C. A small amount of water was added to quench excess NaBH₄ and the mixture was diluted with sat. aq. NaHCO₃ and was filtered through Celite. The filtrate was extracted with EtOAc. The organic phase was dried (Na₂SO₄), filtered, and concentrated, affording the crude title compound as an off-white solid.

### Intermediate 42: Step c

### 4-Isothiocyanato-3-methoxy-benzamide

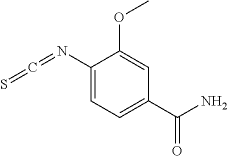

The title compound was prepared using 4-amino-3-methoxy-benzamide (intermediate 42, step b) in place of 3-amino-4-isopropoxy-benzamide according to the procedure described for intermediate 23, step d.

### Intermediate 42: Step d

### 3-Methoxy-4-thioureido-benzamide

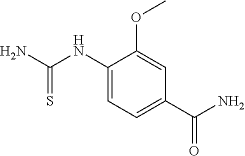

The title compound was prepared using 4-isothiocyanato-3-methoxy-benzamide (intermediate 42, step c) in place of 4-isopropoxy-3-isothiocyanato-benzamide according to the procedure described for intermediate 23, step e. The title compound was obtained as a white solid by filtration of the reaction mixture.

### Intermediate 43: Step a

### 4-Methoxy-2-methyl-benzamide

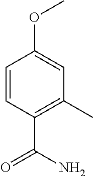

The title compound was prepared as a white powder using 4-methoxy-2-methyl-benzoic acid (Aldrich) in place of 3-methoxy-4-nitro-benzoic acid according to the procedure of intermediate 42, step a.

### Intermediate 43: Step b

### 4-Methoxy-2-methyl-5-nitro-benzamide

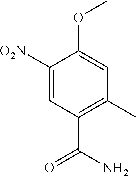

4-Methoxy-2-methyl-benzamide (7.75 g, 46.9 mmol, intermediate 43, step a) was cooled to 0° C. and concentrated sulfuric acid (40 mL) was added followed by potassium nitrate (4.74 g, 46.9 mmol) and the resulting brown suspension was stirred at room temperature for 40 min, then was slowly added to ice. The cream-colored precipitate was collected by vacuum filtration. The solid was dissolved in a mixture of THF and CH₂Cl₂ and was dried over Na₂SO₄, filtered, and concentrated, affording the crude title compound as a white solid.

### Intermediate 43: Step c

### 5-Amino-4-methoxy-2-methyl-benzamide

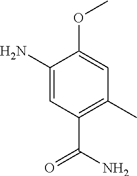

The title compound was prepared using 4-methoxy-2-methyl-5-nitro-benzamide (intermediate 43, step b) in place of 3-methoxy-4-nitro-benzamide according to the procedure of intermediate 42, step b. The crude product was purified by column chromatography (Silica gel, 0-7.5% MeOH—CH₂Cl₂), affording the title compound as a cream-colored solid.

### Intermediate 43: Step d

### 5-Isothiocyanato-4-methoxy-2-methyl-benzamide

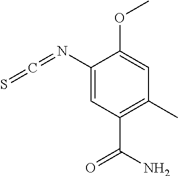

The title compound was prepared using 5-amino-4-methoxy-2-methyl-benzamide (intermediate 43, step c) in place of 3-amino-4-isopropoxy-benzamide according to the procedure described for intermediate 23, step d. During the extraction, precipitated solid made separation of the phases difficult; the solid was collected by vacuum filtration and was combined with the organic extracts. The crude title compound was obtained as a cream colored solid.

### Intermediate 43: Step e

### 4-Methoxy-2-methyl-5-thioureido-benzamide

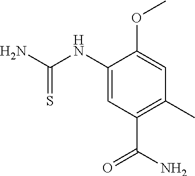

The title compound was prepared using 5-isothiocyanato-4-methoxy-2-methyl-benzamide (intermediate 43, step d) in place of 4-isopropoxy-3-isothiocyanato-benzamide according to the procedure described for intermediate 23, step e. The reaction mixture was concentrated to approximately half its original volume and cooled to 0° C., causing precipitation. The precipitated tan crystalline solid was collected by vacuum filtration and washed with MeOH to afford the title compound.

### Intermediate 44: Step a

### 4-Isobutyl-benzamide

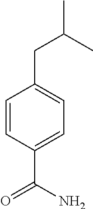

The title compound was prepared as a white powder using 4-isobutyl-benzoic acid (TCI) in place of 3-methoxy-4-nitro-benzoic acid according to the procedure of intermediate 42, step a.

### Intermediate 44: Step b

### 4-Isobutyl-3-nitro-benzamide

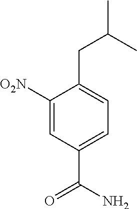

The title compound was prepared using 4-isobutyl-benzamide (intermediate 44, step a) in place of 4-methoxy-2-methyl-benzamide according to the procedure of intermediate 43, step b.

### Intermediate 44: Step c

### 3-Amino-4-isobutyl-benzamide

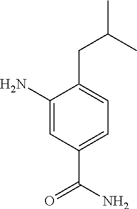

The title compound was prepared using 4-isobutyl-3-nitro-benzamide (intermediate 44, step b) in place of 3-methoxy-4-nitro-benzamide according to the procedure of intermediate 42, step b.

### Intermediate 44: Step d 4-Isobutyl-3-isothiocyanato-benzamide

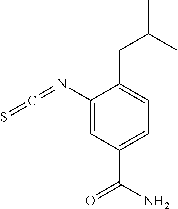

The title compound was prepared using 3-amino-4-isobutyl-benzamide (intermediate 44, step c) in place of 3-amino-4-isopropoxy-benzamide according to the procedure described for intermediate 23, step d. (The reaction was monitored by TLC and several additional portions of thiophosgene were added until the reaction approached complete conversion).

### Intermediate 44: Step e 4-Isobutyl-3-thioureido-benzamide

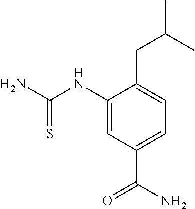

The title compound was prepared using 4-isobutyl-3-isothiocyanato-benzamide (intermediate 44, step d) in place of 4-isopropoxy-3-isothiocyanato-benzamide according to the procedure described for intermediate 23, step e. The reaction mixture was concentrated to approximately half its original volume and cooled to 0° C., causing precipitation. The precipitated white solid was collected by vacuum filtration and washed with MeOH to afford the title compound.

### Intermediate 45: Step a

### 3-Amino-4-methoxy-benzonitrile

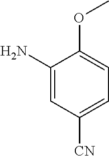

Sodium borohydride (0.159 g, 4.21 mmol) was added slowly to a solution of nickel (II) chloride hexahydrate (0.361 g, 1.40 mmol) in methanol (20 mL) at room temperature and stirred for 0.5 hours. To the resulting solution was added commercially available 4-methoxy-3-nitro-benzonitrile (0.500 g, 2.81 mmol) in methanol (10 mL), followed by sodium borohydride (0.372 g, 9.80 mmol) and stirred for 0.5 hours. The solution was then filtered through Celite, and evaporated. Water was added and the crude product was extracted with ethyl acetate, dried with sodium sulfate and purified via column chromatography with heptanes: ethyl acetate to give the title compound.

### Intermediate 45: Step b

### 3-Isothiocyanato-4-methoxy-benzonitrile

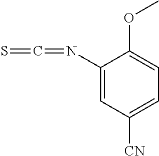

Thiophosgene (0.112 mL, 1.46 mmol) was added to a solution of 3-amino-4-methoxy-benzonitrile (0.216 g, 1.46 mmol, intermediate 45, step a) and sodium bicarbonate (0.368 g, 4.38 mmol) in chloroform (1 mL) and water (10 mL) and stirred at room temperature overnight. An additional 0.1 eq of thiophosgene was added to the reaction mixture and stirred for several hours. Excess ethyl acetate was added and the product was extracted, dried with sodium sulfate and purified via column chromatography with heptanes: ethyl acetate to give the title compound.

### Intermediate 45: Step c

### (5-Cyano-2-methoxy-phenyl)-thiourea

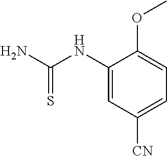

3-Isothiocyanato-4-methoxy-benzonitrile (0.140 g, 0.737 mmol, intermediate 45, step b) was dissolved in a solution of ammonia (2.0 M in methanol, 0.737 mL) and the reaction was heated in a sealed tube at 50° C. for 2 hours. The reaction mixture was then cooled to room temperature and filtered to give the title compound as a solid.

### Intermediate 46

### (6-Methoxy-quinolin-8-yl)-thiourea

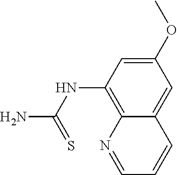

Benzoyl isothiocyanate (0.19 g, 1.15 mmol) was added dropwise to a solution of commercially available 6-methoxy-quinolin-8-ylamine (0.20 g, 1.15 mmol) in acetone (10 mL) and stirred at room temperature for 1 hour until solid precipitated out. The solid was filtered and then dissolved in 10% aqueous sodium hydroxide (5 mL) and heated to 100° C. for 1 hour. The solution was cooled to room temperature and filtered to give the title compound.

### Intermediate 47

### Quinolin-8-yl-thiourea

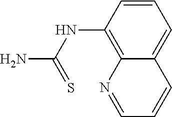

The title compound was prepared using commercially available quinolin-8-ylamine in place of 6-methoxy-quinolin-8-ylamine, according to the procedure described in intermediate 46, except the product was extracted with ethyl acetate, dried with sodium sulfate and evaporated to give the title compound.

### Intermediate 48: Step a

### 4-Ethoxy-3-nitro-benzoic acid ethyl ester

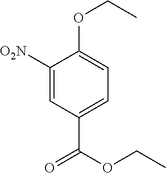

A solution of sodium ethoxide (21% in ethanol, 12.37 mL) was added slowly to a solution of commercially available 4-fluoro-3-nitro-benzoic acid methyl ester (3.00 g, 15.06 mmol) in ethanol (50 mL) at room temperature and stirred for 10 minutes. The reaction mixture was then evaporated, water was added and the product was extracted with ethyl acetate, dried with sodium sulfate and evaporated to give the title compound.

### Intermediate 48: Step b

### 3-Amino-4-ethoxy-benzoic acid ethyl ester

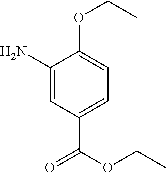

The title compound was prepared using 4-ethoxy-3-nitro-benzoic acid ethyl ester (intermediate 48, step a) in place of 4-methoxy-3-nitro-benzonitrile, according to the procedure described in intermediate 45, step a.

### Intermediate 48: Step c

### 4-Ethoxy-3-isothiocyanato-benzoic acid ethyl ester

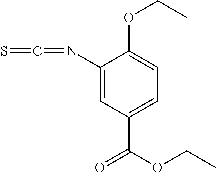

The title compound was prepared using 3-amino-4-ethoxy-benzoic acid ethyl ester (intermediate 48, step b) in place of 3-amino-4-methoxy-benzonitrile, according to the procedure described for intermediate 45, step b.

### Intermediate 48: Step d

### 4-Ethoxy-3-thioureido-benzoic acid ethyl ester

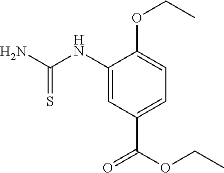

The title compound was prepared using 4-ethoxy-3-isothiocyanato-benzoic acid ethyl ester (intermediate 48, step c) in place of 3-isothiocyanato-4-methoxy-benzonitrile, according to the procedure from intermediate 45, step c, except that ammonia (0.5 M in dioxane) was used in place of ammonia (2.0 M in methanol).

### Intermediate 49

### 4-Ethoxy-3-thioureido-benzoic acid

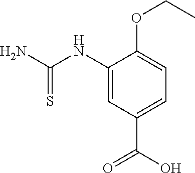

A solution of 1 N aqueous sodium hydroxide (0.932 mL, 0.932 mmol) was added to a solution of 4-ethoxy-3-thioureido-benzoic acid ethyl ester (0.125 g, 0.466 mmol, intermediate 48, step d) in methanol (5 mL) and stirred at room temperature overnight. The resulting solution was evaporated to remove methanol. A solution of 1 N aqueous HCl was added to adjust the pH to 4 and the product was extracted with ethyl acetate, dried with sodium sulfate and evaporated to give the title compound.

### Intermediate 50: Step a

### 1-Isopropoxy-4-methanesulfonyl-2-nitro-benzene

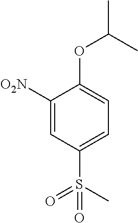

A solution of KHMDS (0.5 M in toluene, 13.68 mL, 6.84 mmol) was added dropwise to a solution of isopropanol (0.52 mL, 6.84 mmol) and THF (30 mL) at 0° C. and stirred for 10 minutes. The resulting solution was added to a solution of commercially available 4-methylsulfonyl-2-nitrofluorobenzene (1.00 g, 4.56 mmol) in THF (10 mL) at 0° C. and stirred for 0.5 hours. Water was added and the product was extracted with ethyl acetate, dried with sodium sulfate and evaporated to give the title compound.

### Intermediate 50: Step b

### 2-Isopropoxy-5-methanesulfonyl-phenylamine

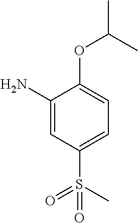

The title compound was prepared using 1-isopropoxy-4-methanesulfonyl-2-nitro-benzene (intermediate 50, step a) in place of 4-methoxy-3-nitro-benzonitrile, according to the procedure described in intermediate 45, step a, except that column chromatography was not done.

### Intermediate 50: Step c

### 1-Isopropoxy-2-isothiocyanato-4-methanesulfonyl-benzene

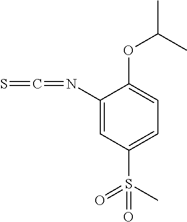

The title compound was prepared using 2-isopropoxy-5-methanesulfonyl-phenylamine (intermediate 50, step b) in place of 3-amino-4-methoxy-benzonitrile, according to the procedure described for intermediate 45, step b, except that column chromatography was not done.

### Intermediate 50: Step d

### (2-Isopropoxy-5-methanesulfonyl-phenyl)-thiourea

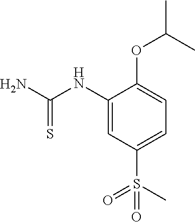

The title compound was prepared using 1-isopropoxy-2-isothiocyanato-4-methanesulfonyl-benzene (intermediate 50, step c) in place of 3-isothiocyanato-4-methoxy-benzonitrile, according to the procedure described for intermediate 45, step c, except that the reaction was done at room temperature.

### Intermediate 51: Step a

### 4-Ethanesulfonyl-2-isothiocyanato-1-methoxy-benzene

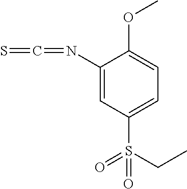

The title compound was prepared using commercially available 5-ethanesulfonyl-2-methoxy-phenylamine in place of 3-amino-4-methoxy-benzonitrile, according to the procedure described for intermediate 45, step b, except that column chromatography was not done.

### Intermediate 51: Step b

### (5-Ethanesulfonyl-2-methoxy-phenyl)-thiourea

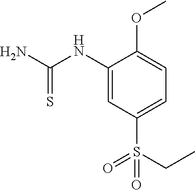

The title compound was prepared using 4-ethanesulfonyl-2-isothiocyanato-1-methoxy-benzene (intermediate 51, step a) in place of 3-isothiocyanato-4-methoxy-benzonitrile, according to the procedure described for intermediate 45, step c, except that ammonia (0.5 M in dioxane) was used in place of ammonia (2.0 M in methanol) and column chromatography was not done.

### Intermediate 52: Step a

### 3-Amino-4-methoxy-benzenesulfonamide

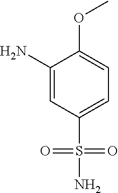

A solution of 30% aqueous ammonium hydroxide (3 mL) was added dropwise to a solution of commercially available 3-amino-4-methoxy-benzenesulfonyl fluoride (0.500 g, 2.44 mmol) at 0° C., then stirred at room temperature overnight. An additional aliquot of 30% aqueous ammonium hydroxide (5 mL) was added and the mixture was heated to 50° C. for several hours. Water was added and the product was extracted with ethyl acetate, dried with sodium sulfate and evaporated to give the title compound.

### Intermediate 52: Step b

### 3-Isothiocyanato-4-methoxy-benzenesulfonamide

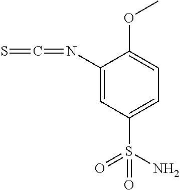

The title compound was prepared using 3-amino-4-methoxy-benzenesulfonamide (intermediate 52, step a) in place of 3-amino-4-methoxy-benzonitrile, according to the procedure described for intermediate 45, step b, except that column chromatography was not done.

### Intermediate 52: Step c

### 4-Methoxy-3-thioureido-benzenesulfonamide

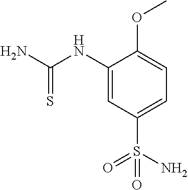

The title compound was prepared using 3-isothiocyanato-4-methoxy-benzenesulfonamide (intermediate 52, step b) in place of 3-isothiocyanato-4-methoxy-benzonitrile, according to the procedure described for intermediate 45, step c, except that ammonia (0.5 M in dioxane) was used in place of ammonia (2.0 M in methanol) and the reaction was concentrated slightly and filtered to give the product as a solid instead of using column chromatography.

### Intermediate 53

### 4-Methoxy-3-thioureido-benzoic acid

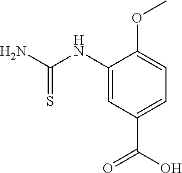

Benzoyl isothiocyanate (0.742 mL, 5.52 mmol) was added dropwise to a solution of commercially available methyl 3-amino-4-methoxybenzoate (1.00 g, 5.52 mmol) in acetone (10 mL) and stirred at room temperature for 1 hour. Water was added and the solution was filtered to give a solid. The solid was filtered and then dissolved in 10% aqueous sodium hydroxide (15 mL) and heated to 100° C. for several hours. The solution was then cooled to room temperature, the pH was adjusted to 6 with 1 N aqueous HCl and the suspension was filtered to give the title compound as a solid.

### Intermediate 54

### 4-Methoxy-3-thioureido-benzoic acid methyl ester

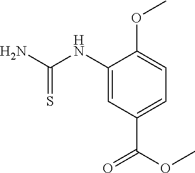

A slurry of 4-methoxy-3-thioureido-benzoic acid (0.500 g, 2.21 mmol, intermediate 53) in methanol (50 mL) and several drops of sulfuric acid were heated to reflux for 6 hours. The solution was then evaporated, saturated aqueous NaHCO₃ was added and the crude product was extracted with ethyl acetate, dried with sodium sulfate and evaporated. The product was purified via column chromatography with a gradient of heptanes: ethyl acetate to ethyl acetate to give the title compound.

### Intermediate 55: Step a

### 4-Fluoro-3-nitro-benzenesulfonamide

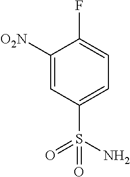

Following the procedure of J. Med. Chem. 2006, 49, 1173, a solution of commercially available 2-fluoronitrobenzene (10.00 g, 70.87 mmol) and chlorosulfonic acid (21 mL) were heated to reflux for 18 hours at 95° C. and then cooled to room temperature. The solution was then added dropwise over a 1 hour period to a solution of iPrOH (225 mL) and concentrated aqueous NH₄OH (54 mL) at −35° C. and stirred for 0.5 hours. The solution was maintained at −35° C. while concentrated aqueous HCl was added until the pH was acidic. The solution was then evaporated to remove some iPrOH, water was added and the solution was evaporated again to remove most of the iPrOH. More water was added, the solution was filtered and the solid was washed with 1 N aqueous HCl and water to give the title compound.

### Intermediate 55: Step b

### 4-Isopropoxy-3-nitro-benzenesulfonamide

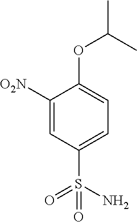

A solution of isopropanol (225 mL) and small chunks of sodium metal (1.92 g, 83.6 mmol) were heated to reflux for 2.5 hours, until the sodium was consumed. The resulting solution was added while still hot to a solution of 4-fluoro-3-nitro-benzenesulfonamide (8.37 g, 38.0 mmol, intermediate 55, step a) in THF/iPrOH (1/1, v/v, 150 mL) over a 10 minute period and stirred at room temperature for 3.5 hours. The reaction mixture was partitioned between EtOAc and brine and 1 N aqueous HCl. The organic phase was then washed with brine, dried with Na₂SO₄ and evaporated to give the title compound.

### Intermediate 55: Step c

### 3-Amino-4-isopropoxy-benzenesulfonamide

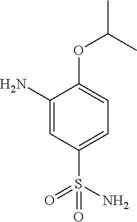

Sodium borohydride (1.88 g, 49.6 mmol) was added slowly to a solution of nickel (II) chloride hexahydrate (3.93 g, 16.5 mmol) in methanol (60 mL) at 0° C. and the resulting black suspension was stirred for 30 min at 23° C. The mixture was cooled to 0° C. and 4-isopropoxy-3-nitro-benzenesulfonamide (8.6 g, 33.0 mmol, intermediate 55, step b) was added followed by sodium borohydride (4.38 g, 115.6 mmol). The resulting black suspension was stirred for 30 min at 23° C. Water was added to the reaction mixture to quench excess NaBH₄, followed by addition of saturated aqueous NaHCO₃. The product was extracted with dichloromethane and the organic phase was washed with brine, dried with Na₂SO₄ and evaporated to give the title compound.

### Intermediate 55: Step d 4-Isopropoxy-3-isothiocyanato-benzenesulfonamide

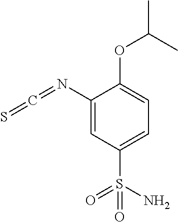

A solution of sodium bicarbonate (16.8 g, 199.5 mmol) in water (400 mL) was added to 3-amino-4-isopropoxy-benzenesulfonamide (15.3 g, 66.5 mmol, intermediate 55, step c) in a mixture of chloroform (200 mL) and water (200 mL). Thiophosgene (6.37 mL, 83.1 mmol) was then added. The biphasic solution was stirred at room temperature for 1.5 h. The phases were separated and the aqueous phase was extracted with CH₂Cl₂. The organic phase was washed with water, dried (Na₂SO₄), filtered, and concentrated, yielding the crude title compound as a tan solid.

### Intermediate 55: Step e

### 4-Isopropoxy-3-thioureido-benzenesulfonamide

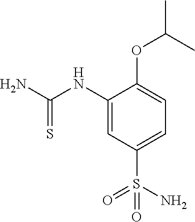

Crude 4-isopropoxy-3-isothiocyanato-benzenesulfonamide (17.8 g, 65.2 mmol, intermediate 55, step d) was treated with a 2 M solution of ammonia in MeOH (250 mL) and the resulting solution was stirred at room temperature for 18 h. The reaction mixture was then concentrated to about half the volume until a large amount of tan solid precipitated. The suspension was cooled to 0° C. for 30 minutes and was filtered. The solid was washed with methanol and ether to give the title compound as a cream colored solid.

### Intermediate 56: Step a

### N-(3-Isothiocyanato-4-methoxy-phenyl)-acetamide

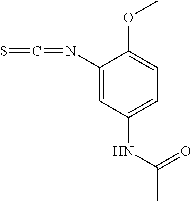

The title compound was prepared using commercially available 3-amino-4-methoxy acetanilide in place of 3-amino-4-methoxy-benzonitrile, according to the procedure described for intermediate 45, step b, except that column chromatography was not done.

### Intermediate 56: Step b

### N-(4-Methoxy-3-thioureido-phenyl)-acetamide

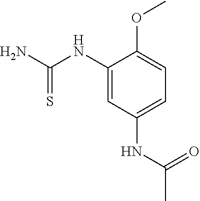

The title compound was prepared using N-(3-isothiocyanato-4-methoxy-phenyl)-acetamide (intermediate 56, step a) in place of 3-isothiocyanato-4-methoxy-benzonitrile, according to the procedure described for intermediate 45, step c, except that the reaction was run at room temperature instead of heating to 50° C., and the reaction mixture was evaporated in place of filtering.

### Intermediate 57

### (5-Methanesulfonyl-2-methoxy-phenyl)-thiourea

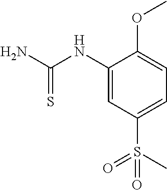

The title compound was prepared using commercially available 5-methanesulfonyl-2-methoxy-phenylamine in place of 6-methoxy-quinolin-8-ylamine, according to the procedure described for intermediate 46 except that after heating in 10% NaOH, the reaction mixture was cooled to room temperature and added to a solution of 6 N aqueous HCl and then the pH was adjusted to 10 with concentrated aqueous NH₄OH. Ethyl acetate was added and the product was extracted into the organic layer, dried with sodium sulfate and evaporated to give the title compound.

### Intermediate 58: Step a

### 4-Isopropoxy-3-nitro-benzoic acid isopropyl ester

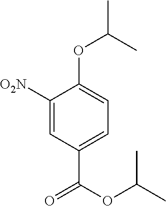

The title compound was prepared using commercially available 4-fluoro-3-nitro-benzoic acid methyl ester in place of 4-methylsulfonyl-2-nitrofluorobenzene, according to the procedure described for intermediate 50, step a, except that 3 equivalents of KHMDS and 3 equivalents of isopropanol were used, and the compound was purified via column chromatography with heptanes: ethyl acetate to give the title compound.

### Intermediate 58: Step b

### 3-Amino-4-isopropoxy-benzoic acid isopropyl ester

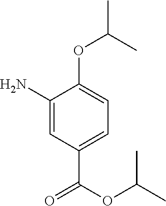

The title compound was prepared using 4-isopropoxy-3-nitro-benzoic acid isopropyl ester (intermediate 58, step a) in place of 4-methoxy-3-nitro-benzonitrile, according to the procedure described in intermediate 45, step a, except that column chromatography was not done.

### Intermediate 58: Step c

### 4-Isopropoxy-3-isothiocyanato-benzoic acid isopropyl ester

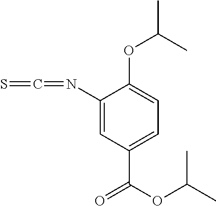

The title compound was prepared using 3-amino-4-isopropoxy-benzoic acid isopropyl ester (intermediate 58, step b) in place of 3-amino-4-methoxy-benzonitrile, according to the procedure described for intermediate 45, step b, except that column chromatography was not done.

### Intermediate 58: Step d 4-Isopropoxy-3-thioureido-benzoic acid isopropyl ester

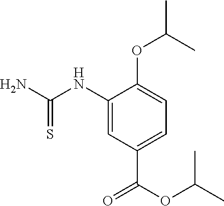

The title compound was prepared using 4-isopropoxy-3-isothiocyanato-benzoic acid isopropyl ester (intermediate 58, step c) in place of 3-isothiocyanato-4-methoxy-benzonitrile, according to the procedure described for intermediate 45, step c, except that the reaction was run at room temperature, ammonia (0.5 M in dioxane) was used in place of ammonia (2.0 M in methanol).

### Intermediate 58: Step e

### 4-Isopropoxy-3-thioureido-benzoic acid

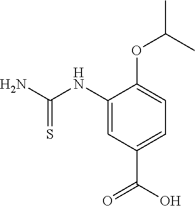

The title compound was prepared using 4-isopropoxy-3-thioureido-benzoic acid isopropyl ester (intermediate 58, step d) in place of 4-ethoxy-3-thioureido-benzoic acid ethyl ester, according to the procedure described in intermediate 49.

### Intermediate 59: Step a

### 4-Ethoxy-3-nitro-benzenesulfonamide

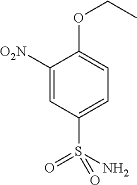

A solution of sodium ethoxide (21 wt. % in ethanol, 5.41 mL, 14.5 mmol) was added slowly to a solution of 4-fluoro-3-nitro-benzenesulfonamide (2.66 g, 12.1 mmol, intermediate 55, step a) in THF (75 mL) at 0° C. and stirred for 5 minutes. The reaction mixture was then evaporated, water was added and the product was extracted with ethyl acetate, dried with sodium sulfate and evaporated to give the title compound.

### Intermediate 59: Step b

### 3-Amino-4-ethoxy-benzenesulfonamide

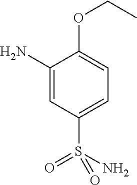

The title compound was prepared using 4-ethoxy-3-nitro-benzenesulfonamide (intermediate 59, step a) in place of 4-methoxy-3-nitro-benzonitrile, according to the procedure described in intermediate 45, step a, except that column chromatography was not done.

### Intermediate 59: Step c

### 4-Ethoxy-3-isothiocyanato-benzenesulfonamide

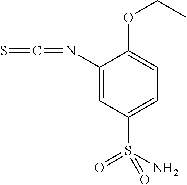

The title compound was prepared using 3-amino-4-ethoxy-benzenesulfonamide (intermediate 59, step b) in place of 3-amino-4-methoxy-benzonitrile, according to the procedure described for intermediate 45, step b, except that column chromatography was not done.

### Intermediate 59: Step d 4-Ethoxy-3-thioureido-benzenesulfonamide

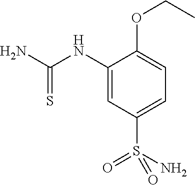

The title compound was prepared using 4-ethoxy-3-isothiocyanato-benzenesulfonamide (intermediate 59, step c) in place of 3-isothiocyanato-4-methoxy-benzonitrile, according to the procedure described for intermediate 45, step c, except that the reaction was run at room temperature, ammonia (0.5 M in dioxane) was used in place of ammonia (2.0 M in methanol).

### Intermediate 60: Step a

### 4-Isopropoxy-3-nitro-benzonitrile

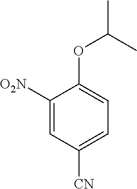

The title compound was prepared using commercially available 4-fluoro-3-nitro-benzonitrile in place of 4-methylsulfonyl-2-nitrofluorobenzene, according to the procedure described in intermediate 50, step a.

### Intermediate 60: Step b

### 3-Amino-4-isopropoxy-benzonitrile

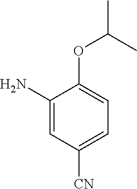

The title compound was prepared using 4-isopropoxy-3-nitro-benzonitrile (intermediate 60, step a) in place of 4-methoxy-3-nitro-benzonitrile, according to the procedure described in intermediate 45, step a.

### Intermediate 60: Step c

### 4-Isopropoxy-3-isothiocyanato-benzonitrile

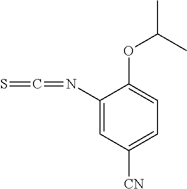

The title compound was prepared using 3-amino-4-isopropoxy-benzonitrile (intermediate 60, step b) in place of 3-amino-4-methoxy-benzonitrile, according to the procedure described in intermediate 45, step b, except column chromatography was not done.

### Intermediate 60: Step d (5-Cyano-2-isopropoxy-phenyl)-thiourea

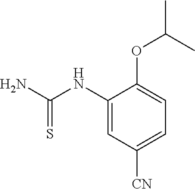

The title compound was prepared using 4-isopropoxy-3-isothiocyanato-benzonitrile (intermediate 60, step c) in place of 3-isothiocyanato-4-methoxy-benzonitrile, according to the procedure described for intermediate 45, step c, except that the reaction was run at room temperature, ammonia (0.5 M in dioxane) was used in place of ammonia (2.0 M in methanol).

### Intermediate 61: Step a

### 4-Ethoxy-3-nitro-benzoic acid

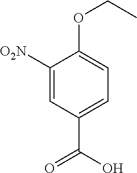

A solution of 2 N aqueous sodium hydroxide (8.36 mL) was added to a solution of 4-ethoxy-3-nitro-benzoic acid ethyl ester (2.00 g, 8.36 mmol, intermediate 48, step a) in ethanol (20 mL) and stirred at room temperature for 0.5 hours. An additional solution of 2 N aqueous sodium hydroxide (8.36 mL) was added and stirred for 0.5 hours. The reaction mixture was then evaporated to remove ethanol and 1 N aqueous HCl was added and the product was extracted with ethyl acetate, dried with sodium sulfate, and evaporated. Dichloromethane was added and the suspension was filtered to give the title compound as a solid.

### Intermediate 61: Step b

### 2-(4-Ethoxy-3-nitro-phenyl)-oxazole

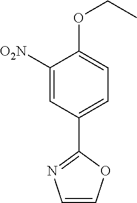

A solution of 4-ethoxy-3-nitro-benzoic acid (0.686 g, 3.48 mmol, intermediate 61, step a) and thionyl chloride (10 mL) was heated to 75° C. for 3 hours and then evaporated to dryness. In a separate flask, NaH (60% in mineral oil) (0.157 g, 3.92 mmol) was added to sulfolane (4 mL) and heated to 50° C. for several minutes. 1,2,3-1H-triazole (0.270 g, 3.92 mmol) was added and the reaction was stirred at 50° C. for 1.5 hours. The acid chloride was then dissolved in sulfolane (4 mL) and heated to 95° C. To this solution was added the triazine solution and the resulting solution was maintained at 95° C. for 1 hour. The reaction was cooled to room temperature, water was added and the crude product was extracted with ethyl acetate, dried with sodium sulfate and evaporated. The product was purified via column chromatography with heptanes: ethyl acetate to give the title compound.

### Intermediate 61: Step c

### 2-Ethoxy-5-oxazol-2-yl-phenylamine

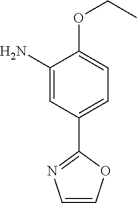

The title compound was prepared using 2-(4-ethoxy-3-nitro-phenyl)-oxazole (intermediate 61, step b) in place of 4-methoxy-3-nitro-benzonitrile, according to the procedure described in intermediate 45, step a.

### Intermediate 61: Step d

### 2-(4-Ethoxy-3-isothiocyanato-phenyl)-oxazole

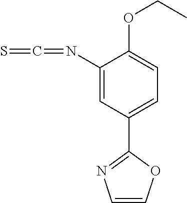

The title compound was prepared using 2-ethoxy-5-oxazol-2-yl-phenylamine (intermediate 61, step c) in place of 3-amino-4-methoxy-benzonitrile, according to the procedure described in intermediate 45, step b, except column chromatography was not done.

### Intermediate 61: Step e

### (2-Ethoxy-5-oxazol-2-yl-phenyl)-thiourea

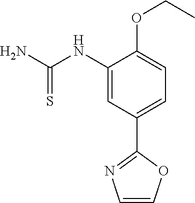

The title compound was prepared using 2-(4-ethoxy-3-isothiocyanato-phenyl)-oxazole (intermediate 61, step d) in place of 3-isothiocyanato-4-methoxy-benzonitrile, according to the procedure described for intermediate 45, step c, except that the reaction was run at room temperature, ammonia (0.5 M in dioxane) was used in place of ammonia (2.0 M in methanol), and the reaction mixture was concentrated instead of filtering.

### Intermediate 62: Step a

### 3-Nitro-4-trifluoromethoxy-benzenesulfonamide

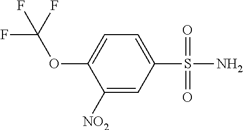

To chlorosulfonic acid (11.3 mL, 170 mmol) was slowly added commercially available 2-trifluoromethoxy-nitrobenzene (8 g, 38.6 mmol). The reaction mixture was heated at 120° C. for 4 h and then cooled down. The above crude mixture was added to a stirred solution of conc. aq. NH₄OH (34.7 mL, 514 mmol, 14.8 M) in iPrOH (100 mL) at −45° C. dropwise over 30 min. The reaction mixture was stirred at −45° C. for 1 h, and 2 N HCl was added to acidify the mixture. Concentration to remove iPrOH was followed by suspension in water, and filtration of the solid. The solid was washed successively with 1 N HCl and water, then air dried to yield the title compound as a white solid.

### Intermediate 62: Step b

### 3-Amino-4-trifluoromethoxy-benzenesulfonamide

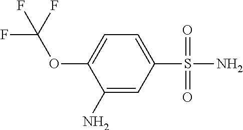

The title compound was prepared using 3-nitro-4-trifluoromethoxy-benzenesulfonamide (intermediate 62, step a) in place of 4-isopropoxy-3-nitro-benzamide according to the procedure of intermediate 23, step c.

### Intermediate 62: Step c

### 3-Isothiocyanato-4-trifluoromethoxy-benzenesulfonamide

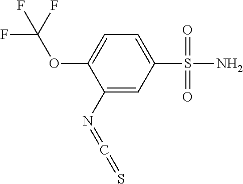

A solution of sodium bicarbonate (3.8 g, 45.2 mmol) in water (50 mL) was added to 3-amino-4-trifluoromethoxy-benzenesulfonamide (3.84 g, 15.0 mmol, intermediate 62, step b) in chloroform (100 mL). Thiophosgene (1.44 mL, 18.7 mmol) was then added. The biphasic solution was stirred at room temperature for 2 h. TLC analysis indicated slight remaining starting material, so an additional 0.5 mL portion of thiophosgene was added and the mixture was stirred for 40 min. The reaction mixture was partially concentrated to get rid of most chloroform. The precipitated solid was filtered, washed with water, and air dried, yielding the crude title compound as an off-white solid.

### Intermediate 62: Step d

### 3-Thioureido-4-trifluoromethoxy-benzenesulfonamide

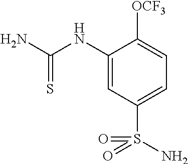

Crude 3-isothiocyanato-4-trifluoromethoxy-benzenesulfonamide (2.2 g, intermediate 62, step c) was dissolved in a 2 M solution of ammonia in MeOH (29.5 mL). The resulting yellow solution was stirred at room temperature for 16 h. The reaction mixture was concentrated and dried under vacuum to afford the title compound as a foamy solid.

### Intermediate 63

### (2-Methoxy-pyridin-3-yl)-thiourea

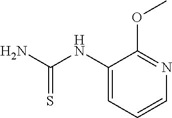

A solution of ammonium thiocyanate (1.0 g, 13 mmol) in acetone (20 mL) was heated to reflux and benzoyl chloride (1.8 g, 13 mmol) was added dropwise and the mixture was heated at reflux for 10 min. A solution of commercially available 2-methoxy-pyridin-3-ylamine (1.6 g, 13 mmol) in acetone (50 mL) was added dropwise and the mixture was heated to reflux overnight. The mixture was poured into water (600 mL) and the resulting suspension was filtered. The collected solid was dissolved in 10% aq. NaOH (20 mL) and the mixture was heated to reflux for 2 h. The mixture was cooled in an ice bath and the pH was adjusted to 4 with conc. aq. HCl, then to pH 9 with conc. aq. NH₄OH. The precipitated solid was collected by vacuum filtration and was air-dried, to yield the title compound.

### Intermediate 64: Step a

### 3-Isothiocyanato-4-methoxypyridine

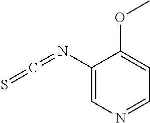

The title compound was prepared according to the procedure of intermediate 23, step d, using

4-methoxypyridin-3-amine in place of 3-amino-4-isopropoxy-benzamide.

### Intermediate 64: Step b

### 1-(4-Methoxypyridin-3-yl)thiourea

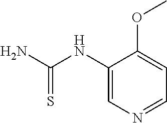

The title compound was prepared using 3-isothiocyanato-4-methoxypyridine (intermediate 64, step a) in place of 4-isopropoxy-3-isothiocyanato-benzamide according to the procedure described for intermediate 23, step e.

### Intermediate 65: Step a

### 4-Ethoxy-3-isothiocyanatopyridine

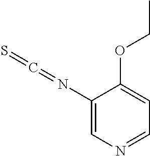

To a stirred mixture of 4-ethoxypyridin-3-amine (1.04 g, 7.53 mmol) and sodium bicarbonate (1.90 g, 22.8 mmol) in chloroform (15 mL) and water (10 mL) at 4° C. was added thiophosgene (0.70 mL, 9.13 mmol) dropwise. The cooling bath was removed and the biphasic mixture was stirred at room temperature for 4 h. TLC analysis indicated some of the starting material was still present. More thiophosgene (0.2 mL, 2.61 mmol) was added and stirring was continued for another 1 h and filtered. The organic layer was separated, and the aqueous layer was extracted with CH₂Cl₂. The combined organic layers were dried (Na₂SO₄) and concentrated to give the crude title compound as a brown solid.

### Intermediate 65: Step b

### 1-(4-Ethoxypyridin-3-yl)thiourea

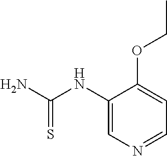

The above crude 4-ethoxy-3-isothiocyanatopyridine (intermediate 65, step a) in MeOH (11 mL) was treated with a 7 N solution of ammonia in MeOH (11 mL) at room temperature for 18 h and filtered. The solid was washed with EtOH and Et₂O, and dried to give the title compound as an off-white solid. The filtrate was concentrated and purified by column chromatography (silica gel, 5-10% MeOH in CH₂Cl₂) to obtain the title compound as a light brown solid.

### Intermediate 66: Step a

### 4-(Ethylthio)-3-nitrobenzenesulfonamide

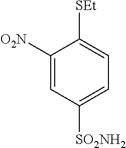

1,8-Diazabicyclo[5.4.0]undec-7-ene (DBU, 1.60 mL, 10.7 mmol) was added to a mixture of 4-fluoro-3-nitro-benzenesulfonamide (2.24 g, 10.2 mmol, intermediate 55, step a) and ethanethiol (0.79 mL, 10.7 mmol) in acetonitrile and the resulting yellow solution was stirred overnight at room temperature. The reaction mixture was concentrated and the residue was partitioned between DCM and 1 N aq. HCl. The separated organic phase was washed with sat. aq. NaHCO₃. The organic phase contained precipitated yellow solid which was collected by vacuum filtration, washed with heptanes, and air-dried to afford the crude title compound.

### Intermediate 66: Step b

### 3-Amino-4-(ethylthio)benzenesulfonamide

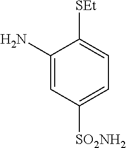

The title compound was prepared using 4-(ethylthio)-3-nitrobenzenesulfonamide (intermediate 66, step a) in place of 3-methoxy-4-nitro-benzamide according to the procedure of intermediate 42, step b.

### Intermediate 66: Step c

### 4-(Ethylthio)-3-isothiocyanatobenzenesulfonamide

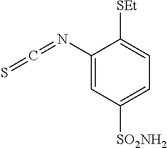

The title compound was prepared using 3-amino-4-(ethylthio)benzenesulfonamide (intermediate 66, step b) in place of 3-amino-4-isopropoxy-benzamide according to the procedure described for intermediate 23, step d, except that the reaction mixture was filtered, and the collected solid was washed with chloroform and air-dried to afford the crude title compound.

### Intermediate 66: Step d 4-(Ethylthio)-3-thioureidobenzenesulfonamide

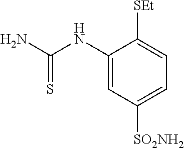

Crude 4-(ethylthio)-3-isothiocyanatobenzenesulfonamide (1.01 g, 3.68 mmol, intermediate 66, step c) was treated with a solution of ammonia in methanol (2 M, 20 mL, 40 mmol) and the resulting yellow solution was stirred at 40° C. for 40 min. The reaction mixture was partially concentrated, until a solid precipitated. The solid was collected by vacuum filtration, washed with cold methanol and air-dried to afford the title compound as a white solid.

### Intermediate 67: Step a

### 1-(2,4-Dimethylthiazol-5-yl)propan-1-one

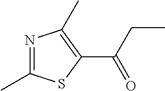

Lithium hexamethyldisilazide (1 M in THF, 21.3 mL, 21.3 mmol) was added to a solution of 1-(2,4-dimethylthiazol-5-yl)ethanone (Alfa, 3.0 g, 19.3 mmol) in THF (20 mL) at −78° C. The resulting yellow solution was stirred at −78° C. for 30 min before addition of iodomethane (1.33 mL, 21.3 mmol). The resulting yellow solution was stirred at −78° C. for 30 min, then at 0° C. for 30 min. Saturated aq. NH₄Cl was added and the mixture was partially concentrated to remove THF. The aqueous residue was extracted with EtOAc and the organic extracts were dried (Na₂SO₄), filtered, and concentrated. The crude product was purified by column chromatography (Silica gel, 0-30% EtOAc-hept), affording the title compound as a light yellow liquid.

### Intermediate 67: Step b

### 2-Bromo-1-(2,4-dimethylthiazol-5-yl)propan-1-one

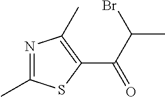

To 1-(2,4-dimethylthiazol-5-yl)propan-1-one (631 mg, 3.73 mmol, intermediate 67, step a) in 1,4-dioxane (6 mL) was added bromine (0.191 mL, 3.73 mmol) in 1,4-dioxane (6 mL). The resulting orange solution was heated in a 50° C. oil bath for 3 days. The reaction mixture was filtered and the collected solid was partitioned between EtOAc and sat. aq. NaHCO₃. The separated aq. phase was extracted with EtOAc and the organic extracts were dried (Na₂SO₄), filtered, and concentrated. The residue was purified by column chromatography (Silica gel, 1-5% Et₂O-DCM), affording the title compound as a colorless liquid.

### Intermediate 68: Step a

### 1-Methylpiperidine-4-carbothioamide

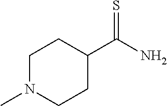

To a suspension of 1-methylpiperidine-4-carboxamide (3.97 g, 27.9 mmol, Amfinecom) in a mixture of toluene (70 mL) and THF (30 mL) was added Lawesson's reagent (6.78 g, 16.8 mmol). The resulting light yellow suspension was heated at reflux for 22 h. The reaction mixture was diluted with DCM and MeOH and was concentrated onto silica gel for purification by column chromatography (Silica gel, 1-8% MeOH in 98:2 DCM:conc. aq. NH₄OH, water layer removed in a separatory funnel), affording the title compound as a yellow solid.

### Intermediate 68: Step b

### 1-(4-Methyl-2-(1-methylpiperidin-4-yl)thiazol-5-yl)ethanone

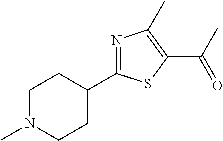

To a suspension of 1-methylpiperidine-4-carbothioamide (1.80 g, 11.4 mmol, intermediate 68, step a) in EtOH (40 mL) was added 3-chloropentane-2,4-dione (1.55 mL, 13.6 mmol) and the mixture was heated at reflux overnight. The reaction mixture was partitioned between EtOAc and sat. aq. NaHCO₃. The aq. phase was extracted with EtOAc. The organic extracts were dried (Na₂SO₄), filtered, and concentrated. The residue was purified by column chromatography (Silica gel, 1-5% MeOH in 98:2 DCM:conc. aq. NH₄OH, water layer removed in a separatory funnel), affording the title compound.

### Intermediate 68: Step c

### 2-bromo-1-(4-methyl-2-(1-methylpiperidin-4-yl)thiazol-5-yl)ethanone.HBr

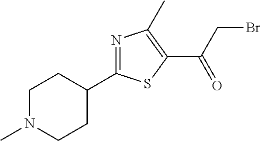

A solution of bromine (0.064 mL, 1.25 mmol) in 48% aq. HBr (0.5 mL) was added to a mixture of 1-(4-methyl-2-(1-methylpiperidin-4-yl)thiazol-5-yl)ethanone (270 mg, 1.13 mmol, intermediate 68, step b) in 48% aq. HBr (2.0 mL). The reaction mixture was heated in a 60° C. oil bath for 75 min. The mixture was concentrated from toluene three times, affording the crude title compound as a thick brown oil which was used without further purification in the next step.

### Examples

### Example 1

### 3-(2′,4′-Dimethyl-[4,5′]bithiazolyl-2-ylamino)-4-isopropoxy-benzamide.HBr

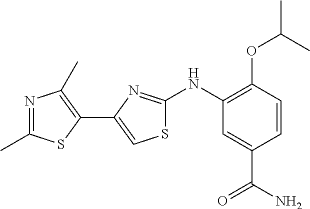

A mixture of 2-bromo-1-(2,4-dimethyl-thiazol-5-yl)-ethanone.HBr (63.0 mg, 0.200 mmol, intermediate 3) and 4-isopropoxy-3-thioureido-benzamide (50.7 mg, 0.200 mmol, intermediate 23, step e) in ethanol (1 mL) was stirred at room temperature for 4 d. The mixture was filtered, washed sequentially with EtOH and heptanes, and air-dried, affording the title compound. ¹H NMR (300 MHz, DMSO-d₆) δ ppm 9.50 (s, 1H), 8.82 (d, J=1.9 Hz, 1 H), 7.74 (br. s., 1H), 7.52 (dd, J=8.3, 1.9 Hz, 1H), 7.04-7.22 (m, 3H), 4.77 (sept, J=6.2 Hz, 1H), 2.67 (s, 3H), 2.57 (m, 3H), 1.34 (d, J=6.0 Hz, 6H). MS m/e 389.0 (M+H).

### Example 2

### 3-(2′-Amino-4′-methyl-[4,5′]bithiazolyl-2-ylamino)-4-isopropoxy-benzamide.HBr

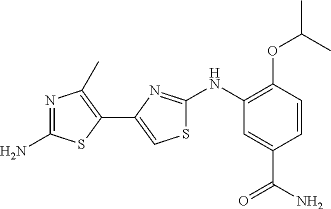

The title compound was prepared using 1-(2-amino-4-methyl-thiazol-5-yl)-2-bromo-ethanone (intermediate 4) in place of 2-bromo-1-(2,4-dimethyl-thiazol-5-yl)-ethanone.HBr as described in Example 1. ¹H NMR (300 MHz, DMSO-d₆) δ ppm 9.52 (s, 1H), 9.20 (br. s., 2H), 8.76 (s, 1H), 7.78 (br. s., 1H), 7.53 (d, J=7.9 Hz, 1H), 6.99-7.16 (m, 3H), 4.72-4.82 (sept, J=6.0 Hz, 1H), 2.44 (s, 3H), 1.33 (d, J=6.0 Hz, 6H). MS m/e 390.1 (M+H).

### Example 3

### 3-(2′-Acetylamino-4′-methyl-[4,5′]bithiazolyl-2-ylamino)-4-isopropoxy-benzamide.HBr

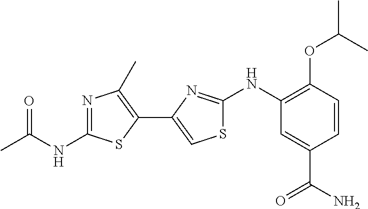

The title compound was prepared using N-[5-(2-bromo-acetyl)-4-methyl-thiazol-2-yl]-acetamide.HBr (intermediate 5) in place of 2-bromo-1-(2,4-dimethyl-thiazol-5-yl)-ethanone.HBr as described in Example 1. The crude product was recrystallized from a mixture of EtOH and water, affording the title compound as a cream-colored solid. ¹H NMR (300 MHz, DMSO-d₆) δ ppm 12.05 (s, 1H), 9.36 (s, 1H), 8.73 (d, J=1.9 Hz, 1H), 7.71 (br. s., 1H), 7.52 (dd, J=8.5, 2.1 Hz, 1H), 7.05-7.14 (m, 2H), 6.88 (s, 1H), 4.76 (sept, J=6.0 Hz, 1H), 2.51 (s, 3H), 2.13 (s, 3H), 1.34 (d, J=6.0 Hz, 6H). MS m/e 432.1 (M+H).

### Example 4

### 3-(5-Bromo-2′,4′-dimethyl-[4,5′]bithiazolyl-2-ylamino)-4-isopropoxy-benzamide

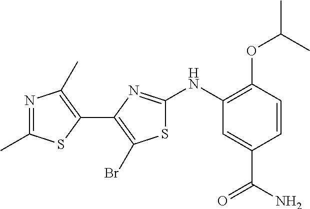

3-(2′,4′-dimethyl-[4,5′]bithiazolyl-2-ylamino)-4-isopropoxy-benzamide.HBr (70 mg, 0.149 mmol, Example 1) was treated with AcOH (2 mL). The suspension was placed in an ice bath and to it was immediately (i.e. before freezing occurred) added a solution of bromine (7.3 μL, 0.14 mmol) in AcOH (3 mL). The resulting orange suspension was stirred at 23° C. for 10 min. The reaction mixture was partitioned between EtOAc and sat. aq. NaHCO₃ (50 mL each) and the aqueous phase was extracted with EtOAc (50 mL). The organic extracts were washed with 0.2 M aq. sodium thiosulfate, then were dried (Na₂SO₄), filtered, and concentrated. The residue was purified by RP-HPLC (10-90% CH₃CN—H₂O, 0.1% TFA) and the resulting TFA salt was converted to the free base by dissolving in sat. aq. NaHCO₃ and extraction with dichloromethane. The title compound was obtained as a white powder. ¹H NMR (300 MHz, DMSO-d₆) δ ppm 9.61 (s, 1H), 8.75 (s, 1H), 7.73 (br. s., 1H), 7.51 (d, J=8.3 Hz, 1H), 7.04-7.15 (m, 2H), 4.76 (sept, J=6.1 Hz, 1H), 2.63 (s, 3H), 2.48 (s, 3H), 1.34 (d, J=6.0 Hz, 6H). MS m/e 466.9; 468.9 (M+H).

### Example 5

### 3-(2′-Amino-4′-methyl-[4,5′]bithiazolyl-2-ylamino)-4-methoxy-benzamide.HBr

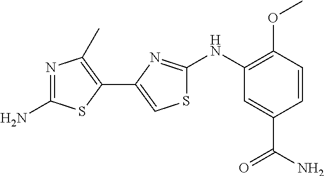

The title compound was prepared using 1-(2-amino-4-methyl-thiazol-5-yl)-2-bromo-ethanone (intermediate 4) in place of 2-bromo-1-(2,4-dimethyl-thiazol-5-yl)-ethanone.HBr and 4-methoxy-3-thioureido-benzamide (intermediate 24) in place of 4-isopropoxy-3-thioureido-benzamide as described in Example 1 (reaction time 18 h). The crude product was recrystallized from a mixture of EtOH and water. ¹H NMR (300 MHz, DMSO-d₆) δ ppm 9.82 (s, 1H), 9.25 (br. s., 2H), 8.79 (s, 1H), 7.80 (br. s., 1H), 7.57 (d, J=7.9 Hz, 1H), 7.00-7.17 (m, 3H), 3.92 (s, 3H), 2.45 (s, 3H). MS m/e 362.1 (M+H).

### Example 6

### 3-(2′,4′-Dimethyl-[4,5′]bithiazolyl-2-ylamino)-4-ethoxy-benzamide.TFA

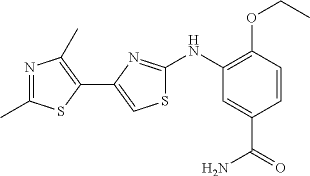

A mixture of 4-ethoxy-3-thioureido-benzamide (25.0 mg, 0.104 mmol, intermediate 25, step c), 2-bromo-1-(2,4-dimethyl-thiazol-5-yl)-ethanone.HBr (32.8 mg, 0.104 mmol, intermediate 3), and EtOH (1 mL) was stirred at room temperature for 3 d. The mixture was partitioned between 0.1 N aq. NaOH and EtOAc, and the aqueous phase was extracted with EtOAc. The organic phase was dried (Na₂SO₄), filtered, and concentrated and the residue was purified by RP-HPLC (10-90% CH₃CN—H₂O, 0.1% TFA) and lyophilized, affording the title compound. ¹H NMR (400 MHz, DMSO-d₆) δ ppm 9.55 (s, 1H), 8.82 (d, J=2.0 Hz, 1H), 7.73 (br. s., 1H), 7.52 (dd, J=8.6, 2.2 Hz, 1H), 7.13 (br. s., 1H), 7.05 (d, J=8.6 Hz, 1H), 6.96 (s, 1H), 4.18 (q, J=7.1 Hz, 2H), 2.60 (s, 3H), 2.54 (s, 3H), 1.40 (t, J=7.0 Hz, 3H). MS m/e 375.0 (M+H).

### Example 7

### 3-(2′-Amino-4′-methyl-[4,5′]bithiazolyl-2-ylamino)-4-ethoxy-benzamide

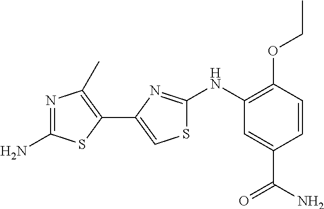

A mixture of 1-(2-amino-4-methyl-thiazol-5-yl)-2-bromo-ethanone (42.7 mg, 0.20 mmol, intermediate 4), 4-ethoxy-3-thioureido-benzamide (48.6 mg, 0.20 mmol, intermediate 25, step c) and EtOH (1 mL) was stirred at 23° C. overnight. The suspension was filtered and the collected solid was treated with sat. aq. NaHCO₃ and extracted with EtOAc. The organic phase was dried (Na₂SO₄), filtered, and concentrated and the residue was purified by flash column chromatography (Silica gel, 1-8% MeOH—CH₂Cl₂), yielding the title compound. ¹H NMR (300 MHz, DMSO-d₆) δ ppm 9.38 (s, 1H), 8.76 (d, J=2.3 Hz, 1H), 7.71 (br. s., 1H), 7.49 (dd, J=8.5, 2.1 Hz, 1H), 6.99-7.15 (m, 2H), 6.94 (s, 2H), 6.56 (s, 1H), 4.17 (q, J=6.8 Hz, 2H), 2.34 (s, 3H), 1.40 (t, J=7.0 Hz, 3H). MS m/e 375.9 (M+H).

### Example 8

### 3-(2′-Acetylamino-4′-methyl-[4,5′]bithiazolyl-2-ylamino)-4-ethoxy-benzamide.TFA

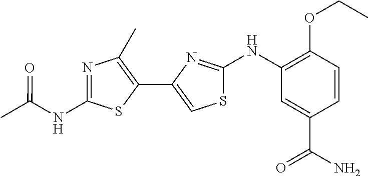

A mixture of N-[5-(2-bromo-acetyl)-4-methyl-thiazol-2-yl]-acetamide.HBr (58.2 mg, 0.163 mmol, intermediate 5) and 4-ethoxy-3-thioureido-benzamide (38.9 mg, 0.163 mmol, intermediate 25, step c) in ethanol (2 mL) was stirred at room temperature for 3 d. The mixture was filtered and the collected solid was purified by RP-HPLC (10-90% CH₃CN—H₂O, 0.1% TFA) affording the title compound. ¹H NMR (300 MHz, DMSO-d₆) δ ppm 12.06 (s, 1H), 9.49 (s, 1H), 8.73 (d, J=2.3 Hz, 1H), 7.72 (br. s., 1H), 7.53 (dd, J=8.3, 2.3 Hz, 1H), 7.02-7.13 (m, 2H), 6.88 (s, 1H), 4.18 (q, J=7.0 Hz, 2H), 2.13 (s, 3H), 1.40 (t, J=7.0 Hz, 3H). (Note that in this and selected other examples, one of the methyl resonances is obscured by the DMSO solvent peak). MS m/e 417.9 (M+H).

### Example 9

### N-[4′-Methyl-2-(2-methyl-benzofuran-7-ylamino)-[4,5′]bithiazolyl-2′-yl]-acetamide

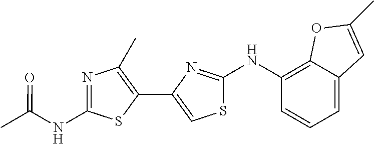

A mixture of (2-methyl-benzofuran-7-yl)-thiourea (103.1 mg, 0.500 mmol, intermediate 26) and N-[5-(2-bromo-acetyl)-4-methyl-thiazol-2-yl]-acetamide.HBr (179.0 mg, 0.500 mmol, intermediate 5) was heated by microwave irradiation (100° C., 10 min, 300 W). The reaction mixture was partitioned between CH₂Cl₂ and sat. aq. NaHCO₃. The separated aq. phase was extracted twice with CH₂Cl₂. The organic phase was dried (Na₂SO₄), filtered, and concentrated and the residue was purified by flash column chromatography (Silica gel, 20-70% EtOAc-Hept) to afford the title compound. ¹H NMR (400 MHz, DMSO-d₆) δ ppm 12.04 (s, 1H), 10.37 (s, 1H), 8.07 (dd, J=7.1, 1.7 Hz, 1H), 7.12-7.21 (m, 2H), 6.90 (s, 1H), 6.61 (s, 1H), 2.14 (s, 3H). MS m/e 385.0 (M+H).

### Example 10

### N-[2-(2,2-Difluoro-benzo[1,3]dioxol-4-ylamino)-4′-methyl-[4,5′]bithiazolyl-2′-yl]-acetamide

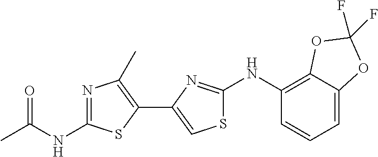

The title compound was prepared using (2,2-difluoro-benzo[1,3]dioxol-4-yl)-thiourea (intermediate 27) in place of (2-methyl-benzofuran-7-yl)-thiourea as described in Example 9. ¹H NMR (400 MHz, DMSO-d₆) δ ppm 12.06 (s, 1H), 10.48 (s, 1H), 8.06 (d, J=8.6 Hz, 1H), 7.21 (t, J=8.3 Hz, 1H), 6.96-7.08 (m, 2H), 2.48 (s, 3H), 2.14 (s, 3H). MS m/e 411.1 (M+H).

### Example 11

### N-[2-(2-Methoxy-5-methyl-phenylamino)-4′-methyl-[4,5′]bithiazolyl-2′-yl]-acetamide

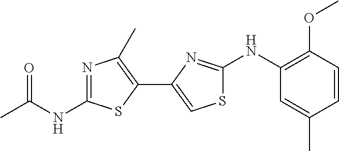

The title compound was prepared using (2-methoxy-5-methyl-phenyl)-thiourea (intermediate 28) in place of (2-methyl-benzofuran-7-yl)-thiourea as described in Example 9. ¹H NMR (400 MHz, DMSO-d₆) δ ppm 12.03 (s, 1H), 9.57 (s, 1H), 8.30 (d, J=1.7 Hz, 1H), 6.91 (d, J=8.1 Hz, 1H), 6.84 (s, 1H), 6.78 (dd, J=8.1, 1.5 Hz, 1H), 3.82 (s, 3H), 2.27 (s, 3H), 2.14 (s, 3H). MS m/e 375.0 (M+H).

### Example 12

### 4-tert-Butoxy-3-(2′,4′-dimethyl-[4,5′]bithiazolyl-2-ylamino)-benzamide

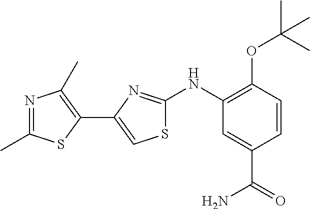

The title compound was prepared using 4-tert-butoxy-3-thioureido-benzamide (intermediate 29, step d) in place of 3-thioureido-4-trifluoromethoxy-benzamide according to the procedure described in Example 17 (reaction time 15 h). ¹H NMR (300 MHz, DMSO-d₆) δ ppm 9.36 (s, 1H), 8.77 (d, J=1.9 Hz, 1H), 7.78 (br. s., 1H), 7.45 (dd, J=8.5, 2.1 Hz, 1H), 7.20 (br. s., 1 H), 7.13 (d, J=8.7 Hz, 1H), 6.94 (s, 1H), 2.58 (s, 3H), 2.53 (s, 3H), 1.38 (s, 9H). MS m/e 403.0 (M+H).

### Example 13

### 3-(2′,4′-Dimethyl-[4,5′]bithiazolyl-2-ylamino)-4-propoxy-benzamide

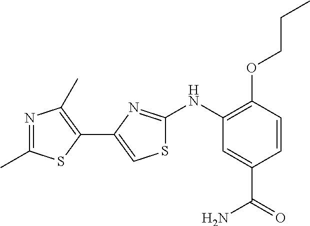

To a suspension of 3-(2′,4′-dimethyl-[4,5′]bithiazolyl-2-ylamino)-4-hydroxy-benzamide (50 mg, 0.144 mmol, intermediate 30, step d) in THF (1 mL) was added in sequence triphenylphosphine (56.8 mg, 0.216 mmol), n-propanol (16.3 μL, 0.216 mmol), and diisopropyl azodicarboxylate (42.6 μL, 0.216 mmol). The resulting light yellow suspension was stirred at room temperature for 20 h. The reaction mixture was partitioned between EtOAc and water and the aqueous phase was extracted with EtOAc. The organic extracts were dried (Na₂SO₄), filtered, and concentrated. The residue was purified by column chromatography (Silica gel, 0-8% MeOH-EtOAc) affording the title compound. ¹H NMR (300 MHz, MeOD) δ ppm 8.91 (d, J=1.9 Hz, 1H), 7.52 (dd, J=8.3, 2.3 Hz, 1H), 7.04 (d, J=8.7 Hz, 1H), 6.85 (s, 1H), 4.12 (t, J=6.6 Hz, 2H), 2.64 (s, 3H), 2.58 (s, 3H), 1.83-1.98 (m, 2H), 1.07 (t, J=7.3 Hz, 3H). MS m/e 389.1 (M+H).

### Example 14

### (±)-4-sec-Butoxy-3-(2′,4′-dimethyl-[4,5′]bithiazolyl-2-ylamino)-benzamide.TFA

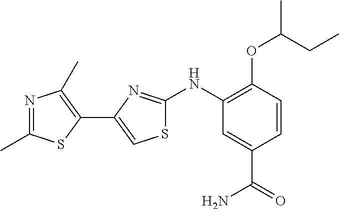

The title compound was prepared using racemic 2-butanol in place of n-propanol by the procedure described in Example 13. An additional reverse phase purification was required (10-90% CH₃CN—H₂O, 0.1% TFA). ¹H NMR (300 MHz, DMSO-d₆) δ ppm 9.43 (s, 1H), 8.80 (d, J=1.9 Hz, 1H), 7.72 (br. s., 1H), 7.51 (dd, J=8.5, 2.1 Hz, 1H), 7.12 (br. s., 1H), 7.07 (d, J=8.7 Hz, 1H), 6.96 (s, 1H), 4.48-4.59 (m, 1H), 2.60 (s, 3H), 2.54 (s, 3H), 1.71-1.85 (m, 1H), 1.54-1.70 (m, 1H), 1.29 (d, J=6.0 Hz, 3H), 0.93 (t, J=7.5 Hz, 3H). MS m/e 403.0 (M+H).

### Example 15

### 4-Cyclopentyloxy-3-(2′,4′-dimethyl-[4,5′]bithiazolyl-2-ylamino)-benzamide

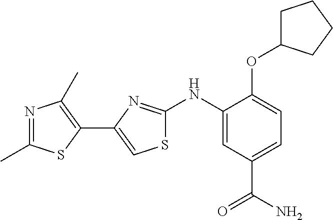

The title compound was prepared using cyclopentanol in place of n-propanol according to the procedure described in Example 13. ¹H NMR (300 MHz, DMSO-d₆) δ 9.36 (s, 1H), 8.75 (d, J=1.88 Hz, 1H), 7.71 (br. s., 1H), 7.51 (dd, J=2.07, 8.48 Hz, 1H), 7.11 (br. s., 1H), 7.02 (d, J=8.67 Hz, 1H), 6.94 (s, 1H), 4.94 (m, 1H), 2.58 (s, 3H), 2.53 (s, 3H), 1.66-2.04 (m, 6H), 1.50-1.63 (m, 2H). MS m/e 415.0 (M+H).

### Example 16

### 3-(2′,4′-Dimethyl-[4,5′]bithiazolyl-2-ylamino)-4-(2,2,2-trifluoro-ethoxy)-benzamide

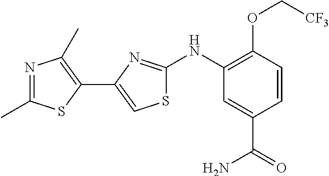

1,1,1-Trifluoro-2-iodoethane (18.3 μL, 0.188 mmol) was added to a suspension of 3-(2′,4′-dimethyl-[4,5′]bithiazolyl-2-ylamino)-4-hydroxy-benzamide (50 mg, 0.144 mmol, intermediate 30, step d) and potassium carbonate (27.9 mg, 0.202 mmol) in dimethylacetamide (0.75 mL). The suspension was heated by microwave irradiation (100° C., 30 min, 300 W). Analysis by TLC indicated incomplete conversion so additional portions of 1,1,1-trifluoro-2-iodoethane and potassium carbonate (amounts as above) were added and heating was continued (100° C., 300 W, 45 min). The reaction mixture was partitioned between EtOAc and water and the aqueous phase was extracted with EtOAc. The organic extracts were dried (Na₂SO₄), filtered, and concentrated. The residue was purified by column chromatography (Silica gel, 0-5% MeOH—CH₂Cl₂) affording the title compound. ¹H NMR (300 MHz, MeOD) δ ppm 9.03 (d, J=2.3 Hz, 1H), 7.53 (dd, J=8.7, 1.9 Hz, 1H), 7.16 (d, J=8.3 Hz, 1H), 6.86 (s, 1H), 4.74 (q, J=8.3 Hz, 2H), 2.64 (s, 3H), 2.58 (s, 3H). MS m/e 428.9 (M+H).

### Example 17

### 3-(2′,4′-Dimethyl-[4,5′]bithiazolyl-2-ylamino)-4-trifluoromethoxy-benzamide

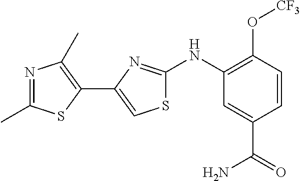

A mixture of 2-bromo-1-(2,4-dimethyl-thiazol-5-yl)-ethanone.HBr (84.6 mg, 0.269 mmol, intermediate 3), 3-thioureido-4-trifluoromethoxy-benzamide (75 mg, 0.269 mmol, intermediate 31, step d), and EtOH (3 mL) was stirred at room temperature for 2 d. The reaction mixture was filtered to collect precipitated solid and the solid was washed with EtOH. The solid was vigorously stirred in a mixture of sat. aq. NaHCO₃ and EtOAc until it dissolved (10 min). The organic phase was dried (Na₂SO₄), filtered, and concentrated, yielding the title compound as a white solid. ¹H NMR (300 MHz, DMSO-d₆) δ ppm 10.21 (s, 1H), 9.09 (d, J=1.5 Hz, 1H), 7.98 (br. s., 1H), 7.38-7.57 (m, 3H), 7.06 (s, 1H), 2.59 (s, 3 H), 2.54 (s, 3H). MS m/e 414.9 (M+H).

### Example 18

### 3-(2′,4′-Dimethyl-[4,5′]bithiazolyl-2-ylamino)-4-methoxy-benzamide.HBr

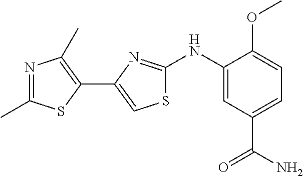

The title compound was prepared using 4-methoxy-3-thioureido-benzamide (intermediate 24) in place of 4-isopropoxy-3-thioureido-benzamide according to the procedure of Example 1 (reaction time 3 d). The crude product was purified by recrystallization from a mixture of EtOH and water, affording the title compound as a tan powder. ¹H NMR (300 MHz, DMSO-d₆) δ 9.77 (s, 1H), 8.85 (d, J=1.88 Hz, 1H), 7.76 (br. s., 1H), 7.56 (dd, J=2.07, 8.48 Hz, 1H), 7.02-7.22 (m, 3H), 3.92 (s, 3H), 2.68 (s, 3H), 2.57 (s, 3H). MS m/e 361.0 (M+H).

### Example 19

### N-[2-(2,3-Dihydro-benzo[1,4]dioxin-5-ylamino)-4′-methyl-[4,5′]bithiazolyl-2′-yl]-acetamide

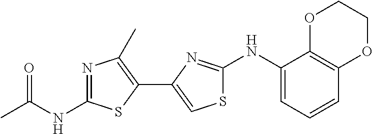

The title compound was prepared using (2,3-dihydro-benzo[1,4]dioxin-5-yl)-thiourea (intermediate 32) in place of (2-methyl-benzofuran-7-yl)-thiourea according to the procedure of Example 9. ¹H NMR (300 MHz, DMSO-d₆) δ 12.04 (s, 1H), 9.63 (s, 1H), 7.89 (dd, J=1.51, 8.29 Hz, 1H), 6.86 (s, 1H), 6.80 (t, J=8.10 Hz, 1H), 6.54 (dd, J=1.51, 8.29 Hz, 1H), 4.25-4.36 (m, 4H), 2.47 (s, 3H), 2.13 (s, 3H). MS m/e 388.8 (M+H).

### Example 20

### N-[2-(5-Diethylsulfamoyl-2-methoxy-phenylamino)-4′-methyl-[4,5]bithiazolyl-2′-yl]-acetamide

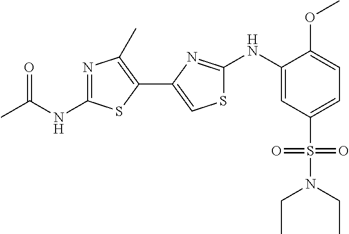

The title compound was prepared using N,N-diethyl-4-methoxy-3-thioureido-benzenesulfonamide (intermediate 33) in place of (2-methyl-benzofuran-7-yl)-thiourea according to the procedure of Example 9 (Silica gel chromatography gradient 30-100% EtOAc-Hept). ¹H NMR (300 MHz, DMSO-d₆) δ 12.05 (s, 1H), 9.97 (s, 1H), 8.97 (d, J=1.88 Hz, 1H), 7.39 (dd, J=2.07, 8.48 Hz, 1H), 7.18 (d, J=8.29 Hz, 1H), 6.96 (s, 1H), 3.96 (s, 3H), 3.21 (q, J=6.91 Hz, 4H), 2.14 (s, 3H), 1.01 (t, J=6.97 Hz, 6H). MS m/e 496.0 (M+H).

### Example 21

### 3-(2′-Amino-4′-methyl-[4,5′]bithiazolyl-2-ylamino)-N,N-diethyl-4-methoxy-benzenesulfonamide.HBr

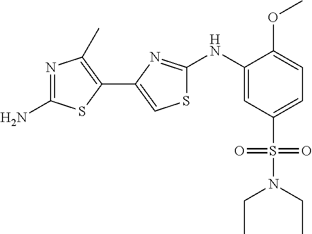

The title compound was prepared using N,N-diethyl-4-methoxy-3-thioureido-benzenesulfonamide (intermediate 33) in place of 4-isopropoxy-3-thioureido-benzamide and 1-(2-amino-4-methyl-thiazol-5-yl)-2-bromo-ethanone.HBr (intermediate 4) in place of 2-bromo-1-(2,4-dimethyl-thiazol-5-yl)-ethanone.HBr according to the procedure of Example 1 (reaction time 1 d). ¹H NMR (400 MHz, DMSO-d₆) δ 10.13 (s, 1H), 9.23 (br. s., 2H), 8.94 (d, J=2.20 Hz, 1H), 7.41 (dd, J=2.20, 8.56 Hz, 1H), 7.20 (d, J=8.56 Hz, 1H), 7.11 (s, 1H), 3.97 (s, 3H), 3.16 (q, J=7.09 Hz, 4H), 2.44 (s, 3H), 1.03 (t, J=7.09 Hz, 6H). MS m/e 454.0 (M+H).

### Example 22

### N²-(2-Methoxy-5-trifluoromethyl-phenyl)-4′-methyl-[4,5′]bithiazolyl-2,2′-diamine.HBr

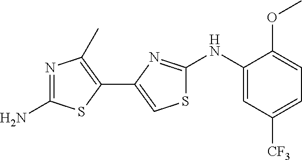

The title compound was prepared using (2-methoxy-5-trifluoromethyl-phenyl)-thiourea (intermediate 34) in place of 4-isopropoxy-3-thioureido-benzamide and 1-(2-amino-4-methyl-thiazol-5-yl)-2-bromo-ethanone (intermediate 4) in place of 2-bromo-1-(2,4-dimethyl-thiazol-5-yl)-ethanone.HBr according to the procedure of Example 1 (reaction time 1 d). ¹H NMR (300 MHz, DMSO-d₆) δ 10.17 (s, 1H), 9.17 (br. s., 2H), 8.91 (s, 1H), 7.34 (d, J=8.67 Hz, 1H), 7.22 (d, J=8.67 Hz, 1H), 7.10 (s, 1H), 3.96 (s, 3H), 2.42 (s, 3H). MS m/e 387.0 (M+H).

### Example 23

### N²-(4-Methoxy-biphenyl-3-yl)-4′-methyl-[4,5′]bithiazolyl-2,2′-diamine.HBr

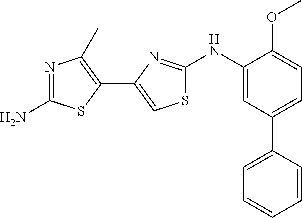

The title compound was prepared using (4-methoxy-biphenyl-3-yl)-thiourea (intermediate 35) in place of 4-isopropoxy-3-thioureido-benzamide and 1-(2-amino-4-methyl-thiazol-5-yl)-2-bromo-ethanone (intermediate 4) in place of 2-bromo-1-(2,4-dimethyl-thiazol-5-yl)-ethanone.HBr according to the procedure of Example 1. ¹H NMR (300 MHz, DMSO-d₆) δ 9.92 (s, 1H), 9.22 (br. s., 2H), 8.75 (d, J=1.88 Hz, 1H), 7.66 (d, J=7.54 Hz, 2H), 7.50 (t, J=7.72 Hz, 2H), 7.26-7.40 (m, 2H), 7.14 (d, J=8.67 Hz, 1H), 7.05 (s, 1H), 3.92 (s, 3H), 2.41 (s, 3H). MS m/e 395.1 (M+H).

### Example 24

### N²-(2-Methoxy-5-methyl-phenyl)-4′-methyl-[4,5′]bithiazolyl-2,2′-diamine.HBr

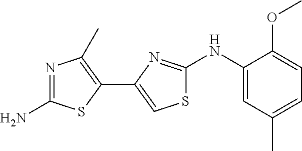

The title compound was prepared using (2-methoxy-5-methyl-phenyl)-thiourea (intermediate 28) in place of 4-isopropoxy-3-thioureido-benzamide and 1-(2-amino-4-methyl-thiazol-5-yl)-2-bromo-ethanone (intermediate 4) in place of 2-bromo-1-(2,4-dimethyl-thiazol-5-yl)-ethanone.HBr according to the procedure of Example 1. ¹H NMR (300 MHz, DMSO-d₆) δ 9.74 (s, 1H), 9.19 (br. s., 2H), 8.16 (s, 1H), 7.00 (s, 1H), 6.89-6.96 (m, 1H), 6.75-6.85 (m, 1H), 3.82 (s, 3H), 2.43 (s, 3H), 2.28 (s, 3H). MS m/e 333.1 (M+H).

### Example 25

### 3-(2′-Amino-4′-methyl-[4,5′]bithiazolyl-2-ylamino)-4-methyl-benzamide.HBr

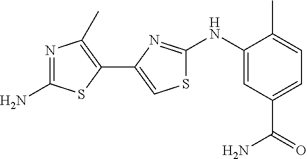

The title compound was prepared using 4-methyl-3-thioureido-benzamide (intermediate 36, step b) in place of 4-isopropoxy-3-thioureido-benzamide and 1-(2-amino-4-methyl-thiazol-5-yl)-2-bromo-ethanone.HBr (intermediate 4) in place of 2-bromo-1-(2,4-dimethyl-thiazol-5-yl)-ethanone.HBr according to the procedure of Example 1 (reaction time 1 d). ¹H NMR (300 MHz, DMSO-d₆) δ 9.61 (s, 1H), 9.15 (br. s., 2H), 8.36 (s, 1H), 7.89 (br. s., 1H), 7.55 (d, J=7.91 Hz, 1H), 7.22-7.34 (m, 2H), 7.00 (s, 1H), 2.40 (s, 3H), 2.30 (s, 3H). MS m/e 346.0 (M+H).

### Example 26

### 3-(2′-Acetylamino-4′-methyl-[4,5′]bithiazolyl-2-ylamino)-4-chloro-benzamide.HBr

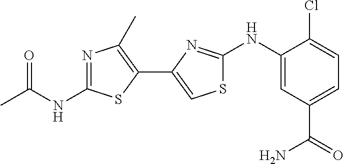

The title compound was prepared using 4-chloro-3-thioureido-benzamide (intermediate 37, step b) in place of 4-isopropoxy-3-thioureido-benzamide and N-[5-(2-bromo-acetyl)-4-methyl-thiazol-2-yl]-acetamide.HBr (intermediate 5) in place of 2-bromo-1-(2,4-dimethyl-thiazol-5-yl)-ethanone.HBr according to the procedure of Example 1 (reaction time 1 d). The crude product was purified by trituration from EtOH/water. ¹H NMR (300 MHz, DMSO-d₆) δ 12.08 (br. s., 1H), 9.85 (br. s., 1H), 8.74 (d, J=1.51 Hz, 1H), 7.98 (br. s., 1H), 7.72 (d, J=1.88 Hz, 1H), 7.52-7.60 (m, 2H), 7.40 (br. s., 1H), 6.97 (s, 1H), 2.49 (s, 3H), 2.13 (s, 3H). MS m/e 408.0; 410.0 (M+H).

### Example 27

### 3-(2′-Amino-[4,5′]bithiazolyl-2-ylamino)-4-methoxy-benzamide.HBr

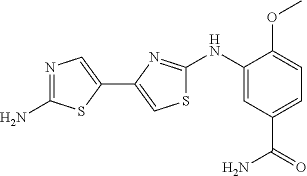

The title compound was prepared using 4-methoxy-3-thioureido-benzamide (intermediate 24) in place of 4-isopropoxy-3-thioureido-benzamide and 1-(2-amino-thiazol-5-yl)-2-bromo-ethanone (intermediate 1) in place of 2-bromo-1-(2,4-dimethyl-thiazol-5-yl)-ethanone.HBr according to the procedure of Example 1 (reaction time 5 d). ¹H NMR (300 MHz, DMSO-d₆) δ ppm 9.81 (s, 1H), 9.23 (br. s., 2H), 8.81 (d, J=2.3 Hz, 1H), 7.82 (br. s., 1H), 7.68 (s, 1H), 7.57 (dd, J=8.7, 1.9 Hz, 1H), 7.24 (s, 1H), 7.04-7.17 (m, 2H), 3.91 (s, 3H). MS m/e 348.0 (M+H).

### Example 28

### 3-(2′,4′-Dimethyl-[4,5′]bithiazolyl-2-ylamino)-4-isobutoxy-benzamide

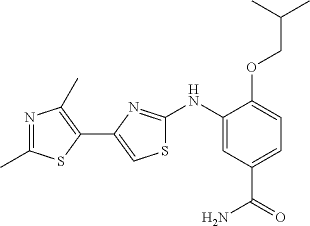

The title compound was prepared using 2-methyl-propan-1-ol in place of n-propanol according to the procedure described in Example 13. ¹H NMR (300 MHz, DMSO-d₆) δ 9.45 (s, 1H), 8.69 (d, J=1.88 Hz, 1H), 7.74 (br. s., 1H), 7.55 (dd, J=2.07, 8.48 Hz, 1H), 7.14 (br. s., 1H), 7.06 (d, J=8.67 Hz, 1H), 6.93 (s, 1H), 3.87 (d, J=6.78 Hz, 2H), 2.58 (s, 3H), 2.52 (s, 3H), 2.11 (sept, J=6.69 Hz, 1H), 0.98 (d, J=6.40 Hz, 6H). MS m/e 403.0 (M+H).

### Example 29

### (2′,4′-Dimethyl-[4,5′]bithiazolyl-2-yl)-(2-methyl-benzofuran-7-yl)-amine

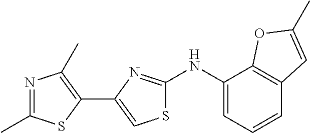

The title compound was prepared using (2-methyl-benzofuran-7-yl)-thiourea (intermediate 26) in place of 3-thioureido-4-trifluoromethoxy-benzamide as described in Example 17 (reaction time 1 d). ¹H NMR (400 MHz, DMSO-d₆) δ 10.42 (s, 1H), 8.10 (dd, J=2.08, 6.97 Hz, 1H), 7.11-7.20 (m, 2H), 6.98 (s, 1H), 6.61 (d, J=0.98 Hz, 1H), 2.60 (s, 3H), 2.52 (s, 3H), 2.49 (s, 3H). MS m/e 342.1 (M+H).

### Example 30

### 2-[3-(2′,4′-Dimethyl-[4,5′]bithiazolyl-2-ylamino)-4-methoxy-phenyl]-acetamide

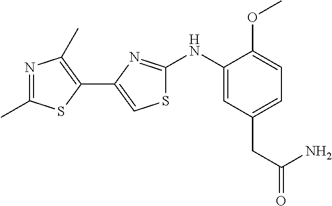

The title compound was prepared using 2-(4-methoxy-3-thioureido-phenyl)-acetamide (intermediate 38, step d) in place of 3-thioureido-4-trifluoromethoxy-benzamide as described in Example 17 (reaction time 1 d). ¹H NMR (400 MHz, DMSO-d₆) δ 9.61 (s, 1H), 8.25 (d, J=1.96 Hz, 1H), 7.38 (br. s., 1H), 6.85-6.98 (m, 4H), 3.83 (s, 3H), 3.34 (s, 2H), 2.59 (s, 3H), 2.53 (s, 3H). MS m/e 375.0 (M+H).

### Example 31

### 6-(2′,4′-Dimethyl-[4,5′]bithiazolyl-2-ylamino)-5-methoxy-2,3-dihydro-isoindol-1-one

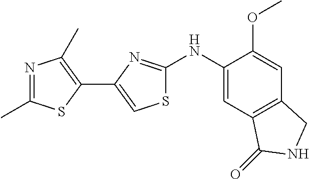

The title compound was prepared using (6-methoxy-3-oxo-2,3-dihydro-1H-isoindol-5-yl)-thiourea (intermediate 39, step d) in place of 3-thioureido-4-trifluoromethoxy-benzamide as described in Example 17 (reaction time 1 d). The crude product was purified by column chromatography (Silica gel, 2-8% MeOH—CH₂Cl₂) affording the title compound as a light yellow solid. ¹H NMR (400 MHz, DMSO-d₆) δ 10.02 (s, 1H), 8.70 (s, 1H), 8.32 (s, 1H), 7.22 (s, 1H), 7.03 (s, 1H), 4.31 (s, 2H), 3.95 (s, 3H), 2.61 (s, 3H), 2.53 (s, 3H). MS m/e 373.1 (M+H).

### Example 32

### 1-Methyl-piperidine-4-carboxylic acid [2-(5-carbamoyl-2-isopropoxy-phenylamino)-4′-methyl-[4,5′]bithiazolyl-2′-yl]-amide

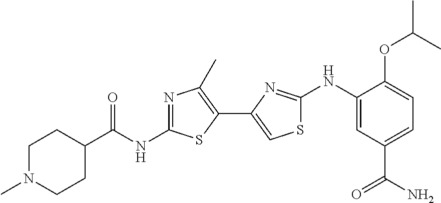

The title compound was prepared using 4-isopropoxy-3-thioureido-benzamide (intermediate 23, step e) in place of 3-thioureido-4-trifluoromethoxy-benzamide and 1-methyl-piperidine-4-carboxylic acid [5-(2-bromo-acetyl)-4-methyl-thiazol-2-yl]-amide (intermediate 2, step b) in place of 2-bromo-1-(2,4-dimethyl-thiazol-5-yl)-ethanone.HBr as described in Example 17 (reaction time 4 d). The crude product was purified by column chromatography (Biotage KP-NH column, 0-8% MeOH-EtOAc) affording the title compound as a white powder. ¹H NMR (400 MHz, DMSO-d₆) δ 12.04 (br. s., 1H), 9.37 (s, 1H), 8.74 (d, J=1.96 Hz, 1H), 7.73 (br. s., 1H), 7.52 (dd, J=1.96, 8.56 Hz, 1H), 7.11 (br. s., 1H) 7.08 (d, J=8.56 Hz, 1H), 6.86 (s, 1H), 4.76 (sept, J=5.99 Hz, 1H), 2.80 (d, J=11.25 Hz, 2H), 2.52 (s, 3H), 2.15 (s, 3H), 1.57-1.91 (m, 6H), 1.34 (d, J=5.87 Hz, 6H). MS m/e 515.2 (M+H).

### Example 33

### 7-(2′,4′-Dimethyl-[4,5′]bithiazolyl-2-ylamino)-2,3-dihydro-benzofuran-5-sulfonic acid amide

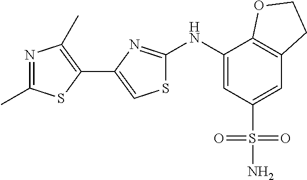

A mixture of 2-bromo-1-(2,4-dimethyl-thiazol-5-yl)-ethanone.HBr (78.8 mg, 0.250 mmol, intermediate 3) and 7-thioureido-2,3-dihydro-benzofuran-5-sulfonic acid amide (68.3 mg, 0.250 mmol, intermediate 40, step b) was heated by microwave irradiation (80° C., 10 min, 300 W). The reaction mixture was filtered and the solid was stirred vigorously in a mixture of EtOAc and sat. aq. NaHCO₃. The separated aq. phase was extracted twice with EtOAc. The organic phase was dried (Na₂SO₄), filtered, and concentrated, affording the title compound as a light yellow powder. ¹H NMR (400 MHz, DMSO-d₆) δ 10.06 (s, 1H), 8.74 (s, 1H), 7.38 (s, 1H), 7.11 (s, 2H), 6.97 (s, 1H), 4.73 (t, J=8.68 Hz, 2H), 3.31 (t, J=8.80 Hz, 2H), 2.59 (s, 3H), 2.53 (s, 3H). MS m/e 409.0 (M+H).

### Example 34

### 3-(2′,4′-Dimethyl-[4,5′]bithiazolyl-2-ylamino)-5-fluoro-4-methoxy-benzamide

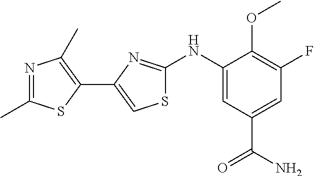

The title compound was prepared using 3-fluoro-4-methoxy-5-thioureido-benzamide (intermediate 41, step d) in place of 3-thioureido-4-trifluoromethoxy-benzamide as described in Example 17 (reaction time 1 d). ¹H NMR (400 MHz, DMSO-d₆) δ 10.04 (s, 1H), 8.86 (s, 1H), 7.85 (br. s., 1H), 7.39 (dd, J=1.96, 11.98 Hz, 1H), 7.36 (br. s., 1H), 7.02 (s, 1H), 3.92 (s, 3H), 2.59 (s, 3H), 2.55 (s, 3H). MS m/e 379.0 (M+H).

### Example 35

### 4-(2′,4′-Dimethyl-[4,5′]bithiazolyl-2-ylamino)-3-methoxy-benzamide

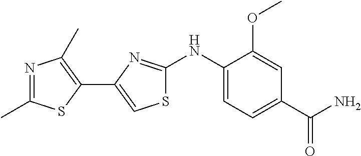

The title compound was prepared using 3-methoxy-4-thioureido-benzamide (intermediate 42, step d) in place of 7-thioureido-2,3-dihydro-benzofuran-5-sulfonic acid amide according to the procedure of Example 33 (reaction temperature 100° C.). ¹H NMR (400 MHz, DMSO-d₆) δ 9.97 (s, 1H), 8.55 (d, J=8.07 Hz, 1H), 7.88 (br. s., 1H), 7.49-7.57 (m, 2H), 7.23 (br. s., 1H), 7.01 (s, 1H), 3.93 (s, 3H), 2.60 (s, 3H), 2.53 (s, 3H). MS m/e 361.2 (M+H).

### Example 36

### 5-(2′,4′-Dimethyl-[4,5′]bithiazolyl-2-ylamino)-4-methoxy-2-methyl-benzamide.TFA

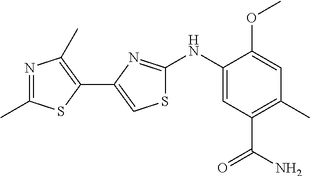

The title compound was prepared using 4-methoxy-2-methyl-5-thioureido-benzamide (intermediate 43, step e) in place of 3-thioureido-4-trifluoromethoxy-benzamide according to the procedure of Example 17 (reaction time 1 d). The crude product was purified by column chromatography (Silica gel, 0-1% MeOH-EtOAc), then by reverse phase HPLC (10-90% CH₃CN—H₂O, 0.1% TFA), affording the title compound as a white solid. ¹H NMR (400 MHz, DMSO-d₆) δ 9.66 (s, 1H), 8.49 (s, 1H), 7.48 (br. s., 1H), 7.21 (br. s., 1H), 6.92 (s, 1H), 6.88 (s, 1H), 3.87 (s, 3H), 2.59 (s, 3H), 2.52 (s, 3H), 2.36 (s, 3H). MS m/e 375.1 (M+H).

### Example 37

### 4-Methoxy-2-methyl-5-[4′-methyl-2′-(4-methyl-piperazin-1-yl)-[4,5]bithiazolyl-2-ylamino]-benzamide.HCl

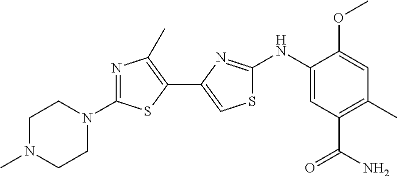

A mixture of 4-methoxy-2-methyl-5-thioureido-benzamide (30.6 mg, 0.128 mmol, intermediate 43, step e), 2-bromo-1-[4-methyl-2-(4-methyl-piperazin-1-yl)-thiazol-5-yl]-ethanone.HBr (51 mg, 0.128 mmol, intermediate 7, step c), and EtOH (0.3 mL) was stirred at room temperature overnight. The mixture was partitioned between sat. aq. NaHCO₃ and EtOAc. The aq. phase was extracted with EtOAc. The organic phase was dried (Na₂SO₄), filtered, and concentrated. The residue was purified by reverse phase HPLC (5-50% CH₃CN—H₂O, 0.1% TFA). The appropriate fractions were concentrated and the residue was concentrated from EtOH/1 N aq HCl three times and once from EtOH, affording the title compound as a tan solid. ¹H NMR (400 MHz, DMSO-d₆) δ 10.77 (br. s., 1H), 9.63 (s, 1H), 8.53 (s, 1H), 7.44 (br. s., 1H), 7.25 (br. s., 1H), 6.88 (s, 1H), 6.73 (s, 1H), 4.00 (d, J=13.45 Hz, 2H), 3.87 (s, 3H), 3.37-3.53 (m, 4H), 3.10-3.23 (m, 2H), 2.82 (d, J=4.40 Hz, 3H), 2.41 (s, 3H), 2.36 (s, 3H). MS m/e 459.1 (M+H).

### Example 38

### 3-(2′,4′-Dimethyl-[4,5′]bithiazolyl-2-ylamino)-4-isobutyl-benzamide.TFA

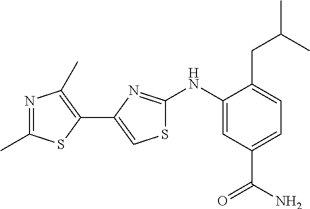

The title compound was prepared using 4-isobutyl-3-thioureido-benzamide (intermediate 44, step e) in place of 4-methoxy-2-methyl-5-thioureido-benzamide and 2-bromo-1-(2,4-dimethyl-thiazol-5-yl)-ethanone.HBr (intermediate 3) in place of 2-bromo-1-[4-methyl-2-(4-methyl-piperazin-1-yl)-thiazol-5-yl]-ethanone.HBr according to the procedure of Example 37 (reaction time 4 d). The crude product was purified by reverse phase HPLC (10-90% CH₃CN—H₂O, 0.1% TFA) affording the title compound as a light yellow solid. ¹H NMR (400 MHz, DMSO-d₆) δ 9.52 (s, 1H), 8.28 (d, J=1.71 Hz, 1H), 7.88 (br. s., 1H), 7.59 (dd, J=1.71, 7.83 Hz, 1H), 7.29 (br. s., 1H), 7.26 (d, J=8.07 Hz, 1H), 6.88 (s, 1H), 2.57-2.61 (m, 5H), 2.49 (s, 3H), 1.80-1.92 (m, 1H), 0.85 (d, J=6.60 Hz, 6H). MS m/e 387.1 (M+H).

### Example 39

### 3-(2′,4′-Dimethyl-[4,5′]bithiazolyl-2-ylamino)-4-methoxy-benzonitrile.TFA

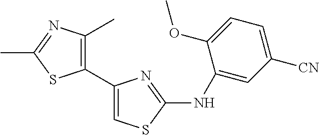

A solution of 2-bromo-1-(2,4-dimethyl-thiazol-5-yl)-ethanone.HBr (0.038 g, 0.121 mmol, intermediate 3) and (5-cyano-2-methoxy-phenyl)-thiourea (0.025 g, 0.121 mmol, intermediate 45, step c) in ethanol (3 mL) was stirred at room temperature overnight. The reaction mixture was then filtered and the solid was washed with ethanol. The solid was purified via reverse phase HPLC with water/acetonitrile/0.1% TFA to give the title compound. ¹H NMR (300 MHz, MeOD) δ 8.94 (d, J=2.26 Hz, 1H), 7.37 (dd, J=1.88, 8.29 Hz, 1H), 7.14 (d, J=8.67 Hz, 1H), 7.08 (s, 1H), 4.01 (s, 3H), 2.81 (s, 3H), 2.66 (s, 3H); MS m/e 342.9 (M+H).

### Example 40

### 3-(2′-Amino-4′-methyl-[4,5′]bithiazolyl-2-ylamino)-4-methoxy-benzonitrile.HBr

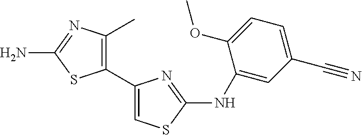

A solution of 1-(2-amino-4-methyl-thiazol-5-yl)-2-bromo-ethanone (0.028 g, 0.121 mmol, intermediate 4) and (5-cyano-2-methoxy-phenyl)-thiourea (0.025 g, 0.121 mmol, intermediate 45, step c) in ethanol (3 mL) was stirred at room temperature overnight. The reaction mixture was then filtered and washed with ethanol and dried to give the title compound. ¹H NMR (300 MHz, DMSO-d₆) δ 10.19 (s, 1H), 9.10 (br. s., 2H), 8.81 (d, J=1.88 Hz, 1H), 7.49 (dd, J=2.07, 8.48 Hz, 1H), 7.22 (d, J=8.29 Hz, 1H), 7.10 (s, 1H), 3.97 (s, 3H), 2.42 (s, 3H); MS m/e 344.0 (M+H).

### Example 41

### (2′,4′-Dimethyl-[4,5′]bithiazolyl-2-yl)-(6-methoxy-quinolin-8-yl)-amine.TFA

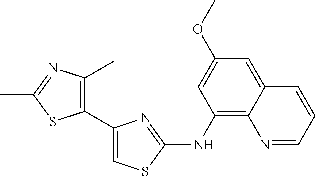

The title compound was prepared using (6-methoxy-quinolin-8-yl)-thiourea (intermediate 46) in place of (5-cyano-2-methoxy-phenyl)-thiourea according to the procedure described in Example 39. ¹H NMR (400 MHz, DMSO-d₆) δ 10.91 (s, 1H), 8.75 (dd, J=1.71, 4.16 Hz, 1H), 8.62 (d, J=2.69 Hz, 1H), 8.27 (dd, J=1.47, 8.31 Hz, 1H), 7.57 (dd, J=4.16, 8.31 Hz, 1H), 7.10 (s, 1H), 6.97 (d, J=2.45 Hz, 1H), 3.93 (s, 3H), 2.64 (s, 3H), 2.53-2.59 (m, 3H); MS m/e 369.1 (M+H).

### Example 42

### (2′,4′-Dimethyl-[4,5′]bithiazolyl-2-yl)-quinolin-8-yl-amine.TFA

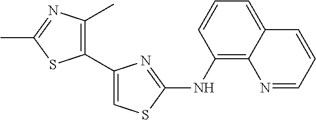

The title compound was prepared using quinolin-8-yl-thiourea (intermediate 47) in place of (5-cyano-2-methoxy-phenyl)-thiourea according to the procedure described in Example 39. ¹H NMR (400 MHz, DMSO-d₆) δ 10.87 (s, 1H), 8.95 (dd, J=1.59, 4.28 Hz, 1H), 8.85 (dd, J=1.34, 7.70 Hz, 1H), 8.41 (dd, J=1.71, 8.31 Hz, 1H), 7.47-7.73 (m, 3H), 7.10 (s, 1H), 2.64 (s, 3H), 2.56 (s, 3H); MS m/e 339.1 (M+H).

### Example 43

### N-[2-(2-Isopropoxy-5-methanesulfonyl-phenylamino)-4′-methyl-[4,5]bithiazolyl-2′-yl]-acetamide.TFA

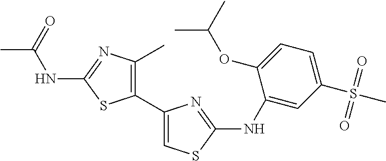

The title compound was prepared using N-[5-(2-bromo-acetyl)-4-methyl-thiazol-2-yl]-acetamide.HBr (intermediate 5) and (2-isopropoxy-5-methanesulfonyl-phenyl)-thiourea (intermediate 50, step d) in place of 2-bromo-1-(2,4-dimethyl-thiazol-5-yl)-ethanone.HBr and (5-cyano-2-methoxy-phenyl)-thiourea, respectively, according to the procedure described in Example 39. ¹H NMR (300 MHz, DMSO-d₆) δ 12.07 (s, 1H), 9.75 (s, 1H), 9.09 (d, J=2.26 Hz, 1H), 7.48 (dd, J=2.26, 8.67 Hz, 1H), 7.27 (d, J=8.67 Hz, 1H), 6.98 (s, 1H), 4.75-4.94 (m, 1H), 3.19 (s, 3H), 2.53 (s, 3H), 2.14 (s, 3H), 1.38 (d, J=6.03 Hz, 6H); MS m/e 467.0 (M+H).

### Example 44

### N²-(2-Isopropoxy-5-methanesulfonyl-phenyl)-4′-methyl-[4,5′]bithiazolyl-2,2′-diamine.TFA

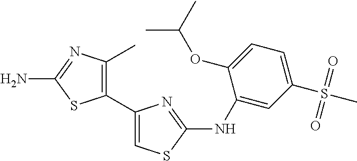

The title compound was prepared using 1-(2-amino-4-methyl-thiazol-5-yl)-2-bromo-ethanone (intermediate 4) and (2-isopropoxy-5-methanesulfonyl-phenyl)-thiourea (intermediate 50, step d) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 9.87 (s, 1H), 9.11 (d, J=2.26 Hz, 1H), 8.76 (br. s., 2H), 7.56 (dd, J=2.26, 8.67 Hz, 1H), 7.35 (d, J=8.67 Hz, 1H), 7.07 (s, 1H), 4.72-5.06 (m, 1H), 3.18-3.25 (m, 3H), 2.50 (s, 3H), 1.45 (d, J=6.03 Hz, 6H); MS ES+ 425.2 (M+H).

### Example 45

### (2′,4′-Dimethyl-[4,5′]bithiazolyl-2-yl)-(2-isopropoxy-5-methanesulfonyl-phenyl)-amine.TFA

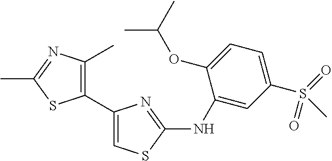

The title compound was prepared using (2-isopropoxy-5-methanesulfonyl-phenyl)-thiourea (intermediate 50, step d) in place of (5-cyano-2-methoxy-phenyl)-thiourea according to the procedure described in Example 39, except the reaction mixture was concentrated instead of filtering. ¹H NMR (300 MHz, MeOD) δ 9.27 (d, J=2.26 Hz, 1H), 7.55 (dd, J=2.26, 8.29 Hz, 1H), 7.21 (d, J=8.67 Hz, 1H), 7.05 (s, 1H), 4.80-4.86 (m, 1H), 3.12 (s, 3H), 2.78 (s, 3H), 2.66 (s, 3H), 1.45 (d, J=6.03 Hz, 6H); MS m/e 424.0 (M+H).

### Example 46

### 3-(2′-Amino-4′-methyl-[4,5′]bithiazolyl-2-ylamino)-4-ethoxy-benzoic acid.HBr

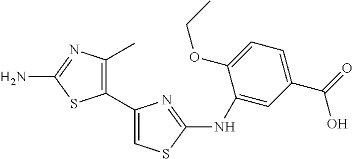

The title compound was prepared using 4-ethoxy-3-thioureido-benzoic acid (intermediate 49) in place of (5-cyano-2-methoxy-phenyl)-thiourea according to the procedure described in Example 40. ¹H NMR (300 MHz, DMSO-d₆) δ 12.31 (br. s., 1H), 9.48 (s, 1H), 8.83 (br. s., 2H), 8.71 (d, J=2.26 Hz, 1H), 7.36 (dd, J=2.07, 8.48 Hz, 1H), 6.86 (d, J=8.67 Hz, 1H), 6.79 (s, 1H), 3.97 (q, J=7.16 Hz, 2H), 2.21 (s, 3H), 1.17 (t, J=6.97 Hz, 3H); MS m/e 377.1 (M+H).

### Example 47

### 3-(2′,4′-Dimethyl-[4,5′]bithiazolyl-2-ylamino)-4-ethoxy-benzoic acid.HBr

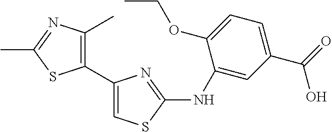

The title compound was prepared using 2-bromo-1-(2,4-dimethyl-thiazol-5-yl)-ethanone.HBr (intermediate 3) and 4-ethoxy-3-thioureido-benzoic acid (intermediate 49) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 9.67 (s, 1H), 9.12 (d, J=1.88 Hz, 1H), 7.59 (dd, J=2.07, 8.48 Hz, 1H), 7.10 (d, J=8.67 Hz, 1H), 7.00 (s, 1H), 4.10-4.31 (m, 2H), 2.61 (s, 3H), 2.56 (s, 3H), 1.43 (t, J=6.97 Hz, 3H); MS m/e 376.0 (M+H).

### Example 48

### 3-(2′-Acetylamino-4′-methyl-[4,5′]bithiazolyl-2-ylamino)-4-ethoxy-benzoic acid.HBr

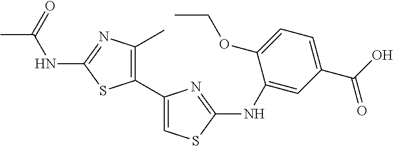

The title compound was prepared using N-[5-(2-bromo-acetyl)-4-methyl-thiazol-2-yl]-acetamide.HBr (intermediate 5) and 4-ethoxy-3-thioureido-benzoic acid (intermediate 49) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 12.52 (br. s., 1H), 12.05 (s, 1H), 9.59 (s, 1H), 9.01 (d, J=1.88 Hz, 1H), 7.59 (dd, J=2.07, 8.48 Hz, 1H), 7.10 (d, J=8.67 Hz, 1H), 6.91 (s, 1H), 4.22 (q, J=7.16 Hz, 2H), 2.54 (s, 3H), 2.13 (s, 3H), 1.43 (t, J=6.97 Hz, 3H); MS m/e 419.1 (M+H).

### Example 49

### N-[2-(5-Ethanesulfonyl-2-methoxy-phenylamino)-4′-methyl-[4,5]bithiazolyl-2′-yl]-acetamide.TFA

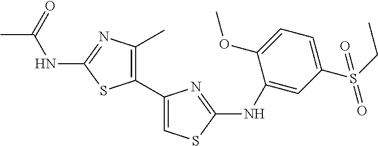

The title compound was prepared using N-[5-(2-bromo-acetyl)-4-methyl-thiazol-2-yl]-acetamide.HBr (intermediate 5) and (5-ethanesulfonyl-2-methoxy-phenyl)-thiourea (intermediate 51, step b) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 12.07 (s, 1H), 10.06 (s, 1H), 9.04 (d, J=2.26 Hz, 1H), 7.35-7.59 (m, 1H), 7.26 (d, J=8.67 Hz, 1H), 6.96 (s, 1H), 3.99 (s, 3H), 3.25 (d, J=7.16 Hz, 2H), 2.52 (s, 3H), 2.14 (s, 3H), 1.17 (t, J=7.35 Hz, 3H); MS m/e 452.9 (M+H).

### Example 50

### (2′,4′-Dimethyl-[4,5′]bithiazolyl-2-yl)-(5-ethanesulfonyl-2-methoxy-phenyl)-amine.TFA

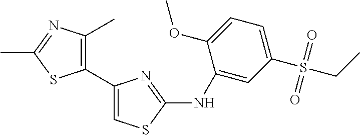

The title compound was prepared using (5-ethanesulfonyl-2-methoxy-phenyl)-thiourea (intermediate 51, step b) in place of (5-cyano-2-methoxy-phenyl)-thiourea according to the procedure described in Example 39, except the reaction mixture was concentrated instead of filtering. ¹H NMR (300 MHz, DMSO-d₆) δ 10.11 (s, 1H), 9.11 (d, J=2.26 Hz, 1H), 7.48 (dd, J=2.26, 8.29 Hz, 1H), 7.26 (d, J=8.67 Hz, 1H), 7.04 (s, 1H), 3.99 (s, 3H), 3.21 (q, J=7.41 Hz, 2H), 2.60 (s, 3H), 2.55 (s, 3H), 1.13 (t, J=7.16 Hz, 3H); MS m/e 410.1 (M+H).

### Example 51

### N²-(5-Ethanesulfonyl-2-methoxy-phenyl)-4′-methyl-[4,5′]bithiazolyl-2,2′-diamine.TFA

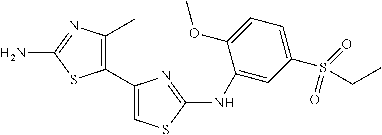

The title compound was prepared using 1-(2-amino-4-methyl-thiazol-5-yl)-2-bromo-ethanone (intermediate 4) and (5-ethanesulfonyl-2-methoxy-phenyl)-thiourea (intermediate 51, step b) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 10.04 (s, 1H), 8.91 (d, J=2.26 Hz, 1H), 8.73 (br. s., 2H), 7.39 (dd, J=2.26, 8.29 Hz, 1H), 7.17 (d, J=8.67 Hz, 1H), 6.91 (s, 1H), 3.89 (s, 3H), 3.10 (q, J=7.28 Hz, 2H), 2.35 (s, 3H), 1.03 (t, J=7.35 Hz, 3H); MS m/e 411.0 (M+H).

### Example 52

### 3-(2′-Amino-4′-methyl-[4,5′]bithiazolyl-2-ylamino)-4-methoxy-benzenesulfonamide.HBr

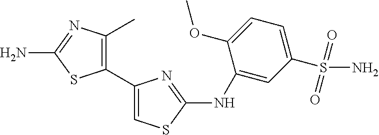

The title compound was prepared using 4-methoxy-3-thioureido-benzenesulfonamide (intermediate 52, step c) in place of (5-cyano-2-methoxy-phenyl)-thiourea according to the procedure described in Example 40. ¹H NMR (300 MHz, DMSO-d₆) δ ppm 10.04 (s, 1H) 9.13 (d, J=6.0 Hz, 1H) 8.94 (d, J=2.3 Hz, 1H) 7.47 (dd, J=8.3, 2.3 Hz, 1H) 7.20 (d, J=8.7 Hz, 1H) 7.14 (br. s., 2H) 7.06 (s, 1H) 3.95 (s, 3H) 2.45 (s, 3H); MS m/e 398.1 (M+H).

### Example 53

### N-[2-(2-Methoxy-5-sulfamoyl-phenylamino)-4′-methyl-[4,5′]bithiazolyl-2′-yl]-acetamide.HBr

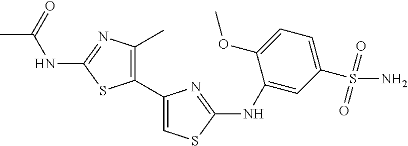

The title compound was prepared using N-[5-(2-bromo-acetyl)-4-methyl-thiazol-2-yl]-acetamide.HBr (intermediate 5) and 4-methoxy-3-thioureido-benzenesulfonamide (intermediate 52, step c) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ ppm 11.87-12.32 (m, 1H) 9.89 (s, 1H) 8.96 (d, J=2.3 Hz, 1H) 7.46 (dd, J=8.3, 2.3 Hz, 1H) 7.19 (d, J=8.7 Hz, 1H) 7.06 (br. s., 2H) 6.93 (s, 1H) 3.95 (s, 3H) 2.54 (s, 3H) 2.14 (s, 3H); MS m/e 440.1 (M+H).

### Example 54

### 3-(2′,4′-Dimethyl-[4,5′]bithiazolyl-2-ylamino)-4-methoxy-benzenesulfonamide.TFA

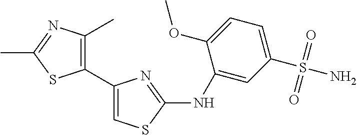

The title compound was prepared using 4-methoxy-3-thioureido-benzenesulfonamide (intermediate 52, step c) in place of (5-cyano-2-methoxy-phenyl)-thiourea according to the procedure described in Example 39, except the reaction mixture was concentrated instead of filtering. ¹H NMR (300 MHz, DMSO-d₆) δ 9.93 (s, 1H), 9.02 (d, J=1.88 Hz, 1H), 7.46 (dd, J=2.26, 8.29 Hz, 1H), 7.19 (d, J=8.67 Hz, 1H), 7.14 (br. s., 2H), 7.01 (s, 1H), 3.95 (s, 3H), 2.61 (s, 3H), 2.55 (s, 3H); MS m/e 397.0 (M+H).

### Example 55

### 3-(2′-Amino-4′-methyl-[4,5′]bithiazolyl-2-ylamino)-4-methoxy-benzoic acid.HBr

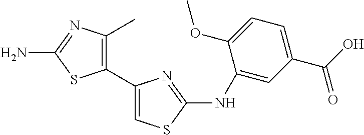

The title compound was prepared using 4-methoxy-3-thioureido-benzoic acid (intermediate 53) in place of (5-cyano-2-methoxy-phenyl)-thiourea according to the procedure described in Example 40. ¹H NMR (300 MHz, DMSO-d₆) δ ppm 12.55 (br. s., 1H) 9.90 (s, 1H) 9.07 (br. s., 2H) 8.98 (d, J=2.3 Hz, 1H) 7.63 (dd, J=8.5, 2.1 Hz, 1H) 7.13 (d, J=8.7 Hz, 1H) 7.03 (s, 1H) 3.94 (s, 3H) 2.46 (s, 3H); MS m/e 363.0 (M+H).

### Example 56

### 3-(2′-Amino-4′-methyl-[4,5′]bithiazolyl-2-ylamino)-N-benzyl-4-methoxy-benzamide.TFA

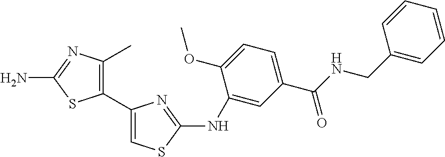

HATU (0.054 g, 0.144 mmol) was added to a solution of 3-(2′-amino-4′-methyl-[4,5]bithiazolyl-2-ylamino)-4-methoxy-benzoic acid (0.050 g, 0.144 mmol, Example 55) and commercially available benzylamine (0.031 mL, 0.288 mmol) in DMF (1 mL) at room temperature and stirred overnight. The reaction mixture was then purified via reverse phase HPLC with water/acetonitrile/0.1% TFA to give the title compound. ¹H NMR (300 MHz, DMSO-d₆) δ 9.78 (s, 1H), 8.63-9.03 (m, 4H), 7.58 (dd, J=2.07, 8.48 Hz, 1H), 7.17-7.38 (m, 5H), 7.10 (d, J=8.29 Hz, 1H), 6.93 (s, 1H), 4.47 (d, J=6.03 Hz, 2H), 3.92 (s, 3H), 2.40 (s, 3H). MS m/e 452.1 (M+H).

### Example 57

### 3-(2′-Acetylamino-4′-methyl-[4,5′]bithiazolyl-2-ylamino)-4-methoxy-benzoic acid.HBr

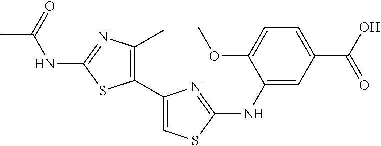

The title compound was prepared using N-[5-(2-bromo-acetyl)-4-methyl-thiazol-2-yl]-acetamide.HBr (intermediate 5) and 4-methoxy-3-thioureido-benzoic acid (intermediate 53) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 12.54 (br. s., 1H), 12.06 (s, 1H), 9.76 (s, 1H), 9.05 (d, J=2.26 Hz, 1H), 7.61 (dd, J=2.07, 8.48 Hz, 1H), 7.12 (d, J=8.67 Hz, 1H), 6.90 (s, 1H), 3.94 (s, 3H), 2.54 (s, 3H), 2.13 (s, 3H); MS m/e 405.1 (M+H).

### Example 58

### 3-(2′-Acetylamino-4′-methyl-[4,5′]bithiazolyl-2-ylamino)-4-methoxy-N-(2-piperidin-1-yl-ethyl)-benzamide.TFA

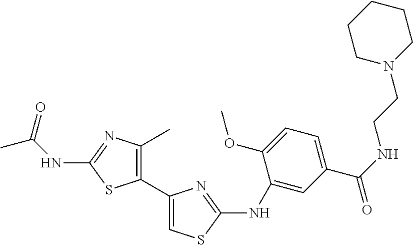

The title compound was prepared using 3-(2′-acetylamino-4′-methyl-[4,5′]bithiazolyl-2-ylamino)-4-methoxy-benzoic acid (Example 57) and commercially available 1-(2-aminoethyl)piperidine in place of 3-(2′-amino-4′-methyl-[4,5′]bithiazolyl-2-ylamino)-4-methoxy-benzoic acid and benzylamine, respectively, according to the procedure described in Example 56. ¹H NMR (300 MHz, DMSO-d₆) δ ppm 11.93 (s, 1H) 9.61 (s, 1H) 8.95 (br. s., 1H) 8.76 (d, J=1.9 Hz, 1H) 8.36 (t, J=5.5 Hz, 1H) 7.36 (dd, J=8.5, 1.7 Hz, 1H) 6.98 (d, J=8.3 Hz, 1H) 6.76 (s, 1H) 3.79 (s, 3H) 3.36-3.53 (m, 4H) 3.00-3.18 (m, 2H) 2.71-2.87 (m, 2H) 1.99 (s, 3H) 1.61-1.74 (m, 2H) 1.39-1.61 (m, 3H) 1.14-1.32 (m, 1H); MS m/e 515.2 (M+H).

### Example 59

### 3-(2′-Acetylamino-4′-methyl-[4,5′]bithiazolyl-2-ylamino)-N-benzyl-4-methoxy-benzamide.TFA

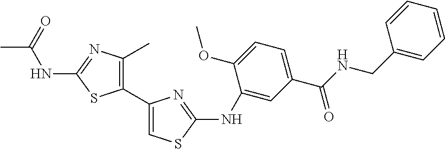

The title compound was prepared using 3-(2′-acetylamino-4′-methyl-[4,5′]bithiazolyl-2-ylamino)-4-methoxy-benzoic acid (Example 57) in place of 3-(2′-amino-4′-methyl-[4,5]bithiazolyl-2-ylamino)-4-methoxy-benzoic acid according to the procedure described in Example 56. ¹H NMR (DMSO-d₆) δ: 11.83 (s, 1H), 9.43 (s, 1H), 8.48-8.63 (m, 2H), 7.33 (dd, J=8.5, 2.1 Hz, 1H), 6.93-7.16 (m, 5H), 6.87 (d, J=8.3 Hz, 1H), 6.56-6.70 (m, 1H), 4.23 (d, J=6.0 Hz, 2H), 3.68 (s, 3H), 1.89 (s, 3H); MS m/e 494.1 (M+H).

### Example 60

### 3-(2′-Amino-4′-methyl-[4,5′]bithiazolyl-2-ylamino)-4-isopropoxy-benzenesulfonamide.TFA

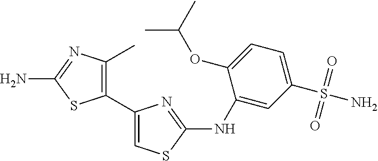

The title compound was prepared using 1-(2-amino-4-methyl-thiazol-5-yl)-2-bromo-ethanone.HBr (intermediate 4) and 4-isopropoxy-3-thioureido-benzenesulfonamide (intermediate 55, step e) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 9.70 (s, 1H), 9.11 (br. s., 2H), 8.92 (d, J=2.26 Hz, 1H), 7.44 (dd, J=2.07, 8.48 Hz, 1H), 7.22 (d, J=8.67 Hz, 1H), 7.11 (s, 2H), 7.03 (s, 1H), 4.63-4.89 (m, 1H), 2.44 (s, 3H), 1.36 (d, J=6.03 Hz, 6H); MS m/e 426.0 (M+H).

### Example 61

### 4-Isopropoxy-3-[4′-methyl-2′-(4-methyl-piperazin-1-yl)-[4,5′]bithiazolyl-2-ylamino]-benzenesulfonamide.TFA

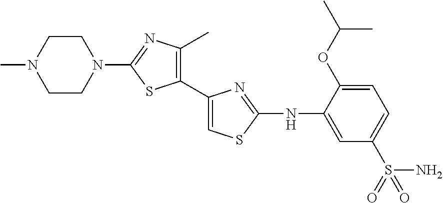

The title compound was prepared using 2-bromo-1-[4-methyl-2-(4-methyl-piperazin-1-yl)-thiazol-5-yl]-ethanone.HBr (intermediate 7, step c) and 4-isopropoxy-3-thioureido-benzenesulfonamide (intermediate 55, step e) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 9.60 (s, 1H), 9.04 (d, J=2.26 Hz, 1H), 7.42 (dd, J=2.26, 8.67 Hz, 1H), 7.21 (d, J=8.67 Hz, 1H), 7.12 (br. s., 2H), 6.81 (s, 1H), 4.80 (sept, J=5.79 Hz, 1H), 3.97-4.07 (m, 2H), 3.46-3.60 (m, 2H), 3.30 (br. s., 4H), 2.86 (s, 3H), 2.42 (s, 3H), 1.36 (d, J=6.03 Hz, 6H); MS m/e 509.0 (M+H).

### Example 62

### 3-(5-Fluoro-2′,4′-dimethyl-[4,5′]bithiazolyl-2-ylamino)-4-isopropoxy-benzenesulfonamide.TFA

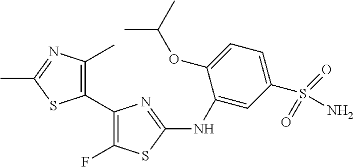

Selectfluor® (0.008 g, 0.023 mmol) was added to a solution of 3-(2′,4′-dimethyl-[4,5]bithiazolyl-2-ylamino)-4-isopropoxy-benzenesulfonamide (0.010 g, 0.023 mmol, Example 64) in dimethylformamide (0.5 mL) and was stirred at room temperature overnight. The reaction mixture was then purified via HPLC eluting with water/acetonitrile/0.1% TFA to give the title compound. ¹H NMR (300 MHz, MeOD) δ 9.02 (d, J=2.26 Hz, 1H), 7.47 (dd, J=2.26, 8.67 Hz, 1H), 7.08 (d, J=8.67 Hz, 1H), 4.73-4.79 (m, 1H), 2.86 (s, 3H), 2.67 (s, 3H), 1.36 (d, J=6.03 Hz, 6H); MS m/e 443.0 (M+H).

### Example 63

### N-[2-(2-Isopropoxy-5-sulfamoyl-phenylamino)-4′-methyl-[4,5′]bithiazolyl-2′-yl]-acetamide.TFA

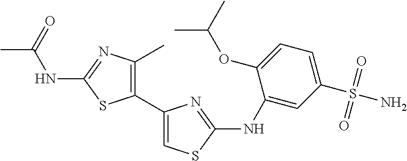

The title compound was prepared using N-[5-(2-bromo-acetyl)-4-methyl-thiazol-2-yl]-acetamide.HBr (intermediate 5) and 4-isopropoxy-3-thioureido-benzenesulfonamide (intermediate 55, step e) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 12.09 (s, 1H), 9.57 (s, 1H), 8.91 (d, J=2.26 Hz, 1H), 7.43 (dd, J=2.26, 8.67 Hz, 1H), 7.20 (d, J=9.04 Hz, 1H), 7.08 (br. s., 2H), 6.94 (s, 1H), 4.57-4.91 (m, 1H), 2.54 (s, 3H), 2.13 (s, 3H), 1.36 (d, J=6.03 Hz, 6H); MS m/e 468.0 (M+H).

### Example 64

### 3-(2′,4′-Dimethyl-[4,5′]bithiazolyl-2-ylamino)-4-isopropoxy-benzenesulfonamide.HBr

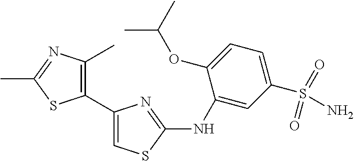

A mixture of 2-bromo-1-(2,4-dimethyl-thiazol-5-yl)-ethanone.HBr (1.07 g, 3.39 mmol, intermediate 3) and 4-isopropoxy-3-thioureido-benzenesulfonamide (0.98 g, 3.39 mmol, intermediate 55, step e) in ethanol (15 mL) was stirred at room temperature for 2 d. The mixture was filtered, washed with cold EtOH, and air-dried, affording the title compound as a white powder. ¹H NMR (300 MHz, DMSO-d₆) δ 9.67 (s, 1H), 9.00 (d, J=2.26 Hz, 1H), 7.43 (dd, J=2.07, 8.48 Hz, 1H), 7.21 (d, J=8.67 Hz, 1H), 7.14 (br. s., 2H), 7.07 (s, 1H), 4.80 (sept, J=6.03 Hz, 1H), 2.65 (s, 3H), 2.56 (s, 3H), 1.36 (d, J=6.03 Hz, 6H); MS m/e 425.1 (M+H).

### Example 65

### 3-(2′-Acetylamino-4′-methyl-[4,5′]bithiazolyl-2-ylamino)-4-methoxy-N-methyl-benzamide.TFA

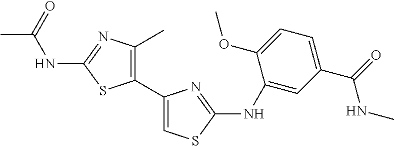

The title compound was prepared using 3-(2′-acetylamino-4′-methyl-[4,5′]bithiazolyl-2-ylamino)-4-methoxy-benzoic acid (Example 57) and methylamine in place of 3-(2′-amino-4′-methyl-[4,5′]bithiazolyl-2-ylamino)-4-methoxy-benzoic acid and benzylamine, respectively, according to the procedure described in Example 56. ¹H NMR (300 MHz, DMSO-d₆) δ ppm 12.06 (s, 1H) 9.67 (s, 1H) 8.79 (d, J=2.3 Hz, 1H) 8.15 (d, J=4.5 Hz, 1H) 7.48 (dd, J=8.7, 1.9 Hz, 1H) 7.08 (d, J=8.7 Hz, 1H) 6.88 (s, 1H) 3.91 (s, 3H) 2.77 (d, J=4.1 Hz, 3H) 2.51 (br. s., 3H) 2.13 (s, 3H); MS m/e 418.2 (M+H).

### Example 66

### 3-(2′-Amino-4′-methyl-[4,5′]bithiazolyl-2-ylamino)-4-methoxy-N-methyl-benzamide.TFA

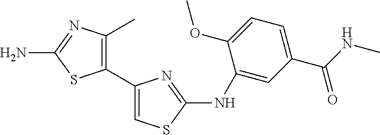

The title compound was prepared using methylamine in place of benzylamine, according to the procedure described in Example 56. ¹H NMR (300 MHz, DMSO-d₆) δ ppm 9.78 (s, 1H) 8.82 (d, J=2.3 Hz, 1H) 8.75 (br. s., 1H) 8.22 (d, J=4.9 Hz, 1H) 7.49 (dd, J=8.7, 2.3 Hz, 1H) 7.08 (d, J=8.7 Hz, 1H) 6.93 (s, 1H) 3.91 (s, 3H) 2.77 (d, J=4.5 Hz, 3H) 2.42 (s, 3H); MS m/e 376.1 (M+H).

### Example 67

### N²-(2-Methoxy-5-nitro-phenyl)-4′-methyl-[4,5′]bithiazolyl-2,2′-diamine.HBr

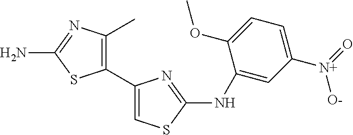

The title compound was prepared using commercially available (2-methoxy-5-nitro-phenyl)-thiourea in place of (5-cyano-2-methoxy-phenyl)-thiourea according to the procedure described in Example 40. ¹H NMR (300 MHz, DMSO-d₆) δ 10.31 (s, 1H), 9.51 (d, J=2.64 Hz, 1H), 9.21 (br. s., 2H), 7.95 (dd, J=3.01, 9.04 Hz, 1H), 7.26 (d, J=9.04 Hz, 1H), 7.14 (s, 1H), 4.01 (s, 3H), 2.46 (s, 3H); MS m/e 364.0 (M+H).

### Example 68

### N-[2-(2-Methoxy-5-nitro-phenylamino)-4′-methyl-[4,5′]bithiazolyl-2′-yl]-acetamide.TFA

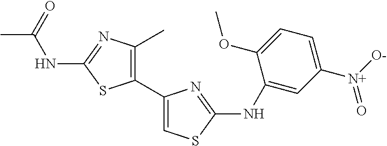

The title compound was prepared using N-[5-(2-bromo-acetyl)-4-methyl-thiazol-2-yl]-acetamide.HBr (intermediate 5) and commercially available (2-methoxy-5-nitro-phenyl)-thiourea in place of 2-bromo-1-(2,4-dimethyl-thiazol-5-yl)-ethanone.HBr and (5-cyano-2-methoxy-phenyl)-thiourea, respectively, according to the procedure described in Example 39. ¹H NMR (300 MHz, CHLOROFORM-d, MeOD) δ 9.46 (d, J=3.01 Hz, 1H), 7.95 (dd, J=2.64, 9.04 Hz, 1H), 7.00 (d, J=9.04 Hz, 1H), 6.72 (s, 1H), 4.07 (s, 3H), 2.69 (s, 3H), 2.28 (s, 3H); MS m/e 406.0 (M+H).

### Example 69

### N-[2-(5-Amino-2-methoxy-phenylamino)-4′-methyl-[4,5′]bithiazolyl-2′-yl]-acetamide

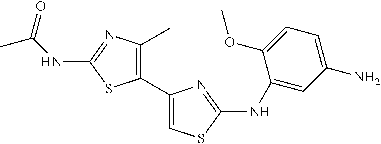

Sodium borohydride (0.007 g, 0.184 mmol) was added slowly to a solution of nickel (II) chloride hexahydrate (0.016 g, 0.062 mmol) in methanol (3 mL) at room temperature and stirred for 0.5 hours. To the resulting solution was added N-[2-(2-methoxy-5-nitro-phenylamino)-4′-methyl-[4,5′]bithiazolyl-2′-yl]-acetamide (0.050 g, 0.123 mmol, Example 68), followed by sodium borohydride (0.016 g, 0.431 mmol) and stirred for 0.5 hour. Water was added and the crude product was extracted with ethyl acetate, dried with sodium sulfate and purified via column chromatography eluting with heptanes:ethyl acetate to give the title compound. ¹H NMR (300 MHz, DMSO-d₆) δ 12.04 (s, 1H), 9.30 (s, 1H), 7.41 (d, J=2.64 Hz, 1H), 6.66-6.91 (m, 2H), 6.25 (dd, J=2.64, 8.67 Hz, 1H), 4.60 (br. s., 2H), 3.71 (s, 3H), 2.49 (s, 3H), 2.13 (s, 3H); MS m/e 376.0 (M+H).

### Example 70

### 3-(2′,4′-Dimethyl-[4,5′]bithiazolyl-2-ylamino)-4-ethoxy-benzenesulfonamide.HCl

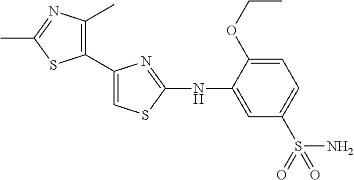

A solution of 4-ethoxy-3-thioureido-benzenesulfonamide (0.131 g, 0.476 mmol, intermediate 59, step d) and 2-bromo-1-(2,4-dimethyl-thiazol-5-yl)-ethanone.HBr (0.150 g, 0.476 mmol, intermediate 3) in ethanol (5 mL) was stirred at room temperature for several hours. The reaction mixture was then evaporated and purified via reverse phase HPLC eluting with water/acetonitrile/0.1% TFA and the pure fractions were evaporated. The compound was dissolved in ethanol and 1 N HCl was added and the solution was evaporated; this process was repeated 3 times to give the title compound. ¹H NMR (400 MHz, DMSO-d₆) δ 9.78 (s, 1H), 9.00 (d, J=2.45 Hz, 1H), 7.44 (dd, J=2.45, 8.56 Hz, 1H), 7.18 (d, J=8.56 Hz, 1H), 7.13 (br. s., 2H), 7.04 (s, 1H), 4.22 (q, J=7.01 Hz, 2H), 2.62 (s, 3H), 2.55 (s, 3H), 1.43 (t, J=6.97 Hz, 3H); MS m/e 410.9 (M+H).

### Example 71

### 3-(2′-Amino-4′-methyl-[4,5′]bithiazolyl-2-ylamino)-4-isopropoxy-benzonitrile.HBr

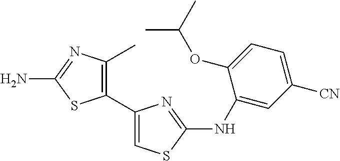

The title compound was prepared using (5-cyano-2-isopropoxy-phenyl)-thiourea (intermediate 60, step d) in place of (5-cyano-2-methoxy-phenyl)-thiourea according to the procedure described in Example 40. ¹H NMR (300 MHz, DMSO-d₆) δ 9.85 (s, 1H), 9.10 (br. s., 2H), 8.80 (d, J=2.26 Hz, 1H), 7.44 (dd, J=2.07, 8.48 Hz, 1H), 7.24 (d, J=8.67 Hz, 1H), 7.11 (s, 1H), 4.85 (sept, J=6.17 Hz, 1H), 2.42 (s, 3H), 1.36 (d, J=6.03 Hz, 6H); MS m/e 372.0 (M+H).

### Example 72

### 3-(2′,4′-Dimethyl-[4,5′]bithiazolyl-2-ylamino)-4-isopropoxy-benzonitrile.HBr

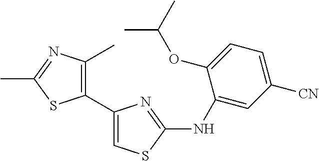

The title compound was prepared using 2-bromo-1-(2,4-dimethyl-thiazol-5-yl)-ethanone.HBr (intermediate 3) and (5-cyano-2-isopropoxy-phenyl)-thiourea (intermediate 60, step d) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 9.80 (s, 1H), 8.92 (d, J=1.88 Hz, 1H), 7.43 (dd, J=2.07, 8.48 Hz, 1H), 7.23 (d, J=8.67 Hz, 1H), 7.10 (s, 1H), 4.77-4.92 (m, 1H), 2.65 (s, 3H), 2.55 (s, 3H), 1.37 (d, J=5.65 Hz, 6H); MS m/e 371.1 (M+H).

### Example 73

### N-[2-(5-Cyano-2-isopropoxy-phenylamino)-4′-methyl-[4,5′]bithiazolyl-2′-yl]-acetamide.HBr

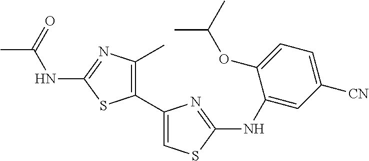

The title compound was prepared using N-[5-(2-bromo-acetyl)-4-methyl-thiazol-2-yl]-acetamide.HBr (intermediate 5) and (5-cyano-2-isopropoxy-phenyl)-thiourea (intermediate 60, step d) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 12.09 (s, 1H), 9.74 (s, 1H), 8.94 (d, J=1.88 Hz, 1H), 7.42 (dd, J=2.07, 8.48 Hz, 1H), 7.22 (d, J=8.67 Hz, 1H), 6.99 (s, 1H), 4.70-4.95 (m, 1H), 2.14 (s, 3H), 1.37 (d, J=6.03 Hz, 6H); MS m/e 414.0 (M+H).

### Example 74

### N-[2-(5-Methanesulfonyl-2-methoxy-phenylamino)-4′-methyl-[4,5]bithiazolyl-2′-yl]acetamide.HBr

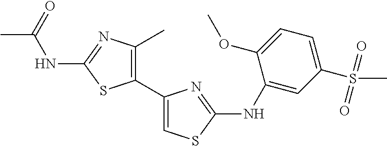

The title compound was prepared using N-[5-(2-bromo-acetyl)-4-methyl-thiazol-2-yl]-acetamide.HBr (intermediate 5) and (5-methanesulfonyl-2-methoxy-phenyl)-thiourea (intermediate 57) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ ppm 12.09 (s, 1H) 10.08 (s, 1H) 9.11 (d, J=2.3 Hz, 1H) 7.51 (dd, J=8.7, 2.3 Hz, 1H) 7.25 (d, J=8.7 Hz, 1H) 6.97 (s, 1H) 3.99 (s, 3 H) 3.19 (s, 3H) 2.53 (s, 3H) 2.14 (s, 3H); MS m/e 438.9 (M+H).

### Example 75

### N²-(5-Methanesulfonyl-2-methoxy-phenyl)-4′-methyl-[4,5′]bithiazolyl-2,2′-diamine.HBr

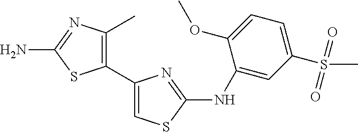

The title compound was prepared using (5-methanesulfonyl-2-methoxy-phenyl)-thiourea (intermediate 57) in place of (5-cyano-2-methoxy-phenyl)-thiourea according to the procedure described in Example 40. ¹H NMR (300 MHz, DMSO-d₆) δ ppm 10.18 (s, 1H) 9.16 (br. s., 2H) 9.03 (d, J=2.3 Hz, 1H) 7.54 (dd, J=8.3, 2.3 Hz, 1H) 7.27 (d, J=8.7 Hz, 1H) 7.10 (s, 1H) 3.99 (s, 3H) 3.15 (s, 3H) 2.45 (s, 3H). MS m/e 397.0 (M+H).

### Example 76

### 3-(2′,4′-Dimethyl-[4,5′]bithiazolyl-2-ylamino)-4-isopropoxy-benzoic acid

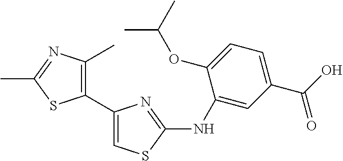

A solution of 2-bromo-1-(2,4-dimethyl-thiazol-5-yl)-ethanone.HBr (0.031 g, 0.098 mmol, intermediate 3) and 4-isopropoxy-3-thioureido-benzoic acid (0.025 g, 0.098 mmol, intermediate 58, step e) in ethanol (2 mL) was stirred at room temperature overnight. The reaction mixture was then filtered and to the solid was added saturated aq. sodium bicarbonate and ethyl acetate. The ethyl acetate was removed with a separatory funnel and the pH of the aqueous layer was adjusted to 3-4. Ethyl acetate was added and the product was extracted, dried with sodium sulfate and evaporated to give the title compound. ¹H NMR (300 MHz, DMSO-d₆) δ 12.49 (br. s., 1H), 9.53 (s, 1H), 9.12 (d, J=1.88 Hz, 1H), 7.57 (dd, J=2.07, 8.48 Hz, 1H), 7.12 (d, J=8.67 Hz, 1H), 6.97 (s, 1H), 4.80 (sept, J=6.31 Hz, 1H), 2.59 (s, 3H), 2.55 (s, 3H), 1.36 (d, J=6.03 Hz, 6H); MS m/e 390.0 (M+H).

### Example 77

### 3-(2′-Amino-4′-methyl-[4,5′]bithiazolyl-2-ylamino)-4-isopropoxy-benzoic acid.TFA

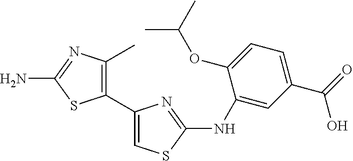

The title compound was prepared using 1-(2-amino-4-methyl-thiazol-5-yl)-2-bromo-ethanone (intermediate 4) and 4-isopropoxy-3-thioureido-benzoic acid (intermediate 58, step e) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 12.50 (br. s., 1H), 9.56 (s, 1H), 8.97 (d, J=2.26 Hz, 1H), 8.74 (br. s., 2H), 7.59 (dd, J=2.07, 8.48 Hz, 1H), 7.13 (d, J=8.67 Hz, 1H), 6.95 (s, 1H), 4.66-4.90 (m, 1H), 2.44 (s, 3H), 1.36 (d, J=6.03 Hz, 6H); MS m/e 391.2 (M+H).

### Example 78

### N-[2-(2-Ethoxy-5-oxazol-2-yl-phenylamino)-4′-methyl-[4,5′]bithiazolyl-2′-yl]-acetamide.TFA

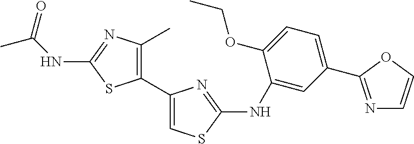

The title compound was prepared using N-[5-(2-bromo-acetyl)-4-methyl-thiazol-2-yl]-acetamide.HBr (intermediate 5) and (2-ethoxy-5-oxazol-2-yl-phenyl)-thiourea (intermediate 61, step e) in place of 2-bromo-1-(2,4-dimethyl-thiazol-5-yl)-ethanone.HBr and (5-cyano-2-methoxy-phenyl)-thiourea, respectively, according to the procedure described in Example 39. ¹H NMR (300 MHz, DMSO-d₆) δ 12.03 (s, 1H), 9.72 (s, 1H), 9.38 (d, J=2.26 Hz, 1H), 8.22 (s, 1H), 7.52-7.71 (m, 1H), 7.30 (s, 1H), 7.14 (d, J=8.29 Hz, 1H), 6.93 (s, 1H), 4.21 (q, J=6.91 Hz, 2H), 2.52 (s, 3H), 2.15 (s, 3H), 1.43 (t, J=6.97 Hz, 3H); MS m/e 442.0 (M+H).

### Example 79

### N²-(2-Ethoxy-5-oxazol-2-yl-phenyl)-4′-methyl-[4,5′]bithiazolyl-2,2′diamine.HBr

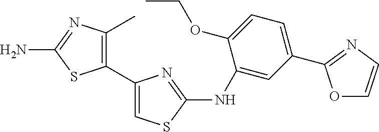

The title compound was prepared using (2-ethoxy-5-oxazol-2-yl-phenyl)-thiourea (intermediate 61, step e) in place of (5-cyano-2-methoxy-phenyl)-thiourea according to the procedure described in Example 40. ¹H NMR (300 MHz, DMSO-d₆) δ 9.81 (s, 1H), 9.14 (d, J=1.88 Hz, 1H), 9.03 (br. s., 2H), 8.18 (s, 1H), 7.61 (dd, J=2.07, 8.48 Hz, 1H), 7.33 (s, 1H), 7.17 (d, J=8.29 Hz, 1H), 7.05 (s, 1H), 4.22 (q, J=7.16 Hz, 2H), 2.47 (s, 3H), 1.44 (t, J=6.78 Hz, 3H). MS m/e 400.0 (M+H).

### Example 80

### (2′,4′-Dimethyl-[4,5′]bithiazolyl-2-yl)-(2-ethoxy-5-oxazol-2-yl-phenyl)-amine.TFA

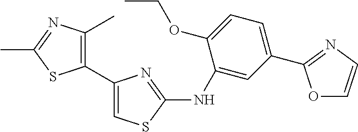

The title compound was prepared using (2-ethoxy-5-oxazol-2-yl-phenyl)-thiourea (intermediate 61, step e) in place of (5-cyano-2-methoxy-phenyl)-thiourea according to the procedure described in Example 39. ¹H NMR (300 MHz, DMSO-d₆) δ 9.73 (s, 1H), 9.24 (d, J=2.26 Hz, 1H), 8.18 (s, 1H), 7.61 (dd, J=2.07, 8.48 Hz, 1H), 7.33 (s, 1H), 7.16 (d, J=8.67 Hz, 1H), 7.01 (s, 1H), 4.22 (q, J=6.91 Hz, 2H), 2.62 (s, 3H), 2.59 (s, 3H), 1.44 (t, J=6.97 Hz, 3H); MS m/e 399.0 (M+H).

### Example 81

### 3-(2′-Amino-4′-methyl-[4,5′]bithiazolyl-2-ylamino)-4-ethoxy-benzoic acid ethyl ester.TFA

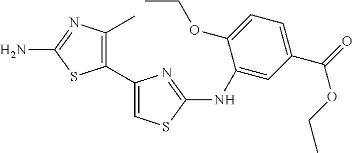

The title compound was prepared using 1-(2-amino-4-methyl-thiazol-5-yl)-2-bromo-ethanone (intermediate 4) and 4-ethoxy-3-thioureido-benzoic acid ethyl ester (intermediate 48, step d) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 9.74 (s, 1H), 9.08 (d, J=2.26 Hz, 1H), 8.64 (br. s., 2H), 7.62 (dd, J=2.07, 8.48 Hz, 1H), 7.13 (d, J=8.67 Hz, 1H), 6.96 (s, 1H), 4.14-4.36 (m, 4H), 2.43 (s, 3H), 1.43 (t, J=6.97 Hz, 3H), 1.31 (t, J=7.16 Hz, 3H); MS m/e 405.1 (M+H).

### Example 82

### 3-(2′,4′-Dimethyl-[4,5′]bithiazolyl-2-ylamino)-4-ethoxy-benzoic acid ethyl ester.HBr

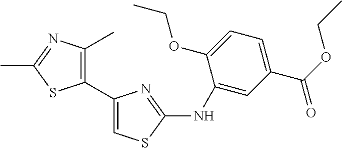

The title compound was prepared using 2-bromo-1-(2,4-dimethyl-thiazol-5-yl)-ethanone.HBr (intermediate 3) and 4-ethoxy-3-thioureido-benzoic acid ethyl ester (intermediate 48, step d) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 9.74 (s, 1H), 9.22 (d, J=2.26 Hz, 1H), 7.62 (dd, J=2.26, 8.67 Hz, 1H), 7.13 (d, J=8.67 Hz, 1H), 7.04 (s, 1H), 4.31 (q, J=6.91 Hz, 2H), 4.23 (q, J=6.78 Hz, 2H), 2.63 (s, 3H), 2.56 (s, 3H), 1.44 (t, J=6.97 Hz, 3H), 1.36 (t, J=7.16 Hz, 3H); MS m/e 404.1 (M+H).

### Example 83

### 3-(2′-Acetylamino-4′-methyl-[4,5′]bithiazolyl-2-ylamino)-4-ethoxy-benzoic acid ethyl ester.HBr

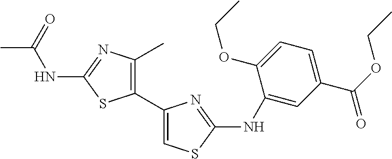

The title compound was prepared using N-[5-(2-bromo-acetyl)-4-methyl-thiazol-2-yl]-acetamide.HBr (intermediate 5) and 4-ethoxy-3-thioureido-benzoic acid ethyl ester (intermediate 48, step d) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 12.04 (s, 1H), 9.66 (s, 1H), 9.16 (d, J=1.88 Hz, 1H), 7.61 (dd, J=2.07, 8.48 Hz, 1H), 7.13 (d, J=8.67 Hz, 1H), 6.94 (s, 1H), 4.33 (q, J=6.91 Hz, 2H), 4.23 (q, J=7.03 Hz, 2H), 2.53 (br. s., 3H), 2.14 (s, 3H), 1.44 (t, J=6.97 Hz, 3H), 1.28 (t, J=7.16 Hz, 3H); MS m/e 447.1 (M+H).

### Example 84

### N-[3-(2′-Amino-4′-methyl-[4,5′]bithiazolyl-2-ylamino)-4-methoxy-phenyl]-acetamide.TFA

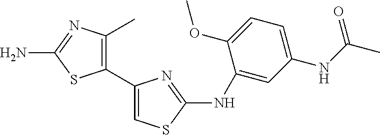

The title compound was prepared using 1-(2-amino-4-methyl-thiazol-5-yl)-2-bromo-ethanone.HBr (intermediate 4) and N-(4-methoxy-3-thioureido-phenyl)-acetamide (intermediate 56, step b) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, MeOD) δ 8.67 (s, 1H), 7.71 (s, 1H), 6.91 (d, J=1.51 Hz, 3H), 6.79 (s, 1H), 3.91 (s, 3H), 2.48 (s, 3H), 2.16 (s, 3H); MS m/e 376.2 (M+H).

### Example 85

### 3-(2′-Acetylamino-4′-methyl-[4,5′]bithiazolyl-2-ylamino)-4-methoxy-benzamide.HBr

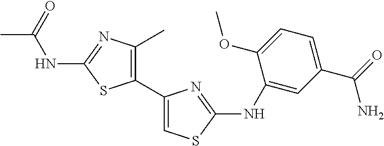

The title compound was prepared using N-[5-(2-bromo-acetyl)-4-methyl-thiazol-2-yl]-acetamide.HBr (intermediate 5) and 4-methoxy-3-thioureido-benzamide (intermediate 24) in place of 1-(2-amino-4-methyl-thiazol-5-yl)-2-bromo-ethanone and (5-cyano-2-methoxy-phenyl)-thiourea, respectively, according to the procedure described in Example 40. ¹H NMR (300 MHz, DMSO-d₆) δ 12.06 (br. s., 1H), 9.65 (s, 1H), 8.76 (d, J=1.88 Hz, 1H), 7.77 (br. s., 1H), 7.55 (dd, J=2.07, 8.48 Hz, 1H), 7.01-7.21 (m, 2H), 6.87 (s, 1H), 3.69-4.02 (m, 3H), 2.52 (br. s., 3H), 2.13 (s, 3H); MS m/e 404.2 (M+H).

### Example 86

### 3-(2′-Amino-4′-methyl-[4,5′]bithiazolyl-2-ylamino)-4-methoxy-benzoic acid methyl ester TFA

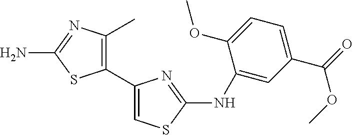

The title compound was prepared using 1-(2-amino-4-methyl-thiazol-5-yl)-2-bromo-ethanone.HBr (intermediate 4) and 4-methoxy-3-thioureido-benzoic acid methyl ester (intermediate 54) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 9.95 (s, 1H), 9.16 (d, J=1.88 Hz, 1H), 8.72 (br. s., 2H), 7.64 (dd, J=2.07, 8.48 Hz, 1H), 7.15 (d, J=8.67 Hz, 1H), 6.96 (s, 1H), 3.96 (s, 3H), 3.84 (s, 3H), 2.45 (s, 3H); MS m/e 377.1 (M+H).

### Example 87

### 3-(2′-Amino-4′-trifluoromethyl-[4,5′]bithiazolyl-2-ylamino)-4-methoxy-benzamide.TFA

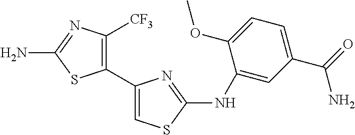

The title compound was prepared using 1-(2-amino-4-trifluoromethyl-thiazol-5-yl)-2-bromo-ethanone (intermediate 6, step c) and 4-methoxy-3-thioureido-benzamide (intermediate 24) in place of 2-bromo-1-(2,4-dimethyl-thiazol-5-yl)-ethanone.HBr and (5-cyano-2-methoxy-phenyl)-thiourea, respectively, according to the procedure described in Example 39, except the reaction mixture was concentrated instead of filtering. ¹H NMR (300 MHz, DMSO-d₆) δ 9.67 (s, 1H), 8.69 (d, J=1.88 Hz, 1H), 7.73 (br. s., 1H), 7.36-7.63 (m, 3H), 6.98-7.17 (m, 2H), 6.89 (s, 1H), 3.91 (s, 3H); MS m/e 416.0 (M+H).

### Example 88

### 3-(2′-Dimethylamino-4′-methyl-[4,5′]bithiazolyl-2-ylamino)-4-isopropoxy-benzamide.TFA

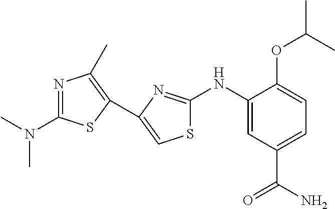

A mixture of 2-bromo-1-(2-dimethylamino-4-methyl-thiazol-5-yl)-ethanone.HBr (39 mg, 0.148 mmol, intermediate 8, step b) and 4-isopropoxy-3-thioureido-benzamide (35 mg, 0.148 mmol, intermediate 23, step e) in EtOH was heated at 100° C. for 40 min. After cooling down, the precipitate was filtered and washed with EtOAc. The solid was purified via reverse phase HPLC with water/acetonitrile/0.1% TFA to give the title compound. ¹H NMR (400 MHz, MeOD) δ 8.97 (d, J=2.20 Hz, 1H), 7.43 (dd, J=2.20, 8.31 Hz, 1H), 6.97 (d, J=8.56 Hz, 1H), 6.81 (s, 1H), 4.71 (sept, J=6.02 Hz, 1H), 3.21 (s, 6H), 2.42 (s, 3H), 1.25-1.43 (m, 6H); MS m/e 417.9 (M+H).

### Example 89

### 4-Isopropoxy-3-(4′-methyl-2′-morpholin-4-yl-[4,5′]bithiazolyl-2-ylamino)-benzamide.TFA

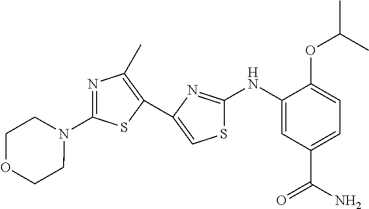

The title compound was prepared using 2-bromo-1-(4-methyl-2-morpholin-4-yl-thiazol-5-yl)-ethanone.HBr (intermediate 9, step b) in place of 2-bromo-1-(2-dimethylamino-4-methyl-thiazol-5-yl)-ethanone.HBr according to the procedure described in Example 88. ¹H NMR (400 MHz, MeOD) δ 8.98 (d, J=1.96 Hz, 1H), 7.42 (dd, J=2.20, 8.31 Hz, 1H), 6.96 (d, J=8.80 Hz, 1H), 6.81 (s, 1H), 4.70 (sept, J=6.11 Hz, 1H), 3.65-3.84 (m, 4H), 3.54 (t, J=5.01 Hz, 4H), 2.41 (s, 3H), 1.23-1.42 (m, 6H); MS m/e 460.0 (M+H).

### Example 90

### 4-Isopropoxy-3-[4′-methyl-2′-(4-methyl-piperazin-1-yl)-[4,5′]bithiazolyl-2-ylamino]-benzamide.TFA

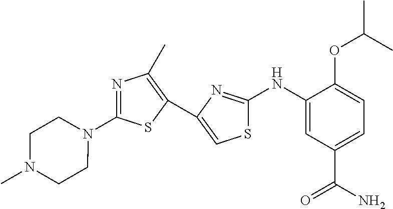

The title compound was prepared using 2-bromo-1-[4-methyl-2-(4-methyl-piperazin-1-yl)-thiazol-5-yl]-ethanone.HBr (intermediate 7, step c), in place of 2-bromo-1-(2-dimethylamino-4-methyl-thiazol-5-yl)-ethanone.HBr according to the procedure described in Example 88. ¹H NMR (400 MHz, MeOD) δ 9.03 (d, J=2.20 Hz, 1H), 7.54 (dd, J=2.20, 8.56 Hz, 1H), 7.09 (d, J=8.80 Hz, 1H), 6.79 (s, 1H), 4.82 (sept, J=6.11, 1H), 3.86 (br. s., 4H), 3.41-3.58 (m, 4H), 3.01 (s, 3H), 2.51 (s, 3H), 1.38-1.46 (m, 6H); MS m/e 473.0 (M+H).

### Example 91

### 4-Isopropoxy-3-(4′-methyl-2′-pyrrolidin-1-yl-[4,5′]bithiazolyl-2-ylamino)-benzamide.TFA

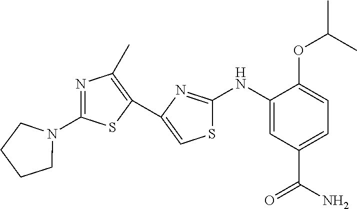

The title compound was prepared using 2-bromo-1-(4-methyl-2-pyrrolidin-1-yl-thiazol-5-yl)-ethanone.HBr (intermediate 10, step b) in place of 2-bromo-1-(2-dimethylamino-4-methyl-thiazol-5-yl)-ethanone.HBr according to the procedure described in Example 88. ¹H NMR (400 MHz, MeOD) δ 9.09 (d, J=2.45 Hz, 1H), 7.54 (dd, J=2.20, 8.56 Hz, 1H), 7.09 (d, J=8.80 Hz, 1H), 6.84 (s, 1H), 4.63-4.91 (m, 1H), 3.55 (br. s., 4H), 2.47 (s, 3H), 2.05-2.25 (m, 4H), 1.45 (d, J=6.11 Hz, 6H); MS m/e 444.1 (M+H).

### Example 92

### 4-Isopropoxy-3-[4′-methyl-2′-(pyridin-4-ylamino)-[4,5′]bithiazolyl-2-ylamino]-benzamide.TFA

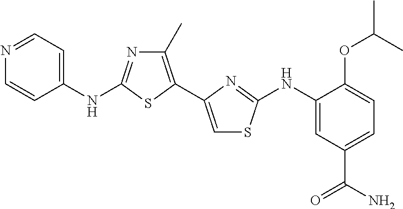

The title compound was prepared using 2-bromo-1-[4-methyl-2-(pyridin-4-ylamino)-thiazol-5-yl]-ethanone.HBr (intermediate 12, step b) in place of 2-bromo-1-(2-dimethylamino-4-methyl-thiazol-5-yl)-ethanone.HBr according to the procedure described in Example 88. ¹H NMR (400 MHz, MeOD) δ 8.90 (d, J=2.20 Hz, 1H), 8.25 (d, J=7.34 Hz, 2H), 7.90 (br. s., 2H), 7.42 (dd, J=2.20, 8.56 Hz, 1H), 6.95 (d, J=8.56 Hz, 1H), 6.69 (s, 1H), 4.69 (sept, J=6.08 Hz, 1H), 2.45 (s, 3H), 1.25-1.43 (m, 6H); MS m/e 467.0 (M+H).

### Example 93

### 4-Methoxy-3-[4′-methyl-2′-(4-methyl-piperazin-1-yl)-[4,5′]bithiazolyl-2-ylamino]-benzamide.TFA

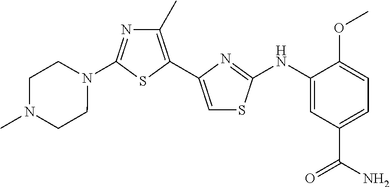

The title compound was prepared using 2-bromo-1-[4-methyl-2-(4-methyl-piperazin-1-yl)-thiazol-5-yl]-ethanone.HBr (intermediate 7, step c) in place of 2-bromo-1-(2-dimethylamino-4-methyl-thiazol-5-yl)-ethanone.HBr and 4-methoxy-3-thioureido-benzamide (intermediate 24) in place of 4-isopropoxy-3-thioureido-benzamide according to the procedure described in Example 88. ¹H NMR (300 MHz, MeOD) δ 9.08 (d, J=2.26 Hz, 1H), 7.49-7.55 (m, 1H), 7.00-7.10 (m, 1H), 6.70 (s, 1H), 3.98 (s, 3H), 3.45 (br. s., 8H), 2.98 (s, 3H), 2.47 (s, 3H); MS m/e 445.0 (M+H).

### Example 94

### 3-(2′-Diethylamino-4′-methyl-[4,5′]bithiazolyl-2-ylamino)-4-isopropoxy-benzamide.TFA

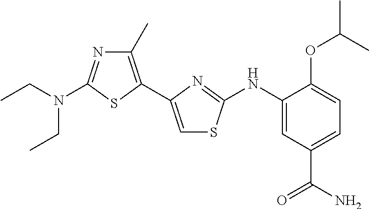

The title compound was prepared using 2-bromo-1-(2-diethylamino-4-methyl-thiazol-5-yl)-ethanone.HBr (intermediate 11, step b) in place of 2-bromo-1-(2-dimethylamino-4-methyl-thiazol-5-yl)-ethanone.HBr according to the procedure described in Example 88. ¹H NMR (400 MHz, MeOD) δ 8.96 (d, J=2.20 Hz, 1H), 7.42 (dd, J=2.20, 8.56 Hz, 1H), 6.96 (d, J=8.80 Hz, 1H), 6.83 (s, 1H), 4.70 (dt, J=6.11, 12.23 Hz, 1H), 3.58 (q, J=7.34 Hz, 4H), 2.44 (s, 3H), 1.11-1.38 (m, 12H); MS m/e 446.0 (M+H).

### Example 95

### 4-Isopropoxy-3-[4′-methyl-2′-(pyridin-3-ylamino)-[4,5′]bithiazolyl-2-ylamino]-benzamide.TFA

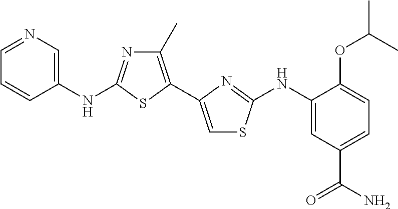

The title compound was prepared using 2-bromo-1-[4-methyl-2-(pyridin-3-ylamino)-thiazol-5-yl]-ethanone.HBr (intermediate 13, step b) in place of 2-bromo-1-(2-dimethylamino-4-methyl-thiazol-5-yl)-ethanone.HBr according to the procedure described in Example 88. MS m/e 467.0 (M+H).

### Example 96

### 4-Isopropoxy-3-[4′-methyl-2′-(pyridin-2-ylamino)-[4,5′]bithiazolyl-2-ylamino]-benzamide.TFA

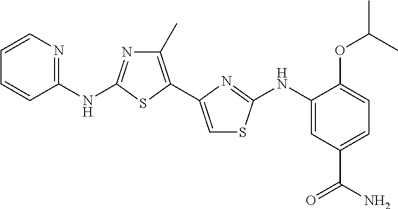

The title compound was prepared using 2-bromo-1-[4-methyl-2-(pyridin-2-ylamino)-thiazol-5-yl]-ethanone.HBr (intermediate 14, step b) in place of 2-bromo-1-(2-dimethylamino-4-methyl-thiazol-5-yl)-ethanone.HBr according to the procedure described in Example 88. MS m/e 467.0 (M+H).

### Example 97

### 4-Isopropoxy-3-[4′-methyl-2′-(3-pyrrolidin-1-yl-5-trifluoromethyl-pyridin-2-yl)-[4,5′]bithiazolyl-2-ylamino]-benzamide.TFA

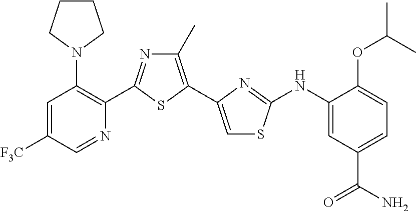

The title compound was prepared using 2-bromo-1-[4-methyl-2-(3-pyrrolidin-1-yl-5-trifluoromethyl-pyridin-2-yl)-thiazol-5-yl]-ethanone.HBr (intermediate 15, step b) in place of 2-bromo-1-(2-dimethylamino-4-methyl-thiazol-5-yl)-ethanone.HBr according to the procedure described in Example 88. ¹H NMR (400 MHz, MeOD) δ 8.86 (d, J=2.20 Hz, 1H), 8.38 (s, 1H), 7.78 (s, 1H), 7.44 (dd, J=2.20, 8.56 Hz, 1H), 6.97-7.02 (m, 2H), 4.68-4.78 (m, 1H), 3.20-3.35 (m, 3H), 3.10-3.18 (m, 1H), 2.65 (s, 3H), 1.93-2.01 (m, 3H), 1.84-1.90 (m, 1H), 1.31-1.36 (m, 6H); MS m/e 605.0 (M+H).

### Example 98

### 4-Isopropoxy-3-(4′-methyl-2′-pyridin-4-yl-[4,5′]bithiazolyl-2-ylamino)-benzamide.TFA

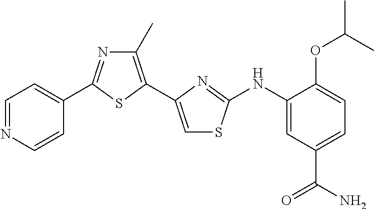

The title compound was prepared using 2-bromo-1-(4-methyl-2-pyridin-4-yl-thiazol-5-yl)-ethanone.HBr (intermediate 22, step b) in place of 2-bromo-1-(2-dimethylamino-4-methyl-thiazol-5-yl)-ethanone.HBr according to the procedure described in Example 88. ¹H NMR (400 MHz, MeOD) δ 9.00 (d, J=2.20 Hz, 1H), 8.54 (dd, J=1.59, 4.52 Hz, 2H), 7.87 (dd, J=1.59, 4.52 Hz, 2H), 7.44 (dd, J=2.20, 8.56 Hz, 1H), 6.95-7.00 (m, 2H), 4.68-4.76 (m, 1H), 2.64 (s, 3H), 1.31-1.36 (m, 6H); MS m/e 452.0 (M+H).

### Example 99

### 2-(5-Carbamoyl-2-isopropoxy-phenylamino)-4′-methyl-[4,5′]bithiazolyl-2′-carboxylic acid ethyl ester.HBr

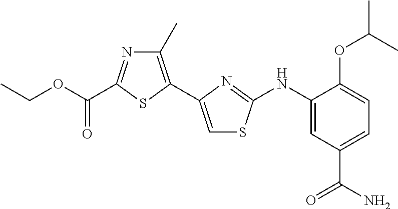

A mixture of 5-(2-bromo-acetyl)-4-methyl-thiazole-2-carboxylic acid ethyl ester (580 mg, 1.97 mmol, intermediate 16, step b) and 4-isopropoxy-3-thioureido-benzamide (500 mg, 1.97 mmol, intermediate 23, step e) in EtOH was stirred at room temperature for 24 h. The mixture was filtered, washed sequentially with EtOH and heptanes, and air-dried, affording the title compound as a white solid. ¹H NMR (300 MHz, MeOD) δ 8.83 (d, J=2.26 Hz, 1H), 7.44 (dd, J=2.26, 8.67 Hz, 1H), 6.95-7.10 (m, 2H), 4.69-4.80 (m, 1H), 4.34 (q, J=7.16 Hz, 2H), 2.62 (s, 3H), 1.28-1.37 (m, 9H); MS m/e 447.1 (M+H).

### Example 100

### 2-(5-Carbamoyl-2-isopropoxy-phenylamino)-4′-methyl-[4,5′]bithiazolyl-2′-carboxylic acid

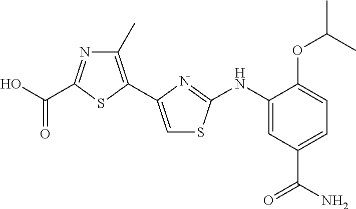

To a solution of 2-(5-carbamoyl-2-isopropoxy-phenylamino)-4′-methyl-[4,5′]bithiazolyl-2′-carboxylic acid ethyl ester.HBr (360 mg, 0.682 mmol, Example 99) in a mixture of THF and MeOH (3 mL each) was added 2 N NaOH (0.68 mL, 1.36 mmol). The reaction mixture was stirred for 1 h and concentrated. The residue was suspended in water (3 mL) and acidified with 1 N HCl. The mixture was filtered, washed with water and air-dried, affording the title compound as a white solid. MS m/e 419.0 (M+H).

### Example 101

### 4-Isopropoxy-3-[4′-methyl-2′-(4-methyl-piperazine-1-carbonyl)-[4,5′]bithiazolyl-2-ylamino]-benzamide.TFA

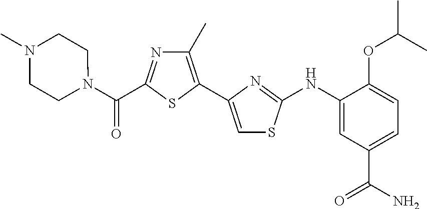

HATU (34 mg, 0.0624 mmol) was added to a THF solution (1 mL) of 2-(5-carbamoyl-2-isopropoxy-phenylamino)-4′-methyl-[4,5′]bithiazolyl-2′-carboxylic acid (20 mg, 0.048 mmol, Example 100) and 1-methyl-piperazine (0.0266 mL, 0.24 mmol) and the mixture was warmed to 70° C. After 4 h at 70° C., the mixture was purified via reverse phase HPLC with water/acetonitrile/0.1% TFA to give the title compound. ¹H NMR (400 MHz, MeOD) δ 8.83 (d, J=2.20 Hz, 1H), 7.43 (dd, J=2.20, 8.56 Hz, 1H), 6.89-6.99 (m, 2H), 4.56-4.86 (m, 1H), 3.53 (d, J=11.74 Hz, 2H), 3.14-3.33 (m, 6H), 2.87 (s, 3H), 2.52 (s, 3H), 1.32 (d, J=6.11 Hz, 6H); MS m/e 501.0 (M+H).

### Example 102

### 3-[2′-(4-Ethyl-piperazine-1-carbonyl)-4′-methyl-[4,5′]bithiazolyl-2-ylamino]-4-isopropoxy-benzamide.TFA

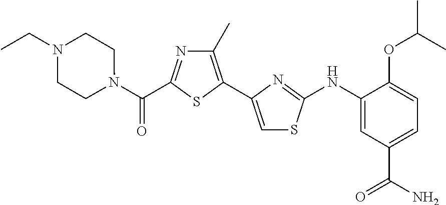

The title compound was prepared using 1-ethyl-piperazine in place of 1-methyl-piperazine according to the procedure described in Example 101. ¹H NMR (400 MHz, MeOD) δ 8.83 (d, J=2.20 Hz, 1H), 7.43 (dd, J=1.96, 8.56 Hz, 1H), 6.95-7.00 (m, 2H), 4.54-4.84 (m, 1H), 3.58 (br. s., 3H), 3.02-3.28 (m, 7H), 2.58 (s, 3H), 1.23-1.35 (m, 9H); MS m/e 515.0 (M+H).

### Example 103

### 4-Isopropoxy-3-[2′-(4-isopropyl-piperazine-1-carbonyl)-4′-methyl-[4,5]bithiazolyl-2-ylamino]-benzamide.TFA

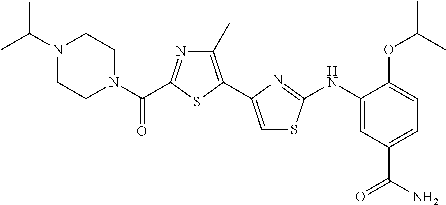

The title compound was prepared using 1-isopropyl-piperazine in place of 1-methyl-piperazine according to the procedure described in Example 101. ¹H NMR (400 MHz, MeOD) δ 8.83 (d, J=1.96 Hz, 1H), 7.43 (dd, J=2.20, 8.56 Hz, 1H), 6.96-7.00 (m, 2H), 4.59-4.80 (m, 1H), 3.40-3.60 (m, 4H), 3.07-3.36 (m, 3H), 2.59 (s, 3H), 1.29-1.34 (m, 12H); MS m/e 529.0 (M+H).

### Example 104

### 4-Isopropoxy-3-[4′-methyl-2′-(4-methyl-[1,4]diazepane-1-carbonyl)-[4,5′]bithiazolyl-2-ylamino]-benzamide.TFA

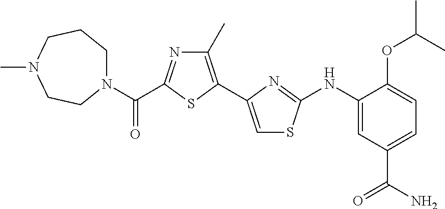

The title compound was prepared using 1-methyl-[1,4]diazepane in place of 1-methyl-piperazine according to the procedure described in Example 101. ¹H NMR (400 MHz, MeOD) δ 8.95 (d, J=1.96 Hz, 1H), 7.51-7.57 (m, 1H), 7.05-7.12 (m, 2H), 4.75-4.95 (m, 1H), 4.14 (br. s., 1H), 3.71-4.00 (m, 3H), 3.58 (br. s., 2H), 3.25-3.43 (m, 1H), 3.01 (br. s., 3H), 2.62-2.87 (m, 4H), 2.31 (br. s., 2H), 1.40-1.49 (m, 6H); MS m/e 515.0 (M+H).

### Example 105

### 3-[2′-(4-Hydroxy-piperidine-1-carbonyl)-4′-methyl-[4,5′]bithiazolyl-2-ylamino]-4-isopropoxy-benzamide.TFA

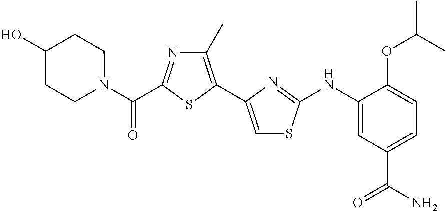

The title compound was prepared using piperidin-4-ol in place of 1-methyl-piperazine according to the procedure described in Example 101. ¹H NMR (400 MHz, MeOD) δ 8.89 (d, J=2.20 Hz, 1H), 7.50-7.63 (m, 1H), 7.08-7.13 (m, 2H), 4.77-4.88 (m, 2H), 3.70-3.98 (m, 2H), 3.30-3.43 (m, 3H), 2.62-2.95 (m, 5H), 1.95-2.04 (m, 1H), 1.59 (br. s., 1H), 1.44 (d, J=6.11 Hz, 6H); MS m/e 502.0 (M+H).

### Example 106

### 2-(5-Carbamoyl-2-isopropoxy-phenylamino)-4′-methyl-[4,5′]bithiazolyl-2′-carboxylic acid dimethylamide.TFA

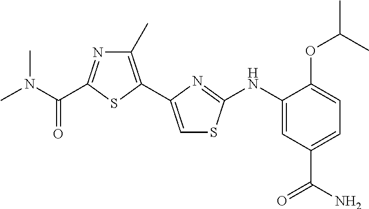

The title compound was prepared using dimethyl amine in THF (2 N) in place of 1-methyl-piperazine according to the procedure described in Example 101. ¹H NMR (400 MHz, MeOD) δ 8.81 (br. s., 1H), 7.56-7.64 (m, 1H), 7.04-7.18 (m, 2H), 4.79-5.02 (m, 1H), 3.58 (br. s., 3H), 3.15 (br. s., 3H), 2.68 (s, 3H), 1.19-1.45 (m, 6H); MS m/e 446.0 (M+H).

### Example 107

### 2-(5-Carbamoyl-2-isopropoxy-phenylamino)-4′-methyl-[4,5′]bithiazolyl-2′-carboxylic acid (2-dimethylamino-ethyl)-methyl-amide.TFA

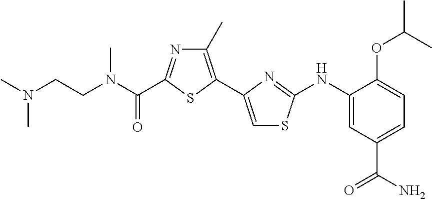

The title compound was prepared using N,N,N′-trimethyl-ethane-1,2-diamine in place of 1-methyl-piperazine according to the procedure described in Example 101. ¹H NMR (400 MHz, MeOD) δ 8.85 (d, J=2.20 Hz, 1H), 7.43 (dd, J=2.20, 8.56 Hz, 1H), 6.94-7.00 (m, 2H), 4.60-4.80 (m, 1H), 4.34 (br. s., 1H), 3.83 (br. s., 1H), 3.43-3.59 (m, 3H), 3.31-3.43 (m, 1H), 2.98-3.13 (m, 1H), 2.92 (br. s., 6H), 2.60 (s, 3H), 1.33 (d, J=6.11 Hz, 6H); MS m/e 503.0 (M+H).

### Example 108

### 3-[2′-(4-Cyclohexyl-piperazine-1-carbonyl)-4′-methyl-[4,5′]bithiazolyl-2-ylamino]-4-isopropoxy-benzamide.TFA

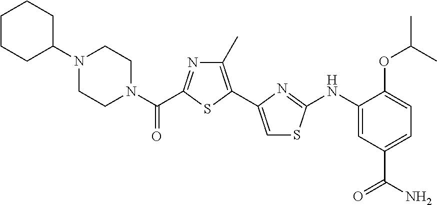

The title compound was prepared using 1-cyclohexyl-piperazine in place of 1-methyl-piperazine according to the procedure described in Example 101. ¹H NMR (400 MHz, MeOD) δ 8.97 (d, J=2.20 Hz, 1H), 7.55 (dd, J=2.20, 8.31 Hz, 1H), 7.10 (t, J=4.40 Hz, 2H), 4.83 (sept, J=6.11 Hz, 2H), 3.65 (br. s., 3H), 3.14-3.44 (m, 6H), 2.71 (s, 3H), 2.11-2.21 (m, 2H), 1.97 (d, J=13.94 Hz, 2H), 1.73 (br. s., 1H), 1.36-1.52 (m, 9H), 1.26-1.34 (m, 2H); MS m/e 569.1 (M+H).

### Example 109

### 4-Isopropoxy-3-{4′-methyl-2′-[4-(5-trifluoromethyl-pyridin-2-yl)-piperazine-1-carbonyl]-[4,5′]bithiazolyl-2-ylamino}-benzamide.TFA

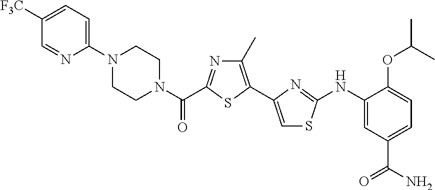

The title compound was prepared using 1-(5-trifluoromethyl-pyridin-2-yl)-piperazine in place of 1-methyl-piperazine according to the procedure described in Example 101. MS m/e 632.0 (M+H).

### Example 110

### 3-[2′-(4-Dimethylamino-piperidine-1-carbonyl)-4′-methyl-[4,5]bithiazolyl-2-ylamino]-4-isopropoxy-benzamide.TFA

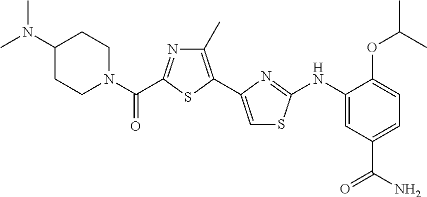

The title compound was prepared using dimethyl-piperidin-4-yl-amine in place of 1-methyl-piperazine according to the procedure described in Example 101. ¹H NMR (400 MHz, MeOD) δ 8.85 (d, J=2.20 Hz, 1H), 7.43 (dd, J=2.20, 8.56 Hz, 1H), 6.93-7.01 (m, 2H), 4.70 (sept, J=6.08 Hz, 1H), 3.41-3.50 (m, 1H), 3.00-3.25 (m, 2H), 2.71-2.95 (m, 8H), 2.58 (s, 3H), 2.09 (br. s., 2H), 1.67 (br. s., 2H), 1.32 (d, J=5.87 Hz, 6H); MS m/e 529.0 (M+H).

### Example 111

### 2-(5-Carbamoyl-2-isopropoxy-phenylamino)-4′-methyl-[4,5′]bithiazolyl-2′-carboxylic acid (1-isopropyl-piperidin-4-yl)-amide.TFA

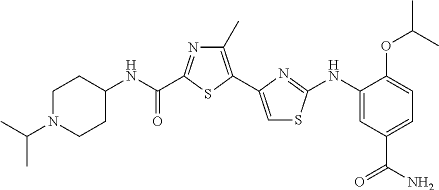

The title compound was prepared using 1-isopropyl-piperidin-4-ylamine in place of 1-methyl-piperazine according to the procedure described in Example 101. ¹H NMR (400 MHz, MeOD) δ 8.88-9.01 (m, 1H), 7.55 (dd, J=2.20, 8.56 Hz, 1H), 7.06-7.13 (m, 2H), 4.82 (sept, J=6.11 Hz, 1H), 4.10-4.20 (m, 1H), 3.49-3.64 (m, 3H), 3.14-3.28 (m, 2H), 2.72 (s, 3H), 2.33 (br. s., 1H), 2.27 (br. s., 1H), 1.87-2.12 (m, 2H), 1.35-1.47 (m, 12H); MS m/e 543.0 (M+H).

### Example 112

### 4-Isopropoxy-3-[2′-(4-isopropyl-[1,4]diazepane-1-carbonyl)-4′-methyl-[4,5]bithiazolyl-2-ylamino]-benzamide.TFA

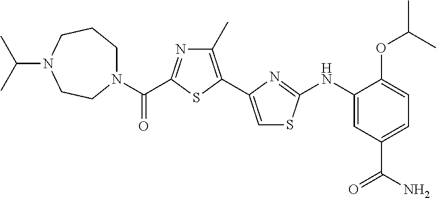

The title compound was prepared using 1-isopropyl-[1,4]diazepane in place of 1-methyl-piperazine according to the procedure described in Example 101. ¹H NMR (400 MHz, CHLOROFORM-d) δ 8.89 (s, 1H), 7.96 (br. s., 1H), 7.48-7.58 (m, 1H), 6.92 (d, J=8.56 Hz, 1H), 6.77 (d, J=6.60 Hz, 2H), 6.49 (br. s., 1H), 4.74 (dt, J=6.02, 12.17 Hz, 1H), 4.05 (br. s., 1H), 3.89 (br. s., 1H), 3.69 (br. s., 2H), 3.41-3.60 (m, 2H), 3.32 (dd, J=4.40, 7.34 Hz, 1H), 2.88-3.05 (m, 1H), 2.65-2.65 (m, 5H), 2.28 (br. s., 1H), 1.38-1.65 (m, 6H), 1.27-1.35 (m, 6H); MS m/e 543.0 (M+H).

### Example 113

### 2-(5-Carbamoyl-2-isopropoxy-phenylamino)-4′-methyl-[4,5′]bithiazolyl-2′-carboxylic acid (1-methyl-piperidin-4-yl)-amide.TFA

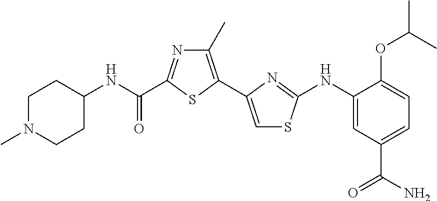

The title compound was prepared using 1-methyl-piperidin-4-ylamine in place of 1-methyl-piperazine according to the procedure described in Example 101. ¹H NMR (400 MHz, CHLOROFORM-d) δ 8.88 (d, J=1.96 Hz, 1H), 7.90 (s, 1H), 7.55 (dd, J=1.96, 8.31 Hz, 1H), 7.42 (d, J=8.31 Hz, 1H), 6.93 (d, J=8.80 Hz, 1H), 6.73-6.80 (m, 1H), 4.73 (sept, J=6.02 Hz, 1H), 4.56 (br. s., 2H), 4.11-4.38 (m, 1H), 3.63 (d, J=11.74 Hz, 2H), 2.90-3.05 (m, 2H), 2.74-2.90 (m, 3H), 2.63 (s, 3H), 2.10-2.38 (m, 2H), 1.38-1.51 (m, 6H); MS m/e 515.0 (M+H).

### Example 114

### 3-[2′-(4-Ethyl-[1,4]diazepane-1-carbonyl)-4′-methyl-[4,5′]bithiazolyl-2-ylamino]-4-isopropoxy-benzamide.TFA

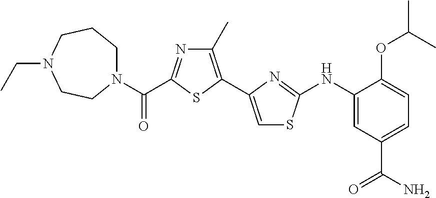

The title compound was prepared using 1-ethyl-[1,4]diazepane in place of 1-methyl-piperazine according to the procedure described in Example 101. ¹H NMR (400 MHz, CHLOROFORM-d) δ 8.80-8.95 (m, 1H), 7.79 (s, 1H), 7.60 (dd, J=2.20, 8.56 Hz, 1H), 6.94 (d, J=8.80 Hz, 1H), 6.78 (s, 1H), 4.74 (sept, J=6.11 Hz, 1H), 4.30-4.46 (m, 1H), 4.26 (t, J=6.24 Hz, 1H), 3.70-3.86 (m, 2H), 2.74-2.86 (m, 2H), 2.49-2.74 (m, 7H), 1.82-2.11 (m, 2H), 1.37-1.65 (m, 6H), 1.08 (q, J=7.09 Hz, 3H); MS m/e 529.0 (M+H).

### Example 115

### 4-Ethoxy-3-[4′-methyl-2′-(4-methyl-piperazine-1-carbonyl)-[4,5′]bithiazolyl-2-ylamino]-benzamide.TFA

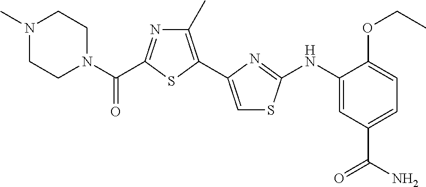

The title compound was prepared using 2-(5-carbamoyl-2-ethoxy-phenylamino)-4′-methyl-[4,5]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid according to the procedure described in Example 101. ¹H NMR (400 MHz, CHLOROFORM-d) δ 8.79 (d, J=2.20 Hz, 1H), 7.70 (s, 1H), 7.54 (dd, J=2.20, 8.31 Hz, 1H), 6.87 (d, J=8.56 Hz, 1H), 6.72 (s, 1H), 4.38 (br. s., 2H), 4.15 (q, J=7.09 Hz, 2H), 3.75 (br. s., 2H), 2.63 (s, 3H), 2.44 (t, J=4.52 Hz, 4H), 2.27 (s, 3H), 1.41-1.50 (m, 3H); MS m/e 487.0 (M+H).

### Example 116

### 4-Ethoxy-3-[2′-(4-isopropyl-[1,4]diazepane-1-carbonyl)-4′-methyl-[4,5]bithiazolyl-2-ylamino]-benzamide.TFA

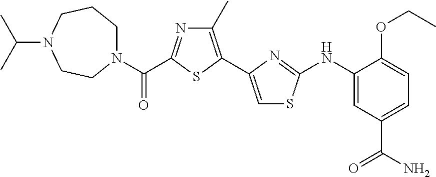

The title compound was prepared using 1-isopropyl-[1,4]diazepane in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid according to the procedure described in Example 101. ¹H NMR (400 MHz, CHLOROFORM-d) δ 8.88 (s, 1H), 7.77 (s, 1H), 7.61 (dd, J=2.08, 8.44 Hz, 1H), 6.94 (d, J=8.56 Hz, 1H), 6.78 (s, 1H), 4.18-4.34 (m, 4H), 3.74-3.82 (m, 2H), 2.94 (td, J=4.16, 6.48 Hz, 1H), 2.74-2.86 (m, 2H), 2.57-2.74 (m, 6H), 1.60-2.00 (m, 1H), 1.50-1.65 (m, 3H), 1.02-1.10 (m, 6H); MS m/e 529.0 (M+H).

### Example 117

### 4-Ethoxy-3-[2′-(4-ethyl-piperazine-1-carbonyl)-4′-methyl-[4,5]bithiazolyl-2-ylamino]-benzamide.TFA

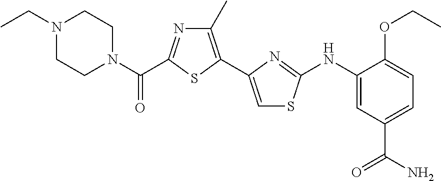

The title compound was prepared using 1-ethyl-piperazine in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5]bithiazolyl-2′-carboxylic acid according to the procedure described in Example 101. ¹H NMR (400 MHz, CHLOROFORM-d) δ 8.86 (d, J=2.20 Hz, 1H), 7.78 (s, 1H), 7.61 (dd, J=2.08, 8.44 Hz, 1H), 6.94 (d, J=8.56 Hz, 1H), 6.79 (s, 1H), 4.46 (br. s., 2H), 4.22 (q, J=7.01 Hz, 2H), 3.83 (br. s., 2H), 2.70 (s, 3H), 2.37-2.63 (m, 6H), 1.46-1.60 (m, 3H), 1.13 (t, J=7.21 Hz, 3H); MS m/e 501.0 (M+H).

### Example 118

### 4-Ethoxy-3-[2′-(4-isopropyl-piperazine-1-carbonyl)-4′-methyl-[4,5]bithiazolyl-2-ylamino]-benzamide.TFA

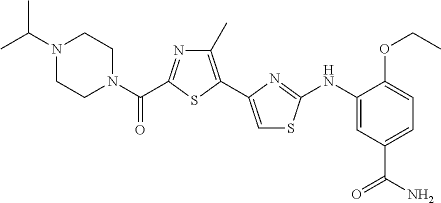

The title compound was prepared using 1-isopropyl-piperazine in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid, according to the procedure described in Example 101. ¹H NMR (400 MHz, CHLOROFORM-d) δ 8.86 (d, J=2.20 Hz, 1H), 7.81 (s, 1H), 7.60 (dd, J=2.08, 8.44 Hz, 1H), 6.93 (d, J=8.56 Hz, 1H), 6.78 (s, 1H), 4.43 (br. s., 2H), 4.21 (q, J=7.01 Hz, 2H), 3.80 (br. s., 2H), 2.66-2.81 (m, 4H), 2.50-2.66 (m, 4H), 1.52 (t, J=6.97 Hz, 3H), 1.00-1.17 (m, 6H); MS m/e 515.1 (M+H).

### Example 119

### 4-Ethoxy-3-[4′-methyl-2′-(4-methyl-[1,4]diazepane-1-carbonyl)-[4,5′]bithiazolyl-2-ylamino]-benzamide.TFA

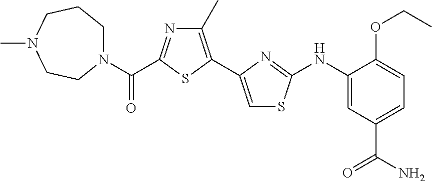

The title compound was prepared using 1-methyl-[1,4]diazepane in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid, according to the procedure described in Example 101. ¹H NMR (400 MHz, CHLOROFORM-d) δ 8.88 (d, J=1.96 Hz, 1H), 7.76 (s, 1H), 7.61 (dd, J=2.20, 8.56 Hz, 1H), 6.94 (d, J=8.56 Hz, 1H), 6.79 (s, 1H), 4.33-4.38 (m, 1H), 4.19-4.31 (m, 3H), 3.76-3.85 (m, 2H), 2.67-2.81 (m, 5H), 2.60-2.67 (m, 2H), 2.40 (d, J=4.89 Hz, 3H), 2.00-2.08 (m, 2H), 1.43-1.57 (m, 3H); MS m/e 501.0 (M+H).

### Example 120

### 4-Ethoxy-3-[2′-(4-ethyl-[1,4]diazepane-1-carbonyl)-4′-methyl-[4,5]bithiazolyl-2-ylamino]-benzamide.TFA

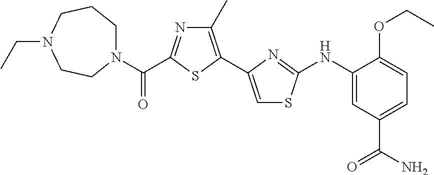

The title compound was prepared using 1-ethyl-[1,4]diazepane in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid according to the procedure described in Example 101. ¹H NMR (400 MHz, CHLOROFORM-d) δ 8.81-8.94 (m, 1H), 7.77 (s, 1H), 7.61 (dd, J=2.08, 8.44 Hz, 1H), 6.94 (d, J=8.31 Hz, 1H), 6.78 (s, 1H), 4.18-4.31 (m, 2H), 3.75-3.85 (m, 2H), 2.75-2.86 (m, 2H), 2.53-2.75 (m, 7H), 1.95-2.07 (m, 2H), 1.88 (br. s., 2H), 1.51-1.62 (m, 3H), 1.03-1.20 (m, 3H); MS m/e 515.0 (M+H).

### Example 121

### 2-(5-Carbamoyl-2-ethoxy-phenylamino)-4′-methyl-[4,5′]bithiazolyl-2′-carboxylic acid (1-methyl-piperidin-4-yl)-amide.TFA

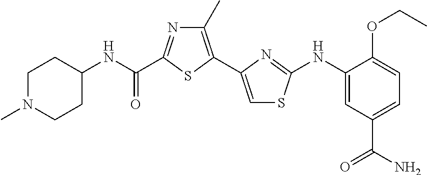

The title compound was prepared using 1-methyl-piperidin-4-ylamine in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid, according to the procedure described in Example 101. ¹H NMR (400 MHz, CHLOROFORM-d) δ 8.83 (d, J=2.20 Hz, 1H), 7.77 (s, 1H), 7.62 (dd, J=2.20, 8.56 Hz, 1H), 7.11 (d, J=8.31 Hz, 1H), 6.95 (d, J=8.56 Hz, 1H), 6.80 (s, 1H), 4.22 (q, J=6.85 Hz, 2H), 3.96 (br. s., 1H), 2.84 (br. s., 2H), 2.69 (s, 3H), 2.31 (s, 3H), 2.10-2.20 (m, 2H), 2.04 (dd, J=3.42, 12.72 Hz, 2H), 1.60-1.75 (m, 2H), 1.45-1.60 (m, 3H); MS m/e 501.0 (M+H).

### Example 122

### 2-(5-Carbamoyl-2-ethoxy-phenylamino)-4′-methyl-[4,5′]bithiazolyl-2′-carboxylic acid (1-isopropyl-piperidin-4-yl)-amide.TFA

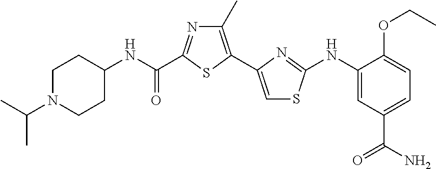

The title compound was prepared using 1-isopropyl-piperidin-4-ylamine in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid according to the procedure described in Example 101. ¹H NMR (400 MHz, CHLOROFORM-d) δ 8.84 (d, J=1.96 Hz, 1H), 7.77 (s, 1H), 7.62 (dd, J=2.08, 8.44 Hz, 1H), 7.11 (d, J=8.31 Hz, 1H), 6.95 (d, J=8.56 Hz, 1H), 6.79 (s, 1H), 4.22 (q, J=6.85 Hz, 2H), 3.95 (br. s., 1H), 2.82-2.92 (m, 2H), 2.72-2.82 (m, 1H), 2.69 (s, 3H), 2.27-2.38 (m, 2H), 2.00-2.10 (m, 2H), 1.62 (br. s., 2H), 1.43-1.54 (m, 3H), 1.07 (d, J=6.60 Hz, 6H); MS m/e 529.0 (M+H).

### Example 123

### 2-(5-Carbamoyl-2-ethoxy-phenylamino)-4′-methyl-[4,5′]bithiazolyl-2′-carboxylic acid (2-dimethylamino-ethyl)-methyl-amide.TFA

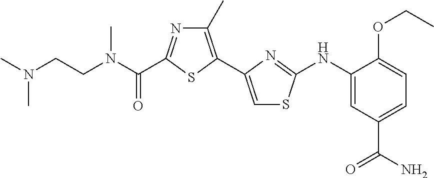

The title compound was prepared using N,N,N′-trimethyl-ethane-1,2-diamine in place of 1-methyl-piperazine and 2-(5-carbamoyl-2-ethoxy-phenylamino)-4′-methyl-[4,5′]bithiazolyl-2′-carboxylic acid (intermediate 17, step b) in place of 2-(5-carbamoyl-2-isopropoxy-phenylamino)-4′-methyl-[4,5′]bithiazolyl-2′-carboxylic acid according to the procedure described in Example 101. ¹H NMR (400 MHz, CHLOROFORM-d) δ 8.87 (d, J=1.96 Hz, 1H), 7.77 (s, 1H), 7.61 (dd, J=2.20, 8.56 Hz, 1H), 6.94 (d, J=8.56 Hz, 1H), 6.79 (s, 1H), 4.15-4.26 (m, 2H), 3.55-3.71 (m, 2H), 3.15 (br. s., 2H), 2.69 (s, 3H), 2.61 (br. s., 3H), 2.30 (s, 6H), 1.53 (t, J=6.97 Hz, 3H); MS m/e 489.0 (M+H).

### Example 124

### 4-Isopropoxy-3-(4′-methyl-2′-morpholin-4-ylmethyl-[4,5′]bithiazolyl-2-ylamino)-benzamide.TFA

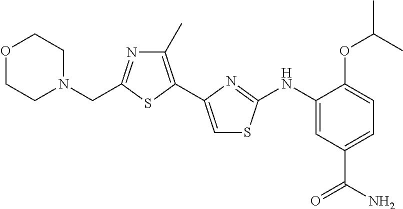

A mixture of 3-(2′-chloromethyl-4′-methyl-[4,5′]bithiazolyl-2-ylamino)-4-isopropoxy-benzamide (41 mg, 0.097 mmol, intermediate 18, step b) and morpholine (85 mg, 0.97 mmol) in CH₃CN was heated at 85° C. for 30 min. After cooling down, the mixture was purified via reverse phase HPLC with water/acetonitrile/0.1% TFA to give the title compound. ¹H NMR (400 MHz, CHLOROFORM-d) δ 8.88 (d, J=2.20 Hz, 1H), 8.10 (br. s., 1H), 7.56 (dd, J=2.08, 8.44 Hz, 1H), 6.95 (d, J=8.80 Hz, 1H), 6.53-6.80 (m, 1H), 4.69-4.80 (m, 1H), 4.50 (s, 2H), 3.98 (t, J=4.65 Hz, 4H), 3.34 (br. s., 4H), 2.66 (s, 3H), 1.40-1.52 (m, 6H); MS m/e 474.0 (M+H).

### Example 125

### 4-Isopropoxy-3-[2′-(4-isopropyl-piperazin-1-ylmethyl)-4′-methyl-[4,5]bithiazolyl-2-ylamino]-benzamide.TFA

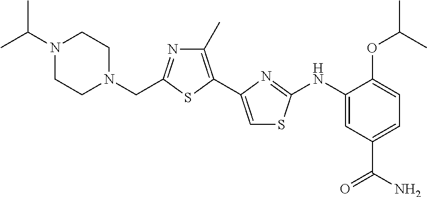

The title compound was prepared using 4-isopropyl-piperazine in place of morpholine according to the procedure described in Example 124. ¹H NMR (400 MHz, CHLOROFORM-d) δ 8.84 (d, J=1.96 Hz, 1H), 7.82 (s, 1H), 7.51 (dd, J=2.20, 8.56 Hz, 1H), 6.94 (d, J=8.80 Hz, 1H), 6.69 (s, 1H), 4.75 (sept, J=6.02 Hz, 1H), 3.89 (s, 2H), 3.37-3.59 (m, 7H), 2.72-2.95 (m, 2H), 2.63 (s, 3H), 1.21-1.51 (m, 12H); MS m/e 515.1 (M+H).

### Example 126

### 4-Isopropoxy-3-[4′-methyl-2′-(4-methyl-[1,4]diazepan-1-ylmethyl)-[4,5′]bithiazolyl-2-ylamino]-benzamide.TFA

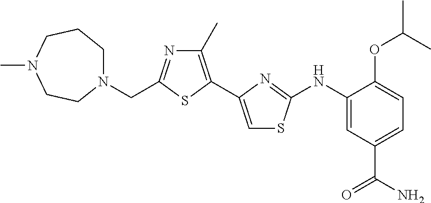

The title compound was prepared using 1-methyl-[1,4]diazepane in place of morpholine according to the procedure described in Example 124. ¹H NMR (400 MHz, CHLOROFORM-d) δ 9.06 (d, J=2.20 Hz, 1H), 7.75 (s, 1H), 7.42 (dd, J=2.20, 8.31 Hz, 1H), 6.92 (d, J=8.80 Hz, 1H), 6.68 (s, 1H), 6.25 (br. s., 2H), 4.74 (sept, J=6.11 Hz, 1H), 3.99 (s, 2H), 3.25-3.73 (m, 4H), 2.92 (s, 3H), 2.61 (s, 3H), 1.70-2.40 (m, 6H), 1.40-1.51 (m, 6H); MS m/e 501.2 (M+H).

### Example 127

### 3-[2′-(4-Hydroxy-piperidin-1-ylmethyl)-4′-methyl-[4,5′]bithiazolyl-2-ylamino]-4-isopropoxy-benzamide.TFA

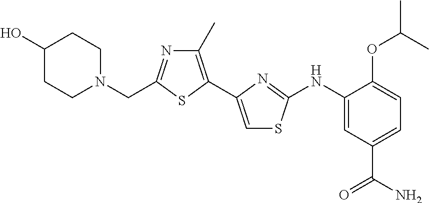

The title compound was prepared using piperidin-4-ol in place of morpholine according to the procedure described in Example 124. ¹H NMR (400 MHz, CHLOROFORM-d) δ 8.91 (d, J=1.96 Hz, 1H), 7.77 (s, 1H), 7.54 (dd, J=2.08, 8.44 Hz, 1H), 6.93 (d, J=8.80 Hz, 1H), 6.73 (s, 1H), 4.74 (sept, J=6.11 Hz, 1H), 4.41 (br. s., 2H), 4.04 (br. s., 1H), 3.20-3.45 (m, 4H), 2.65 (s, 3H), 2.15-2.25 (m, 2H), 1.84-1.97 (m, 2H), 1.35-1.51 (m, 6H); MS m/e 488.2 (M+H).

### Example 128

### 4-Isopropoxy-3-(4′-methyl-2′-piperidin-1-ylmethyl-[4,5′]bithiazolyl-2-ylamino)-benzamide.TFA

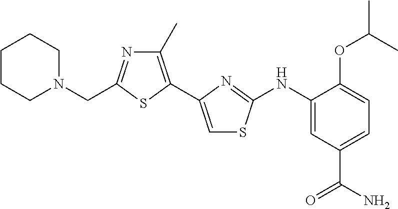

The title compound was prepared using piperidine in place of morpholine according to the procedure described in Example 124. ¹H NMR (400 MHz, CHLOROFORM-d) δ 8.85 (d, J=1.96 Hz, 1H), 7.85 (s, 1H), 7.56 (dd, J=2.08, 8.44 Hz, 1H), 6.90-6.96 (m, 1H), 6.60-6.77 (m, 1H), 4.69-4.80 (m, 1H), 4.49 (s, 2H), 3.27-3.56 (m, 4H), 2.66 (m, 3H), 1.85-2.04 (m, 6H), 1.36-1.51 (m, 6H); MS m/e 472.2 (M+H).

### Example 129

### 3-(2′-Dimethylaminomethyl-4′-methyl-[4,5′]bithiazolyl-2-ylamino)-4-isopropoxy-benzamide.TFA

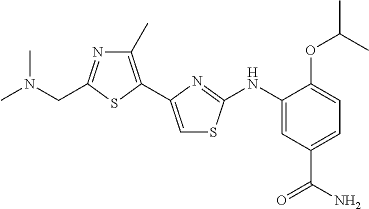

The title compound was prepared using dimethyl amine (2 N in THF) in place of morpholine according to the procedure described in Example 124. ¹H NMR (400 MHz, CHLOROFORM-d) δ 8.78-8.91 (m, 1H), 7.79 (s, 1H), 7.53-7.59 (m, 1H), 6.92-6.97 (m, 1H), 6.74-6.78 (m, 1H), 4.75 (sept, J=6.02 Hz, 1H), 4.49 (s, 2H), 2.90 (s, 6H), 2.68 (s, 3H), 1.40-1.50 (m, 6H); MS m/e 432.1 (M+H).

### Example 130

### 4-Isopropoxy-3-(4′-methyl-2′-pyrrolidin-1-ylmethyl-[4,5′]bithiazolyl-2-ylamino)-benzamide.TFA

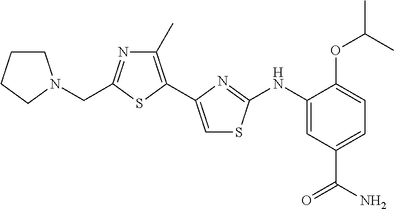

The title compound was prepared using pyrrolidine in place of morpholine according to the procedure described in Example 124. ¹H NMR (400 MHz, CHLOROFORM-d) δ 8.86 (d, J=2.20 Hz, 1H), 7.79 (s, 1H), 7.56 (dd, J=2.20, 8.56 Hz, 1H), 6.90-6.96 (m, 1H), 6.74 (s, 1H), 4.70-4.81 (m, 1H), 4.55 (s, 2H), 3.37-3.59 (m, 4H), 2.66 (s, 3H), 2.00-2.13 (m, 4H), 1.40-1.51 (m, 6H); MS m/e 458.2 (M+H).

### Example 131

### 4-Isopropoxy-3-[4′-methyl-2′-(4-methyl-piperazin-1-ylmethyl)-[4,5′]bithiazolyl-2-ylamino]-benzamide.TFA

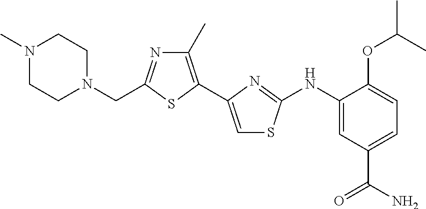

The title compound was prepared using 1-methyl-piperazine in place of morpholine according to the procedure described in Example 124. ¹H NMR (400 MHz, CHLOROFORM-d) δ 8.77 (d, J=2.20 Hz, 1H), 7.82 (s, 1H), 7.63 (dd, J=2.08, 8.44 Hz, 1H), 6.96 (d, J=8.80 Hz, 1H), 6.68 (s, 1H), 4.75 (sept, J=6.08 Hz, 1H), 3.83 (s, 2H), 2.40-2.80 (m, 8H), 2.31 (s, 3H), 2.02 (s, 3H), 1.36-1.55 (m, 6H); MS m/e 487.2 (M+H).

### Example 132

### 4-Isopropoxy-3-(2′-methanesulfonyl-4′-methyl-[4,5′]bithiazolyl-2-ylamino)-benzamide

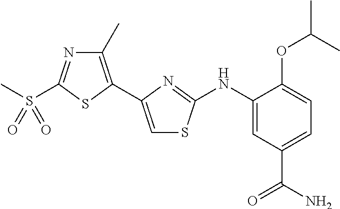

To a solution of 4-isopropoxy-3-(4′-methyl-2′-methylsulfanyl-[4,5′]bithiazolyl-2-ylamino)-benzamide.HBr (250 mg, 0.594 mmol, intermediate 19, step d) in THF (3 mL) was added OXONE® (1.46 g, 2.38 mmol) in water (3 mL). The reaction mixture was stirred for 3 h and quenched with 1.5 M Na₂S₂O₃ (10 mL) and saturated NaHCO₃ solution (10 mL). The solution was extracted with EtOAc and the organic layer was dried (Na₂SO₄), filtered, concentrated and purified through column chromatography to yield the title compound. MS m/e 453.1 (M+H).

### Example 133

### 3-(2′-{[(2-Dimethylamino-ethyl)-methyl-amino]-methyl}-4′-methyl-[4,5]bithiazolyl-2-ylamino)-4-isopropoxy-benzamide.TFA

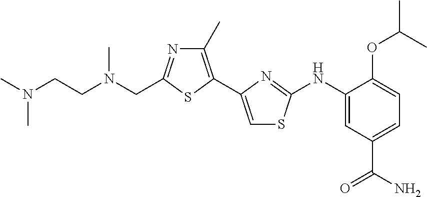

The title compound was prepared using N,N,N′-trimethyl-ethane-1,2-diamine in place of morpholine according to the procedure described in Example 124. ¹H NMR (400 MHz, CHLOROFORM-d) δ 9.02 (d, J=2.20 Hz, 1H), 7.84 (s, 1H), 7.49 (dd, J=2.08, 8.44 Hz, 1H), 6.93 (d, J=8.56 Hz, 1H), 6.69 (s, 1H), 4.74 (sept, J=6.17 Hz, 1H), 3.95 (s, 2H), 3.27-3.35 (m, 2H), 3.00-3.15 (m, 2H), 2.86-3.00 (m, 6H), 2.62 (s, 3H), 2.45 (s, 3H), 1.38-1.51 (m, 6H); MS m/e 489.2 (M+H).

### Example 134

### 1-{5-[3-(2′,4′-Dimethyl-[4,5′]bithiazolyl-2-ylamino)-4-methoxy-phenyl]-thiophen-2-yl}-ethanone

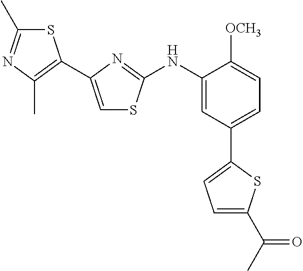

A mixture of (5-bromo-2-methoxy-phenyl)-(2′,4′-dimethyl-[4,5′]bithiazolyl-2-yl)-amine (60 mg, 0.126 mmol, intermediate 20, step c), 5-acetyl-thiophene-2-boronic acid (32 mg, 0.189 mmol), PdCl₂(dppf).CH₂Cl₂ (20 mg, 0.025 mmol) and K₂CO₃ (69.5 mg, 0.503 mmol) in 1,4-dioxane (1 mL) and H₂O (1 mL) was heated at 90° C. for 5 h. After cooling down the reaction mixture was partitioned between water and EtOAc, the organic layer was dried (Na₂SO₄), filtered, concentrated and purified through column chromatography to afford the title compound. ¹H NMR (400 MHz, CHLOROFORM-d) δ 8.78 (d, J=2.20 Hz, 1H), 7.80 (s, 1H), 7.67 (d, J=3.91 Hz, 1H), 7.21-7.35 (m, 2H), 6.91 (d, J=8.31 Hz, 1H), 6.64 (s, 1H), 3.96 (s, 3H), 2.72 (s, 3H), 2.63 (s, 3H), 2.57 (s, 3H); MS m/e 442.1 (M+H).

### Example 135

### (2′,4′-Dimethyl-[4,5′]bithiazolyl-2-yl)-[5-(3,5-dimethyl-isoxazol-4-yl)-2-methoxy-phenyl]-amine

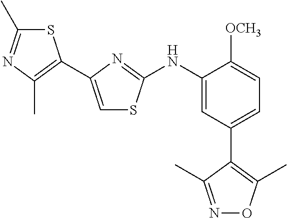

A mixture of (5-bromo-2-methoxy-phenyl)-(2′,4′-dimethyl-[4,5′]bithiazolyl-2-yl)-amine (60 mg, 0.126 mmol, intermediate 20, step c), 3,5-dimethyl-4-(4,4,5,5-tetramethyl-[1,3,2]dioxaborolan-2-yl)-isoxazole (33.7 mg, 0.151 mmol), Pd(PPh₃)₄ (14.5 mg, 0.013 mmol) and 2 M Na₂CO₃ (0.189 mL, 0.377 mmol) in DME (1.5 mL) was heated at 95° C. for 6 h. After cooling down the reaction mixture was partitioned between water and EtOAc, the organic layer was dried (Na₂SO₄), filtered, concentrated and purified through column chromatography to afford the title compound. ¹H NMR (400 MHz, CHLOROFORM-d) δ 8.16 (d, J=2.20 Hz, 1H), 7.84 (s, 1H), 6.96 (d, J=8.31 Hz, 1H), 6.87 (dd, J=2.08, 8.19 Hz, 1H), 6.62 (s, 1H), 3.96 (s, 3H), 2.63-2.70 (m, 3H), 2.58 (s, 3H), 2.46 (s, 3H), 2.34 (s, 3H); MS m/e 413.1 (M+H).

### Example 136

### (2′,4′-Dimethyl-[4,5′]bithiazolyl-2-yl)-[2-methoxy-5-(1-methyl-1H-pyrazol-4-yl)-phenyl]-amine

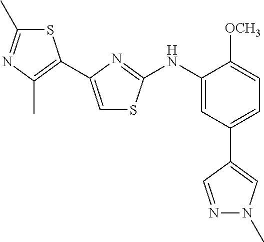

The title compound was prepared using 1-methyl-4-(4,4,5,5-tetramethyl-[1,3,2]dioxaborolan-2-yl)-1H-pyrazole in place of 3,5-dimethyl-4-(4,4,5,5-tetramethyl-[1,3,2]dioxaborolan-2-yl)-isoxazole according to the procedure described in Example 135. ¹H NMR (400 MHz, CHLOROFORM-d) δ 8.42 (d, J=1.96 Hz, 1H), 7.70-7.86 (m, 2H), 7.63 (s, 1H), 7.09 (dd, J=2.20, 8.31 Hz, 1H), 6.88 (d, J=8.56 Hz, 1H), 6.61 (s, 1H), 3.72-4.01 (m, 6H), 2.69 (s, 3H), 2.63 (s, 3H); MS m/e 398.1 (M+H).

### Example 137

### (2′,4′-Dimethyl-[4,5′]bithiazolyl-2-yl)-[2-methoxy-5-(1H-pyrazol-3-yl)-phenyl]-amine

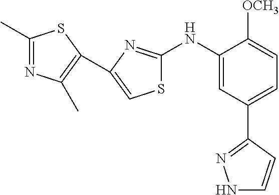

The title compound was prepared using 1H-pyrazole-3-boronic acid in place of 5-acetyl-thiophene-2-boronic acid according to the procedure described in Example 134. ¹H NMR (400 MHz, CHLOROFORM-d) δ 8.57 (d, J=1.71 Hz, 1H), 7.92 (br. s., 1H), 7.63 (d, J=2.20 Hz, 1H), 7.39 (d, J=7.09 Hz, 1H), 6.91 (d, J=8.31 Hz, 1H), 6.54-6.67 (m, 2H), 3.92 (s, 3H), 2.66 (s, 3H), 2.60 (s, 3H); MS m/e 384.1 (M+H).

### Example 138

### (2′,4′-Dimethyl-[4,5′]bithiazolyl-2-yl)-[2-methoxy-5-(1H-pyrazol-4-yl)-phenyl]-amine

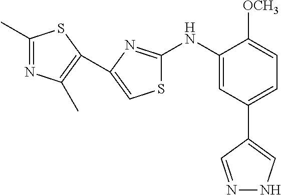

The title compound was prepared using 1H-pyrazole-4-boronic acid in place of 5-acetyl-thiophene-2-boronic acid according to the procedure described in Example 134. ¹H NMR (400 MHz, MeOD) δ 8.65 (d, J=1.96 Hz, 1H), 7.82 (br. s., 2H), 7.09 (dd, J=1.96, 8.31 Hz, 1H), 6.89 (d, J=8.56 Hz, 1H), 6.73 (s, 1H), 3.83 (s, 3H), 2.57 (s, 3H), 2.47 (s, 3H); MS m/e 384.1 (M+H).

### Example 139

### (2′,4′-Dimethyl-[4,5′]bithiazolyl-2-yl)-[2-methoxy-5-(1-methyl-1H-imidazol-2-yl)-phenyl]-amine

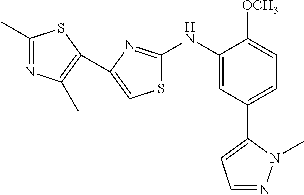

The title compound was prepared using 1-methyl-5-(4,4,5,5-tetramethyl-[1,3,2]dioxaborolan-2-yl)-1H-pyrazole in place of 5-acetyl-thiophene-2-boronic acid according to the procedure described in Example 134. ¹H NMR (400 MHz, CHLOROFORM-d) δ 8.37 (d, J=2.20 Hz, 1H), 7.89 (s, 1H), 7.52 (d, J=1.96 Hz, 1H), 7.05 (dd, J=2.20, 8.31 Hz, 1H), 6.96 (d, J=8.31 Hz, 1H), 6.61 (s, 1H), 6.34 (d, J=1.96 Hz, 1H), 3.95 (s, 3H), 3.98 (s, 3H), 2.66 (s, 3H), 2.58 (s, 3H); MS m/e 398.1 (M+H).

### Example 140

### (2′,4′-Dimethyl-[4,5′]bithiazolyl-2-yl)-(2-methoxy-5-vinyl-phenyl)-amine

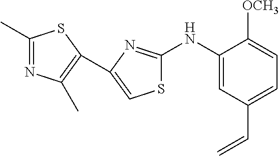

The title compound was prepared using 4,4,5,5-tetramethyl-2-vinyl-[1,3,2]dioxaborolane in place of 5-acetyl-thiophene-2-boronic acid according to the procedure described in Example 134. ¹H NMR (400 MHz, CHLOROFORM-d) δ 8.35 (d, J=1.96 Hz, 1H), 7.75 (s, 1H), 7.00 (dd, J=1.96, 8.31 Hz, 1H), 6.84 (d, J=8.31 Hz, 1H), 6.70 (dd, J=10.88, 17.48 Hz, 1H), 6.60 (s, 1H), 5.74 (d, J=17.61 Hz, 1H), 5.20 (d, J=10.76 Hz, 1H), 3.91 (s, 3H), 2.68 (s, 3H), 2.63 (s, 3H); MS m/e 344.1 (M+H).

### Example 141

### (2′,4′-Dimethyl-[4,5′]bithiazolyl-2-yl)-(2-methoxy-5-pyridin-3-yl-phenyl)-amine

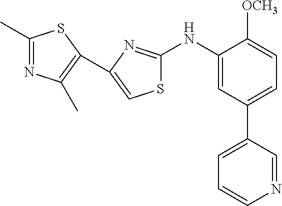

The title compound was prepared using 3-pyridyl-boronic acid in place of 5-acetyl-thiophene-2-boronic acid according to the procedure described in Example 134. ¹H NMR (400 MHz, CHLOROFORM-d) δ 8.92 (br. s., 1H), 8.58 (d, J=2.20 Hz, 2H), 7.95 (dt, J=1.83, 7.83 Hz, 1H), 7.87 (s, 1H), 7.36 (dd, J=4.65, 7.83 Hz, 1H), 7.14-7.31 (m, 1H), 6.99 (d, J=8.31 Hz, 1H), 6.62 (s, 1H), 3.96 (s, 3H), 2.68 (s, 3H), 2.61 (s, 3H); MS m/e 395.1 (M+H).

### Example 142

### (2′,4′-Dimethyl-[4,5′]bithiazolyl-2-yl)-(2-methoxy-5-thiazol-2-yl-phenyl)-amine.TFA

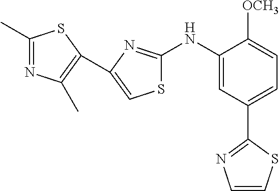

A mixture of (5-bromo-2-methoxy-phenyl)-(2′,4′-dimethyl-[4,5′]bithiazolyl-2-yl)-amine (50 mg, 0.126 mmol, intermediate 20, step c), thiazole-2-zinc bromide in THF (2 M, 1.26 mL, 0.631 mmol), and PdCl₂(dppf).CH₂Cl₂ (20 mg, 0.025 mmol) in THF (0.6 mL) was degassed under N₂ and then heated at 76° C. for 5 h. After cooling down the reaction mixture was purified via reverse phase HPLC with water/acetonitrile/0.1% TFA to afford the title compound. ¹H NMR (400 MHz, CHLOROFORM-d) δ 8.71 (d, J=2.20 Hz, 2H), 8.05 (d, J=3.67 Hz, 1H), 7.75 (dd, J=2.20, 8.56 Hz, 1H), 7.45 (d, J=3.42 Hz, 1H), 7.07 (d, J=8.56 Hz, 1H), 6.80 (s, 1H), 4.00 (s, 3H), 2.93 (s, 3H), 2.70 (s, 3H); MS m/e 401.0 (M+H).

### Example 143

### (2′,4′-Dimethyl-[4,5′]bithiazolyl-2-yl)-[2-methoxy-5-(3-methyl-3H-imidazol-4-yl)-phenyl]-amine

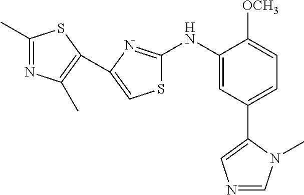

The title compound was prepared using (2′,4′-Dimethyl-[4,5′]bithiazolyl-2-yl)-[2-methoxy-5-(4,4,5,5-tetramethyl[1,3,2]dioxaborolan-2-yl)-phenyl]-amine (intermediate 21) in place of 5-acetyl-thiophene-2-boronic acid and 5-bromo-1-methyl-1H-imidazole in place of (5-bromo-2-methoxy-phenyl)-(2′,4′-dimethyl-[4,5′]bithiazolyl-2-yl)-amine, according to the procedure described in Example 134. ¹H NMR (400 MHz, CHLOROFORM-d) δ 8.39 (s, 1H), 7.83 (br. s., 1H), 7.38 (d, J=8.31 Hz, 1H), 7.12 (s, 1H), 6.93-7.03 (m, 2H), 6.61 (s, 1H), 3.97 (s, 3H), 3.84 (s, 3H), 2.67 (s, 3H), 2.59 (s, 3H); MS m/e 398.1 (M+H).

### Example 144

### 3-(2′,4′-Dimethyl-[4,5′]bithiazolyl-2-ylamino)-4-trifluoromethoxy-benzenesulfonamide.HCl

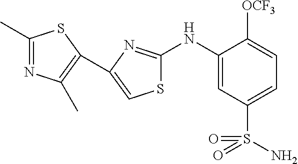

A mixture of 2-bromo-1-(2,4-dimethyl-thiazol-5-yl)-ethanone.HBr (300 mg, 0.951 mmol, intermediate 3) and 3-thioureido-4-trifluoromethoxy-benzenesulfonamide (300 mg, 0.951 mmol, intermediate 62, step d) in EtOH (10 mL) was stirred at room temperature for 6 h. The mixture was basified with 2 N NH₃/MeOH and silica gel (300 mesh, ˜5 g) was added. The mixture was concentrated and purified through solid loading on column chromatography (70% EtOAc/heptane) to afford the free base of the title compound. This material was suspended in MeOH (3 mL) and 1 N HCl/ether (10 mL) was added dropwise. The mixture was stirred for 30 min and was filtered, washed with EtOAc and dried under vacuum to afford the title compound as a white solid. ¹H NMR (400 MHz, DMSO-d₆) δ 10.47 (s, 1H), 9.24 (d, J=1.96 Hz, 1H), 7.63 (d, J=8.31 Hz, 1H), 7.38-7.56 (m, 3H), 7.21 (s, 1H), 2.68 (s, 3H), 2.58 (s, 3H); MS m/e 451.0 (M+H).

### Example 145

### 4-Isopropoxy-3-[4′-methyl-2′-(pyrazin-2-ylamino)-[4,5′]bithiazolyl-2-ylamino]-benzamide.TFA

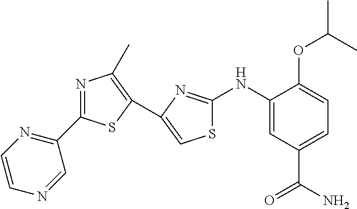

The title compound was prepared using 2-bromo-1-(4-methyl-2-pyrazin-2-yl-thiazol-5-yl)-ethanone (Maybridge) in place of 2-bromo-1-(2-dimethylamino-4-methyl-thiazol-5-yl)-ethanone.HBr, according to the procedure described in Example 88. ¹H NMR (400 MHz, MeOD) δ 9.19 (s, 1H), 8.81 (d, J=2.20 Hz, 1H), 8.45-8.51 (m, 2H), 7.44 (dd, J=2.20, 8.56 Hz, 1H), 6.98 (d, J=8.80 Hz, 1H), 6.94 (s, 1H), 4.66-4.77 (m, 1H), 2.63 (s, 3H), 1.32 (d, J=6.11 Hz, 6H); MS m/e 453.0 (M+H).

### Example 146

### N-[4′-Methyl-2-(quinolin-8-ylamino)-[4,5′]bithiazolyl-2′-yl]-acetamide.TFA

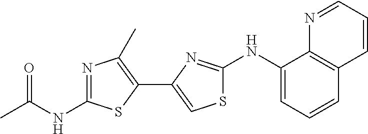

A mixture of quinolin-8-yl-thiourea (0.28 g, 1.4 mmol, intermediate 47) and N-[5-(2-bromo-acetyl)-4-methyl-thiazol-2-yl]-acetamide.HBr (0.4 g, 1.1 mmol, intermediate 5) in EtOH (20 mL) was heated to reflux overnight. The mixture was concentrated and the residue was purified by reverse phase HPLC to afford the title compound. ¹H NMR (400 MHz, DMSO-d₆) δ 12.08 (s, 1H), 10.81 (s, 1H), 8.95 (dd, J=1.71, 4.16 Hz, 1H), 8.84 (dd, J=1.22, 7.58 Hz, 1H), 8.41 (dd, J=1.47, 8.31 Hz, 1H), 7.53-7.67 (m, 3H), 7.02 (s, 1H), 2.53 (s, 3H), 2.15 (s, 3H). MS m/e 382.1 (M+H).

### Example 147

### N-[2-(2-Methoxy-pyridin-3-ylamino)-4′-methyl-[4,5′]bithiazolyl-2′-yl]-acetamide.TFA

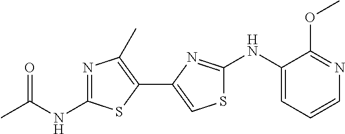

A mixture of (2-methoxy-pyridin-3-yl)-thiourea (0.5 g, 2.7 mmol, intermediate 63), N-[5-(2-bromo-acetyl)-4-methyl-thiazol-2-yl]-acetamide.HBr (0.75 g, 2.1 mmol, intermediate 5) and Et₃N (1.14 mL, 8.1 mmol) in EtOH (100 mL) was heated at reflux overnight. The mixture was concentrated and the residue was purified by reverse phase HPLC, and recrystallized (1/1 CH₂Cl₂/MeOH, v/v) to afford the title compound. ¹H NMR (400 MHz, DMSO-d₆) δ 12.08 (s, 1H), 9.95 (s, 1H), 8.76 (d, J=7.58 Hz, 1H), 7.76 (d, J=5.13 Hz, 1H), 7.01 (dd, J=5.01, 7.70 Hz, 1H), 6.95 (s, 1H), 3.98 (s, 3H), 2.48 (s, 3H), 2.14 (s, 3H). MS m/e 362.1 (M+H).

### Example 148

### N-[2-(2-Hydroxy-pyridin-3-ylamino)-4′-methyl-[4,5′]bithiazolyl-2′-yl]-acetamide.HBr

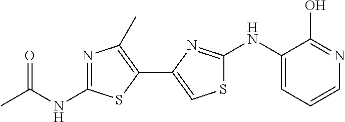

A mixture of (2-methoxy-pyridin-3-yl)-thiourea (0.08 g, 0.44 mol, intermediate 63) and N-[5-(2-bromo-acetyl)-4-methyl-thiazol-2-yl]-acetamide.HBr (0.12 g, 0.34 mol, intermediate 5) in EtOH (20 mL) was heated at 80° C. overnight in a sealed tube. The reaction mixture was cooled to room temperature and was filtered and washed with EtOH, to afford the title compound. ¹H NMR (400 MHz, DMSO-d₆) δ 12.07 (br. s., 1H), 11.86 (br. s., 1H), 10.04 (s, 1H), 8.45 (dd, J=1.47, 7.34 Hz, 1H), 6.97-7.03 (m, 1H), 6.93 (s, 1H), 6.27 (t, J=6.97 Hz, 1H), 2.48 (s, 3H), 2.14 (s, 3H). MS m/e 348.1 (M+H).

### Example 149

### N-[4′-Methyl-2-(naphthalen-1-ylamino)-[4,5′]bithiazolyl-2′-yl]-acetamide.HCl

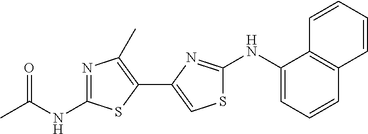

A mixture of commercially available naphthalen-1-yl-thiourea (0.283 g, 1.4 mmol) and N-[5-(2-bromo-acetyl)-4-methyl-thiazol-2-yl]-acetamide.HBr (1.1 g, 3.1 mmol, intermediate 5) in EtOH (20 mL) was heated at reflux for 12 h. The mixture was concentrated and the residue was purified by reverse phase HPLC. The eluant was concentrated and the residue was dissolved in THF and added to 1 N HCl in Et₂O. The precipitate was collected by filtration and lyophilized to yield the title compound. ¹H NMR (400 MHz, DMSO-d₆) δ 12.08 (br. s., 1H), 10.25 (br. s., 1H), 8.26-8.33 (m, 1H), 8.19 (d, J=7.58 Hz, 1H), 7.92-7.99 (m, 1H), 7.69 (d, J=8.07 Hz, 1H), 7.49-7.61 (m, 3H), 6.89 (s, 1H), 2.47 (s, 3H), 2.14 (s, 3H). MS m/e 381.1 (M+H).

### Example 150

### 3-(2′-Amino-4′-trifluoromethyl-[4,5′]bithiazolyl-2-ylamino)-4-isopropoxy-benzamide

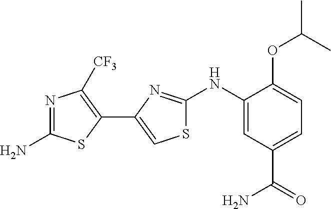

The title compound was prepared using 1-(2-amino-4-trifluoromethyl-thiazol-5-yl)-2-bromo-ethanone (intermediate 6, step c) in place of 1-(2-amino-4-methyl-thiazol-5-yl)-2-bromo-ethanone and 4-isopropoxy-3-thioureido-benzamide (intermediate 23, step e) in place of 4-ethoxy-3-thioureido-benzamide as described in example 7. ¹H NMR (300 MHz, MeOD) δ 8.91 (d, J=2.26 Hz, 1H), 7.51 (dd, J=2.26, 8.67 Hz, 1H), 7.06 (d, J=9.04 Hz, 1H), 6.89 (s, 1H), 4.74-4.84 (m, 1H), 1.42 (d, J=6.03 Hz, 6H). MS m/e 444.0 (M+H).

### Example 151

### N-(4-Methoxypyridin-3-yl)-2′,4′-dimethyl-[4,5′-bithiazol]-2-amine

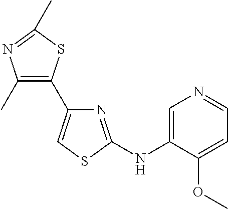

A mixture of 2-bromo-1-(2,4-dimethyl-thiazol-5-yl)-ethanone.HBr (60.5 mg, 0.192 mmol, intermediate 3), 1-(4-methoxypyridin-3-yl)thiourea (35.2 mg, 0.192 mmol, intermediate 64, step b), and EtOH (2 mL) was stirred at room temperature overnight. The reaction mixture was diluted with sat. aq. NaHCO₃ and extracted with EtOAc. The organic phase was dried (Na₂SO₄), filtered, and concentrated. The crude product was purified by column chromatography (Silica gel, 0-4% MeOH-EtOAc), affording the title compound as a cream-colored solid. ¹H NMR (400 MHz, DMSO-d₆) δ 9.81 (s, 1H), 9.47 (s, 1H), 8.16 (d, J=5.38 Hz, 1H), 7.10 (d, J=5.38 Hz, 1H), 6.97 (s, 1H), 3.94 (s, 3H), 2.59 (s, 3H), 2.51 (s, 3H). MS m/e 319.0 (M+H).

### Example 152

### 4-Isopropoxy-3-((2′,4′,5-trimethyl-[4,5′-bithiazol]-2-yl)amino)benzenesulfonamide

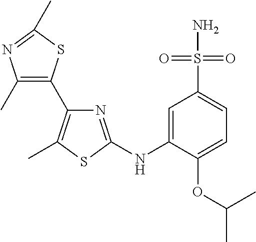

The title compound was prepared using 4-isopropoxy-3-thioureidobenzenesulfonamide (intermediate 55, step e) and 2-bromo-1-(2,4-dimethylthiazol-5-yl)propan-1-one (intermediate 67, step b) according to the procedure of example 151. ¹H NMR (400 MHz, DMSO-d₆) δ 9.41 (s, 1H), 8.90 (d, J=2.20 Hz, 1H), 7.38 (dd, J=2.20, 8.56 Hz, 1H), 7.18 (d, J=8.80 Hz, 1H), 7.11 (s, 2H), 4.75-4.83 (m, 1H), 2.62 (s, 3H), 2.37 (s, 3H), 2.28 (s, 3H), 1.35 (d, J=6.11 Hz, 6H). MS m/e 438.9 (M+H).

### Example 153

### 4-Isopropoxy-3-((2′,4′,5-trimethyl-[4,5′-bithiazol]-2-yl)amino)benzamide

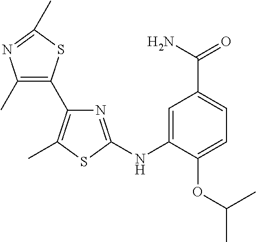

The title compound was prepared using 4-isopropoxy-3-isothiocyanato-benzamide (intermediate 23, step e) and 2-bromo-1-(2,4-dimethylthiazol-5-yl)propan-1-one (intermediate 67, step b) according to the procedure of example 151. ¹H NMR (400 MHz, DMSO-d₆) δ 9.19 (s, 1H), 8.72 (d, J=2.20 Hz, 1H), 7.70 (br. s., 1H), 7.46 (dd, J=2.08, 8.44 Hz, 1H), 7.09 (br. s., 1H), 7.05 (d, J=8.56 Hz, 1H), 4.74 (sept, J=5.99 Hz, 1H), 2.62 (m, 3H), 2.36 (s, 3H), 2.26 (s, 3H), 1.33 (d, J=5.87 Hz, 6H). MS m/e 403.0 (M+H).

### Example 154

### 3-((2′,4′-Dimethyl-[4,5′-bithiazol]-2-yl)amino)-4-(ethylthio)benzenesulfonamide

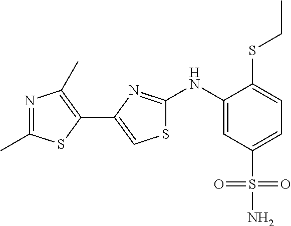

The title compound was prepared using 4-(ethylthio)-3-thioureidobenzenesulfonamide (intermediate 66, step d) according to the procedure of example 151 (chromatography eluent 50-100% EtOAc-Hept). ¹H NMR (400 MHz, DMSO-d₆) δ 9.71 (s, 1H), 8.43 (d, J=1.96 Hz, 1H), 7.58 (d, J=8.31 Hz, 1H), 7.53 (dd, J=1.96, 8.31 Hz, 1H), 7.34 (s, 2H), 6.98 (s, 1H), 3.01 (q, J=7.34 Hz, 2H), 2.57 (s, 3H), 2.50 (s, 3H), 1.24 (t, J=7.21 Hz, 3H). MS m/e 426.9 (M+H).

### Example 155

### N-(4-Methoxypyridin-3-yl)-4′-methyl-2′-(4-methylpiperazin-1-yl)-[4,5′-bithiazol]-2-amine.TFA

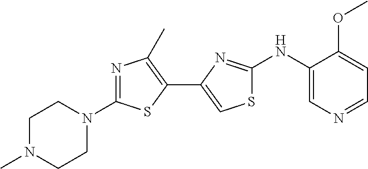

The title compound was prepared using 1-(4-methoxypyridin-3-yl)thiourea (intermediate 64, step b) and 2-bromo-1-[4-methyl-2-(4-methyl-piperazin-1-yl)-thiazol-5-yl]-ethanone.HBr (intermediate 7, step c) according to the procedure of example 151, except that the crude product was purified by RP-HPLC (10-40% CH₃CN—H₂O, 0.1% TFA). ¹H NMR (400 MHz, DMSO-d₆) δ 10.58 (br. s., 1H), 10.01 (br. s., 1H), 9.74 (s, 1H), 8.55 (d, J=6.36 Hz, 1H), 7.63 (d, J=6.11 Hz, 1H), 6.97 (s, 1H), 4.18 (s, 3H), 3.06-4.14 (m, 8H), 2.87 (s, 3H), 2.42 (s, 3H). MS m/e 403.0 (M+H).

### Example 156

### 4-Isopropoxy-3-((4′-methyl-2′-(1-methylpiperidin-4-yl)-[4,5′-bithiazol]-2-yl)amino)benzenesulfonamide.TFA

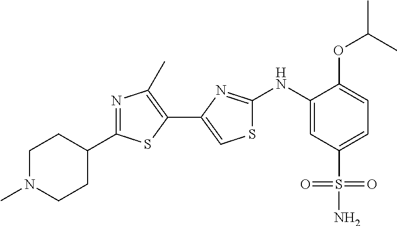

The title compound was prepared using 4-isopropoxy-3-thioureidobenzenesulfonamide (intermediate 55, step e) and 2-bromo-1-(4-methyl-2-(1-methylpiperidin-4-yl)thiazol-5-yl)ethanone.HBr (intermediate 68, step c) according to the procedure of example 151, except that the crude product was purified by RP-HPLC (10-90% CH₃CN—H₂O, 0.1% TFA). ¹H NMR (400 MHz, DMSO-d₆) δ 9.66 (s, 1H), 9.38 (br. s., 1H), 9.07 (m, 1H), 7.43 (dd, J=2.32, 8.68 Hz, 1H), 7.22 (d, J=8.31 Hz, 1H), 7.12 (br. s., 2H), 7.06 (s, 1H), 4.75-4.86 (m, 1H), 3.51-3.59 (m, 1H), 3.04-3.28 (m, 4H), 2.82 (m, 3H), 2.56 (s, 3H), 2.23-2.32 (m, 2H), 1.81-1.98 (m, 2H), 1.36 (d, J=5.87 Hz, 6H). MS m/e 508.2 (M+H).

### Example 157

### 4-Isopropoxy-3-((4′-methyl-2′-(1-methylpiperidin-4-yl)-[4,5′-bithiazol]-2-yl)amino)benzamide.TFA

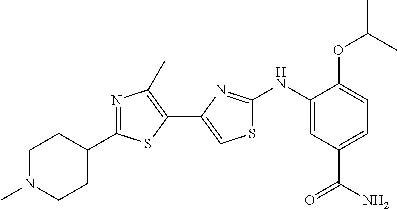

The title compound was prepared using 4-isopropoxy-3-isothiocyanato-benzamide (intermediate 23, step e) and 2-bromo-1-(4-methyl-2-(1-methylpiperidin-4-yl)thiazol-5-yl)ethanone.HBr (intermediate 68, step c) according to the procedure of example 151, except that the crude product was purified by RP-HPLC (10-90% CH₃CN—H₂O, 0.1% TFA). ¹H NMR (400 MHz, DMSO-d₆) δ 9.45 (s, 1H), 9.39 (br. s., 1H), 8.84-8.89 (m, 1H), 7.73 (br. s., 1H), 7.49-7.55 (m, 1H), 7.06-7.15 (m, 2H), 7.01 (s, 1H), 4.73-4.82 (m, 1H), 3.49-3.58 (m, 2H), 3.21-3.28 (m, 1H), 3.04-3.16 (m, 2H), 2.79-2.85 (m, 3H), 2.56 (s, 3H), 2.21-2.32 (m, 2H), 1.83-1.97 (m, 2H), 1.34 (d, J=6.11 Hz, 6H). MS m/e 472.3 (M+H).

### Example 158

### N-(4-Ethoxypyridin-3-yl)-4′-methyl-2′-(1-methylpiperidin-4-yl)-[4,5′-bithiazol]-2-amine.TFA

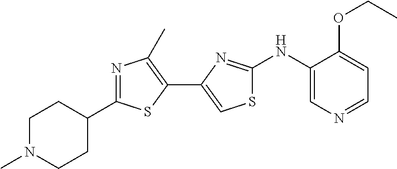

The title compound was prepared using 1-(4-ethoxypyridin-3-yl)thiourea (intermediate 65, step b) and 2-bromo-1-(4-methyl-2-(1-methylpiperidin-4-yl)thiazol-5-yl)ethanone.HBr (intermediate 68, step c) according to the procedure of example 151, except that the crude product was purified by RP-HPLC (10-90% CH₃CN—H₂O, 0.1% TFA). ¹H NMR (400 MHz, DMSO-d₆) δ 10.48 (br. s., 1H), 9.78 (s, 1H), 9.53 (br. s., 1H), 8.55 (d, J=5.87 Hz, 1H), 7.64 (d, J=6.60 Hz, 1H), 7.24 (s, 1H), 4.45-4.58 (q, J=6.85 Hz, 2H), 3.51-3.58 (m, 2H), 3.05-3.28 (br. s., 3H), 2.79-2.87 (m, 3H), 2.57 (s, 3H), 2.23-2.35 (m, 2H), 1.84-1.98 (m, 2H), 1.51 (t, J=6.97 Hz, 3H). MS m/e 472.3 (M+H).

### Cloning, Expression and Purification

Cloning of human proMMP9

Amino acid numbering for all human proMMP9 constructs was based on UniProtKB/Swiss-Prot P14780, full-length human matrix metalloproteinase-9 precursor, proMMP9(1-707) (SEQ ID NO:1). One construct, proMMP9(20-445) (SEQ ID NO:2), was based on the previously published crystal structure (Acta Crystallogr D Biol Crystallogr 58(Pt 7): 1182-92). The construct lacked the signal peptide at the N-terminus and also lacked the four hemopexin-like domains at the C-terminus. An N-terminal truncated construct was also designed with an N-terminus truncation after the first observable electron density in the previously published proMMP9 structure and a single amino acid was removed from the C-terminus to produce proMMP9(29-444) (SEQ ID NO:3). Other truncated constructs were also synthesized without the three fibronectin type-II domains (ΔFnII), amino acids 216-390. The ΔFnII constructs were proMMP9(29-444;ΔFnII) (SEQ ID NO:4), proMMP9(67-444;ΔFnII) (SEQ ID NO:5) and proMMP9(20-445;ΔFnII) (SEQ ID NO:6). Binding studies with the proMMP9 proteins without the FnII domains showed that compounds bound with similar affinity compared to the wild-type protein (data not shown).

In order to make the constructs with the FnII domains deleted, proMMP9(29-444;ΔFnII) (SEQ ID NO:4), proMMP9(67-444;ΔFnII) (SEQ ID NO:5) and proMMP9(20-445;ΔFnII) (SEQ ID NO:6), plasmids encoding the different proMMP9 truncations were used as templates for PCR to create two fragments of DNA corresponding to amino acid pairs including: 29-215/391-444, 67-215/391-444, and 20-215/391-445, respectively. Overlapping PCR was used to join the fragments. The 5′ primers had an Nde1 site and a start methionine and the 3′ primers had a stop codon and a Bgl2 site. The final PCR products were cloned into the TOPO TA cloning vector (Invitrogen) and the sequences were confirmed. Subsequently the vectors were digested with Nde1 and Bgl2 and the sequences were subcloned into Nde1 and BamH1 sites of the T7 expression vector pET11a (Novagen).

Expression of Truncated Forms of Human proMMP9

For expression in E. coli, all of the truncated proMMP9 constructs were transformed into BL21(DE3) RIL cells (Stratagene). Cells were initiated for an overnight culture from glycerol stocks in LB+Ampicillin (100 μg/ml) @ 37° C. shaking at 220 rpms. The overnight culture was subcultured 1:100 in LB+Ampicillin (100 ug/ml) and maintained at 37° C. shaking at 220 rpms. Samples were taken and A600 readings were monitored until an OD of 0.6 was achieved. The culture was induced with 1 mM IPTG and maintained under present growth conditions. Cultures were harvested 3 hours post induction at 6000×g for 10 min. Pellets were washed in 1×PBS with protease inhibitors and stored at −80° C.

Purification of truncated forms of human proMMP9

To purify the truncated proMMP9 proteins from E. coli, cell pellets were suspended in 25 mM Na₂HPO₄ pH 7, 150 mM NaCl, 10 mL/gram cell pellet. The cells were homogenized in a Dounce homogenizer, and then processed twice through a microfluidizer (Microfluidics International Corporation, model M-110Y). The lysate was centrifuged at 32,000×g for 45 minutes at 4° C. The supernatant was discarded. The pellet was suspended in 25 mM Na₂HPO₄ pH 7, 150 mM NaCl, 10 mM DTT, 1 mM EDTA, 10 mL/gram cell pellet. The pellet was homogenized in a Dounce homogenizer, and then centrifuged at 32,000×g for 45 minutes at 4° C. The supernatant was discarded. The pellet was suspended in 7 M urea, 25 mM Tris pH 7.5, 10 mM DTT, 1 mM EDTA, 6.5 mL/gram cell pellet, and then solubilized in a Dounce homogenizer and stirred for approximately 16 hours at ambient temperature. The solubilized protein solution was adjusted to pH 7.5, centrifuged at 45,000×g, 45 minutes at 4° C., and the supernatant, containing the denatured proMMP9, was filtered to 0.8 micron. A 5 mL HiTrap Q Sepharose HP column (GE Healthcare) was prepared according to manufacturer's instructions using Buffer A: 7 M urea, 25 mM Tris pH 7.5 and Buffer B: 7 M urea, 25 mM Tris pH 7.5, 1.0 M NaCl. The protein solution was applied to the HiTrap at 2.5 mL/minute. The column was washed to baseline absorbance with approximately 3.5 CV Buffer A. The proMMP9 was eluted in a 12 CV linear gradient from 0% Buffer B to 12% Buffer B. Fractions were collected, analyzed on SDS-PAGE (Novex) and pooled based on purity. The pooled protein was re-natured by drop-wise addition to a solution, stirring and at ambient temperature, of 20 mM Tris pH 7.5, 200 mM NaCl, 5 mM CaCl₂, 1 mM ZnCl₂, 0.7 M L-arginine, 10 mM reduced and 1 mM oxidized glutathione, and was stirred for approximately 16 hours at 4° C. The refolded protein was concentrated to approximately 2.5 mg/mL in Jumbo Sep centrifugal concentrators (Pall) with 10,000 MWCO membranes. The concentrated protein solution was dialyzed at 4° C. for approximately 16 hours against 20 mM Tris pH 7.5, 150 mM NaCl. The dialyzed protein solution was clarified by filtration to 0.8 micron, concentrated to 2 mg/mL as before, centrifuged at 45,000×g for 15 minutes at 4° C. and filtered to 0.2 micron. It was purified on a HiLoad 26/60 Superdex 200 column (GE Healthcare) equilibrated in 20 mM Tris pH 7.5, 200 mM NaCl. Fractions were analyzed by SDS-PAGE and pooled based on purity. The pooled protein was concentrated in a Jumbo Sep concentrator as before and centrifuged at 16,000×g for 10 minutes at 4° C. The protein concentration was determined using Bio-Rad Protein Assay (Bio-Rad Laboratories, Inc.) with bovine serum albumin as a standard. The supernatant was aliquoted, frozen in liquid nitrogen and stored at −80° C.

Full-Length Human proMMP9

Full-length proMMP9(1-707) (SEQ ID NO:1) was expressed in HEK293 cells or in COS-1 cells as a secreted protein using a pcDNA3.1 expression vector. When expressed as a secreted protein in HEK293 cells or COS-1 cells, there is cotranslational removal of the signal peptide, amino acids 1-19 of full-length proMMP9(1-707) (SEQ ID NO:1). The final purified proMMP9(1-707) (SEQ ID NO:1) protein lacks the signal peptide.

Prior to transfection with the proMMP9(1-707) (SEQ ID NO:1) construct, the HEK293 cells were suspension adapted (shake flasks) in a serum free media (Freestyle 293) supplemented with pluronic acid (F-68) at a final concentration of 0.1%. Once cells reached a density of 1.2×10⁶/mL they were transiently transfected using standard methods. Transient transfection of COS-1 cells was done in flasks with adherent cell cultures and serum free media. For both HEK293 and COS-1 cells, the conditioned media was collected for purification of the proMMP9(1-707) (SEQ ID NO:1) protein. 1.0 M HEPES pH 7.5 was added to 9 L of conditioned media for a final concentration of 50 mM. The media was concentrated to 600 mL in a Kvicklab concentrator fitted with a hollow fiber cartridge of 10,000 MWCO (GE Healthcare). This was clarified by centrifugation at 6,000×g, 15 minutes, at 4° C. and then further concentrated to 400 mL in Jumbo Sep centrifugal concentrators (Pall) with 10,000 MWCO membranes. The concentrated protein was dialyzed against 50 mM HEPES pH 7.5, mM CaCl₂, 0.05% Brij 35, overnight at 4° C. and then dialysis was continued for several hours at 4° C. in fresh dialysis buffer. The dialyzed protein was centrifuged at 6,000×g, 15 minutes, at 4° C., and filtered to 0.45 micron. 12 mL of Gelatin Sepharose 4B resin (GE Healthcare) was equilibrated in 50 mM HEPES pH 7.5, 10 mM CaCl₂, 0.05% Brij 35 in a 2.5 cm diameter Econo-Column (Bio-Rad Laboratories). The filtered protein solution was loaded onto the Gelatin Sepharose resin using gravity flow at approximately 3 mL/minute. The resin was washed with 10 CV 50 mM HEPES pH 7.5, 10 mM CaCl₂, 0.05% Brij 35 and eluted with 30 mL 50 mM HEPES pH 7.5, 10 mM CaCl₂, 0.05% Brij 35, 10% DMSO, collected in 5 mL fractions. Fractions containing protein, confirmed by A280 absorbance, were dialyzed, in 500 times the volume of the fractions, against 50 mM HEPES pH 7.5, 10 mM CaCl₂, 0.05% Brij 35, overnight at 4° C. Dialysis was continued for an additional 24 hours in two fresh buffer changes. The dialyzed fractions were analyzed on SDS-PAGE and pooled based on purity. The pooled protein was concentrated to 1.2 mg/mL in Jumbo Sep centrifugal concentrators with 10,000 MWCO membranes. Protein concentration was determined with DC™ protein assay (Bio-Rad Laboratories, Inc.). The protein was aliquoted, frozen in liquid nitrogen and stored at −80° C.

Full-Length Rat proMMP9

Amino acid numbering for full-length rat proMMP9 was based on UniProtKB/Swiss-Prot P50282, full-length rat matrix metalloproteinase-9 precursor, proMMP9(1-708) (SEQ ID NO:11). The full-length rat proMMP9 was produced with the same methods as described for full-length human proMMP9. In brief, full-length rat proMMP9(1-708) (SEQ ID NO:11) was expressed in HEK293 cells as a secreted protein using a pcDNA3.1 expression vector. When expressed in HEK293 cells and secreted into the media, there is cotranslational removal of the signal peptide, so the final purified full-length rat proMMP9(1-708) (SEQ ID NO:11) protein lacks the signal peptide.

Human proMMP13

The sequence for proMMP13 was amino acids 1-268 from UniProtKB/Swiss-Prot P45452, proMMP13(1-268) (SEQ ID NO:7). The expression construct included a C-terminal Tev cleavage sequence flanking recombination sequences for use in the Invitrogen Gateway system. The construct was recombined into an entry vector using the Invitrogen Gateway recombination reagents. The resulting construct was transferred into a HEK293 expression vector containing a C-terminal 6×-histidine tag. Protein was expressed via transient transfection utilizing HEK293 cells and secreted into the media. When expressed in HEK293 cells and secreted into the media, there is cotranslational removal of the signal peptide, amino acids 1-19 of proMMP13(1-268) (SEQ ID NO:7). The final purified proMMP13(1-268) (SEQ ID NO:7) protein lacks the signal peptide. HEK293 media were harvested and centrifuged. Media were loaded on GE Healthcare H isTrap FF columns, washed with buffer A (20 mM Tris pH 7.5, 200 mM NaCl, 2 mM CaCl₂, 10 mM imidazole), and eluted with buffer B (20 mM Tris pH 7.5, 200 mM NaCl, 2 mM CaCl₂ 200 mM imidazole). The eluted protein was loaded on a Superdex 200 column equilibrated with buffer C (20 mM HEPES pH 7.4, 100 mM NaCl, 0.5 mM CaCl₂). Fractions containing proMMP13(1-268) (SEQ ID NO:7) were pooled and concentrated to >2 mg/mL.

### Human Catalytic MMP3

Catalytic MMP3 was amino acids 100-265 of human MMP3 from UniProtKB/Swiss-Prot P08254, MMP3(100-265) (SEQ ID NO:8). The corresponding nucleotide sequence was subcloned into a pET28b vector to add a C-terminal 6×-Histidine tag and the construct was used for expression in E. coli. The protein was purified to >95% purity from 4.5 M urea solubilized inclusion bodies by standard techniques. Aliquots of purified protein were stored at −70° C. Purified recombinant human catalytic MMP3 is also available from commercial sources (e.g., Calbiochem®, 444217).

### Biological Assays

### ThermoFluor® Assays

### Generalized ThermoFluor® Methods

The ThermoFluor® (TF) assay is a 384-well plate-based binding assay that measures thermal stability of proteins (Biomol Screen 2001, 6, 429-40; Biochemistry 2005, 44, 5258-66). The experiments were carried out using instruments available from Johnson & Johnson Pharmaceutical Research & Development, LLC. TF dye used in all experiments was 1,8-anilinonaphthalene-8-sulfonic acid (1,8-ANS) (Invitrogen: A-47).

Compounds were arranged in a pre-dispensed plate (Greiner Bio-one: 781280), wherein compounds were serially diluted in 100% DMSO across 11 columns within a series. Columns 12 and 24 were used as DMSO reference and contained no compound. For multiple compound concentration-response experiments, the compound aliquots (50 mL) were robotically predispensed directly into black 384-well polypropylene PCR microplates (Abgene: TF-0384/k) using a Cartesian Hummingbird liquid handler (DigiLab, Holliston, Mass.). Following compound dispense, protein and dye solutions were added to achieve the final assay volume of 3 μL. The assay solutions were overlayed with 1 μL of silicone oil (Fluka, type DC 200: 85411) to prevent evaporation.

Assay plates were robotically loaded onto a thermostatically controlled PCR-type thermal block and then heated from 40 to 90° C. at a ramp-rate of 1° C./min for all experiments. Fluorescence was measured by continuous illumination with UV light (Hamamatsu LC6) supplied via fiber optics and filtered through a band-pass filter (380-400 nm; >6 OD cutoff). Fluorescence emission of the entire 384-well plate was detected by measuring light intensity using a CCD camera (Sensys, Roper Scientific) filtered to detect 500±25 nm, resulting in simultaneous and independent readings of all 384 wells. A single image with 20-sec exposure time was collected at each temperature, and the sum of the pixel intensity in a given area of the assay plate was recorded vs temperature and fit to standard equations to yield the Tₘ (J Biomol Screen 2001, 6, 429-40).

Thermodynamic parameters necessary for fitting compound binding for each proMMP were estimated by differential scanning calorimetry (DSC) and from ThermoFluor® data. The heat capacity of unfolding for each protein was estimated from the molecular weight and from ThermoFluor® dosing data. Unfolding curves were fit singly, then in groups of 12 ligand concentrations the data were fit to a single K_{D} for each compound.

ThermoFluor® with proMMP9(67-444;ΔFnII) (SEQ ID NO:5)

The protein sample preparations had to include a desalting buffer exchange step via a PD-10 gravity column (GE Healthcare). The desalting buffer exchange was performed prior to diluting the protein to the final assay concentration of 3.5 μM proMMP9(67-444;ΔFnII) (SEQ ID NO:5). The concentration of proMMP9(67-444;ΔFnII) (SEQ ID NO:5) was determined spectrophotometrically based on a calculated extinction coefficient of ε₂₈₀=33900 M⁻¹cm⁻¹, a calculated molecular weight of 22.6 kDa, and calculated pI of 5.20. ThermoFluor® reference conditions were defined as follows: 80 μg/mL (3.5 μM) proMMP9(67-444;ΔFnII) (SEQ ID NO:5), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(67-444;ΔFnII) (SEQ ID NO:5) are as follows: Tₘ (° C.)=63 (+/−0.1), Δ_{U}H_{(Tm)} (cal mol⁻¹)=105000(+/−5000), Δ_{U}S_{(Tm)} (cal mol⁻¹ K⁻¹)=450, Δ_{U}Cₚ (cal mol⁻¹ K⁻¹)=2000.

ThermoFluor® with proMMP9(20-445;ΔFnM (SEQ ID NO:6)

The protein sample preparations included a desalting buffer exchange step via a PD-10 gravity column (GE Healthcare). The desalting buffer exchange was performed prior to diluting the protein to the final assay concentration of 2.8 μM proMMP9(20-445;ΔFnII) (SEQ ID NO:6). The concentration of proMMP9(20-445;ΔFnII) (SEQ ID NO:6) was determined spectrophotometrically based on a calculated extinction coefficient of ε₂₈₀=39880 M⁻cm⁻¹, a calculated molecular weight of 28.2 kDa, and calculated pI of 5.5. ThermoFluor® reference conditions were define as follows: 80 μg/mL (2.8 μM) proMMP9(20-445;ΔFnII) (SEQ ID NO:6), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(20-445;ΔFnII) (SEQ ID NO:6) are as follows: Tₘ (° C.)=72 (+/−0.1), Δ_{U}H_{(Tm)} (cal mol⁻¹)=160000(+/−5000), Δ_{U}S_{(Tm)} (cal mol⁻¹ K⁻¹)=434, Δ_{U}Cₚ (cal mol⁻¹ K⁻)=2400.

ThermoFluor® with proMMP13(1-268) (SEQ ID NO: 7)

The proMMP13(1-268) (SEQ ID NO:7) protein sample preparations included a desalting buffer exchange step via a PD-10 gravity column (GE Healthcare). The desalting buffer exchange was performed prior to diluting the protein to the final assay concentration of 3.5 μM. The concentration of proMMP13(1-268) (SEQ ID NO:7) was estimated spectrophotometrically based on a calculated extinction coefficient of ε₂₈₀=37000 M⁻¹cm⁻¹, a calculated molecular weight of 30.8 kDa, and calculated pI of 5.33. ThermoFluor® reference conditions were defined as follows: 100 μg/mL proMMP13(1-268) (SEQ ID NO:7), 25 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP13(1-268) (SEQ ID NO:7) are as follows: Tₘ (° C.)=67 (+/−0.1), Δ_{U}H_{(Tm)} (cal mol⁻¹)=107000(+/−5000), Δ_{U}S_{(Tm)} (cal mol⁻¹ K⁻¹)=318, Δ_{U}Cₚ (cal mol⁻¹ K⁻¹)=2600.

Thermofluor data for representative compounds of Formula I is shown in Table 1.

TABLE 1

| | proMMP9 | proMMP9 | proMMP13 |
|---|---|---|---|
| | (20-445; ΔFnII) | (67-444; ΔFnII) | (1-268) |
| | (SEQ ID NO: 6) | (SEQ ID NO: 5) | (SEQ ID NO: 7) |
| Example | binding, Kd (μM) | binding, Kd (μM) | binding, Kd (μM) |
| 1 | 0.026 | 0.17 | 0.14 |
| 2 | 0.021 | 0.16 | 0.081 |
| 3 | 0.41 | 0.30 | 0.30 |
| 4 | 1.2 | 3.1 | 3.2 |
| 5 | 0.27 | 0.56 | 4.9 |
| 6 | 0.20 | 0.58 | 2.0 |
| 7 | 0.089 | 0.34 | 1.4 |
| 8 | 1.3 | 0.56 | 39 |
| 9 | ND | ND | ND |
| 10 | ND | ND | ND |
| 11 | 9.8 | 1.1 | 30 |
| 12 | 1.9 | 15 | ND |
| 13 | 0.83 | 5.1 | 7 |
| 14 | 0.57 | 7.0 | ND |
| 15 | 7.9 | >95 | 13 |
| 16 | 0.24 | 2.1 | ND |
| 17 | 0.082 | 0.73 | 12 |
| 18 | 0.34 | 0.84 | 14 |
| 19 | 17 | 11 | 62 |
| 20 | 21 | 6.7 | ND |
| 21 | 9.6 | 2.9 | 1.7 |
| 22 | 6.4 | 1.6 | ND |
| 23 | 12 | 15 | 0.98 |
| 24 | 8.6 | 2.3 | 25 |
| 25 | 2.7 | 6.8 | 31 |
| 26 | 8.9 | 3.0 | 84 |
| 27 | 1.0 | 2.8 | ND |
| 28 | 11 | >95 | 56 |
| 29 | 61 | >95 | >95 |
| 30 | 18 | 4.9 | >95 |
| 31 | 9.0 | 33 | 49 |
| 32 | 0.72 | 0.25 | 1.9 |
| 33 | 13 | >95 | 2.9 |
| 34 | 7.8 | 12 | 28 |
| 35 | 14 | 3.9 | ND |
| 36 | 0.85 | 27 | 11 |
| 37 | 0.57 | 7.2 | ND |
| 38 | 0.55 | 192 | ND |
| 39 | 1.1 | 1.4 | >95 |
| 40 | 0.99 | 0.31 | 1.5 |
| 41 | 84 | 61 | ND |
| 42 | >95 | 52 | ND |
| 43 | 0.59 | 0.58 | 0.84 |
| 44 | 0.072 | 0.24 | 0.07 |
| 45 | 0.24 | 0.98 | 0.20 |
| 46 | 1.4 | 1.2 | 5.5 |
| 47 | 1.8 | 0.68 | 2.4 |
| 48 | 23 | 3.7 | 63 |
| 49 | 4.1 | 2.5 | 6.1 |
| 50 | 1.3 | 2.6 | 2.6 |
| 51 | 1.1 | 3.0 | 1.9 |
| 52 | 0.87 | 1.6 | ND |
| 53 | 1.5 | 1.2 | ND |
| 54 | 1.0 | 1.6 | 2.0 |
| 55 | 2.5 | 1.7 | 9.8 |
| 56 | 1.3 | 2.3 | ND |
| 57 | 11 | 1.7 | >95 |
| 58 | 19 | 6.5 | >95 |
| 59 | 2.0 | 2.7 | 20 |
| 60 | 0.058 | 0.11 | 0.07 |
| 61 | 0.069 | 0.14 | 0.11 |
| 62 | 0.30 | 0.67 | 0.16 |
| 63 | 0.52 | 0.79 | 1.3 |
| 64 | 0.10 | 0.26 | 0.14 |
| 65 | 3.0 | 2.8 | 7.8 |
| 66 | 2.8 | 1.8 | 2.6 |
| 67 | 0.56 | 0.087 | ND |
| 68 | 0.32 | 0.55 | 8.4 |
| 69 | 4.8 | 1.1 | 25 |
| 70 | 0.25 | 0.41 | 1.1 |
| 71 | 0.28 | 0.095 | 0.042 |
| 72 | 8.6 | 1.2 | ND |
| 73 | 4.3 | 0.33 | ND |
| 74 | 1.8 | 0.5 | 2.4 |
| 75 | 1.1 | 0.6 | 1.5 |
| 76 | 0.57 | 0.78 | 0.37 |
| 77 | 0.35 | 0.45 | ND |
| 78 | 15 | 0.48 | 2.9 |
| 79 | 1.2 | 0.052 | 0.24 |
| 80 | 23 | 1.8 | 3.6 |
| 81 | 0.52 | 0.62 | 0.37 |
| 82 | 15 | 3.5 | 13 |
| 83 | 3.9 | 0.49 | 1.5 |
| 84 | 19 | 9.7 | ND |
| 85 | 3.5 | 0.75 | 43 |
| 86 | 1.2 | 0.20 | 3.4 |
| 87 | 0.40 | 0.38 | 3.0 |
| 88 | 0.10 | 0.65 | 0.95 |
| 89 | 0.063 | 0.39 | 0.71 |
| 90 | 0.037 | 0.17 | 0.68 |
| 91 | 0.14 | 0.52 | ND |
| 92 | 0.045 | 0.22 | ND |
| 93 | 0.74 | 2.5 | 16 |
| 94 | 0.31 | 1.7 | 6.1 |
| 95 | 0.22 | 2.8 | 4.1 |
| 96 | 0.28 | 3.8 | 16 |
| 97 | 1.9 | >95 | ND |
| 98 | >95 | 0.67 | 2.3 |
| 99 | 0.27 | 1.1 | 2.4 |
| 100 | 0.76 | 0.32 | 1.4 |
| 101 | 0.29 | 1.1 | 0.51 |
| 102 | 0.70 | 0.73 | 0.66 |
| 103 | 0.48 | 1.0 | 0.45 |
| 104 | 0.46 | 0.61 | 0.53 |
| 105 | 0.24 | 2.9 | 1.3 |
| 106 | 1.2 | 0.50 | 2.2 |
| 107 | 0.20 | 0.52 | 0.54 |
| 108 | 0.31 | >95 | 0.32 |
| 109 | 3.8 | >95 | >95 |
| 110 | 0.13 | 0.48 | 0.69 |
| 111 | 0.086 | 0.29 | 0.36 |
| 112 | 1.4 | 2.2 | >57 |
| 113 | 1.5 | 1.9 | >57 |
| 114 | 0.69 | 0.96 | 1.8 |
| 115 | 2.8 | 3.8 | 6.4 |
| 116 | 2.7 | 1.4 | 6.7 |
| 117 | 0.68 | 2.0 | 6.9 |
| 118 | 0.64 | 1.2 | 5.5 |
| 119 | 3.3 | 2.2 | 6.2 |
| 120 | 2.5 | 1.6 | 5.6 |
| 121 | 2.4 | 1.2 | 8.7 |
| 122 | 0.83 | 0.67 | 4.9 |
| 123 | 0.28 | 1.0 | 1.8 |
| 124 | 0.65 | 3.7 | 6.1 |
| 125 | 0.58 | 3.2 | 4.9 |
| 126 | 1.1 | 4.7 | 11 |
| 127 | 0.73 | 3.5 | 8.6 |
| 128 | 0.50 | 2.7 | 5.2 |
| 129 | 0.72 | 4.7 | 5.1 |
| 130 | 0.55 | 3.0 | 4.9 |
| 131 | 0.77 | 3.3 | 5.6 |
| 132 | 4.3 | 21 | 11 |
| 133 | 0.97 | 4.0 | 3.8 |
| 134 | 18 | >95 | ND |
| 135 | 31 | 40 | ND |
| 136 | 38 | 8.2 | ND |
| 137 | 11 | 2.2 | ND |
| 138 | 1.7 | 0.48 | 0.83 |
| 139 | 75 | 11 | 8.1 |
| 140 | >95 | 45 | 26 |
| 141 | >95 | 15 | 4.3 |
| 142 | >95 | 58 | 6.7 |
| 143 | 62 | 16 | 6.0 |
| 144 | 1.0 | 2.0 | 3.6 |
| 145 | 0.13 | 0.65 | ND |
| 146 | 4.6 | 1.3 | ND |
| 147 | 12 | 7.6 | ND |
| 148 | >8 | >8 | ND |
| 149 | 5.0 | 4.4 | 40 |
| 150 | 0.16 | 1.3 | 0.22 |
| 151 | 3.7 | 0.57 | ND |
| 152 | 5.7 | 6.4 | ND |
| 153 | 1.6 | 4.5 | ND |
| 154 | 2.2 | 2.0 | ND |
| 155 | 1.9 | 0.34 | ND |
| 156 | ND | ND | ND |
| 157 | ND | ND | ND |
| 158 | ND | ND | ND |

### Enzyme Assays

proMMP9/MMP3 P126 Activation Assay

Compounds were assessed for inhibition of proMMP9 activation by catalytic MMP3, MMP3(100-265) (SEQ ID NO:8) using full-length proMMP9(1-707) (SEQ ID NO:1) purified from HEK293 cells and a peptide (Mca-PLGL-Dpa-AR-NH₂, BioMol P-126) that fluoresces upon cleavage by catalytic MMP9. The assay buffer employed was 50 mM Hepes, pH 7.5, 10 mM CaCl₂, 0.05% Brij-35. DMSO was included at a final concentration of 2%, arising from the test compound addition. On the day of assay, proMMP9(1-707) (SEQ ID NO:1) purified from HEK293 cells and MMP3(100-265) (SEQ ID NO:8) were diluted to 400 nM in assay buffer. The reaction volume was 50 μL. In 96-well black plates (Costar 3915), 44 μL of assay buffer was mixed with 1.0 μL of test compound, 2.5 μL of 400 nM proMMP9(1-707) (SEQ ID NO:1) purified from HEK293 cells and the reaction was initiated with 2.5 μL of 400 nM MMP3(100-265) (SEQ ID NO:8).The plate was sealed and incubated for 80 min at 37° C. Final concentrations were 20 nM proMMP9(1-707) (SEQ ID NO:1) purified from HEK293 cells and 20 nM MMP3(100-265) (SEQ ID NO:8), and concentrations of test compounds were varied to fully bracket the IC₅₀. Immediately following the 80 min incubation, 50 μL of 40 μM P-126 substrate was added (freshly diluted in assay buffer), and the resulting activity associated with catalytic MMP9 was kinetically monitored at 328 nm excitation, 393 nm emission for 10-15 min at 37° C., using a Spectramax Gemini XPS reader (Molecular Devices). Reactivity of residual MMP3 towards P-126 substrate was minimal under these conditions. Initial velocities were plotted by use of a four-parameter logistics equation (GraphPad Prism® software) for determination of IC₅₀.

### ProMMP13/Plasmin P126 Activation Assay

Compounds were assessed for inhibition of proMMP13 activation by plasmin using a peptide (Mca-PLGL-Dpa-AR-NH2, BioMol P-126) that fluoresces upon cleavage by catalytic MMP13. The assay buffer employed was 50 mM Hepes, pH 7.5, 10 mM CaCl₂, 0.05% Brij-35. DMSO was included at a final concentration of 2%, arising from the test compound addition. On the day of assay, proMMP13(1-268) (SEQ ID NO:7) purified from HEK293 cells and plasmin were diluted to 160 nM and 320 nM, respectively, in assay buffer. The reaction volume was 50 μL. In 96-well black plates (Costar 3915), 44 μL of assay buffer was mixed with 1.0 μL of test compound, 2.5 μL of 160 nM proMMP13(1-268) (SEQ ID NO:7), and the reaction was initiated with 2.5 μL of 320 nM plasmin. The plate was sealed and incubated for 40 min at 37° C. Final concentrations were 8 nM proMMP13(1-268) (SEQ ID NO:7) and 16 nM plasmin, and concentrations of test compounds were varied to fully bracket the IC₅₀. Immediately following the 40 min incubation, 50 μL of 40 μM P-126 substrate was added (freshly diluted in assay buffer), and the resulting activity associated with catalytic MMP13 was kinetically monitored at 328 nm excitation, 393 nm emission for 10-15 min at 37° C., using a Spectramax Gemini XPS reader (Molecular Devices). Plasmin was not reactive towards P-126 substrate under these conditions. Initial velocities were plotted by use of a four-parameter logistics equation (GraphPad Prism® software) for determination of IC₅₀.

### ProMMP9/MMP3 DQ Gelatin Activation Assay

Compounds were assessed for inhibition of proMMP9 activation by catalytic MMP3 using a quenched fluorescein gelatin substrate (DQ gelatin, Invitrogen D12054) that fluoresces upon cleavage by activated MMP9. The assay buffer employed was 50 mM Hepes, pH 7.5, 10 mM CaCl₂, 0.05% Brij-35. DMSO was included at a final concentration of 0.2%, arising from the test compound addition. On the day of assay, full-length proMMP9(1-707) (SEQ ID NO:1) from COS-1 cells and catalytic MMP3(100-265) (SEQ ID NO:8) were diluted to 60 nM and 30 nM, respectively, in assay buffer. Test compounds in DMSO were diluted 250-fold in assay buffer at 4× the final concentration. The reaction volume was 12 μL, and all reactions were conducted in triplicate. In 384-well half-volume plates (Perkin Elmer ProxiPlate 384 F Plus, 6008260), 4 μL of test compound in assay buffer was mixed with 4 μL of 60 nM full-length proMMP9(1-707) (SEQ ID NO:1) from COS-1 cells. The plate was sealed and incubated for 30 min at 37° C. Final concentrations were 20 nM full-length proMMP9(1-707) (SEQ ID NO:1) from COS-1 cells and 10 nM MMP3(100-265) (SEQ ID NO:8), and concentrations of test compounds were varied to fully bracket the IC₅₀. Immediately following the 30 min incubation, 4 μL of 40 μg/ml DQ gelatin substrate was added (freshly diluted in assay buffer), and incubated for 10 min at room temperature. The reaction was stopped by the addition of 4 μL of 50 mM EDTA, and the resulting activity associated with catalytic MMP9 was determined at 485 nm excitation, 535 nm emission using an Envision fluorescent reader (Perkin Elmer). Reactivity of residual MMP3 towards DQ gelatin was minimal under these conditions. Percent inhibition of test compounds were determined from suitable positive (DMSO only in assay buffer) and negative (EDTA added prior to reaction initiation) controls. Plots of % inhibition vs. test compound concentration were fit to a four-parameter logistics equation (GraphPad Prism® software) for determination of IC₅₀.

Enzyme assay data for representative compounds of Formula I is shown in Table 2.

TABLE 2

| | proMMP9/ | ProMMP9/ | ProMMP13/ |
|---|---|---|---|
| | MMP3 P126 | MMP3 DQ gel | Plasmin P126 |
| | Activation Assay, | Activation Assay, | Activation Assay, |
| Example | IC₅₀ (μM) | IC₅₀ (μM) | IC₅₀ (μM) |
| 1 | 0.16 | ND | ND |
| 2 | 0.16 | ND | ND |
| 3 | 0.13 | ND | ND |
| 4 | 9.0 | ND | ND |
| 5 | 0.85 | ND | ND |
| 6 | 0.16 | ND | ND |
| 7 | 0.26 | ND | ND |
| 8 | 0.10 | ND | ND |
| 9 | 3.6 | ND | ND |
| 10 | 5.6 | ND | ND |
| 11 | ND | 9.0 | ND |
| 12 | 2.5 | ND | ND |
| 13 | 6.1 | ND | ND |
| 14 | 4.7 | ND | ND |
| 15 | >20 | ND | ND |
| 16 | 1.3 | ND | ND |
| 17 | 0.46 | ND | ND |
| 18 | 0.69 | ND | ND |
| 19 | ND | ND | ND |
| 20 | ND | ND | ND |
| 21 | ND | ND | ND |
| 22 | ND | ND | ND |
| 23 | ND | ND | ND |
| 24 | ND | ND | ND |
| 25 | 4.6 | ND | ND |
| 26 | >20 | ND | ND |
| 27 | 5.2 | ND | ND |
| 28 | >20 | ND | ND |
| 29 | 3.8 | ND | ND |
| 30 | 4.1 | ND | ND |
| 31 | ~7 | ND | ND |
| 32 | 0.15 | ND | ND |
| 33 | 7.3 | ND | ND |
| 34 | 9.0 | ND | ND |
| 35 | 1.7 | ND | ND |
| 36 | 0.67 | ND | ND |
| 37 | 3.6 | ND | ND |
| 38 | 0.8 | ND | ND |
| 39 | 0.76 | ND | ND |
| 40 | 0.9 | ND | ND |
| 41 | ~13 | ND | ND |
| 42 | 9.1 | ND | ND |
| 43 | 0.76 | ND | ND |
| 44 | 0.41 | ND | ND |
| 45 | 0.83 | ND | ND |
| 46 | 1.1 | ND | ND |
| 47 | 0.31 | ND | ND |
| 48 | 1.4 | ND | ND |
| 49 | 3.0 | ND | ND |
| 50 | 4.1 | ND | ND |
| 51 | 4.1 | ND | ND |
| 52 | 1.2 | ND | ND |
| 53 | 1.9 | ND | ND |
| 54 | 3.4 | ND | ND |
| 55 | 0.74 | ND | ND |
| 56 | 12 | ND | ND |
| 57 | 1.9 | ND | ND |
| 58 | ND | ND | ND |
| 59 | 7.5 | ND | ND |
| 60 | 0.35 | ND | ND |
| 61 | 0.38 | ND | ND |
| 62 | 1.5 | ND | ND |
| 63 | 0.75 | ND | ND |
| 64 | 0.18 | ND | 3.3 |
| 65 | 1.8 | ND | ND |
| 66 | 4.9 | ND | ND |
| 67 | 0.45 | ND | ND |
| 68 | 0.097 | ND | ND |
| 69 | 1.4 | ND | ND |
| 70 | 0.46 | ND | ND |
| 71 | 0.87 | ND | ND |
| 72 | 1.0 | ND | ND |
| 73 | 0.52 | ND | ND |
| 74 | 2.5 | ND | ND |
| 75 | 1.6 | ND | ND |
| 76 | 0.43 | ND | ND |
| 77 | 0.41 | ND | ND |
| 78 | ND | ND | ND |
| 79 | 0.55 | ND | ND |
| 80 | 0.65 | ND | ND |
| 81 | 0.64 | ND | ND |
| 82 | ND | ND | ND |
| 83 | 0.48 | ND | ND |
| 84 | 9.8 | ND | ND |
| 85 | 0.27 | ND | ND |
| 86 | 0.61 | ND | ND |
| 87 | 1.2 | ND | ND |
| 88 | 0.38 | ND | ND |
| 89 | 0.33 | ND | ND |
| 90 | 0.10 | ND | ND |
| 91 | 0.30 | ND | ND |
| 92 | 0.34 | ND | ND |
| 93 | ND | ND | ND |
| 94 | 0.40 | ND | ND |
| 95 | 0.41 | ND | ND |
| 96 | ~5 | ND | ND |
| 97 | >20 | ND | ND |
| 98 | 0.62 | ND | ND |
| 99 | 0.59 | ND | ND |
| 100 | 0.22 | ND | ND |
| 101 | 0.17 | ND | ND |
| 102 | 0.13 | ND | ND |
| 103 | 0.097 | ND | ND |
| 104 | 0.13 | ND | ND |
| 105 | ND | ND | ND |
| 106 | 0.91 | ND | ND |
| 107 | 0.15 | ND | ND |
| 108 | 1.7 | ND | ND |
| 109 | 5.7 | ND | ND |
| 110 | 0.20 | ND | ND |
| 111 | 0.081 | ND | ND |
| 112 | 0.52 | ND | ND |
| 113 | 0.57 | ND | ND |
| 114 | 0.31 | ND | ND |
| 115 | 0.88 | ND | ND |
| 116 | 0.50 | ND | ND |
| 117 | 0.67 | ND | ND |
| 118 | 1.1 | ND | ND |
| 119 | 0.52 | ND | ND |
| 120 | 0.48 | ND | ND |
| 121 | 0.37 | ND | ND |
| 122 | 0.25 | ND | ND |
| 123 | 0.44 | ND | ND |
| 124 | 0.67 | ND | ND |
| 125 | 0.78 | ND | ND |
| 126 | 0.77 | ND | ND |
| 127 | 0.65 | ND | ND |
| 128 | 1.3 | ND | ND |
| 129 | 1.2 | ND | ND |
| 130 | 0.70 | ND | ND |
| 131 | 0.73 | ND | ND |
| 132 | 0.72 | ND | ND |
| 133 | 0.66 | ND | ND |
| 134 | >20 | ND | ND |
| 135 | ~5 | ND | ND |
| 136 | ~6 | ND | ND |
| 137 | 4.7 | ND | ND |
| 138 | 0.73 | ND | ND |
| 139 | 5.2 | ND | ND |
| 140 | 5.5 | ND | ND |
| 141 | ~12 | ND | ND |
| 142 | 11 | ND | ND |
| 143 | ~6 | ND | ND |
| 144 | 1.6 | ND | ND |
| 145 | 0.78 | ND | ND |
| 146 | ND | 5.9 | ND |
| 147 | ND | 20 | ND |
| 148 | ND | 6.0 | ND |
| 149 | ND | 27 | ND |
| 150 | 1.2 | ND | ND |
| 151 | 0.41 | ND | ND |
| 152 | 4.7 | ND | ND |
| 153 | 1.4 | ND | ND |
| 154 | 2.6 | ND | ND |
| 155 | 0.29 | ND | ND |
| 156 | 0.067 | ND | ND |
| 157 | 0.033 | ND | 0.86 |
| 158 | 0.29 | ND | ND |

### Cell-based Assays

Activation of proMMP9 in rat Synoviocyte Cultures

A primary synoviocytes line was derived from the periarticular tissue of arthritic rats. Arthritis was induced in female Lewis rats following an i.p. administration of streptococcal cell wall peptidoglycan polysaccharides (J Exp Med 1977; 146:1585-1602). Rats with established arthritis were sacrificed, and hind-limbs were severed, immersed briefly in 70% ethanol, and placed in a sterile hood. The skin was removed and the inflamed tissue surrounding the tibia-tarsal joint was harvested using a scalpel. Tissue from six rats was pooled, minced to approximately 8 mm³ pieces, and cultured in Dulbecco's Modified Eagle's Medium (DMEM) containing 15% fetal calf serum (FCS). In the following weeks, cells migrated out of the tissue piece, proliferated, and formed a monolayer of adherent cells. The synoviocytes were lifted from culture plates with 0.05% trypsin and passaged weekly at 1:4 ratios in DMEM containing 10% FCS. Synoviocytes were used at passage 9 to investigate the ability of Example 64 to inhibit the maturation of MMP9 to active form.

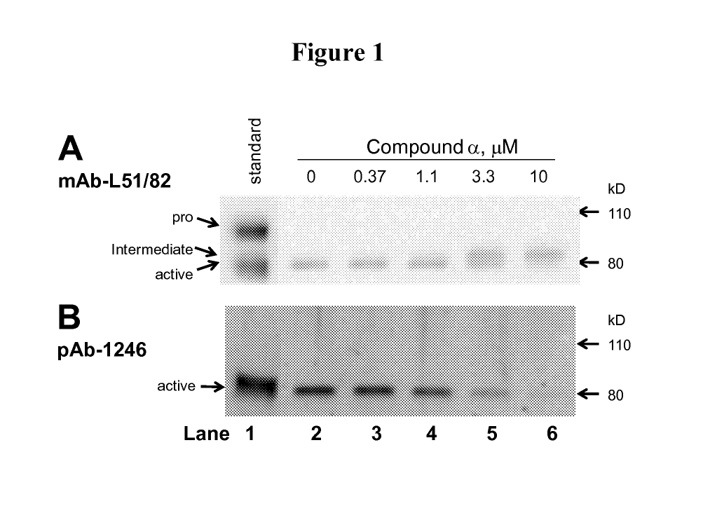

Rat synoviocytes spontaneously expressed and activated MMP9 when cultured in collagen gels and stimulated with tumor necrosis factor-alpha (TNFα) (FIG. 1 and Table 3). Eight volumes of an ice-cold solution of 3.8 mg/mL rat tail collagen (Sigma Cat #C3867-1VL) were mixed with 1 volume of 1 M sodium bicarbonate and 1 volume of 10× Roswell Park Memorial Institute medium. The pH of the mixture was adjusted to pH 7 with 1 N sodium hydroxide and equal volumes of the pH-adjusted collagen solution were mixed with DMEM containing 0.8 million synoviocytes per mL. One half mL volumes were dispensed into Costar 24-well culture dishes and placed for one hr at 37° C. and 5% CO₂, during which time the collagen solution formed a gel. Individual gels were dislodged into wells of 12-well Costar plates containing 1 mL/well of DMEM adjusted to contain 0.05% BSA and 100 ng/mL mouse TNFα (R&D Systems Cat #410-MT-010). The plates were agitated 10 seconds to ensure that the collagen gels did not adhere to the well bottoms. After overnight culture at 37° C. and 5% CO₂, wells were adjusted to contain an additional 0.5 mL of DMEM containing 0.05% BSA and Example 64 at 4× the final desired concentration (final culture volumes were 2 mL). The plates were cultured an additional 48 hrs, at which time 1 mL of conditioned media were harvested into fresh eppendorf tubes containing 40 μL/mL of a 50% slurry of gelatin-conjugated sepharose (GE Healthcare Cat #17-0956-01). Samples were rotated for 2 hrs at 4° C. before centrifugation 1 min×200 g. Supernatants were discarded. The gelatin-sepharose pellets were washed once with 1 mL of ice cold DMEM, resuspended in 50 μL of 2× reducing Leamli buffer and heated 5 min at 95° C. Fifteen μL of eluted proteins were resolved on 4-12% NuPAGE gels and transferred to 0.45 μm pore-sized nitrocellose blots. Next, blots were incubated in blocking buffer (5% milk in Tris-buffered saline containing 0.1% Tween-20) for 1 hr at RT and probed overnight (4° C.) with blocking buffer containing 1 μg/mL primary antibodies. Blots were next probed 1 hr at RT with 1/10,000 dilutions of goat anti-mouse IgG-HRP or goat anti-rabbit IgG-HRP (Santa Cruz) in blocking buffer and developed using SuperSignal® West Fempto Maximum Sensitivity Substrate. Chemiluminesence signal was analyzed using a ChemiDoc imaging system (BioRad Laboratories) and Quantity One® image software. Electrophoretic mobility was estimated based on the mobility of standards (Novex Sharp Pre-Stained Protein Standards P/N 57318).

Mouse mAb-L51/82 (UC Davis/NIH NeuroMab Facility, Antibody Incorporated) was used to detect pro and processed forms of MMP9. Synoviocyte-conditioned media contained an approximately 80 kD form of MMP9 (FIG. 1A, lane 2). In the presence of 0.37-10 μM Example 64 (FIG. 1A, lanes 3-6), the 80 kD active MMP9 form was reduced in a dose dependent fashion, and a form of approximately 86 kD appeared. The 86 kD form was predominant in the presence of 10 μM Example 64 (FIG. 1A, lane 6). Lane 1 was loaded with a standard containing 3 ng of full-length rat proMMP9(1-708) (SEQ ID NO:11) and 3 ng of full-length rat proMMP9(1-708) (SEQ ID NO:11) converted to catalytic rat MMP9 by catalytic MMP3. The electrophoretic mobility of the 80 kD form present in synoviocyte conditioned medium was the same as the active MMP9 standard. The 86 kD form produced by synoviocytes in the presence of Example 64 demonstrated greater mobility than the full-length rat proMMP9(1-708) (SEQ ID NO:11) standard which ran with a mobility of approximately 100 kD. The 86 kD form demonstrated a mobility similar to an incompletely processed intermediate form described previously that retains the cysteine switch and lacks catalytic activity (J Biol Chem; 1992; 267:3581-4).

ProMMP9 is activated when cleaved between R106 and F107 (J Biol Chem; 1992; 267:3581-4). A rabbit polyclonal antibody (pAb-1246) was generated to the active MMP9 N-terminal neoepitope using an approach similar to that reported previously (Eur J Biochem; 1998; 258:37-43). Rabbits were immunized and boosted with a peptide, human MMP9(107-113) (SEQ ID NO:9) conjugated to keyhole limpet hemocyanin, and antibodies were affinity purified from serum using FQTFEGD-conjugated agarose affinity resin and 100 mM glycine (pH 2.5) elution. To resolve N-terminal neoepitope antibodies from antibodies directed to other epitopes within the sequence, eluted antibody was dialyzed in PBS and cross-absorbed by mixing with a peptide, human proMMP9(99-113) (SEQ ID NO:10), that was conjugated to agarose. The unbound fraction containing N-terminal neoepitope antibodies was recovered and was designated pAb-1246.

FIG. 1B, lane 1 demonstrated that pAb-1246 bound the 80 kD active MMP9 standard, but did not recognize the 100 kD proMMP9 standard. pAb-1246 detected 80 kD active MMP9 in synoviocyte conditioned medium, and Example 64 caused a dose-dependent reduction in active MMP9 (FIG. 1B, lanes 2-6). Band chemiluminescence intensities were measured directly and reported in Table 3. The production of active MMP9 was inhibited by Example 64 with an IC₅₀ of approximately 1.1 μM. pAb-1246 did not recognize the 86 kD form, providing further evidence that this likely represented an intermediate form whose further maturation was blocked by Example 64.

TABLE 3
Example 64 blocked production of active MMP9 by rat
synoviocytes ^{a}

| | | Signal of 80 kD band | |
|---|---|---|---|
| Example 64, μM | | (INT*mm²) ^{b} | % Inhibition ^{c} |
| 0 | | 84384 | 0 |
| 0.37 | μM | 74381 | 12 |
| 1.1 | μM | 45381 | 46 |
| 3.3 | μM | 11554 | 86 |
| 10 | μM | 2578 | 97 |

^{a} Rat synoviocytes embedded in collagen gels were stimulated 72 hrs with TNFα. Cultures were supplemented with the indicated concentrations of Example 64 for the final 48 hrs and conditioned media were assessed for the 80 kD active form of MMP9 by Western blotting with pAb-1246 developed against the N-terminal activation neoepitope.
^{b} Chemiluminesence captured during a 30 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c} Percent signal reduction relative to the signal generated by synoviocytes cultured in the absence of Example 64.

Activation of proMMP9 by Human Fetal Lung Fibroblast Cultures Example 64 was assessed additionally for ability to block the maturation of proMMP9 to active MMP9 in cultures of human fetal lung fibroblasts (HFL-1, American Type Culture Collection #CCL-153). Unlike rat synoviocytes, HFL-1 cells were unable to process proMMP9 to the active form without addition of neutrophil elastase. Elastase did not directly cause processing of recombinant proMMP9 (data not shown). Rather, the function of elastase in this assay may be to inactivate tissue inhibitors of matrix metalloproteinases (TIMPs) that repress endogenous pathways of MMP9 activation (Am J Respir Grit Care Med; 1999; 159:1138-46).

HLF-1 were maintained in monolayer culture in DMEM with 10% FCS and were used between passage numbers 5-15. HLF-1 were embedded in collagen gels as described for rat SCW synoviocytes (vida supra). Half mL gels containing 0.4 million cells were dislodged into wells of 12 well Costar plates containing 1 mL/well of DMEM adjusted to contain 0.05% BSA and 100 ng/mL human TNFα (R&D Systems Cat #210-TA/CF). After overnight culture (37° C. and 5% CO₂) wells were adjusted to contain an additional 0.5 mL of DMEM containing 0.05% BSA and with or without 13.2 μM Example 64 (final concentration was 3.3 μM Example 64). Next, cultures were adjusted to contain 30 nM human elastase (Innovative Research). The plates were cultured an additional 72 hrs, at which time MMP9 secreted into the conditioned media was bound to gelatin-sepharose and evaluated by Western blot analysis as described for the rat synoviocyte cultures (vida supra). mAb-51/82 detected three forms of MMP9 in HFL-1 cultures.

These included a form of approximately 100 kD with mobility similar to recombinant rat proMMP9, an approximately 80 kD form with mobility similar to rat active MMP9, and an approximately 86 kD intermediate form. The band intensities are provided in Table 4. In the absence of Example 64, most of the MMP9 was present as the 80 kD form. In the presence of Example 64, the 80 kD form was a minor fraction of the total signal while nearly half of the signal were contributed each by the 100 kD and 86 kD forms. The total signal of the three bands was similar with or without Example 64. These data indicate that the 100 kD and 86 kD forms of MMP9 were effectively stabilized by Example 64 and the formation of the 80 kD form was suppressed.

TABLE 4
Example 64 blocked processing of MMP9 by HFL-1 cells ^{a}

| Example 64, | Signal (INT*mm²) ^{b} | | | | Percent of total signal | | |
|---|---|---|---|---|---|---|---|
| 3.3 μM | 100 kD | 86 kD | 80 kD | Total | 100 kD | 86 kD | 80 kD |
| − | 17190 | 24858 | 61925 | 103973 | 16 | 24 | 60 |
| + | 42107 | 43147 | 6092 | 91346 | 46 | 47 | 7 |

^{a} Human fetal lung fibroblasts (HFL-1) embedded in collagen gels were stimulated 90 hrs with TNFα. Cultures were supplemented with or without 3.3 μM Example 64 and with 30 nM elastase for the final 72 hrs and conditioned media were assessed for the MMP9 forms by Western blotting with mAb-L51/82.
^{b} Chemiluminesence captured during a 150 s exposure was analyzed using a ChemiDoc imaging system (BioRad Laboratories) and Quantity One ® image software. Signals were measured within uniform sized boxes drawn to circumscribe the bands and were the product of the average intensity (INT) and the box area (mm²). Values given have been corrected for background signal.

A second experiment was performed to determine if the 80 kD form was mature active MMP9 and to determine the potency of Example 64 as an inhibitor of MMP9 maturation in this assay. HFL-1 cells embedded in collagen gels were cultured as described above in the presence of TNFα overnight and the cultures were then adjusted to contain 30 nM elastase and graded concentrations of Example 64 for an additional 72 hrs at which time MMP9 secreted into the conditioned media was bound to gelatin-sepharose and evaluated by Western blot analysis for active MMP9 using pAb-1246 raised against the N-terminal neoepitope of active MMP9 (Table 5). In the absence of Example 64, pAb-1246 readily detected MMP9 with an electrophoretic mobility of approximately 80 kD. Example 64 effectively inhibited the ability of HFL-1 cultures to process proMMP9 to active MMP9. Inhibition occurred over a dose range with an IC₅₀ of approximately 0.3 μM Example 64.

TABLE 5
Example 64 blocked production of active MMP9 by human fetal
lung fibroblasts ^{a}

| | | Signal of 80 kD band | |
|---|---|---|---|
| Example 64, μM | | (INT*mm²) ^{b} | % Inhibition ^{c} |
| 0 | | 168781 | 0 |
| 0.12 | μM | 168211 | 0 |
| 0.37 | μM | 45996 | 73 |
| 1.1 | μM | 1747 | 99 |
| 3.3 | μM | 152 | 100 |
| 10 | μM | 0 | 100 |

^{a} Human fetal lung fibroblasts (HFL-1) embedded in collagen gels were stimulated 90 hrs with TNFα. Cultures were supplemented with the indicated concentrations of Example 64 and 30 nM elastase for the final 72 hrs and conditioned media were assessed for active MMP9 by Western blotting with pAb-1246 developed against the N-terminal activation neoepitope.
^{b} Chemiluminesence captured during a 10 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c} Percent signal reduction relative to the signal generated by HFL-1 cells cultured in the absence of Example 64.

### In Vivo Studies

Expression and Activation of proMMP9 In Vivo is Associated with Rat SCW-Arthritis

MMP9 protein expression was reportedly increased in the synovial fluid of patients with rheumatoid arthritis (Clinical Immunology and Immunopathology; 1996; 78:161-71). A preliminary study was performed to assess MMP9 expression and activation in a rat model of arthritis.

A polyarthritis can be induced in female Lewis rats following i.p. administration of streptococcal cell wall (SCW) proteoglycan-polysaccharides (PG-PS) (J Exp Med 1977; 146:1585-1602). The model has an acute phase (days 3-7) that is complement and neutrophil-dependent and that resolves. A chronic erosive phase begins at about day ten and is dependent on the development of specific T cell immunity to the PG-GS, which resists digestion and remains present in synovial macrophages for months. Like rheumatoid arthritis, SCW-induced arthritis is reduced by TNF inhibitors, and the dependence of SCW-induced arthritis on macrophages (Rheumatology; 2001; 40:978-987) and the strong association of rheumatoid arthritis severity with synovial-tissue macrophage counts (Ann Rheum Dis; 2005; 64:834-838) makes SCW-arthritis an attractive model for testing potential therapeutic agents. SCW PG-PS 10S (Beckton Dickinson Cat#210866) suspended in saline was vortexed for 30 seconds and sonicated for 3 min with a probe type sonicator prior to injection. Female Lewis (LEW/N) rats, 5-6 weeks of age (80-100 g) were injected (i.p.) with SCW PG-PS (15 μg of rhamnose/gram BW) in the lower left quadrant of the abdomen using a 1 mL syringe fitted with a 23-gauge needle. Control (disease-free) rats were treated in a similar manner with sterile saline. Control rats were sacrificed on day 5 and groups of SCW-injected rats were sacrificed on day 5 when acute inflammation was maximal or on day 18 when chronic inflammation was established.

Hind-limbs were skinned, severed just above the tibia-tarsus joint and below the metatarsals, and the tibia-tarsus joints (ankles) were weighed, snap frozen and pulverized on dry ice using a hammer and anvil. The pulverized tissue was suspended in 3 volumes (w:v) of ice-cold homogenization buffer containing 50 mM Tris pH 7.5, 150 mM NaCl, 5 mM EDTA, 1% Triton X100, 0.05% Brij 30, 10% dimethylsulfoxide and Complete EDTA-free Protease Inhibitor Cocktail (Roche Diagnostics). The suspended tissue was homogenized sequentially with a Kinematica AG Polytron and a Dounce homogenizer. Homogenates were centrifuged at 16,000×g for 10 min at 4° C. and the soluble fractions were saved. Dimethylsulfoxide was removed from a portion of each soluble fraction using PD MiniTrap™ G-25 desalting columns (GE Healthcare). Homogenates (0.25 mL), free of DMSO, were diluted with an equal volume of binding buffer (i.e., homogenization buffer without dimethylsufoxide) and adjusted to contain 50 μL of a 50% slurry of gelatin-conjugated sepharose. Following 2 hours of rotation at 4° C. the beads were washed twice in binding buffer and eluted in 100 μL 2×-reducing Laemmli buffer with heating to 95° C. for 5 minutes. Eluates (20 μL) were resolved on 4-12% NuPAGE gels, transferred to 0.45 μm pore-sized nitrocellose and immunoblotted for detection of proMMP9, active MMP9, and other processed forms using mAb-L51/82 and pAb-1246 as described above for detection of MMP9 forms in synoviocyte and HFL-1 cell conditioned media.

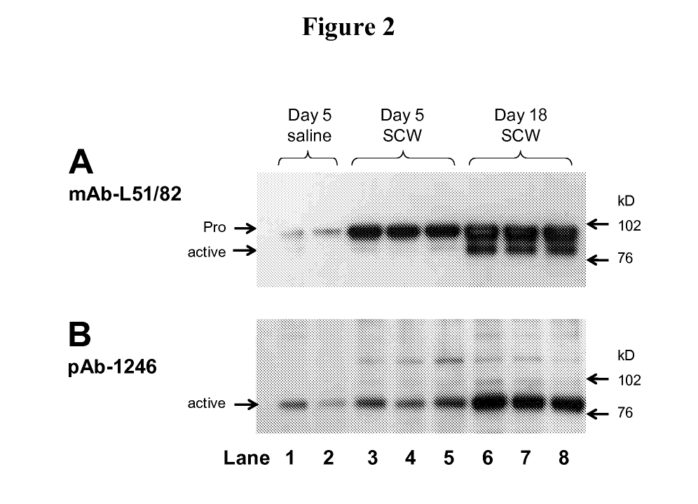

In healthy ankles of rats administered saline, mAb-L51/82 detected small amounts of an approximately 100 kD (proMMP9) and an approximately 80 kD form of MMP9 (FIG. 2A, lanes 1 and 2). proMMP9 was increased markedly in ankle homogenates 5 and 18 days after SCW-administration (FIG. 2A, lanes 3-5 and 6-8, respectively). The 80 kD MMP9 was increased mildly 5 days after SCW-administration (FIG. 2A, lanes 3-5) and was increased markedly 18 days after SCW-administration (FIG. 2A, lanes 6-8). In healthy ankles of rats administered saline, mAb-1246 detected small amounts active MMP9 at 80 kD (FIG. 2B, lanes 1 and 2). The 80 kD active MMP9 was increased mildly 5 days after SCW-administration (FIG. 2A, lanes 3-5) and was increased markedly 18 days after SCW-administration (FIG. 2A, lanes 6-8).

Efficacy of Example 64 in Rats with SCW Arthritis

Having shown that active MMP9 is increased in rats with SCW-induced arthritis, we next sought to determine the ability of Example 64 to reduce disease severity and to reduce active MMP9.

Example 64 Reduced Ankle Swelling of Rats with SCW-Induced Arthritis

To induce arthritis, Female Lewis (LEW/N) rats, 5-6 weeks of age (80-100 g) were injected (i.p.) with SCW PG-PS as described above. Eighteen days later, arthritis was well established. Calipers were used to measure the width (anterior to posterior surface) of the left and right hind ankles of each rat. Each ankle was measured 3 times and averaged, and treatment groups were randomized based on ankle thickness (Table 6). Commencing on day 18, randomized groups of arthritic rats (n=5 rats/group) received vehicle or 5, 20, or 50 mg/kg Example 64 BID by oral gavage. Vehicle consisted of an aqueous mixture containing 2% (v:v) N-methylpyrrolidone, 5% (v:v) glycerine, and 20% (w:v) captisol. Treatment continued daily through the morning of day 26.

By day 18 mean ankle thickness was increased an average of >4.4 mm compared to disease free rats. Rats treated with vehicle alone continued to gradually develop a more severe arthritis based on ankle thickness measurements over the eight-day treatment period (Table 6). Treatment with Example 64 induced a dose-dependent decrease in ankle thickness measurements. By day 26, the disease associated increase in ankle thickness had been reduced 27, 37, and 46 percent by 5, 20, and 50 mg/kg Example 64, respectively.

TABLE 6
Ankle thickness of rats with SCW-arthritis dosed with vehicle vs. Example 64

| | | Ankle thickness | | | |
|---|---|---|---|---|---|
| | | (mm) ^{a} | | Day 26 Δ mm | |
| Treatment | | Day 18 | Day 26 | (vs. group 1) | % Inh |
| Group 1: | mean (n = 4) | 7.20 | 7.26 | 0 | 100 |
| Sterile Saline | SD | 0.043 | 0.012 | | |
| Vehicle | p-value ^{b} | 0.0000 | 0.0001 | | |
| Day 18-26 | | | | | |
| Group 2: | mean (n = 5) | 11.86 | 12.31 | 5.04 | 0 |
| PG-PS (15 μg/gramBW) | SD | 0.77 | 1.26 | | |
| Vehicle | p-value* | na | na | | |
| Day 18-26 | | | | | |
| Group 3: | mean (n = 5) | 11.79 | 10.93 | 3.67 | 27 |
| PG-PS (15 μg/gramBW) | SD | 0.56 | 0.21 | | |
| Example 64 (5 mg/kg) | p value* | 0.88 | 0.043 | | |
| Day 18-26 | | | | | |
| Group 4: | mean (n = 5) | 11.76 | 10.42 | 3.15 | 37 |
| PG-PS (15 μg/gramBW) | SD | 0.73 | 0.93 | | |
| Example 64 (20 mg/kg) | p-value* | 0.85 | 0.028 | | |
| Day 18-26 | | | | | |
| Group 5: | mean (n = 5) | 11.68 | 9.99 | 2.73 | 46 |
| PG-PS (15 μg/gramBW) | SD | 0.62 | 0.73 | | |
| Example 64 (50 mg/kg) | p-value* | 0.71 | 0.0075 | | |
| Day 18-26 | | | | | |

^{a} Calipers were used to measure the width (anterior to posterior surface) of the left and right hind ankles of each rat. Each ankle was measured 3 times and averaged.
^{b} Student's t-test vs. group 2

Hind paw inflammation clinical scores were assigned based on swelling and erythema. By day 18, nearly all rats induced with SCW PG-PS had a clinical score of 8 based on an 8-point scale (Table 7). Treatment with Example 64 induced a dose dependent decrease in clinical score measurements with significant effects emerging at the 20 mg/kg dose (Table 7).

TABLE 7
Clinical Scores of rats with SCW-arthritis dosed with vehicle vs. Example 64

| | | Clinical Scores (0-8) ^{a} | | Δ Day 18 vs. |
|---|---|---|---|---|
| Treatment | | Day 18 | Day 26 | day 26 |
| Group 1: | mean (n = 4) | 0 | 0 | 0 |
| Sterile Saline | SD | 0 | 0 | |
| Vehicle | p-value ^{b} | | <0.0001 | |
| Day 18-26 | | | | |
| Group 2: | mean (n = 5) | 7.80 | 7.80 | 0 |
| PG-PS (15 μg/gramBW) | SD | 0.45 | 0.45 | |
| Vehicle | p-value | | na | |
| Day 18-26 | | | | |
| Group 3: | mean (n = 5) | 8.00 | 6.80 | −1.20 |
| PG-PS (15 μg/gramBW) | SD | 0.00 | 1.09 | |
| Example 64 (5 mg/kg) | p-value | | 0.095 | |
| Day 18-26 | | | | |
| Group 4: | mean (n = 5) | 8.00 | 5.20 | −2.80 |
| PG-PS (15 μg/gramBW) | SD | 0.00 | 1.79 | |
| Example 64 (20 mg/kg) | p-value | | 0.014 | |
| Day 18-26 | | | | |
| Group 5: | mean (n = 5) | 7.80 | 4.40 | −3.40 |
| PG-PS (15 μg/gramBW) | SD | 0.45 | 1.67 | |
| Example 64 (50 mg/kg) | p-value | | 0.0023 | |
| Day 18-26 | | | | |

^{a} Hind paw inflammation clinical scores were assigned based on swelling and erythema as follows: 1 = ankle involvement only; 2 = involvement of ankle and proximal 1/2 of tarsal joint; 3 = involvement of the ankle and entire tarsal joint down to the metatarsal joints; and 4 = involvement of the entire paw including the digits. Scores of both hind-paws were summed for a maximal score of 8.
^{b} Student's t-test vs. group 2

Example 64 Reduced Active MMP9 in Ankles of Rats with SCW-Induced Arthritis Demonstrated by Western Blot Analysis

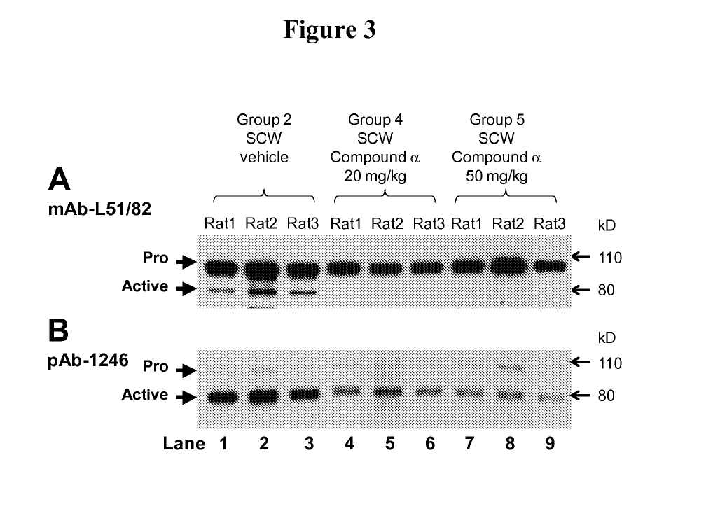

Rats in the study reported in Tables 4 and 5 were sacrificed on Day 26 four hours after the AM dose. Ankles harvested from the right-hind-limbs were processed by the method described above. Pro and active MMP9 were abundantly present in ankles of SCW-induced vehicle-treated rats (FIGS. 3A and 3B, lanes 1-3). Treatment of rats with Example 64 did not reduce the abundance of proMMP9 (FIG. 3A, lanes 4-9). However, treatment of rats with Example 64 resulted in a notable reduction in the active 80 kD form of MMP9 detected with pAb-1246 (FIG. 3B, lanes 4-9 vs. 1-3) and with mAb-L51/82 (FIG. 3A, lanes 4-9 vs. 1-3).

Example 64 Reduced MMP9 Mediated Gelatinase Activity in the Livers of Rats with SCW Arthritis

In situ zymography provides an alternative approach to assess active MMP9 in tissues (Frederiks). Tissue sections are overlain with fluorescene-conjugated gelatin wherein the conjugation is sufficiently dense to cause the fluorescene to be dye-quenched (DQ). Proteolytic degradation of the DQ-gelatin releases the fluorescene from the quenching effect giving rise to bright green fluorescence at the site of degradation. Because in situ zymography requires the use of frozen sections, calcified tissues are problematic. However, an additional feature of the SCW arthritis model is the development of hepatic granulomatous disease (J Immunol; 1986; 137:2199-2209), and MMP9 reportedly plays a role in macrophage recruitment in the granulomas response to mycobacteria (Infect Immun; 2006; 74:6135-6144). Consequently, granulomatous livers from SCW-treated rats were assessed for active MMP9 by in situ zymography.

As described above, Female Lewis (LEW/N) rats, 5-6 weeks of age (80-100 g) were injected (i.p.) with saline or SCW PG-PS. On day 28, when the granulomatous response was well established, animals were sacrificed and livers were frozen in OCT cryo-sectioning medium and 10 μm sections were cut on a Cryome HM 500 M cryotome and mounted on glass microscope slides. Sections were air dried briefly. MMP9 was confirmed as the source of the gelatinase activity in the liver by treating liver sections with monoclonal antibodies directed against the active site of the two major gelatinases MMP9 and MMP2. Liver sections overlain with 50 μL of 100 μg/mL neutralizing mouse monoclonal antibodies directed against MMP9 (Calbiochem, clone 6-6B), or MMP2 (Millipore, clone CA-4001), or with PBS for 1 hr at room temperature. Tissues were rinsed once with PBS, blotted, and briefly air dried and then overlain with DQ-gelatin (Invitrogen) dissolved to 1 mg/mL in deionized water and then diluted 1:10 in 1% wt/vol low gelling point agarose type VII (Sigma) in PBS. The sections were covered with coverslips, incubated in the dark at room temperature for 20 min, and imaged on an Olympus IX80 inverted microscope fitted with fluorescence optics, using SlideBook™ imaging software (Intelligent Imaging Innovations, Inc., Philadelphia, Pa.; version 5.0). Fluorescence intensity was determined (Table 8). When compared to a saline-treated rat, gelatinase activity was abundantly expressed in granulomatous liver sections obtained from a rat with SCW arthritis. The activity in the granulomatous liver sections was almost completely inhibited by treatment with anti-MMP9 monoclonal antibody but not by treatment with anti-MMP2 monoclonal antibody.

TABLE 8
Indentification of MMP9 as the gelatinase
responsible for signals detected by in
situ zymography in SCW-granulomatous livers

| Disease | Section | Intensity (RLU × 10⁶) | |
|---|---|---|---|
| induction | treatment | Mean | SD |
| Saline-healthy | PBS | 11.4 | 2.91 |
| SCW- | PBS | 109 | 19.3 |
| granulomatous | Anti-MMP9 | 1.02 | 0.17 |
| | Anti-MMP2 | 128 | 36.2 |

Key:
RLU = relative light units; SCW = Streptococcal cell wall peptidoglycan-polysaccharide equivalent to 15 μg rhamnose/gram BW.

Next, liver in situ zymography was used to assess the relative presence of active MMP9 in rats dosed with vehicle vs. Example 64. Female Lewis (LEW/N) rats, 5-6 weeks of age (80-100 g) were injected (i.p.) with saline or SCW PG-PS. Commencing on day 25, randomized groups of rats (n=3 rats/group) received vehicle or 20 or 50 mg/kg Example 64 BID by oral gavage. Vehicle consisted of an aqueous mixture containing 2% (v:v) N-methylpyrrolidone, 5% (v:v) glycerine, and 20% (w:v) captisol. Treatment continued daily through the morning of day 28. Four hrs after the AM dose on day 28, rats were sacrificed and livers assessed for active MMP9 by in situ zymography (Table 9). Gelatinase activity was increased markedly in SCW-induced rats, but activity was reduced by approximately 80% in animals treated with 50 mg/kg Example 64.

TABLE 9
In situ zymography determination of gelatinase activity in livers
of SCW-induced rats dosed with vehicle vs. Example 64

| | | | | | | t-test vs. |
|---|---|---|---|---|---|---|
| | Intensity (RLU × 10⁶) | | | | | SCW- |
| Treatment | Rat 1 | Rat 2 | Rat 3 | Mean | SD | vehicle |
| Saline | 3.3 | 1.1 | 1.6 | 2.0 | 1.15 | 0.001 |
| Vehicle | | | | | | |
| Day 25-28 | | | | | | |
| SCW | 65.1 | 43.4 | 58.9 | 55.8 | 11.17 | 1 |
| Vehicle | | | | | | |
| Day 25-28 | | | | | | |
| SCW | 43.0 | 69.0 | 53.7 | 55.2 | 13.06 | 0.96 |
| Example 64 | | | | | | |
| (20 mg/kg) | | | | | | |
| Day 25-28 | | | | | | |
| SCW | 3.2 | 25.6 | 4.5 | 11.1 | 12.57 | 0.010 |
| Example 64 | | | | | | |
| (50 mg/kg) | | | | | | |
| Day 25-28 | | | | | | |

Key: RLU = relative light units; SCW = Streptococcal cell wall peptidoglycan-polysaccharide equivalent to 15 μg rhamnose/gram BW.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. Compounds of Formula I 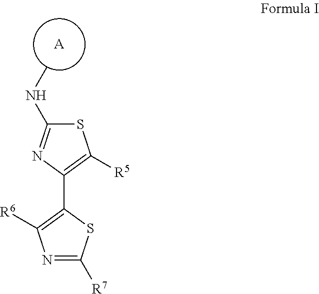
wherein:
A is a ring selected from the group consisting of 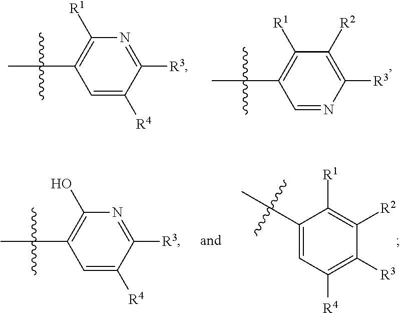
or R¹ and R² may be taken together with the ring to which they are attached to make a fused ring system selected from the group consisting of: quinolinyl, isoquinolinyl, quinazolinyl, quinoxalinyl, benzimidazolyl, benzothiazolyl, napthalyl, benzofuranyl, benzo[1,3]dioxol-4-yl, 2,3-dihydro-benzo[1,4]dioxin-5-yl, 2,3-dihydro-benzofuranyl, benzothiophenyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, benzo[1,3]dioxolyl, 2,3-dihydro-benzo[1,4]dioxinyl, 2,3-dihydro-benzofuranyl, benzothiophenyl, benzotriazolyl, indolyl, indolinyl, and indazolyl are optionally substituted with one methyl group or up to two fluorine atoms;
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;
R² is H, CH₃, or F;
R³ is Cl, SO₂NH₂, SO₂CH₃, CO₂H, CONH₂, NO₂, —CN, CH₃, CF₃, or H;
R⁴ is NH₂, NHC₍₁₋₃₎alkyl, N(C₍₁₋₃₎alkyl)₂, C₍₁₋₃₎alkyl, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)C₍₁₋₄₎alkyl, C₍₁₋₄₎alkyl, F, Cl, Br, —CN, OC₍₁₋₄₎alkyl;
R⁵ is H, F, Br, Cl, CF₃, or CH₃;
R⁶ is H, CH₃, or CF₃;
R⁷ is H, CF₃, CH₂CF₃, Cl, Br, C₍₁₋₆₎alkyl, 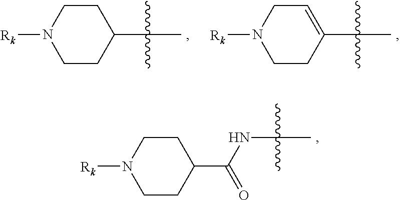
CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², or C₍₁₋₆₎alkylNA¹A²; wherein said is 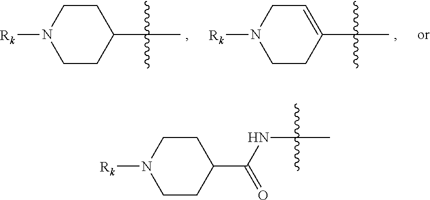
optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, C₍₃₋₆₎cycloalkyl, 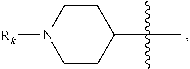
C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of: 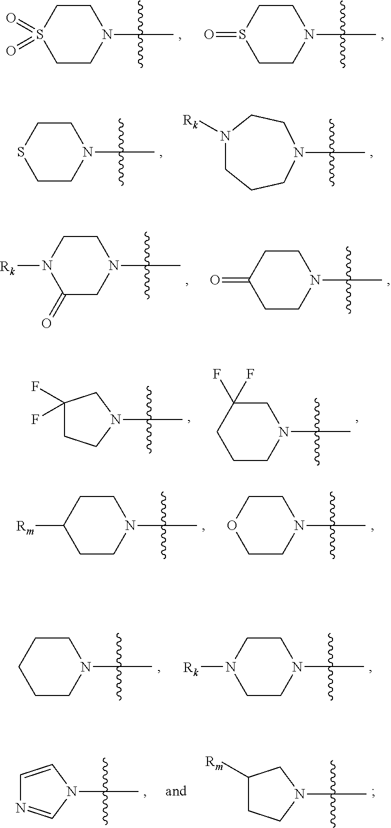
wherein any said A¹ and A² ring, except imidazolyl, may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₆₎alkyl, COC₍₁₋₄₎alkyl, SO₂C₍₁₋₄₎alkyl, trifluoropyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH;
and solvates, hydrates, tautomers, and pharmaceutically acceptable salts thereof.

2. A compound of claim 1 wherein:
A is a ring selected from the group consisting of 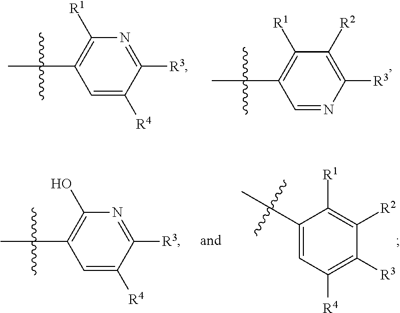
or R¹ and R² may be taken together with the ring to which they are attached to make a fused ring system selected from the group consisting of: quinolinyl, isoquinolinyl, quinazolinyl, benzimidazolyl, napthalyl, benzofuranyl, benzo[1,3]dioxol-4-yl, 2,3-dihydro-benzo[1,4]dioxin-5-yl, 2,3-dihydro-benzofuranyl, and indazolyl, wherein said quinolinyl, isoquinolinyl, quinazolinyl, benzimidazolyl, napthalyl, benzofuranyl, benzo[1,3]dioxolyl, 2,3-dihydro-benzo[1,4]dioxinyl, 2,3-dihydro-benzofuranyl, and indazolyl are optionally substituted with one methyl group or up to two fluorine atoms;
R² is H, or F;
R⁴ is NH₂, CH₃, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)C₍₁₋₄₎alkyl, C₍₁₋₄₎alkyl, F, Cl, Br, —CN, or OCH₃;
R⁷ is H, CF₃, CH₂CF₃, Cl, Br, C₍₁₋₆₎alkyl, 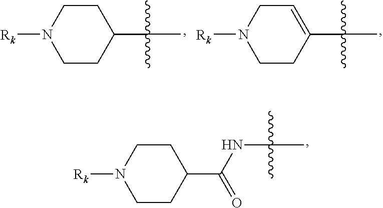
CO₂H, CO₂C₍₁₋₄₎alkyl, SO₂C₍₁₋₄₎alkyl, pyridinyl, pyrimidinyl, pyrazinyl, NA¹A², C(O)NA¹A², SO₂NA¹A², SONA¹A², C(O)N(CH₃)C₍₂₋₆₎alkylNA¹A², C(O)NHC₍₂₋₆₎alkylNA¹A², NHC(O)C₍₁₋₆₎alkylNA¹A², N(CH₃)C(O)C₍₁₋₆₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², NHC₍₂₋₆₎alkylNA¹A², N(CH₃)C₍₂₋₆₎alkylNA¹A², or CH₂NA¹A²;
A¹ is H, or C₍₁₋₃₎alkyl;
A² is H, C₍₁₋₆₎alkyl, C₍₃₋₆₎cycloalkyl, 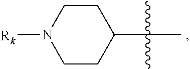
C₍₂₋₆₎alkylOH, C₍₂₋₆₎alkylOCH₃, SO₂C₍₁₋₄₎alkyl, C(O)Ph, C(O)C₍₁₋₄₎alkyl, pyrazinyl, or pyridyl; wherein said pyridyl may be optionally substituted with two substituents selected from the group consisting of CF₃ and pyrrolidinyl; or A¹ and A² are taken together with their attached nitrogen to form a ring selected from the group consisting of: 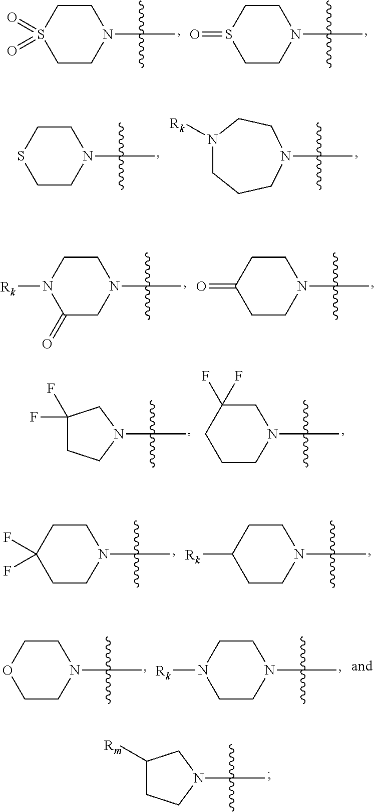
wherein any said A¹ and A² ring may be optionally substituted with up to four methyl groups on two or more ring carbon atoms or optionally substituted with up to two CF₃ groups on any two ring carbon atoms;
Rₖ is selected from the group consisting of H, CH₂CF₃, CH₂CH₂CF₃, C₍₁₋₃₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, CH₃, F, or OH;
and solvates, hydrates, tautomers, and pharmaceutically acceptable salts thereof.

3. A compound of claim 2 wherein:
A is a ring selected from the group consisting of 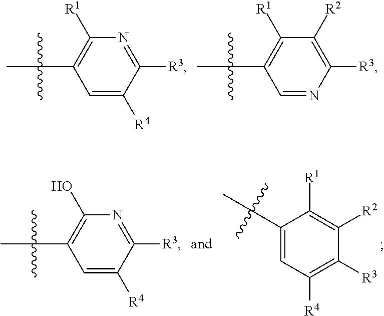
or R¹ and R² may be taken together with the ring to which they are attached to make a fused ring system selected from the group consisting of: quinolinyl, napthalyl, benzofuranyl, benzo[1,3]dioxol-4-yl, 2,3-dihydro-benzo[1,4]dioxin-5-yl, and 2,3-dihydro-benzofuranyl, wherein said quinolinyl, napthalyl, benzofuranyl, benzo[1,3]dioxolyl, 2,3-dihydro-benzo[1,4]dioxinyl, and 2,3-dihydro-benzofuranyl are optionally substituted with one methyl group or up to two fluorine atoms;
R^{d} is C(O)CH₃, C₍₁₋₄₎alkyl, F, or Cl;
R⁵ is H, F, CH₃, or Br;
R⁷ is H, CF₃, CH₂CF₃, C₍₁₋₄₎alkyl, 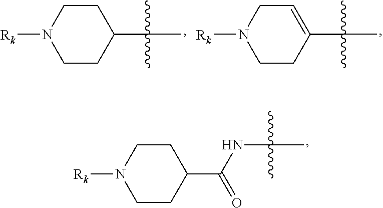
CO₂H, CO₂C₍₁₋₄₎alkyl, SO₂C₍₁₋₄₎alkyl, pyridinyl, pyrimidinyl, pyrazinyl, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₆₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², or CH₂NA¹A²;
A¹ is H, or C₍₁₋₃₎alkyl;
A² is H, C₍₁₋₃₎alkyl, 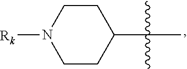
SO₂C₍₁₋₄₎alkyl, C(O)Ph, C(O)C₍₁₋₄₎alkyl, pyrazinyl, or pyridyl; wherein said pyridyl may be optionally substituted with two substituents selected from the group consisting of CF₃ and pyrrolidinyl; or A¹ and A² are taken together with their attached nitrogen to form a ring selected from the group consisting of: 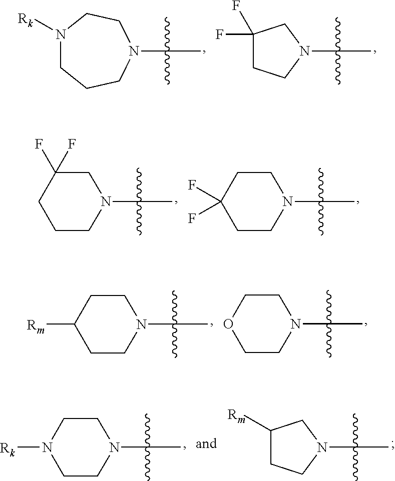
Rₖ is selected from the group consisting of H, CH₂CF₃, CH₂CH₂CF₃, C₍₁₋₃₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, F, or OH; and solvates, hydrates, tautomers, and pharmaceutically acceptable salts thereof.

4. A compound of claim 3 wherein:
R¹ is C₍₁₋₄₎alkoxy, C₍₁₋₄₎alkyl, SC₍₁₋₄₎alkyl, Cl, OC₍₃₋₆₎cycloalkyl, OCH₂CF₃, or OCF₃;
R³ is SO₂NH₂, SO₂CH₃, CO₂H, CONH₂, —CN, CH₃, or H;
R⁴ is NH₂, CH₃, —CN, —CH═CH₂, —CONH₂, —CO₂H, —NO₂, —CONHC₍₁₋₄₎alkyl, CH₂CONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHCH₂CH₂-piperidinyl, —CONHCH₂Ph, or R⁴ is selected from the group consisting of: phenyl, pyridyl, pyrazolyl, imidazolyl, oxazolyl, isoxazolyl, thiazolyl, and thiophenyl, wherein said phenyl, pyridyl, pyrazolyl, imidazolyl, oxazolyl, isoxazolyl, thiazolyl, and thiophenyl are optionally substituted with one R^{d}; provided that R⁴ is not NHC(O)CH₃, if R⁷ is NHC(O)CH₃; further provided that R⁴ is neither phenyl, thiophenyl, nor pyridyl, if R⁷ is CH₃; or R⁴ may be H, provided that R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C(O)CH₃, C₍₁₋₄₎alkyl, F, or Cl;
R⁷ is C₍₁₋₄₎alkyl, 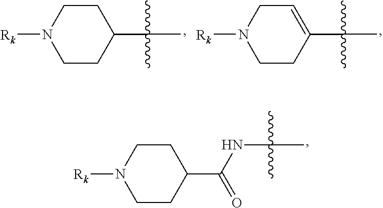
CO₂H, CO₂C₍₁₋₄₎alkyl, SO₂C₍₁₋₄₎alkyl, pyridyl, pyrimidinyl, pyrazinyl, NA¹A², C(O)NA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², or CH₂NA¹A²;
A¹ is H, or C₍₁₋₃₎alkyl;
A² is H, C₍₁₋₃₎alkyl, 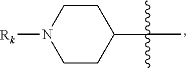
SO₂C₍₁₋₄₎alkyl, C(O)Ph, C(O)C₍₁₋₄₎alkyl, pyrazinyl, or pyridyl; wherein said pyridyl may be optionally substituted with two substituents selected from the group consisting of CF₃ and pyrrolidinyl; or A¹ and A² are taken together with their attached nitrogen to form a ring selected from the group consisting of: 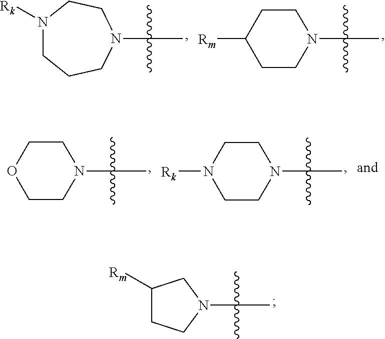
R^{k} is selected from the group consisting of H, C₍₁₋₃₎alkyl, COC₍₁₋₄₎alkyl, SO₂C₍₁₋₄₎alkyl, trifluoromethylpyridyl, and C₍₃₋₆₎cycloalkyl;
Rₘ is H, OCH₃, CH₂OH, N(C₍₁₋₄₎alkyl)₂, NH₂, F, or OH;
and solvates, hydrates, tautomers, and pharmaceutically acceptable salts thereof.

5. A compound of claim 4 wherein:
A is a ring selected from the group consisting of 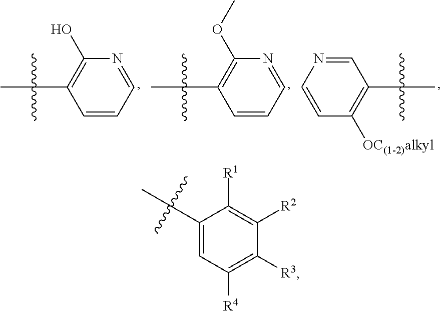
quinolin-8-yl, 6-methoxy-quinolin-8-yl, napthal-1-yl, 2-methyl-benzofuran-7-yl, 2,2-difluoro-benzo[1,3]dioxol-4-yl, 2,3-dihydro-benzo[1,4]dioxin-5-yl, 5-methoxy-2,3-dihydro-isoindolon-6-yl, and 5-sulfonic acid amide-2,3-dihydro-benzofuran-7-yl;
wherein
R¹ is C₍₁₋₄₎alkoxy, C₍₁₋₄₎alkyl, SCH₂CH₃, OCF₃, Cl, OCH₂CF₃, or O-cyclopentyl;
R³ is H, CONH₂, or CH₃;
R⁴ is NH₂, CH₃, —CN, —CH═CH₂, —CONH₂, —CO₂H, —NO₂, —CONHCH₃, CH₂CONH₂, —NHCOCH₃, —CO₂CH₃, —CO₂CH₂CH₃, CF₃, SO₂C₍₁₋₂₎alkyl, —SO₂NH₂, —SO₂N(CH₂CH₃)₂, —CONHCH₂CH₂-piperidinyl, —CONHCH₂Ph, or R⁴ is selected from the group consisting of: 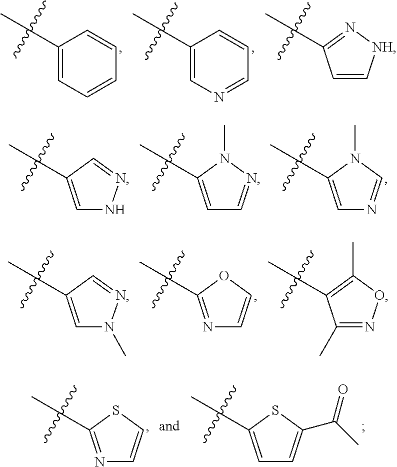
provided that R⁴ is not NHC(O)CH₃, if R⁷ is NHC(O)CH₃; further provided that R⁴ is neither phenyl, nor pyridyl, if R⁷ is CH₃; or R⁴ may be H, provided that R³ is CONH₂; 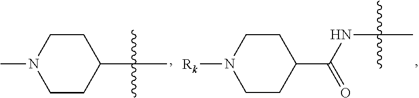
R⁷ is CH₃, CO₂H, CO₂C₍₁₋₂₎alkyl, SO₂CH₃, pyridinyl, NA¹A², C(O)NA¹A², CH₂NHCH₂CH₂NA¹A², CH₂N(CH₃)CH₂CH₂NA¹A², C(O)N(CH₃)CH₂CH₂NA¹A², or CH₂NA¹A²; wherein
A is H, or C₍₁₋₂₎alkyl;
A² is H, C₍₁₋₃₎alkyl, C(O)CH₃, 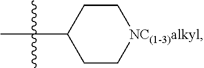
pyrazinyl, or pyridyl, wherein said pyridyl is optionally substituted with up to two substituents selected from the group consisting of CF₃ and pyrrolidinyl; alternatively, A¹ and A² may be taken together with their attached nitrogen, to form a ring selected from the group consisting of 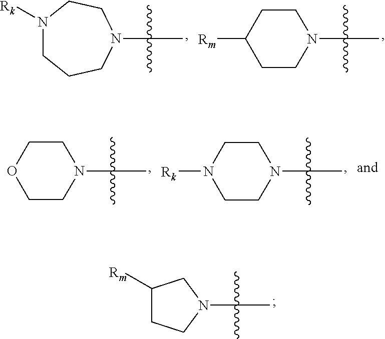
Rₖ is selected from the group consisting of C₍₁₋₃₎alkyl, trifluoromethylpyridyl, and cyclohexyl;
Rₘ is H, N(CH₃)₂, or OH;
and solvates, hydrates, tautomers, and pharmaceutically acceptable salts thereof.

6. A compound of claim 1, wherein:
A is 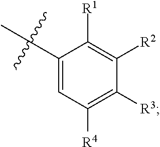
R² is OCH(CH₃)₂;
R² is H;
R³ is H;
R⁴ is —CONH₂, —CO₂H, or —SO₂NH₂;
R⁵ is H; and
R⁶ is CH₃;
and solvates, hydrates, tautomers, and pharmaceutically acceptable salts thereof.

7. A compound which is selected from the group consisting of: 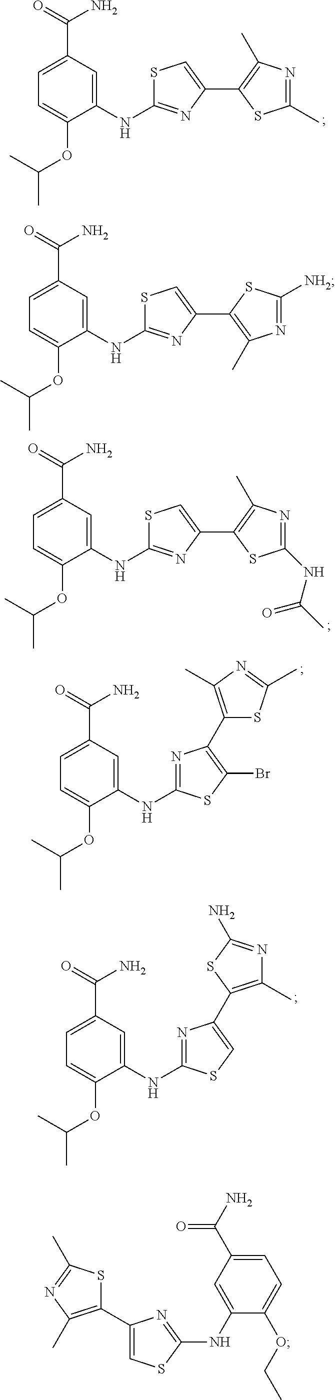 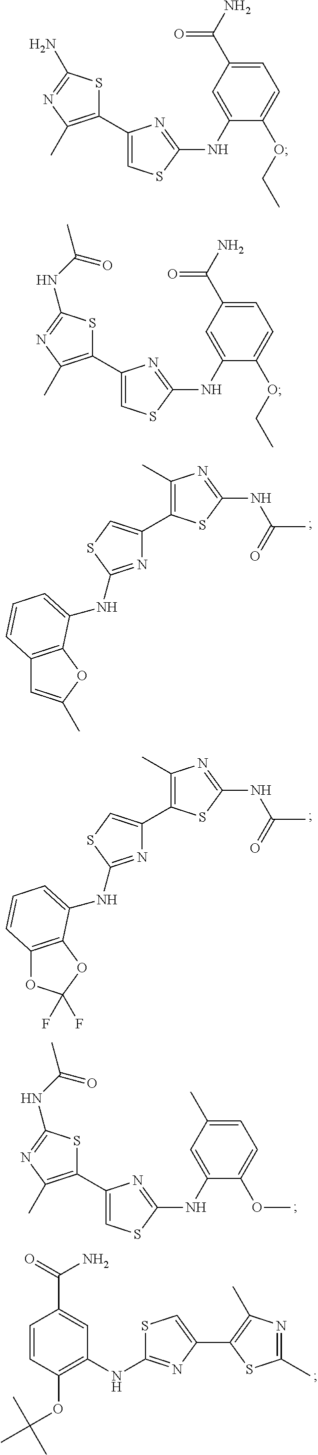 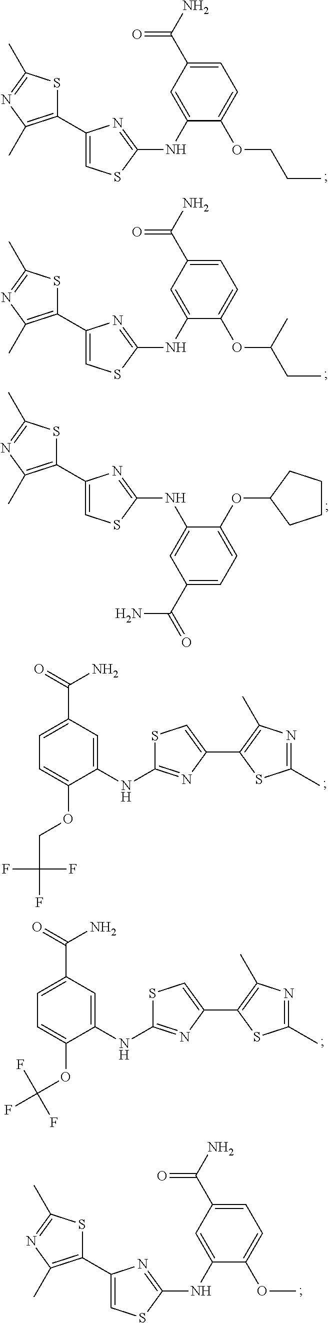 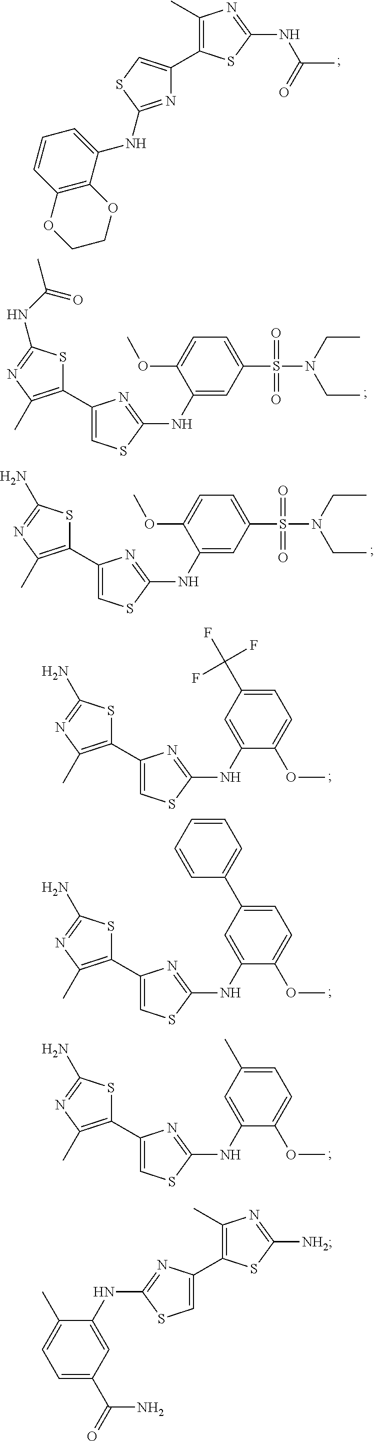 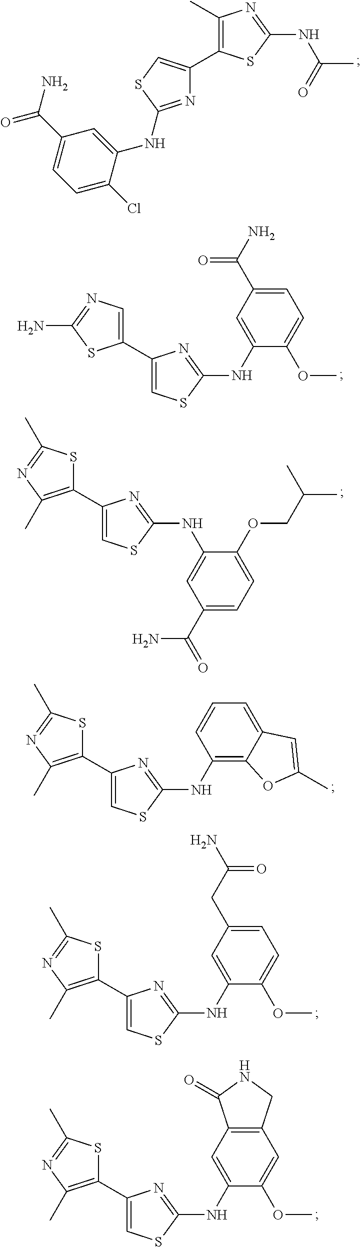 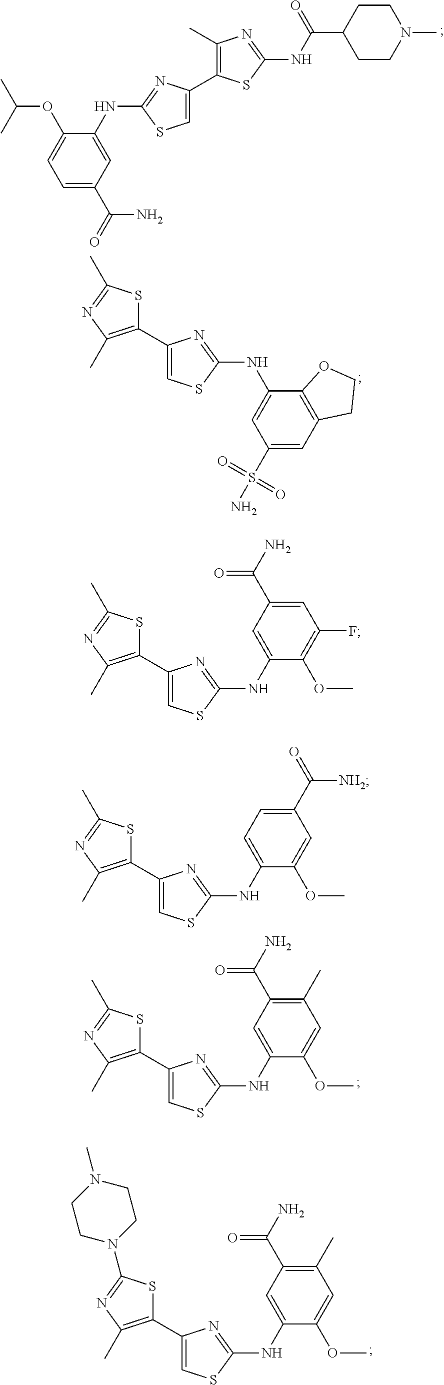 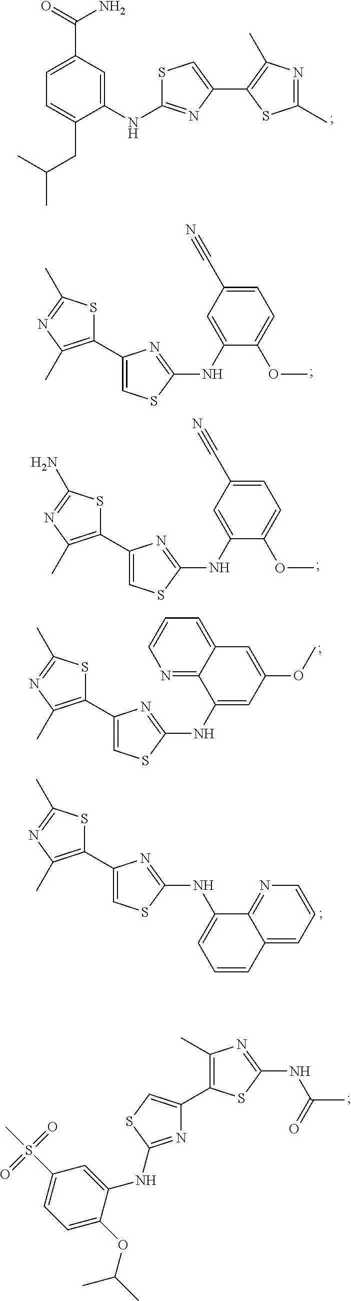 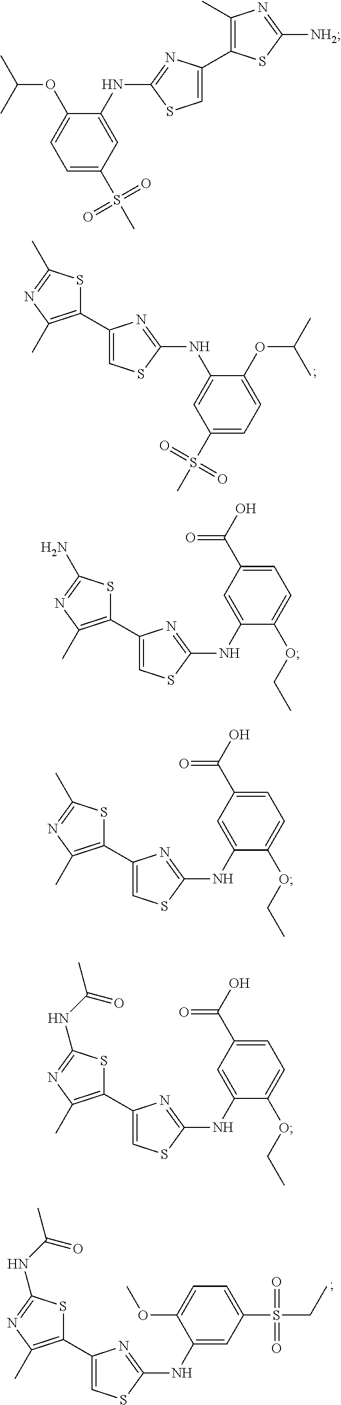 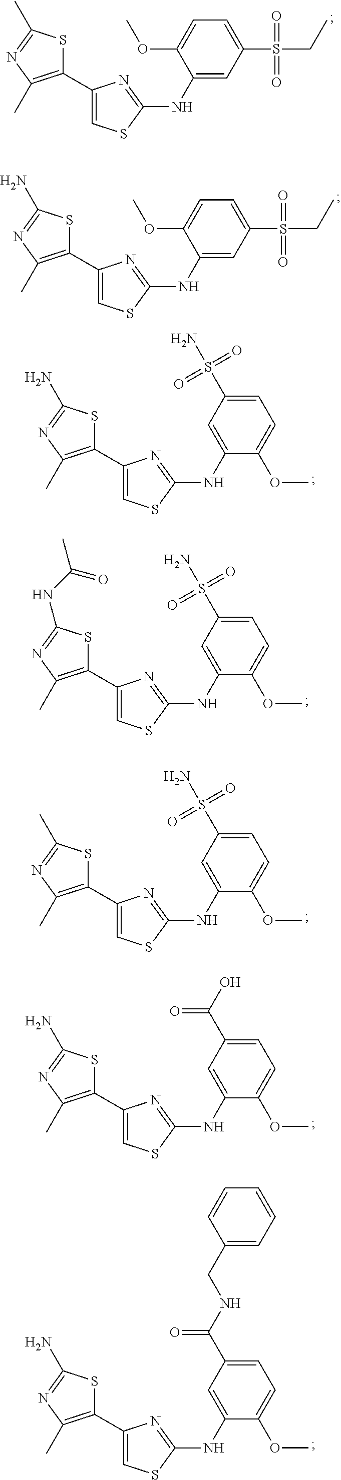 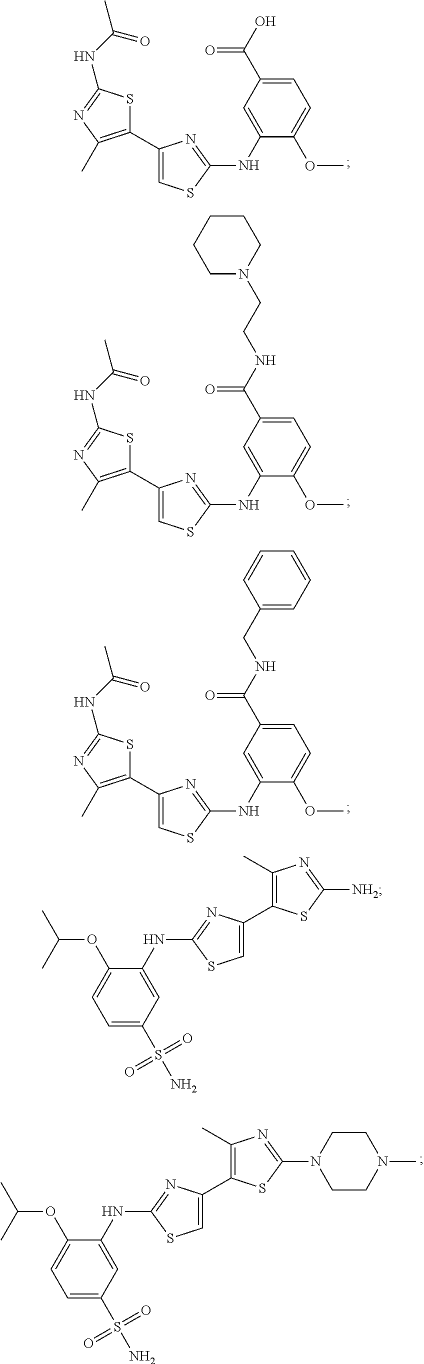 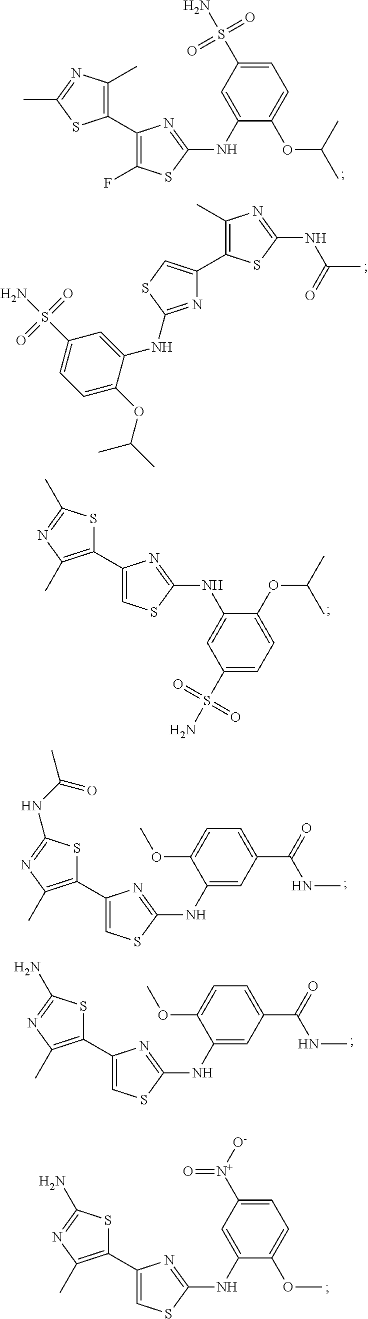 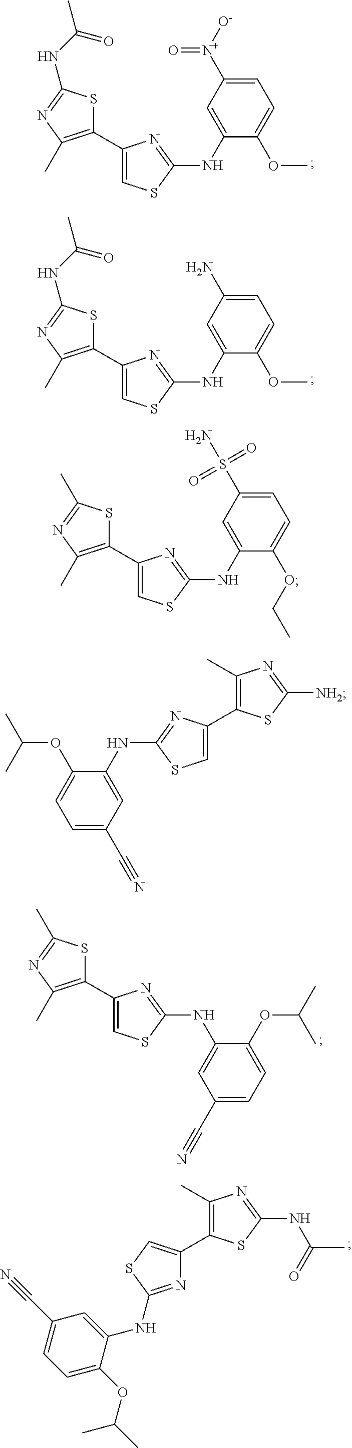 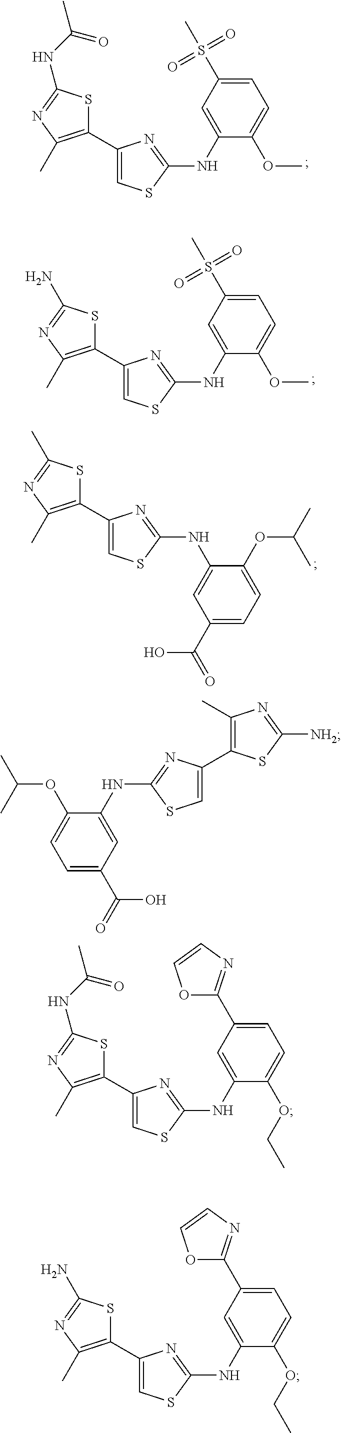 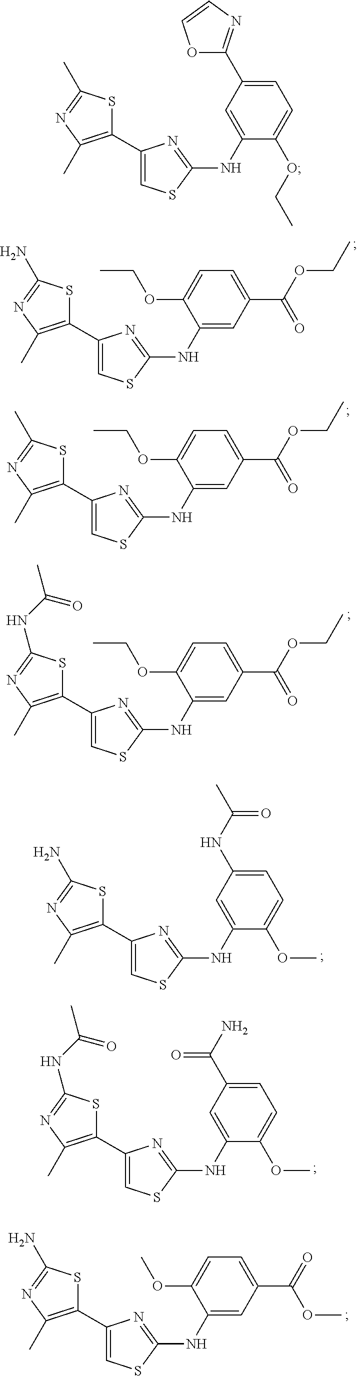 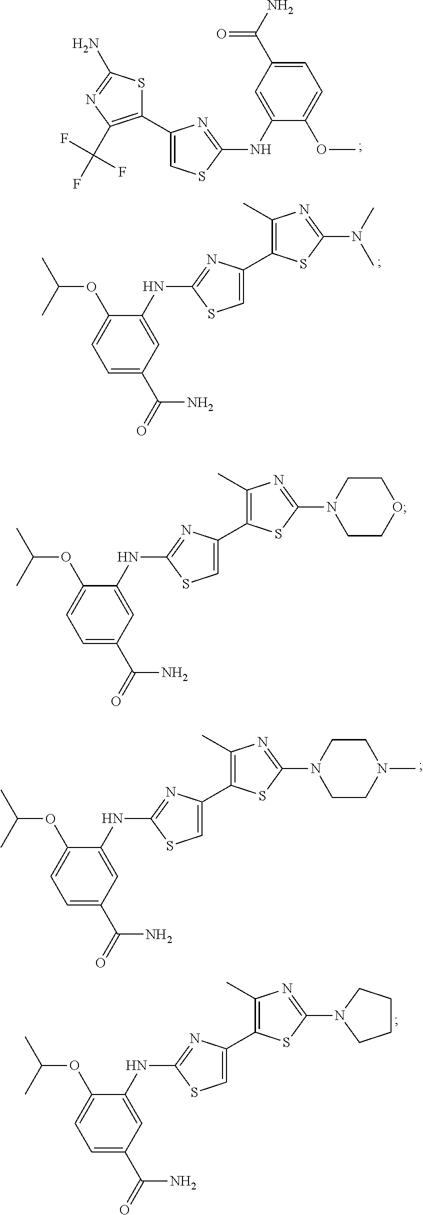 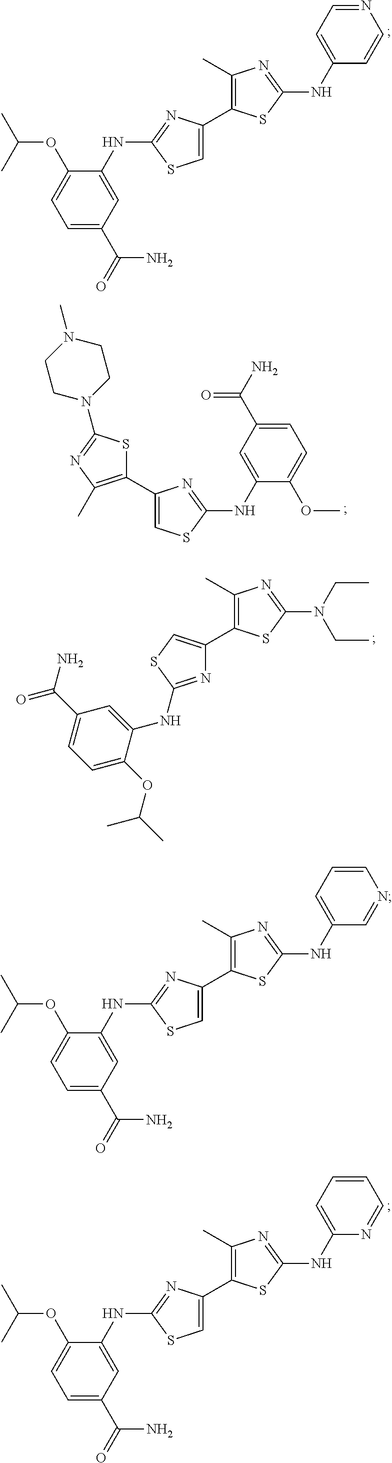 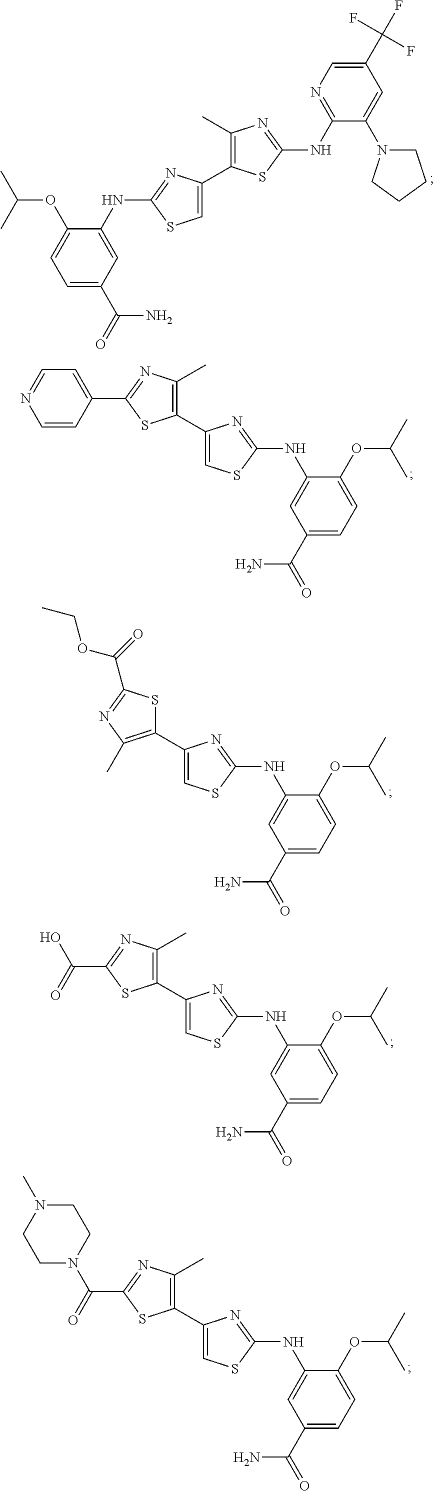 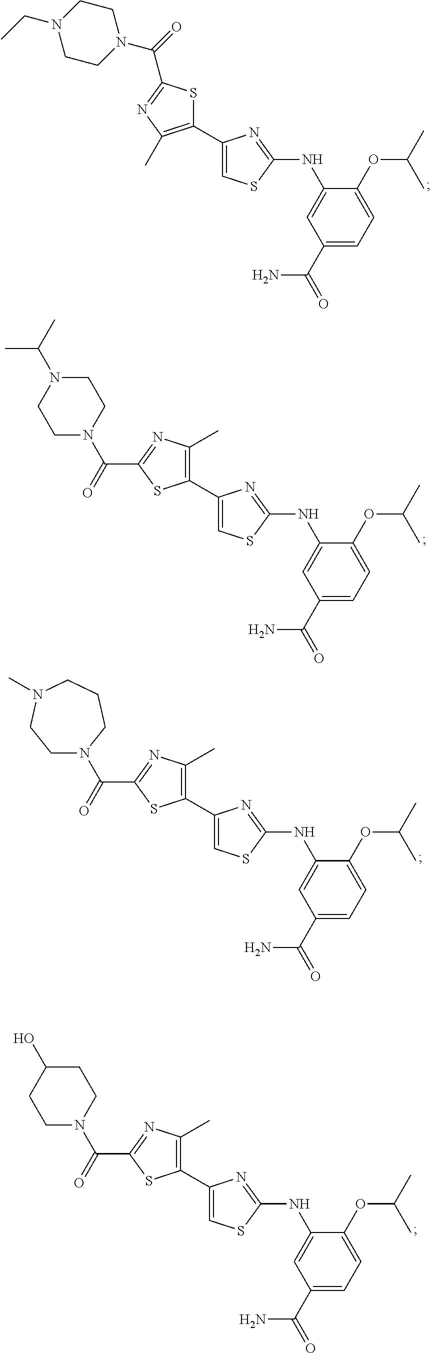 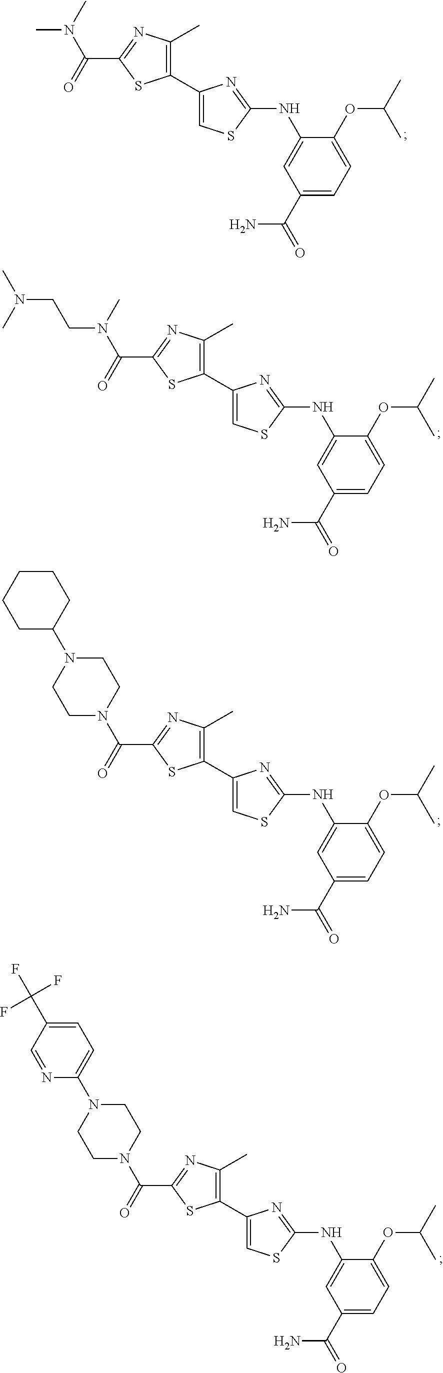 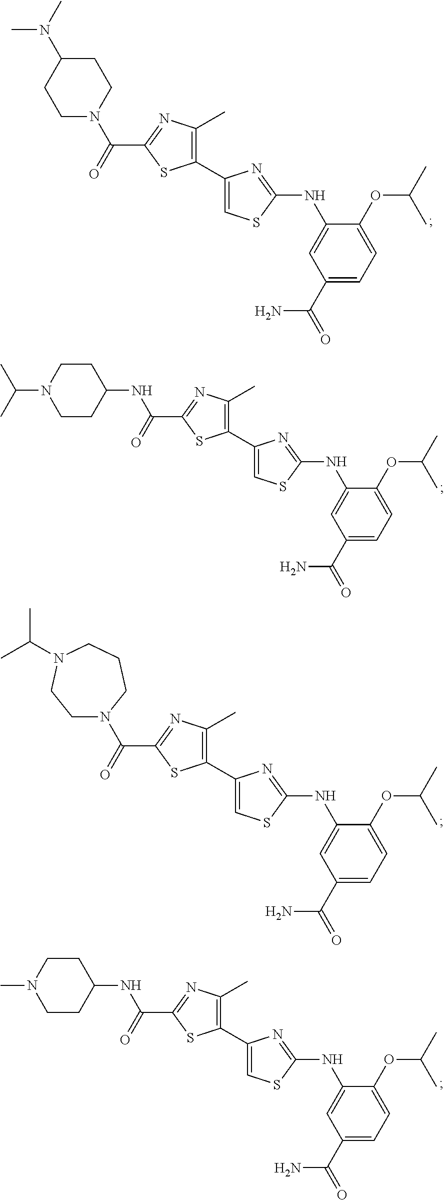 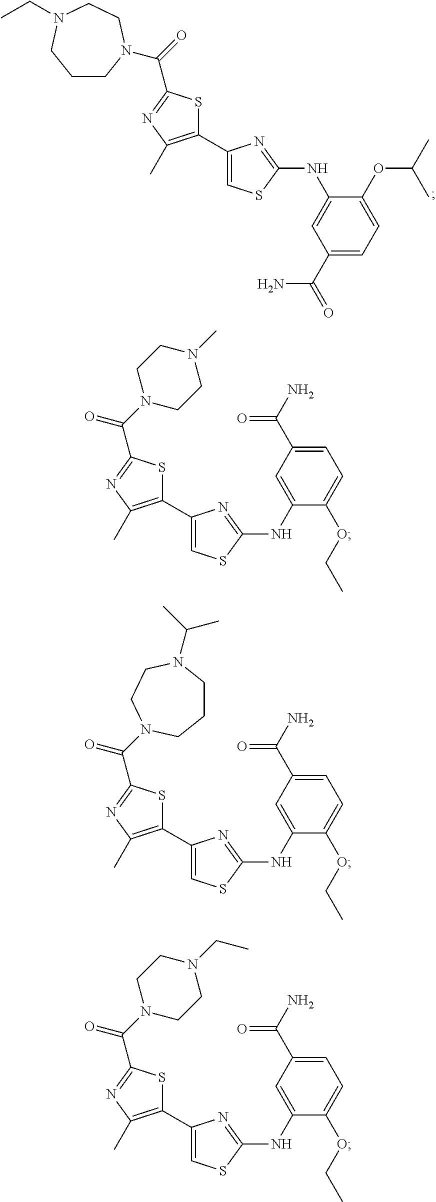 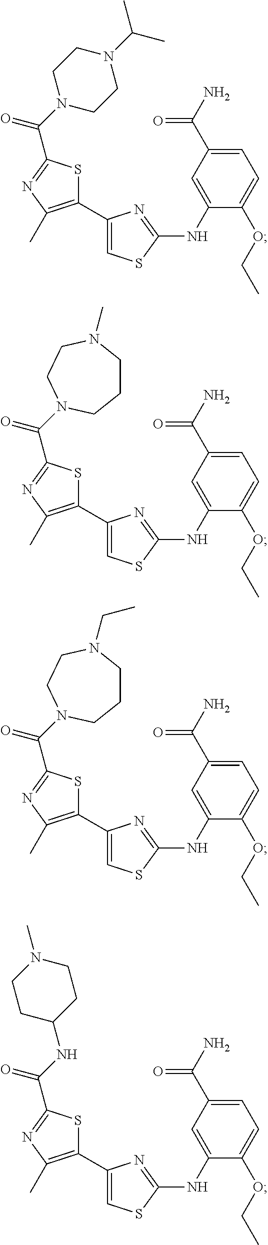 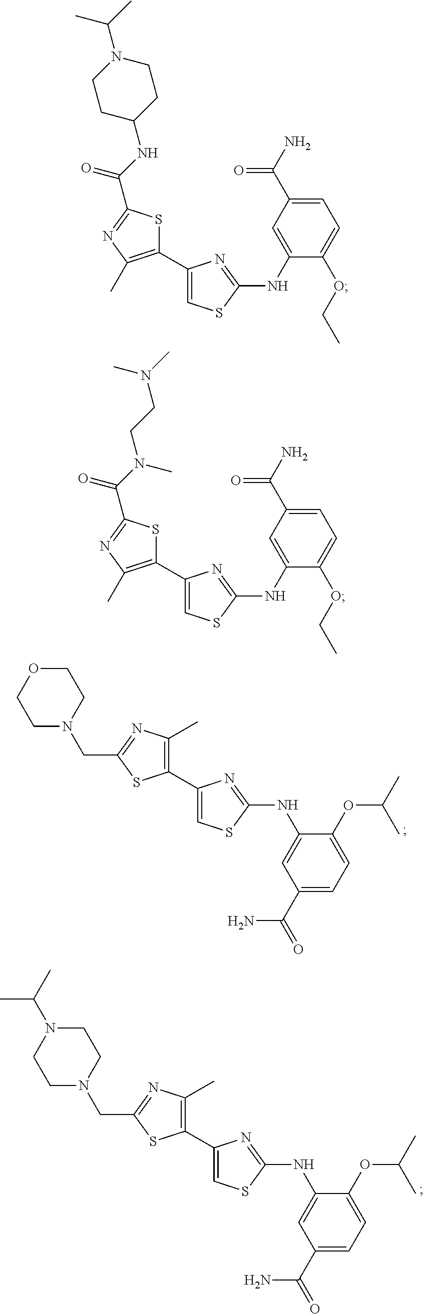 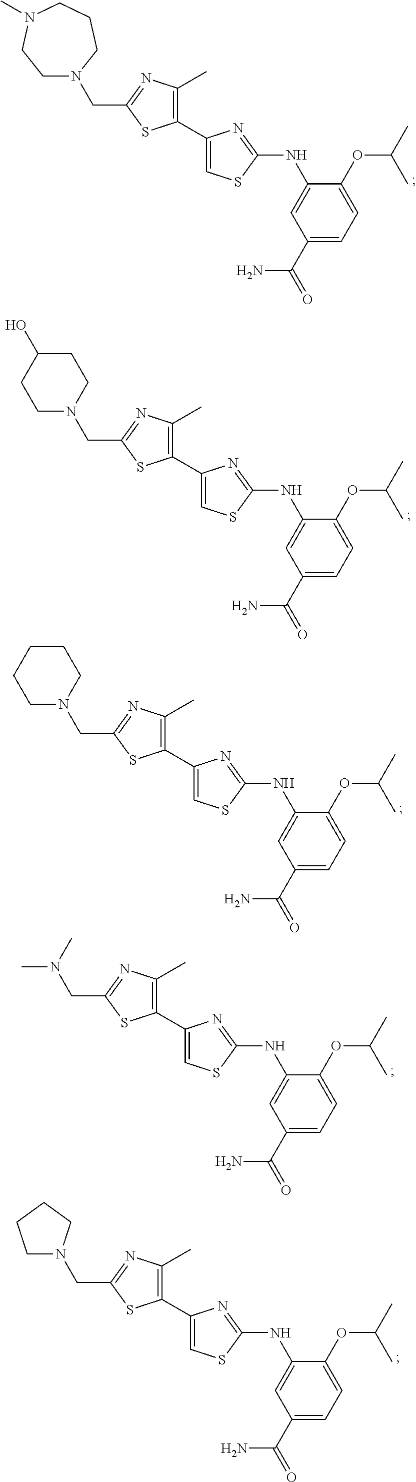 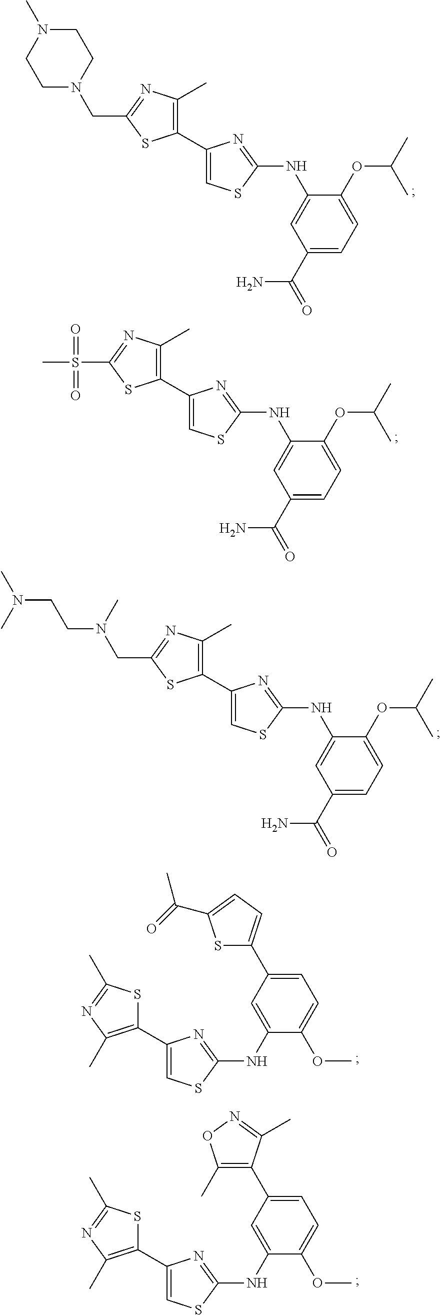 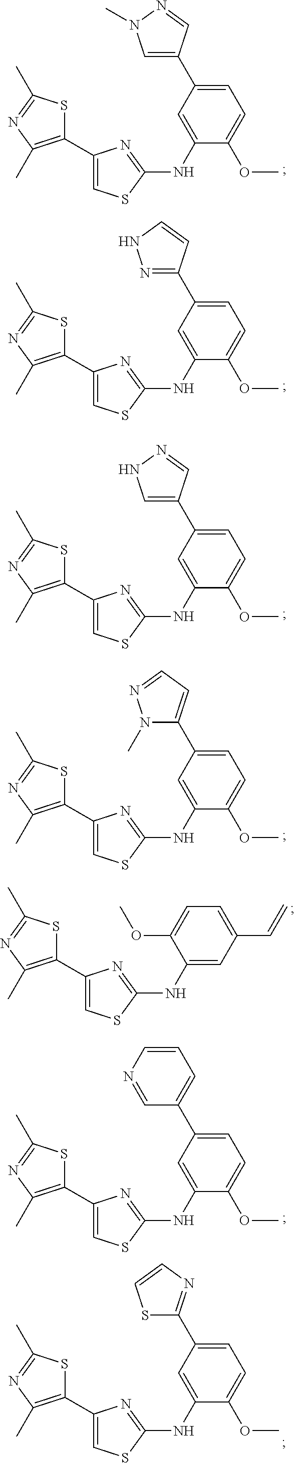 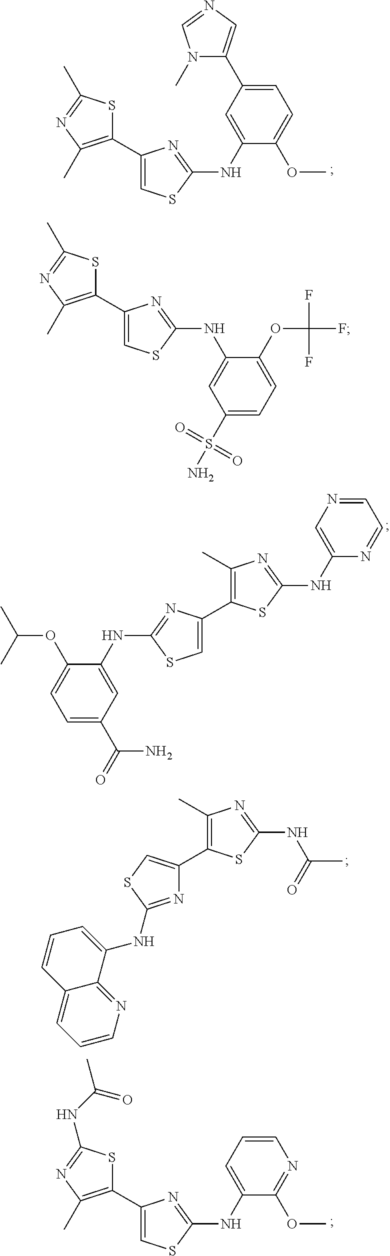 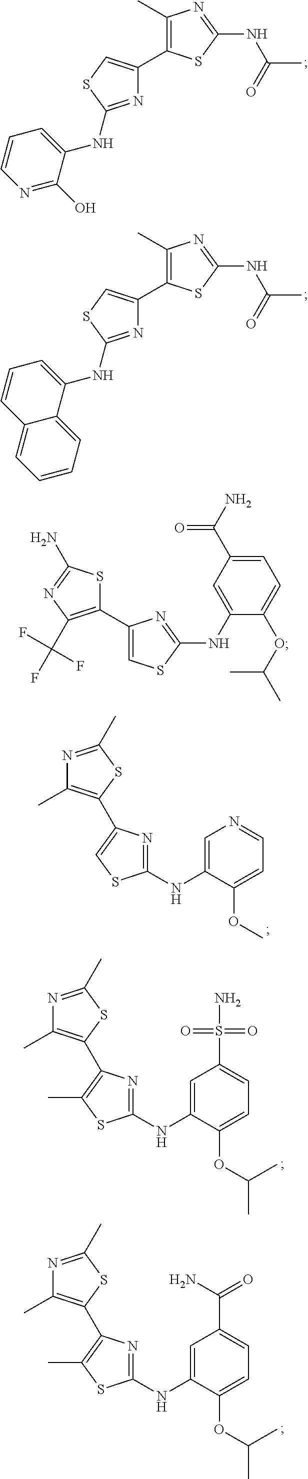 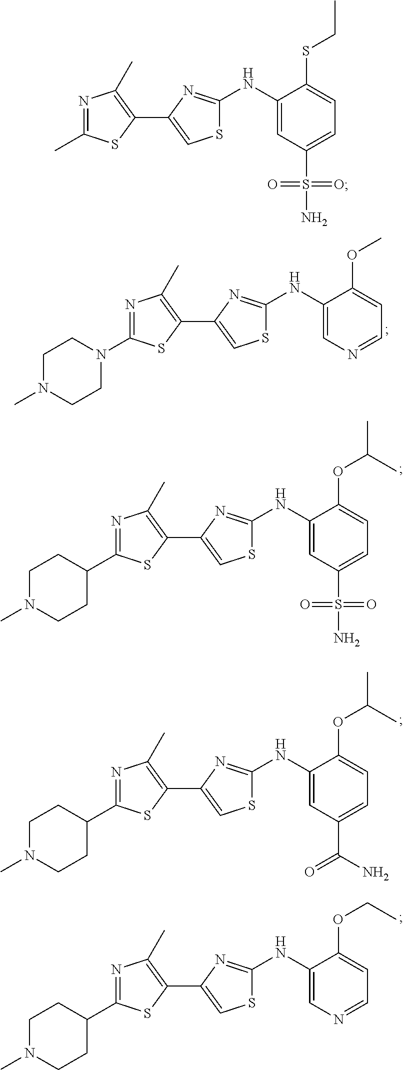
and solvates, hydrates, tautomers, and pharmaceutically acceptable salts thereof.

8. A pharmaceutical composition, comprising a compound of claim 1 and a pharmaceutically acceptable carrier.

9. A pharmaceutical composition, comprising a compound listed in the Examples section of this specification and a pharmaceutically acceptable carrier.

10. A method for preventing, treating or ameliorating an MMP9 mediated syndrome, disorder or disease comprising administering to a subject in need thereof an effective amount of a compound of claim 1 or a form, composition or medicament thereof.

11. A method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound of Formula I or a form, composition or medicament thereof.

12. A method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound of Formula I or a form, composition or medicament thereof.

13. The method of claim 12, wherein said syndrome, disorder or disease is a neoplastic disorder, which is ovarian cancer.

14. The method of claim 12, wherein said syndrome, disorder or disease is a cardiovascular disease, wherein said cardiovascular disease is selected from the group consisting of: atherosclerotic plaque rupture, aneurysm, vascular tissue morphogenesis, coronary artery disease, and myocardial tissue morphogenesis.

15. The method of claim 14, wherein said cardiovascular disease is atherosclerotic plaque rupture.

16. The method of claim 12, wherein said syndrome, disorder or disease is rheumatoid arthritis.

17. The method of claim 12, wherein said syndrome, disorder or disease is asthma.

18. The method of claim 12, wherein said syndrome, disorder or disease is chronic obstructive pulmonary disease.

19. The method of claim 12, wherein said syndrome, disorder or disease is inflammatory bowel syndrome.

20. The method of claim 12, wherein said syndrome, disorder or disease is abdominal aortic aneurism.

21. The method of claim 12, wherein said syndrome, disorder or disease is osteoarthritis.

22. The method of claim 12, wherein said syndrome, disorder or disease is idiopathic pulmonary fibrosis.

23. A method of inhibiting MMP9 activity in a mammal by administration of an effective amount of at least one compound of claim 1.

24. A method for preventing, treating or ameliorating an MMP13 mediated syndrome, disorder or disease comprising administering to a subject in need thereof an effective amount of a compound of claim 1 or a form, composition or medicament thereof.

25. A method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound of Formula I or a form, composition or medicament thereof.

26. A method of inhibiting MMP13 activity in a mammal by administration of an effective amount of at least one compound of claim 1.
